(19) United States
(12) Patent Application Publication
(10) Pub. No.: US 2024/0130661 A1
(43) Pub. Date: Apr. 25, 2024

(54) VIRTUAL AND AUGMENTED REALITY DEVICES TO DIAGNOSE AND TREAT COGNITIVE AND NEUROPLASTICITY DISORDERS

(71) Applicant: The Regents of the University of California, Oakland, CA (US)

(72) Inventor: Mayank R. Mehta, Marina Del Rey, CA (US)

(21) Appl. No.: 18/392,437

(22) Filed: Dec. 21, 2023

Related U.S. Application Data
(63) Continuation of application No. PCT/US2022/034946, filed on Jun. 24, 2022.
(60) Provisional application No. 63/214,563, filed on Jun. 24, 2021.

(51) Int. Cl. A61B 5/378; A61B 5/00; A61B 5/0205; A61B 5/374; G06F 3/01

(52) U.S. Cl. CPC A61B 5/378; A61B 5/02055; A61B 5/374; A61B 5/4088; A61B 5/7267; A61B 5/744; G06F 3/015

(57) ABSTRACT

Virtual and augmented reality devices and methods are provided for measuring and controlling an electrical activity of the brain, including for manipulation of cortico-hippocampal rhythms to treat neurological and neuropsychiatric disorders.

### CROSS-REFERENCE TO RELATED APPLICATIONS

This application is a continuation of International Patent Application No. PCT/US22/34946, filed Jun. 24, 2022, which claims the benefit of U.S. Provisional Application No. 63/214,563, filed Jun. 24, 2021, the entire contents of which are incorporated herein by reference.

### STATEMENT REGARDING FEDERALLY SPONSORED RESEARCH OR DEVELOPMENT

This invention was made with government support under Grant Number MH092925, awarded by the National Institutes of Health. The government has certain rights in the invention.

### BACKGROUND

Embodiments of the present disclosure generally relate to virtual and augmented reality devices for controlling the electrical activity of the brain, including for driving cortico-hippocampal activity, brain rhythms and neuro plasticity, with or without active engagement (e.g., movement) from the user, and for early diagnosis of neurocognitive disorders.

### BRIEF SUMMARY

Systems, methods, and computer program products of the present invention for controlling an electrical activity of the brain and diagnosing abnormalities are disclosed. In various embodiments, a first visual stimulus is presented to the user within the virtual environment. The first visual stimulus has a high spatial frequency. A second visual stimulus is presented to the user within the virtual environment. The second visual stimulus has a low spatial frequency. In various embodiments, these stimuli may be controlled by the user's movements (e.g., in VR) or they change autonomously (e.g., in AR).

In various embodiments, at least one electrical activity of the brain is measured by at least one sensor. The measured at least one electrical activity of the brain is provided to a learning system and determining therefrom an updated first visual stimuli and an updated second visual stimuli adapted to induce a change in the at least one electrical activity of the brain. The updated first visual stimuli and the updated second visual stimuli to the user within the virtual environment.

In various embodiments, the first visual stimulus is presented on a floor of the virtual environment. In various embodiments, the second visual stimulus is presented on a wall of the virtual environment. In various embodiments, the second visual stimulus is presented on one or more of: a forward surface, peripheral surfaces, and a rear surface. In various embodiments, the first visual stimulus comprises a virtual platform and a virtual floor. In various embodiments, the virtual platform comprises a different shape and/or pattern from the virtual floor. In various embodiments, the first visual stimulus and the second visual stimulus comprises a size based on a visual acuity of the user. In various embodiments, a size of the first visual stimulus corresponds to the visual acuity of the user. In various embodiments, a size of the second visual stimulus is greater than the visual acuity of the user. In various embodiments, the first or the second stimulus moves autonomously and/or due to the movement caused by the user, with varying degree.

### BRIEF DESCRIPTION OF THE DRAWINGS

FIG. 1A illustrates a perspective view of an exemplary virtual reality (VR) and/or Augmented Reality (AR) system according to embodiments of the present disclosure.

FIG. 1B illustrates a front view of the exemplary VR system according to embodiments of the present disclosure.

FIG. 2 illustrates a cross-sectional view of the exemplary VR system according to embodiments of the present disclosure.

FIGS. 3A-3I illustrate various visual stimuli according to embodiments of the present disclosure.

FIG. 4 depicts an exemplary computing node according to embodiments of the present disclosure.

FIGS. 5A-5H the emergence of distinct ˜4-Hz eta oscillation during running in VR. FIG. 5A shows the LFP, raw (gray), filtered in theta (6-10 Hz, green) and filtered in eta (2.5-5.5 Hz, brown) bands during high-speed running (>15 cm s−1) on track (top) and at low speeds (<15 cm s−1, bottom) recorded on the same tetrodes on the same day in the RW (a) and VR. A power spectra is shown of these LFPs computed during the entire RW (blue) session at high and low speeds (including stops). FIG. 5B shows the LFP, raw (gray), filtered in theta (6-10 Hz, green) and filtered in eta (2.5-5.5 Hz, brown) bands during high-speed running (>15 cm s⁻¹) on track (top) and at low speeds (<15 cm s⁻¹, bottom) recorded on the same tetrodes on the same day in the VR. A power spectra is shown of these LFPs computed during the entire VR (red) session at high and low speeds (including stops). FIG. 5C shows a spectrogram (bottom, frequency versus time) of example LFPs during RW across several run and stop epochs. FIG. 5D shows a spectrogram (bottom, frequency versus time) of example LFPs during VR across several run and stop epochs. For FIGS. 5C and 5D, color bar denotes the power range in decibels (dB). White dashed lines indicate onset of the running epochs. The linear speed of a rat is shown above the spectrograms in black, along with the eta (brown) and theta (green) amplitude envelopes (scale bar shown in FIG. 5D also applies to FIG. 5C). Highlighted in gray are periods of the LFP data shown in FIGS. 5A and 5B. FIG. 5E shows the distributions of the eta amplitude index (Methods) across electrodes in VR (0.063±0.002, red) was significantly greater (P<10⁻¹⁰, χ²=540.5, Kruskal-Wallis test) than in RW (−0.013±0.003, blue). FIG. 5F shows similar distributions to FIG. 5E but for theta. Theta amplitude index in VR (0.115±0.001, red) was significantly greater (P<10⁻¹⁰, χ²=414.9, Kruskal-Wallis test) than in RW (0.056±0.002, blue). FIG. 5G shows a population-averaged power index (between run and stop; Methods) for tetrodes recorded on the same day in RW and VR (n=150, obtained from four rats and 39 sessions) that showed significant and sustained eta in RW or VR. FIG. 5H shows distributions of amplitude envelope correlations (AECs) computed as partial correlation (running speed as a controlling variable) between the LFP eta and theta amplitude envelopes across running epochs in RW (0.28±0.0019) were significantly greater (P<10⁻¹0, χ²=1864.8, Kruskal-Wallis test) than in VR (0.18±0.0013). Shades in FIG. 5G show s.e.m. θ, theta; η, eta.

FIGS. 6A-6N show additional examples of ˜4 Hz eta oscillation during running in VR, but not in RW. The data were recorded from rat #1 and rat #2. Similar format as FIG. 5A, FIGS. 6A, 6B, 6H, and 6I show traces of LFP, raw (grey), filtered in theta (6-10 Hz, cyan) and eta (2.5-5.5 Hz, magenta) bands during high-speed (above 15 cm/s) running on track (top, FIG. 6I) and at low-speeds (below 15 cm/s) (bottom, ii) recorded on the same tetrodes in the same day RW (FIGS. 6A, 6H) and VR (FIGS. 6B, 6I). FIG. 6C (rat #1) and FIG. 6J (rat #2) show amplitude envelope distribution during high—(30-60 cm/s) and low—(5-15 cm/s) speed runs for the theta (left panel) and eta (right panel) bands in RW. Theta amplitude was significantly (rat #1, p<10⁻¹⁰, X²=822.14; rat #2, p<10⁻¹⁰, χ²=218.0, KW test) larger at high speeds than low speeds, whereas eta amplitude was slightly smaller at high speeds (rat #1, p=10⁻¹⁰, X²=49.5; rat #2, p<10⁻¹⁰, X²=359.5, KW test). FIG. 6D (rat #1) and FIG. 6K (rat #2) are similar to FIG. 6C, but for VR showing large and significant increase in both eta (rat #1, p<10⁻¹⁰, X²=7942.7; rat #2, p<10⁻¹⁰, X²=279.76, KW test) and theta (rat #1, p<10⁻¹⁰, X²=5542.9; rat #2, p<10⁻¹⁰, X²=259.14, KW test) amplitudes at higher speeds. FIGS. 6E, 6F, 6L and 6M show power spectra of the example LFPs in RW (blue) and VR (red) during running (FIGS. 6E and 6L) and immobility (FIGS. 6F and 6M). FIG. 6G (rat #1) and FIG. 6N (rat #2) show the power index, during run compared to stop, showing prominent peaks in both eta and theta bands in VR (red) and only in theta band in RW (blue). (*** p<10⁻¹⁰).

FIGS. 7A-7C show additional examples of ˜4 Hz eta oscillation during running in VR. The data were recorded from rat #5, rat #6, and rat #7. Similar to the format of FIG. 5, FIGS. 7A, 7B, and 7C show traces (left) of LFP, raw (grey), filtered in theta (6-10 Hz, green) and eta (2.5-5.5 Hz, brown) bands during high-speed (above 15 cm/s) running on track (top) and at low-speeds (below 15 cm/s) (bottom) recorded in the VR. The middle, bottom panel shows power spectra of the example LFPs in VR during running (red) and immobility (black), and the top panel shows the power index, during run compared to stop, showing prominent peaks in both eta and theta bands in VR (red). The right panel shows the amplitude envelope distribution during high—(30-60 cm/s) and low—(5-15 cm/s) speed runs for the theta (top panel) and eta (bottom panel) bands in VR. Theta amplitude was significantly (rat #5, p<10⁻¹⁰, X²=1430.5; rat #6, p<10⁻¹⁰, X²=4357.1; rat #7, p<10⁻¹⁰, X²=2661.0, KW test) larger at high speeds than low speeds, whereas eta amplitude was slightly smaller at high speeds (rat #5, p<10⁻¹⁰, X²=3434.0; rat #6, p<10⁻¹⁰, X²=12250.0; rat #7, p<10⁻¹⁰, X²=1997.3, KW test).

FIGS. 8A-8J show Differential effect of speed on eta amplitude and theta frequency in RW and VR. FIG. 8A shows the running speed of the rat (top, black) and the corresponding LFP (same format as in FIG. 5A) in VR. Both theta and eta amplitudes increase with speed. FIG. 8b shows the same tetrode measured in RW on the same day showing speed-dependent increase in theta, but not eta, amplitude. FIGS. 8C and 8D shows the eidividual LFP eta-cycle amplitude and corresponding speed in VR (FIG. 8C) and RW (FIG. 8D) for the entire session in FIGS. 8A and 8B. The broken axis separates two speed ranges—below (outlined) and above 10 cm/s. Each small dot indicates one measurement. The square dots show mean and s.e.m. in each bin in RW (blue) and VR (red). A log speed scale was used for the speed range below 10 cm/s. Linear regression fits are shown separately for both speed ranges (black lines). FIG. 8E shows the population averaged theta amplitude, showing strong increase with running speed in RW. Population averaged theta amplitude in VR first decreased at low speeds (0 vs 10 cm/s) and then increased comparable to RW. FIG. 8F is the same as FIG. 8E, but the theta frequency showed significant increase with running speed in RW, but in VR the frequency dropped at very low speeds (0 vs 10 cm/s), and then became speed-independent. FIG. 8G is the same as FIG. 8E, but with a decrease in eta amplitude with increasing running speed for RW, sharp drop in eta amplitude at low speeds (0 vs 10 cm/s), and steady increase in amplitude at higher speeds in VR. FIG. 7H is the same as FIG. 8E, but with no clear dependence of eta frequency on running speed in both RW and VR. FIG. 8I shows that individual eta-cycle amplitudes are positively correlated with speed above 10 cm/s across tetrodes in VR (0.09±0.001, p<10⁻¹⁰), but not in RW (−0.107±0.005, p<10⁻¹⁰). 7.5% and 43.1% of all tetrode showed significant increase in eta amplitude with running speed in RW and VR, respectively (p<0.05). FIG. 8J shows individual eta-cycle amplitudes are negatively correlated with speed below 10 cm/s across tetrodes in VR (−0.095±0.003, p<10⁻¹⁰), but not in RW (0.002±0.003, p=0.1). Shaded areas and error bars denote s.e.m.

FIGS. 9A-9D show additional example spectrograms. FIGS. 9A, 9B, 9C, and 9D have the same format as FIGS. 5A-5D. A pronounced increase in theta and eta amplitudes can be seen during running in VR. Theta peak is pronounced in the RW, too, but eta band power increases less reliably in RW than in VR.

FIGS. 10A-10P show the speed dependence of theta and eta amplitude and frequency. Two more example data are shown having similar formats as in FIG. 3. FIGS. 10A-10H show LFP theta and eta amplitude (left two columns) and frequency (right two columns) as a function of running speed. Individual theta- and eta-cycle amplitudes (FIG. 10A, FIG. 10B, FIG. 10E, and FIG. 10F) and frequencies (FIG. 10C, FIG. 10D, FIG. 10G, FIG. 10H) from a single LFP in the same day RW (FIGS. 10A-10D) and VR (FIGS. 10E-10H) recordings for an example electrode are shown as a function of speed. FIGS. 10A and 10E show LFP theta-cycle (cyan) amplitudes and corresponding speeds in RW (FIG. 10A) and VR (FIG. 10E). FIGS. 10B and 10F show speed modulation of eta-cycle (magenta) amplitudes in RW (FIG. 10B) and VR (FIG. 10F). FIGS. 10C and 10G show speed modulation of theta-cycle frequency in RW (FIG. 10C) and VR (FIG. 10G). FIGS. 10D and 10H show LFP eta-cycle frequency speed modulation in RW (FIG. 10D) and VR (FIG. 10H). Mean and s.e.m. are shown for both RW (blue) and VR (red). Because of a near exponential distribution of the amount of data as a function of low speeds, a log speed scale was used for low speeds (outlined parts in (FIGS. 10F and 10G)). Linear regression fits are plotted (black lines). For FIGS. 10F and 10G, the broken x-axis separates two speed ranges—below (outlined) and above 10 cm/s. Dependence of theta and eta amplitude and frequency on speed above 10 cm/s (FIG. 10I, FIG. 10J, FIG. 10K, and FIG. 10L) and within a 0-10 cm/s range (FIG. 10M, FIG. 10N, FIG. 10O, FIG. 10P). FIG. 10I shows theta-cycle amplitude is similarly correlated with speed in both RW (0.20±0.005, p<10⁻¹⁰) and VR (0.22±0.004, p<10⁻¹⁰) across all tetrodes. FIG. 10J shows eta-cycle amplitude is positively correlated with speed VR (0.075±0.004, p<10⁻¹⁰), but anti-correlated in RW (−0.11±0.005, p<10⁻¹⁰) with significant difference between RW and VR (p<10⁻¹⁰, X²=676.44). k, Theta-cycle frequency and speed showed significant correlation in RW (n=991, 0.16±0.004, p<10⁻¹⁰), but not in VR (n=1222, −0.0076±0.0027, P=0.002) with significant difference between RW and VR (p<10⁻¹⁰, X²=604.22). FIG. 10L shows no significant correlation between eta frequency and speed in both RW (5.42e-04±0.0035, p=0.8) and VR (−0.008±0.0027, p=0.0022). FIG. 10M shows theta-cycle amplitude is similarly correlated with speed in both RW (0.044±0.0023, p<10⁻¹⁰) and VR (0.021±0.002, p<10⁻¹⁰) across the tetrodes. FIG. 10N shows eta-cycle amplitude is negatively correlated with speed in VR (−0.09±0.003, p<10⁻¹⁰), but not in RW 0.0022±0.003, p=0.1) with significant difference between RW and VR (p<10⁻¹⁰, X²=654.93). FIG. 10O shows significant anti-correlation between theta frequency and speed were seen in VR (n=1222, −0.074±0.0012, p=0), but not in RW (n=991, 0.012±0.0019, p<10⁻¹⁰) with significant difference between RW and VR (p<10⁻¹⁰, X²=920.35). FIG. 10P shows no significant correlation between eta frequency and speed in both RW (−0.01±0.0015, p<10⁻¹⁰) and VR (−0.016±0.0009, p<10⁻¹⁰) (***, P<0.001).

FIGS. 11A-11D show running speeds in the linear track in RW and VR. FIG. 11A shows running speed (means±SD) of the rats as a function of position on a 2.2-m-long linear track for RW (blue) and VR (red). Although the rats were faster in RW, their behavior was similar, reliably reducing speed before reaching the end of the track (n=49 sessions in RW, n=121 sessions in VR). FIG. 11B shows average speeds in RW (69.42±0.27) were significantly greater (p<10⁻¹⁰, χ²=2502.3, KW test) than in VR (47.00±0.15). FIG. 11 shows the CV of running speeds in RW (0.59±0.0023) were significantly greater (p<10⁻¹⁰, χ²=2933.6, KW test) than in VR (0.35±0.0012). FIG. 11D shows that average speeds in RW (147.13±0.02) were significantly greater (p<10⁻¹⁰, χ²=2872.7, KW test) than in VR (81.48±0.0046). Shaded areas in a denote s.e.m.

FIGS. 12A-12I show eta-theta phase-phase coupling but not eta-theta amplitude-amplitude coupling is far greater in VR than in RW during running. FIG. 12A is traces showing co-existence of eta and theta. Traces of LFP, raw (grey), filtered in theta (6-10 Hz, green) and eta (2.5-5.5 Hz, brown) bands are shown during high-speed (above 15 cm/s) running on track. FIG. 12B shows distributions of the eta-theta amplitude envelope correlation (AEC) during running (>5 cm/s, 0.27±0.0019) and stops at goal location (0.16±0.0017) in RW were significantly different (p<10⁻¹⁰, χ²=2178.2, KW test). Shaded area indicates significant correlations. FIG. 12C is the same as in FIG. 12B but in VR. Distributions of theta-to-eta amplitude envelope correlation (AEC) during runs in track (>5 cm/s, 0.18±0.0013) and stops at goal location (−0.02±0.0011) in VR were significantly different (p<10⁻¹⁰, χ²=2499.4, KW test). FIG. 12D shows phase locking values (PLV) computed as the mean vector length of the differences between instantaneous LFP theta and eta phases (See methods). The distribution of the LFP eta-to-theta PLV across the tetrodes was significantly smaller (p<10-10, χ²=331.56, KW test) in RW (0.039±0.0006) than in VR (0.063±0.0017). FIG. 12E shows distributions of eta-to-theta phase differences in RW (blue) and VR (red) for tetrodes with significant PLV. f, Eta-to-theta PLV for the same tetrodes in RW versus in VR recorded in the same day sessions, showed that 72% of tetrodes had greater eta-theta PLV in VR than RW. FIG. 12G and FIG. 12H shows the relationship between SPW amplitude and polarity and (FIG. 12G) eta-to-theta amplitude envelope correlation (AEC) (for positive SPW n=279, r=−0.05, p=0.397, for negative SPW, n=617, r=0.155, p<10⁻¹), and (FIG. 12H) phase locking value (PLV) (for positive SPW n=279, r=0.18, p=0.005, for negative SPW, n=617, r=−0.3128, p<10⁻¹⁰) in VR. Eta-theta phase-phase coupling is larger for tetrodes with larger magnitude SPW, for both +ve and −ve polarity SPW. The picture is reversed for the AEC. Number indicates max value. FIG. 12I shows a scatter plot between eta-to-theta AEC and PLV in VR (r=−0.343, p<10⁻¹⁰).

FIG. 13 shows that prominent eta band peak appears only during running in VR on tetrodes with small SPW, independent of the planer position of the electrodes. LFP power spectra for simultaneously recorded tetrodes are shown during running (red) and immobility (grey) in VR. Power spectra of the same tetrodes during running in RW are also shown (blue). Average z-scored sharp-waves computed from the baseline session preceding the VR session are shown for each tetrode (grey inset). Tetrode numbers are shown at left bottom corner of the power spectra. Center: Pictures of the bilateral cannulae with tetrode numbers (red). These are not sequential here because the numbers are determined by their sequential position in the electrode interface board.

FIGS. 14A-14F show that theta is weakest and eta is strongest in the CA1 cell layer. FIG. 14A shows LFP from three simultaneously recorded tetrodes (same color scheme as FIG. 5A) in a VR session during high-speed (>30 cm s⁻¹) run. FIG. 14B shows LFP power index (same as in FIG. 5F) for these electrodes (red). FIG. 14C shows the average z-scored (mean±s.e.m.) ripple traces (red, centered at the peak of the ripple powers) and associated SPWs (black) for the corresponding electrodes computed during the baseline session preceding the task. The eta band signal (brown) is the highest in the middle row, which has the smallest SPW amplitude, whereas the theta band signal (green) shows the opposite pattern. SO (stratum oriens), SP (stratum pyramidale), SR (stratum radiatum) and SLM (stratum lacunosum moleculare) indicate the presumed depth of the electrodes based on SPW properties. FIG. 14D is a density plot of the z-scored SPW peak amplitude and polarity during rest versus normalized (norm.) theta power during run in VR. Theta and SPW amplitudes were significantly correlated for both the positive polarity SPW (n=361, r=0.24, P<10⁻⁵; Spearman's rank correlation, here and subsequently, unless specified otherwise) and the negative polarity SPW (n=737, r=−0.24, P<10⁻¹⁰). FIG. 14E is similar to FIG. 14D but for the eta power, which shows the opposite to theta pattern (for positive SPW, n=279, r=−0.53 and P<10⁻¹⁰; for negative SPW, n=472, r=0.11 and P=0.04). FIG. 14F shows distribution of correlation (corr.) values between the absolute value of z-scored SPW peak amplitude during rest and eta normalized power during run were significantly negative (−0.34±0.04, P<10⁻¹⁰, n=70), but the same for theta were significantly positive (0.20±0.03, P<10⁻⁵, n=85). Only the sessions with at least four electrodes in the hippocampus were used. Shades in c show s.e.m. θ, theta; η, eta.

FIGS. 15A-15L show enhanced TR and eta and theta modulation of interneurons in VR. FIG. 15A shows the magnitude of the theta phase locking of interneurons in VR (0.29±0.014, n=174 from seven rats) is significantly greater than in RW (0.16±0.014, n=34 from four rats) (χ²=16.35, P<0.001, Kruskal-Wallis test) by 81%. Boxes show the 25th and 75th percentiles in the RW and VR groups; the central line shows the median; the whiskers show data in the 1.5× interquartile range outside of the 25th to 75th percentiles. FIG. 15B shows the same as FIG. 15A but for eta, which shows significantly greater (150%) phase locking of interneurons in VR (0.05±0.003) compared to RW (0.02±0.001) (χ²=14.25, P<0.001, Kruskal-Wallis test). FIG. 15C shows cumulative distribution of the log-transformed Rayleigh's Z of theta modulation for interneurons shows significantly modulated (shaded area) cells in VR (n=174, 100%) and in RW (n=33, 97.05%) at P<0.05 (dashed line). FIG. 15D is the same as FIG. 15C but for eta band, showing that 31.37% more cells are significantly modulated in VR (n=116, 66.66%) than in RW (n=12, 35.29%). FIG. 15E shows the relationship between preferred theta and eta phases of interneurons in RW (n=34, r=−0.44, P=0.011; circ-circ corr, circular statistics; Rayleigh statistics). The corresponding distributions (circ. (mean±s.d.)) of preferred theta (137.82±1.32°, MVL=0.48, green) and eta (291.67±1.39°, MVL=0.21, brown) phases are specified. A reference theta/eta cycle is plotted in black (LFP positive polarity is downward). FIG. 15F is the same as in e but for VR (n=174, r=0.35, P=1.8×10⁻⁶¹). The corresponding distributions of preferred theta (155.4±1.38°, MVL=0.42, green) and eta (219.41±1.35°, MVL=0.29, brown) phases in VR are shown. The distributions are significantly different for theta (P=0.02, V=0.132, Kuiper's test) and eta (P=0.05, V=0.339, Kuiper's test) preferred phases between RW and VR. FIG. 15G shows no significant correlation between eta and theta DoMs of interneurons was seen in RW (n=34, r=0.28, P=0.11, partial correlation factoring out number of spikes). FIG. 15H is the same as FIG. 15G but in VR, showing strong positive correlation (n=174, r=0.75, P<10⁻¹⁰, partial correlation factoring out number of spikes). FIG. 15I shows corrected auto-correlations ordered according to the increasing TR1 values for RW. The auto-correlograms are normalized by their first theta peak values as for the place cells. FIG. 15J is the same as FIG. 15I but for VR, showing more theta peaks—that is, greater rhythmicity—than in RW. FIG. 15K shows that the population average of auto-correlations show greater theta rhythmicity (TR) in VR compared to RW. FIG. 15L is histograms of the TR1 distributions in VR (−0.118±0.004) are 82% greater (P<10⁻¹⁰, χ²=47.31, Kruskal-Wallis test) than in RW (−0.215±0.009). θ, theta; η, eta; MVL, mean vector length.

FIGS. 16A-16L show enhanced theta rhythmicity but not eta modulation of CA1 place cells in VR. FIG. 16A shows the magnitude of theta phase locking in RW (0.2±0.005, n=407) and VR (0.196±0.006, n=499) are not significantly different (p=0.07, χ²=3.26). Boxes show 25th and 75th percentiles in RW and VR groups, central line shows the median and the whiskers data in the 1.5× inter-quartile range outside of 25th to 75 the percentiles. FIG. 16B is the same as FIG. 16A, but for eta phase locking in RW (0.089±0.003) and VR (0.08±0.002) also showing no significant difference (p=0.076, χ²=4.93). FIG. 16C shows cumulative distribution of log-transformed Rayleigh's Z computed for theta modulation of the place cells. This was significant (shaded area) at 0.05 level for a majority of cells in RW (n=297, 72.9%) and VR (n=360, 72.1%). FIG. 16D is the same as FIG. 16C but for eta band in RW (n=60, 14.7%) and VR (n=100, 20.04%). FIG. 16E shows the relationship between preferred theta and eta phases of place fields in RW (n=407, r=0.16, p=0.0014, circ-circ corr.). The corresponding distributions (circ. (mean±std.)) of preferred theta (223.65±1.40° o, MVL=0.50, green) and eta (241.53±1.39°, MVL=0.40, brown) phases of place fields in RW are shown. A reference theta/eta cycle is plotted in black (LFP positive polarity is downward). FIG. 16F is the same as FIG. 16E but for VR (n=499, r=0.15, p<0.001). The corresponding distributions of preferred theta (231.71±1.32°, MVL=0.28, green) and eta (166.34±1.30°, MVL=0.06, brown) phases in VR are shown. The preferred distributions are significantly different for theta (p=0.02, V=0.132, Kuiper's test, 5) and eta (p=0.01, V=0.175, Kuiper's test) between RW and VR. FIG. 16G shows no significant correlation was found between theta and eta depth of modulation of spikes (DoMs), defined as the magnitude of phase locking (see methods), within the place fields in RW (n=407, r=−0.025, p=0.605, partial Spearman correlation factoring out number of spikes). FIG. 16H is similar to FIG. 16G but for VR showed significant positive correlation between eta and theta DoMs (n=499, r=0.19, p<10-5). FIG. 16I shows autocorrelograms of spike trains (corrected by the overall autocorrelation decay, see methods) ordered according to the increasing TR1 values for the place fields in RW. The autocorrelograms are normalized by the amplitude of their theta peak to allow easy comparison. FIG. 16J is the same as 5i, but for VR, showing more theta peaks, i.e., greater rhythmicity, than in RW. FIG. 16K shows the population average of autocorrelations shows greater theta rhythmicity in VR than in RW. FIG. 16L shows the distribution of the theta rhythmicity (TR1) index was significantly greater (p<10-10, χ²=123.3) in VR (−0.151±0.07) than RW (−0.275±0.158).

FIGS. 17A-17J show a Model fit of autocorrelograms of interneurons in RW and VR. FIGS. 17A and 17B show examples of interneurons' autocorrelograms (grey) with TR1 values along with fits using GMM in RW (FIG. 17A, top two rows, left, blue) and in VR (FIG. 17B, bottom two rows, left, red). The distribution of spikes' theta (middle column) and eta (right column) phases are given. FIG. 17C shows histograms of ACG rhythmicity decay in RW (blue, n=36, 0.64±0.04) and VR (red, n=157, 1.4±0.04) are significantly different (p<10-10, χ²=81.37). FIG. 17D shows histograms of ACG decay constant in RW (blue, 8.4±0.39) and VR (red, 9.8±0.07) are shown (p<10⁻⁵, χ²=19.65). FIG. 17E shows histograms of ACG theta period in RW (0.12±0.015) and VR (0.138±0.02) are significantly different (p<10⁻¹⁰, χ²=82.63). FIG. 17F shows histograms of ACG peak widths in RW (0.65±0.002) and VR (0.54±0.0012) are significantly different (p=1.1*10⁻⁷, χ²=23.71). FIG. 17G and FIG. 17H shows heat maps of ACGs of spike trains sorted by increasing TR1 for putative interneurons recorded during running in RW (FIG. 17G) and VR (FIG. 17H). The ACGs are normalized by their first theta peak values. FIG. 17I shows the population average of autocorrelations shows greater theta rhythmicity in VR than in RW. FIG. 17J shows histograms of the TR1 distributions having a significant difference between RW (median=−0.09) and VR (median=−0.07) (p<10⁻¹⁰, χ²=57.02).

FIGS. 18A-18L show theta rhythmicity index of the putative pyramidal cells and interneurons in RW and VR. FIGS. 18A-18F show data from putative pyramidal cells. FIG. 18A shows TR1 distributions in VR (−0.1±0.0075, n=355) is much greater (p<10⁻¹⁰, ↔²=72.83) than RW (−0.17±0.014, n=268). FIG. 18B shows the difference of third-to-second peak of theta (TR2) in VR (−0.21±0.007, n=153) is much greater (p<10⁻¹⁰, χ²=51.83) than RW (−0.32±0.0081, n=186). FIG. 18C shows the difference of fourth-to-third theta peak (TR3) in VR (−0.21±0.008, n=201) is much greater (p<10⁻⁹, χ²=33.58) than in RW (−0.36±0.013, n=114). FIG. 18D is the s as FIG. 18A but model corrected ACG estimates show TR1 in VR (−0.14±0.0091, n=357) is much greater (p<10⁻¹⁰, χ²=54.44) than in RW (−0.21±0.014, n=274). FIG. 18E shows the difference of third-to-second peak (TR2) difference index (p=2.5188e-05, χ²=17.75) between RW (−0.32±0.0153, n=252) and VR (−0.276±0.009, n=155). FIG. 18F shows the difference of fourth-to-third peak (TR3) difference index (p=0.002, χ²=9.58) between RW (−0.35±0.014, n=142) and VR (−0.27±0.009, n=76). FIGS. 18G-18L are similar to FIGS. 18A-18D but for interneurons. FIG. 18G shows a significant difference of TR1 distributions (p<10⁻¹⁰, χ²=47.56) cells between RW (−0.09±0.004, n=33) and VR (−0.05±0.0033, n=149). FIG. 18H shows the difference of third-to-second peak (TR2) difference index (p=0.49, χ²=0.47) between RW (−0.0767±0.0031, n=33) and VR (−0.078±0.0035, n=149). FIG. 18I shows the difference of fourth-to-third peak (TR3) difference index (p=0.13, χ²=2.18) between RW (−0.066±0.006, n=33) and VR (−0.05±0.007, n=148). FIG. 18J shows a significant difference of TR1 distributions (p=4.8071e-12, χ²=47.76) of putative pyramidal cells between RW (−0.215±0.0097, n=33) and VR (−0.116±0.0048, n=149). FIG. 18K shows a difference of third-to-second peak (TR2) difference index (p<10-5, χ²=16.29) between RW (−0.21±0.0075, n=33) and VR (−0.153±0.0098, n=149). FIG. 18L shows a difference of fourth-to-third peak (TR3) difference index (p=2.3933e-06, χ²=22.25) between RW (−0.21±0.0187, n=23) and VR (−0.115±0.0076, n=130). (***, P<0.001).

FIGS. 19A-19H show the relationship between theta rhythmicity and theta and eta phase locking of place cells and interneurons in RW and VR. FIGS. 19A and 19B show the quantified relationship between TR1 and eta (r=−0.1, p=0.28, partial Pearson correlation with number of spikes as controlling variable) and theta (r=0.0032, p=0.95) phase locking of place cells in RW. FIGS. 19C and 19D show the place cells with higher TR1 showed increasingly more eta (r=0.22, p<10⁻⁵), but not theta (r=0.06, p=0.2), phase locking in VR. FIG. 19E and FIG. 19F show that no systematic relationship was found between TR1 and eta (r=−0.1, p=0.28) (FIG. 19E) or theta (r=−0.15, p=0.41) (FIG. 19F) phase locking in RW for interneurons. FIGS. 19G and 19H show that interneurons with higher TR1 showed increasingly more eta (r=0.19, p=0.02) (FIG. 19G) and theta (r=0.22, p=0.01) (FIG. 19H) phase locking in VR.

FIGS. 20A-20J show Model based estimate of theta rhythmicity of place fields in RW and VR. FIGS. 20A and 20B show examples of place cell ACG (grey shaded area) along with fits using a Gaussian mixture model (GMM, see methods) in RW (FIG. 20A, left column, blue) and in VR (FIG. 20B, left column, red). The theta (middle column) and eta (right column) phase distributions for these example place fields. FIG. 20C shows histograms of ACG rhythmicity decay in RW (blue, 0.59±0.0439, n=312) and VR (red, 1.13±0.059, n=326 are significantly different (p<10⁻¹⁰, χ²=107.92). Thus, ACG in VR decayed nearly half as much as RW. FIG. 20D shows histograms of ACG decay constant in RW (blue, 1.06±0.08) and VR (red, 3.81±0.16) show that the ACG decayed more than twice as slowly than in RW (p<10⁻¹⁰, X²=427.37) indicating sustained theta rhythmicity. FIG. 20E shows histograms of ACG theta period in VR (0.13±0.0006 sec.) is significantly greater (p<10⁻¹⁰, χ²=427.38) than in RW (0.11±0.0006 sec.). FIG. 20F shows histograms of ACG theta peak widths in VR (0.56±0.0065 sec.) are significantly smaller (p<10⁻¹⁰, χ²=89.37) than in RW (0.66±0.007 sec.), showing greater theta rhythmicity. FIGS. 20G and 20H show heat maps of the GMM estimates of ACGs, sorted by increasing TR1 for the place fields recorded during running in RW (FIG. 20G) and VR (FIG. 20H). The ACGs are normalized by their first theta peak values for easy comparison. FIG. 20I shows the population average of ACGs have greater theta rhythmicity in VR than in RW. FIG. 20J shows histograms of GMM corrected estimates of the TR1 distributions show VR (median=−0.12) is 75% greater than RW (median=−0.21) (p<10⁻¹⁰, χ²=135.54).

FIGS. 21A-21C show theta rhythmicity is greater in VR than RW even when factoring out place field width and number of spike contribution. FIG. 21A shows place fields are broader (p<10⁻¹⁰, χ²=64.1158) in VR (55.3±1.27 cm) than in RW (42.2±1.08 cm). FIG. 21B shows theta rhythmicity index TR1 increases as a function of the place field width in RW (blue, r=0.34, p<10⁻¹⁰) and VR (red, r=0.33, p<10⁻¹⁰). Linear regression fits are shown. TR1 is consistently greater in VR than in RW across all place field widths. FIG. 21C shows TR1 increases with the number of spikes in place field, in RW (blue, r=0.22, p<10⁻¹⁰) and VR (red, r=0.20, p<10⁻⁵). Linear regression fits are shown (blue—RW, red—VR).

FIGS. 22A-22D show Relationship between number of spikes and eta phase locking of place cells and interneurons in RW and VR. FIGS. 22A and 22B show the logarithm of number of spikes generated by a cell was inversely correlated with the depth of modulation of eta band phase locking in the RW (r=−0.45, p<0.01) and VR (r=−0.3, p<0.01). FIGS. 22C and 22D show that the interneurons have a similar relationship with eta in VR (r=−0.4, p<0.01). This was not as clear in the RW (r=−0.04, p=0.82), potentially due to the smaller number of interneurons. Thus, even though far greater fraction of interneurons, show significant eta phase locking compared to pyramidal neurons, the numerical value of eta-DOM index is smaller for the interneurons than pyramidal neurons.

FIGS. 23A-23F show eta oscillations in VR is present across different landmarks (distal visual cues) and their configurations (symmetric vs. asymmetric) during high speed running. The data were recorded from the rat #5. FIGS. 23A-23C show top-down schematic view of the VR mazes showing elevated linear track centered in a 300 cm×300 cm room. The distal visual cues indicate type of the task—(FIG. 23A) asymmetric, symmetric (FIG. 23B) and alternative asymmetric (FIG. 23C) VR rooms. FIGS. 23D-23F show power spectra (top) and power indices (bottom) across all the tetrodes recorded (n=10) in (FIG. 23D) assymetric (left column), (FIG. 23E) symmetric (middle column) and (FIG. 23F) alternative asymmetric (right column) VR rooms. Asymmetric and symmetric results are from the same session recorded in consequent 15 trials. The green and brown shaded areas indicate theta and eta frequency ranges, correspondingly. Shades in d and e show s.e.m.

FIGS. 24A-24E show eta rhythm is present in several two-dimensional VR tasks. Rats were trained to run in two dimensional VR along different paths, each involving different amount of angular movement: FIG. 24A shows linear paths between two fixed locations with 180 degree turns at the ends and small angular movements in between, FIG. 24B shows running along the perimeter of a triangular path with 120 degree turns at the ends and small angular movements in between, and FIG. 24C shows random-foraging in two dimensional plane with very little straight line paths and nearly constant angular motion. All three experiments were done in two dimensional VR with different sets of visual cues than in 1D VR. Rat's running speed varied across different experiments, with slowest average speed in 2D random foraging, followed by the linear path. FIG. 24D shows the population averaged eta amplitude in VR as a function of running speed is similar to the computations in FIG. 15 in 1D VR with no angular motion in the middle or at the end of the track (see methods). These data are from n=65 electrodes in linear paths, n=125 electrodes in triangular paths and n=180 in 2D random paths. Despite these major differences in vestibular, visual and locomotion cues, all of these experiments showed a comparable sharp drop in eta amplitude from zero to low speeds (0 vs 10 cm/s) and steady increase in amplitude at higher speeds. The data for random and linear paths are go only up to 30 cm/s, because rats never ran at higher speeds. FIG. 24E is the same as FIG. 24D but for theta amplitude, which shows a steady increase of the amplitude with running speed for all three different trajectories. These results are similar to the speed-dependence of eta and theta on 1D track without any vestibular or rotational cues. Shades in FIGS. 24D and 24E show s.e.m.

FIGS. 25A-25F show the synchronicity of eta and theta oscillations within and across the hemispheres. FIGS. 25A and 25B show scatter plots between phase locking values (PLV) and mean phase differences of the eta FIG. 25A and theta FIG. 25B oscillations recorded in pairs of tetrodes within the same cannulae. FIG. 25C shows a density plot of relationship between eta and theta PLV computed in pairs of tetrodes within the same cannulae. FIGS. 25D and 25E show scatter plots between PLV and mean phase differences of the eta FIG. 25D and theta FIG. 25E oscillations recorded in pairs of tetrodes across the different cannulae. FIG. 25F shows a density plot of relationship between eta and theta PLV computed in pairs of tetrodes across the different cannulae. The PLVs are computed during running in the linear track.

FIGS. 26A-26H show hippocampal response to a revolving bar of light. FIG. 26A shows a schematic of the experimental setup and FIG. 26B shows a top-down view. The rat's head is at the center of a cylinder. A green-striped bar of light (13° wide) revolves around the rat at a fixed distance in two directions (clockwise (CW) or counterclockwise (CCW)). Rat's putative field of view is 270°, with the area (dark gray) behind him being invisible to him. FIG. 26C shows Raster plots. Trial number (y-axis on the left) and firing rates (y-axis on the right) of six CA1 neurons as a function of the angular position of the bar (x-axis, 0° in front of the rat and ±180° behind). Bold arrows underneath show the direction of revolution (top panels, Counterclockwise (CCW); bottom panels, Clockwise (CW)). FIG. 26D shows the cumulative distribution function (CDF) of strength of tuning (z-scored sparsity, see methods) for 1191 active CA1 putative pyramidal cells (response with higher tuning chosen between CCW and CW, FIGS. 26D-26F. The actual data shows significantly greater (KS-test p=1.26×10⁻⁸⁹) tuning than the shuffled data (Gray line for FIGS. 26D-26G). 39% of neurons showed significant (z>2, vertical black line) tuning. FIG. 26E shows the distribution of tuned cells as a function of the preferred angle (angle of maximal firing). There were twice as many tuned cells at forward angles than behind. FIG. 26F shows the median z-scored sparsity and its variability (SEM, shaded area, here and subsequently) of tuned cells as a function of their preferred angle. (Correlation coefficient r=−0.28 p=1.5×10⁻⁹). FIG. 26G shows the median value of the full width at quarter maxima across the ensemble of tuned responses increased as a function of preferred angle of tuning. (r=+0.15 p=1.3×10+). FIG. 26H shows the CDF of firing rate modulation index within versus outside the preferred zone (see methods) for tuned cells was significantly different (Two-sample KS test p=1.9×10⁻⁵⁰) than untuned cells.

FIGS. 27A-27D show the relationship between different properties of SAC. FIG. 27A shows (top) SAC quantified by z-scored sparsity is significantly correlated (r=0.82, p<10⁻¹⁵⁰) with, but significantly greater than the z-scored direction selectivity index (DSI) (41% z>2 for sparsity vs 31% for DSI, KS-test p=9.3×10⁻¹⁰); (Bottom) Cumulative histogram (cdf) of z-scored metric of sparsity and DSI. FIG. 27B is similar to FIG. 27A, wherein (1-(circular variance)) is significantly correlated (r=0.84, p<10⁻¹⁵⁰) but significantly weaker (33% z>2 for (1−circular variance)) than sparsity. (KS-test p=7×10⁻⁶). FIG. 27C is similar to FIG. 27A as coherence is significantly correlated (r=0.89 p<10⁻¹⁵⁰) but significantly weaker (26% z>2 for coherence KS-test p=6.3×10⁻¹⁶) than sparsity. FIG. 27D is similar to FIG. 27A, but mutual information is significantly correlated (r=0.47 p=8.6×10⁻¹³²) but significantly smaller than sparsity (37% z>2 for mutual information, KS-test p=7.2×10⁻⁵)

FIGS. 28A-28C show the Unimodality of SAC. The majority of uni-directional (FIG. 28A) as well as bidirectional tuning curves (FIG. 28B) were unimodal with only one significant peak (top row), whereas untuned responses (FIG. 28C) did not have significant peaks, as expected. Both tuned responses were used for the bi-directional cells, and only the tuned response was used for the uni-directional cells. Significant troughs, i.e., off-responses were not found for unidirectional or bidirectional cells (bottom row). Significance of a peak (or trough) was determined with the spike train shuffling analysis, similar to that performed to compute the z-scored sparsity. A peak (trough) was determined to be significant if it was larger (smaller) than the median value of peaks in all shuffles and had a height of at least 20% of the range of firing rate variation in the shuffle data. These criteria resulted in zero significant peaks for some tuned responses.

FIGS. 29A-29M show trial-to-trial variability and co-fluctuation of simultaneously recorded cells: For each cell, in each trial, computed the mean firing rate (MFR), mean vector length (MVL) and mean vector angle (MVA) of SAC were calculated (see methods). FIG. 29A shows trial to trial variation of firing rate (top) was significantly (T-test p=3.4×10⁻¹²) higher for untuned cells (gray, mean coefficient of variation (CV)=1.22), compared to tuned cells (maroon, mean CV=1.02), when using all trials. FIG. 29B (top) shows the difference in variability was not significantly correlated with SAC tuning strength (after factoring out firing rate, partial correlation r=−0.04, p=0.14). FIG. 29A (bottom) shows the rate-variability was qualitatively similar between tuned and untuned cells when using only the responsive trials (firing rate above 0.5 Hz, T-test p=0.2), and FIG. 29B (bottom) shows uncorrelated (partial correlation after factoring out mean firing rate p=0.85) with the degree of SAC. FIG. 29C shows the variance of mean vector length, which is a measure of the non-uniformity of spiking as a function of the stimulus angle, was significantly greater for untuned cells (T-test p=0.002) than tuned cells, and FIG. 29D shows that this was inversely related to SAC tuning strength (r=−0.19, p=7.3×10⁻¹⁰). FIG. 29E shows the circular standard deviation of MVA, which signifies the instability of SAC tuning from trial to trial, was significantly (p=4.1×10⁻⁹⁴) smaller (11%) for tuned than untuned cells, and FIG. 29F shows that this strongly anti-correlated with SAC (r=−0.77 p=7.4×10⁻¹⁹²). FIG. 29G shows this standard deviation of MVA was inversely correlated with MVL for tuned (r=−0.15 p=0.004), and for untuned cells (r=−0.12 p=0.003). FIG. 29H shows that it was also positively correlated with the location of tuning (r=0.18 p=3.5×10⁻⁴), with lower variation for cells tuned to the front angles (abs. avg. MVA around 0°) than behind (±180°). Standard deviation of MVA was uncorrelated with location of tuning for untuned cells (p=0.64). FIG. 29I shows two simultaneously recorded cells showing SAC in the CCW direction. FIG. 29J shows data for trial numbers 53 to 59, showing mostly uncorrelated rate variability. FIG. 29K shows that only 17% of tuned cell-pairs showed significant (z>2) co-fluctuation of mean firing rates across trials, while 7% of cell pairs had significantly opposing fluctuations (z<2). (see methods). FIG. 29L shows that only 9% of cell pairs showed significant co-fluctuation of SAC. SAC and firing rate co-fluctuations were computed between simultaneously recorded cell-pairs of tuned or untuned cells in only trials when the rat was stationary (see methods). CCW and CW tuning curves were treated as separate responses throughout these analyses. FIG. 29M shows the strength of rate co-fluctuation was positively correlated with overlap between the two tuning curves, quantified as the correlation coefficient between their SAC (r=0.178 p=0.004).

FIGS. 30A-30C show the continuity of stability and sparsity measures. FIG. 30A shows across all neurons, the z-scored sparsity, i.e., degree of tuning, and stability varied continuously, with no clear boundary between tuned and untuned neurons. FIG. 30B shows the same distribution as FIG. 30A, with colorcoding of stable and tuned responses separated. FIG. 30C shows a detailed breakdown of SAC properties that had significant sparsity (i.e., tuned) or significant stability and whether these were observed in both directions (e.g., bidirectional stable) or only one direction (e.g., unidirectional tuned). If unidirectional, whether CW or CCW direction was significant. Nearly all cells that were significantly tuned in a given direction were also stable in that direction.

FIGS. 31A-31K show the directionality, stability and ensemble decoding of SAC. FIG. 31A shows an example of a bidirectional cell, showing significant (z>2) tuning (maroon) in both C CW and CW directions. FIG. 31B is similar to FIG. 31A, but for a uni-directional cell, showing significant tuning in only one direction (CW here). CCW (blue) and CW (red) trials have been grouped together for ease of visualization, but experimentally were presented in alternating blocks of four trials each. FIG. 31C shows example cells showing stable responses (lavender) with multiple peaks that did not have significant sparsity (z<2) (bi-directional stable, left; unidirectional stable (CCW), right). FIG. 31D shows relative percentages of cells. FIG. 31E shows the percentage of tuned responses as a function of the absolute preferred angle, for bidirectional and unidirectional populations are significantly different from each other (twosample KS test p=0.04). FIG. 31F shows the correlation coefficient of CCW and CW responses for different populations of cells, (two sample KS test green, bidirectional, p=3.3×10⁻²⁷, orange, unidirectional p=7.0×10⁻²⁷, lavender, untuned stable, p=4.4×10⁻⁴. Dashed curves indicate respective shuffles. FIG. 31G shows the firing rate modulation index for uni-directionally tuned cells (see methods), for angles around the response peak (preferred zone) was significantly different from zero (t-test p=4.1×10⁻³⁵), but not outside preferred zone (t-test p=0.35). FIG. 31H shows an example of the decoding of 10 randomly chosen trials (gray) using all tuned cells in the CCW direction (maroon); all other trials were used to build the population-encoding matrix. FIG. 31I is the same as FIG. 31H, but using the untuned-stable responses (lavender). FIG. 31J shows the median error between stimulus angle and decoded angle over 30 instantiations of 10 trials each for actual and shuffle data. The decoding errors for tuned (median=17.6°) and untuned stable (median=45.2°) are significantly less than that of shuffle (non-parametric rank sum test p<10⁻¹⁵⁰ for both populations). Green dashed line indicates width of the visual cue; black dashed line indicates median error expected by chance. FIG. 31K shows a sample iteration showing decoding error decreases with increase in the number of responses used for decoding, for populations of all (black), tuned (maroon) and untuned stable (lavender) cells, but not for untuned unstable cells (gray).

FIG. 32 shows additional examples of tuned cells. For clarity, the CCW (blue) and CW (red) trials are stacked separately in all raster plot figures, even though these alternated every four trials. First five examples are of bi-directionally tuned cells (green y-axis); next four examples are of uni-directionally tuned cells (orange-yellow y-axis).

FIG. 33 shows additional examples of bi-directionally stable but untuned cells. These cells did not have significant sparsity (z<2) in either direction but had significant stability.

FIGS. 34A-34F show the firing rate differences between CW and CCW revolution direction: FIG. 34A shows that the firing rate of unidirectional cells in tuned versus untuned directions is significantly higher (paired t-test p=4.5×10⁻¹⁰) in the tuned direction. FIG. 34B is the same as FIG. 34A, for bidirectional cells showing higher firing rate (paired t-test, p=2.0×10⁻⁶) in the revolution direction with better tuning. FIG. 34C shows the cumulative histogram of ratio between firing rate in untuned to tuned direction was less than one for 67% (65%) of cells. FIG. 34D is the same as FIG. 34C, but for bidirectional cells (other/better since both directions are tuned) showing 65% of firing rate ratios were less than one. FIG. 34E shows that to remove the contribution of firing rate to sparsity, the strength of tuning (z-score sparsity) difference was computed with spike thinning procedures (similar to FIG. 35, see methods) ensuring equal firing rate in both directions. The difference in tuning strength (z-scored sparsity) was not significantly correlated with firing rate ratio for unidirectional (r=−0.09 p=0.16) as well as (FIG. 34F) bidirectional (r=0.005 p=0.95) populations. For bi-directionally tuned cells, SAC with higher z-scored sparsity was labeled as the “better” response, and the SAC with lower z-scored sparsity was called “other” response.

FIGS. 35A-35D show that t The relative number of bidirectional cells increases with mean firing rate, but not the fraction of tuned cells. To remove the effect of firing rate on z-scored sparsity computation, we randomly subsampled spike trains to have a firing rate of 0.5 Hz (see methods). FIG. 35A shows the fraction of cells with significant sparsity, i.e. fraction tuned, increased with the firing rate for the actual data (r=0.11 p=2.2×10⁻⁶), but after spike thinning, there was no correlation (r=0.01, p=0.77). Thus, the true probability of being tuned was independent of the firing rate of neurons. FIG. 35B shows the proportion of bidirectional and uni-directional tuned neurons is comparable (10% vs 13%) with and without spike thinning. FIG. 35C shows the fraction of bi-directional cells compared to uni-directional cells increases with original firing rate, even after spike train thinning. FIG. 35D shows that a spike thinning procedure reduces the sparsity of the tuning curves, as expected, due to loss of signal. After spike thinning, sparsity was significantly correlated in both directions of revolution (r=0.39, p=3.8×10⁻³¹) and this was not due to the rate changes because sparsity was uncorrelated with firing rates (r=0.01, p=0.72 for CCW sparsity and firing rate, r=0.02, p=0.54 for CW sparsity and firing rate).

FIGS. 36A-36K show the p Population vector stability and decoding of visual cue angle. FIG. 36A shows the stability for CCW tuned responses (n=310). Color indicates correlation coefficient between two non-overlapping groups of trials' population responses (see methods). The maximum correlation values were pre-dominantly along the diagonal. Maxima along x-axis and y-axis were significantly correlated (Circular correlation coefficient r=0.97, p<10⁻¹⁵⁰). FIG. 36B is the same as FIG. 36A, but using untuned stable cells (n=266) showed significant ensemble stability (r=0.91, p<10⁻¹⁵⁰). FIG. 36C shows the same as FIG. 36A, but using untuned and unstable cells (n=306). This was not significantly different than chance (r=−0.16, p=0.09). FIG. 36D is the same as FIG. 36A, using tuned cells with their spike trains circularly shifted in blocks of four trials, showed no significant stability (r=1.1×10⁻³, p=0.99). FIGS. 36E-36H are the same as FIGS. 36A-36D, but for CW data. FIG. 36I shows that decoding CW direction has similar results as in CCW direction (shown earlier in FIG. 31). Similar analysis as shown in FIG. 31 for the stimulus movement in CW direction. (Left) Decoding cue angle in 10 trials of CW cue movement, using all other CW trials to build a population-encoding matrix. Gray trace indices movement of visual bar, colored trace is the decoded angle. (Right) Same as left, for shuffle data. FIG. 36J is the same as FIG. 36I, but using the untuned-stable cells in CW movement direction. FIG. 36K shows the median error between stimulus angle and decoded angle over 10 instantiations of decoding 10 trials each for actual and cell ID shuffle data. Green dashed line indicates width of the visual cue; black dashed line indicates median error expected by chance.

FIGS. 37A and 37B show retrospective coding of SAC cells versus prospective coding in place cells. FIG. 37A (Top) shows that a bidirectional cell responds with a latency after the stimulus goes past the angular position of the bar of light depicted by the green stripped bar. Bottom-Population overlap is above the 45° line, indicating retrospective response. FIG. 37B is the same as FIG. 37A but for a prospective response, where the neuron responds before the stimulus arrives in the receptive field. Such prospective responses are seen in place fields during navigation in the real world, where the population overlap is maximal below the 45° line. Prospective coding was seen in purely visual virtual reality, but those cells encoded prospective distance, not position.

FIGS. 38A-38K show the retrospective nature of stimulus angle coding. FIG. 38A shows that for bidirectional tuned cells, the peak angle in the CW (y-axis) direction was greater than that in the CCW (x-axis). FIG. 38B shows a histogram of difference (CW−CCW, restricted to ±50°) of the peak angles in two directions of a cell was significantly (t-test, p=0.003) positive indicating a retrospective shift. For bidirectional cells: FIG. 38C shows stack plots of normalized population responses of cells, sorted according to the peak angle in the CCW (left). The corresponding responses of cells in the CW direction (right). FIG. 38D shows an example cell showing retrospective latency between the CCW (blue) and CW (red) tuning curves, corresponding to the horizontal white boxes in FIG. 38C. FIG. 38E shows the cross correlation between the CCW and CW responses in FIG. 38D had a maximum at positive latency (+27°). FIG. 38F shows cell wise cross correlations between CW and CCW tuning curves, sorted according to their peak-lag. Majority (80%) of lags were positive, i.e., retrospective. The ensemble median lag of 19.9°±49.8° was significantly positive (Circular median test at 0°, p=4.8×10⁻¹⁶). FIG. 38G shows the firing rate, averaged across the entire ensemble of bidirectional cells at −30° in the CCW direction was misaligned with the ensemble averaged responses in the CW direction at the same angle (top), but better aligned with the ensemble averaged responses in the CW direction at −10° (bottom, vertical boxed in FIG. 38C), showing retrospective response. FIG. 38H shows a population vector overlap of SAC across all cells. At all angles, these population vector correlation coefficients had a peak at a positive lag (CW peak-CCW peak, median=+54.3°±25.3° t-test p=0.007) showing a retrospective shift. Black marker (+) indicates the correlation coefficient between the population responses at black boxes, i.e. the population response in FIG. 38G. FIG. 38I is the same as FIG. 38C for uni-directional cells with CCW tuned cells (top row) and CW tuned cells (bottom row) sorted according to their SAC peak in the tuned direction. FIG. 38J is the same as in FIG. 38F. Cross correlations from the uni-directional tuned cells were combined and sorted according to the peak-lag. Majority (67%) of the cross correlations had a significantly positive lag (median latency=19.9°±86.1°, circular median test at 0°, p=1.8×10⁻¹⁰). FIG. 38K is the same as FIG. 38H for unidirectional cell population vector cross-correlation. For all angles the population vector cross correlation coefficients had a peak at a positive lag (CW peak-CCW peak, median=+56.2°±23.7° t-test p=0.001) showing retrospective coding, which was not significantly different from the retrospective lag in bidirectional cells (KS-test, p=0.28).

FIGS. 39A and 39B show a photodiode experiment to measure the latency introduced by the equipment: Instead of a rat, a photodiode was placed where the rat sat. FIG. 39A shows the signal from the photodiode (purple trace) synchronized with bar position (black) was extracted, and FIG. 39B shows the cross correlation computed between the CW and CCW tuning curves of photodiode response. The cross correlation had maxima at a latency of −2.8°, which corresponds to a temporal lag of 38.9 ms. This was much smaller than the latency between neural spike trains (median latency 22.7°, corresponding to a temporal latency of 315.3 ms before accounting for the recording apparatus latency). For all the latency numbers reported in the main text, this small latency introduced by the recording apparatus was removed.

FIGS. 40A-40D show significant retrospective SAC in the untuned stable cells but not unstable cells. FIG. 40A shows the average strength of tuning in CCW and CW direction is inversely related to the peak angular lag between the two SAC for bidirectional (r=−0.19 p=0.04) as well as unidirectional cells (r=−0.16 p=0.02). FIG. 40B shows the absolute difference between strengths of tuning between CCW and CW directions was not significantly correlated with the peak angular lag in their cross correlation for bidirectional (r=0.13 p=0.14) or unidirectional cells (r=0.03 p=0.64). This analysis was restricted to cells with retrospective lags, which were in majority. FIG. 40C shows untuned stable cells too show significant retrospective bias, quantified using the cross correlation between the tuning curves in CCW and CW directions (median lag=−13.6° circular t-test p=0.02). FIG. 40D shows that the results observed in FIG. 40C were not observed for the untuned unstable population (median=−4.6°, circular t-test p=0.39).

FIGS. 41A-41G show the dependence of SAC tuning on stimulus pattern, color, movement predictability and time. FIG. 41A shows the response of the same cell has similar SAC for green striped pattern (left) and green-checkered pattern (right). FIG. 41B is similar to FIG. 41A, but for changes of stimulus color, green and blue, and pattern (horizontal vs vertical stripe). FIG. 41C is the same as FIG. 41A, but for changes to predictability of the stimulus, termed “systematic” (left) for predictable movement of the stimulus, as compared to “random” (right, see methods). FIG. 41D is the same as FIG. 41A, but for the same cell's response to the same systematic stimulus across 2 days. FIG. 41E shows firing rate remapping, quantified by FR change index (mean±SEM), was significantly (p<8×10⁻⁶) smaller for the actual data (dark-pink) than for shuffle data (gray) for all conditions. FIG. 41F is similar to FIG. 41E, but correlation coefficient between the tuning curves across different conditions (mean values: pattern=0.48, color=0.39, predictability=0.28, day=0.19. All correlations were significantly greater (t-test p<7.7×10-9) than shuffle. FIG. 41G is the same as FIG. 41E, using angular lag in cross correlation to quantify amount of shift between tuning curves across the two conditions (pattern=48°, color=59°, predictability=63°, day=74°). All were significantly lesser (t-test p<0.003) than shuffle. All example cells here are chosen from CW condition for clarity.

FIGS. 42A-42K show Additional properties of SAC invariance. FIG. 42A (Row 1) shows that for same cells recorded in response to the movement of a green striped and green checkered bars of light, mean firing rate during stationary epochs (running speed<5 cm/sec), was significantly correlated (r=0.48 p=2×10⁻⁵). Preferred angles of SAC between the two stimulus patterns were also significantly correlated (circular correlation r=0.32 p=5×10⁻³). Solid red dots denote preferred angles of cells tuned (sparsity (z)>2) in both conditions; gray dots are for cells with significant tuning in one of the conditions. FIG. 42A (Row 2) is the same as FIG. 42A (Row 1), but for responses to changes of stimulus color, green and blue. Firing rate (r=0.45 p=1×10⁻⁴) and preferred angle (r=0.36 p=0.01) were correlated. FIG. 42A (Row 3) is the same as FIG. 42A (Row 1), but for changes to predictability of the stimulus, also called “random” vs “systematic”. Firing rate (r=0.55 p=2×10⁻¹³) and preferred angle (r=0.27 p=0.01) were significantly correlated between systematic and random stimuli movement. FIG. 42A (Row 4) is the same as FIG. 42A (Row 1), but for responses recorded across 2 days. Firing rate (r=0.28 p=3.2×10⁻⁵) and preferred angle (r=0.22 p=0.006) were correlated. FIG. 42B is similar to FIG. 41, the population remapping indices were computed based on sparsity difference, preferred angle difference and peak value of cross correlation. The sparsity difference did not show a systematic pattern but the other two metrics showed increasing remapping going from pattern (correlation=0.69, angle difference=49.1°) to color (correlation=0.64, angle difference=63.3°) to predictability (correlation=0.60, angle difference=68°) and across days (angle difference=77.9°). FIG. 42C shows the percentage of tuned responses in the random stimulus experiments, showing, comparable bi-directionality (10% here vs 13% for systematically moving bar). FIG. 42D shows that for same cells recorded in random and systematic stimulus experiments, the distributions of firing rates and SAC, quantified by zscored sparsity, were not significantly different (cyan-systematic, purple-random, KS-test for z-scored sparsity p=0.14, for firing rate p=0.27). FIG. 42E shows cross correlation between CCW and CW tuning curves showing lagged response for the majority of bidirectional cells in the random condition. FIG. 42F is the same as FIG. 42E, but for unidirectional cells. FIG. 42G shows the cross correlation of tuning curves (for tuned cells in the random stimulus experiment) between fast and slow moving stimulus was calculated from the subsample of data for a particular speed in CW and CCW direction separately and stacked together after flipping the CCW data, and was not significantly biased from zero (Circular median test at 0°, p=0.56). FIG. 42H shows an example cell with similar SAC for data within 1 second of stimulus direction change (left), or an equivalent, late subsample (right). FIG. 42I shows the firing rate (KS-test p=0.73) and sparsity (KS-test p=0.87) were not significantly different for these two subsamples of experimental recordings. FIG. 42J shows that in the randomly moving stimulus experiments, a stimulus speed modulation index was computed (see methods) and that this distribution was not significantly biased away from zero. FIG. 42K shows that the modulation index was z-scored (see methods), and only 5.2% of cells had significant firing rate modulation beyond z of ±2.

FIGS. 43A and 43B show comparable retrospective coding in systematic and randomly revolving bar experiments. FIG. 43A shows cross-correlations between CCW and CW tuning curves were averaged across all the bidirectional cells (green curves) for the systematic (latency for peak=25.7°) and random (16.7°) condition and showed a similar pattern of retrospective coding (two sample KS-Test to ascertain if the distribution of latencies was significantly different, p=0.75). Unidirectional cells showed similar pattern for systematic (19.7°) and random (31.8°) conditions, but correlations were weaker than bidirectional cells. FIG. 43B shows the cumulative distributions under systematic and random conditions comparable number of cells had positive latency (80% each) for bidirectional cells, and (67% and 68%) unidirectional cells respectively.

FIGS. 44A-44J show that SAC cells to are place cells and stimulus distance encoding cells. FIG. 44A shows two cells recorded on the same day having significant SAC in the revolving bar of experiment, and FIG. 44B shows the spatial selectivity during free foraging in two-dimensional maze. Top panel shows the position of the rat (grey dots) when the spikes occurred from that neuron (red dots). Bottom panel shows the firing probability or rate at each position. FIG. 44C shows the strength of SAC and spatial selectivity measured by z-scored sparsity were significantly correlated (r=+0.22, p=0.014). FIG. 44D is a schematic of the stimulus distance experiment. The same green stripped bar moved between −225 cm to +675 cm in 10 seconds, towards and away from the rat at a fixed angle (0°). FIG. 44E shows Raster plots and firing rates of a bidirectional cells with significant tuning to the approaching (pink, top) as well as receding (dark blue, bottom) movement of the bar of light. Trial number (y-axis on the left) and firing rates (y-axis on the right). FIG. 44F is the same as FIG. 44E, but for an unidirectional cell, tuned for stimulus distance only during the approaching stimulus movement. FIG. 44G is a pie chart depicting fraction of cell tuned (bidirectional and unidirectional) as well as untuned but stable, similar to FIG. 31. FIG. 44H shows the stimulus distance tuning is higher during approaching epochs, even after down sampling spike trains to have same firing rate (t-test actual p=4.6×10⁴, shuffle p=0.7). FIG. 44I shows that for same cells recorded in angular and linear stimulus movement experiments, tuning was positively correlated (r=0.36 p=5×10⁻⁴). FIG. 44J shows population vector overlap computed using all cells, between responses in approaching and receding stimulus movement shows retrospective response, with maxima at values above the diagonal, similar to FIG. 38H.

FIGS. 45A-45I show the relationship between SAC cells place cells and stimulus distance tuned cells. FIG. 45A shows the mean firing rates of cells was significantly correlated (r=0.43 p=4.5×10⁻¹⁰) between the SAC and spatial exploration experiments. FIG. 45B shows that a majority of cells active during the SAC experiments were also active during random foraging in real world. FIG. 45C shows that almost all of the SAC cells were also spatially selective during spatial exploration. FIG. 45D shows that between the approaching and receding directions, the mean firing rates, computed when the rats were immobile, were highly correlated (r=0.96 p=4×10⁻⁸¹) and not significantly different (t-test p=0.93). FIG. 45E shows the firing rates, computed when rats were stationary, during the stimulus angle and stimulus distance experiments were significantly correlated (r=0.22 p=0.008). FIG. 45F shows population vector decoding of the stimulus distance (similar to stimulus angle decoding, FIG. 31), was significantly better than chance. (KS-test p=5.5×10⁻¹⁰ for approaching and p=4.7×10⁻⁹ for receding data). Approaching stimulus decoding error (mean=204 cm) was significantly lesser than that for receding (mean=231 cm) (KS-test p=4.2×10⁻⁵). These errors were 63% and 74% of the error expected from shuffled data, which was greater than that for SAC decoding, where the error was 33% of the shuffles, when controlling for the number of cells. FIG. 45G shows that more than twice as many cells were unidirectional tuned for approaching (coming closer) movement direction, as compared to receding (moving away). FIG. 45H shows that for bidirectional cells, location of peak firing in the approaching and receding direction shows bimodal response, with most cells preferring either the locations close to the rat, i.e., 0 cm or far away, ˜500 cm. Unidirectional cells preferred locations close to the rat. FIG. 45I shows a population vector overlap, (FIG. 44H), was further quantified by comparing the values along the diagonal for actual tuning curves, with the spike train shuffles. The actual overlap was significantly above two standard deviations of the shuffles for distances close to the rat (around 0) and far away (beyond 400 cm).

FIGS. 46A-46D show that rewards and reward related licking are uncorrelated with SAC. FIG. 46A shows example cells showing SAC from FIG. 26, with reward times overlaid (black dots), showing random reward dispensing at all stimulus angles. FIG. 46B shows the average rate of rewards was uncorrelated with visual stimulus angle (circular test for uniformity p=0.99). FIG. 46C shows that rat consumption of rewards, estimated by the reward tube lick rate, was measured by an infrared detector attached to the reward tube¹⁸. As expected, lick rate increased after reward delivery by ˜4 fold and remained high for about five seconds (green shaded area). This duration is termed the “reward zone”. FIG. 46D shows that lick rate inside the reward zone (green) was significantly larger than that outside (red, KS-test p=2.3×10⁻⁵⁴). Inside as well as outside reward-zone lick rates were uncorrelated with visual stimulus angle (circular test for uniformity p=0.99 for both).

FIGS. 47A-47G show behavioral controls of SAC: To ascertain whether systematic changes in behavior caused SAC, a ‘behavioral clamp’ approach was used and tuning strength estimated using only the subset of data where the hypothesized behavioral variable was held constant. FIG. 47A shows example SAC tuned cells maintained tuning even if used only the data used was when the rat was stationary (running speed<5 cm/sec, blue, left). FIG. 47B shows that this was comparable to a random subsample of behavior, obtained by shuffling the indices of spikes and behavior when the animal was stationary (orange, middle) (see methods). 38% of cells were SAC tuned (sparsity z>2) when using only the stationary data which is substantially greater than chance, whereas 42% were significantly tuned in the equivalent, random subsample and this difference was significant (KS-test p=0.02). FIG. 47C is similar to FIG. 47B, but using only the data when the rat's head was immobile (head movement velocity<10 mm/sec). 43% and 42% of cells were significant tuned in actual behavioral clamp and equivalent subsample, and these were not significantly different (KS-test p=0.93). FIG. 47D is similar to FIG. 47B, but using only the data beyond 5 seconds after reward dispensing, called non-reward. 43% SAC were tuned for non-reward, 43% for equivalent subsample, KS-test p=0.56. FIG. 47E shows that using a subsample of data, from when the rat's head was within the central 20 percentile of head positions (typically <10°), rat was stationary and there were no rewards in the last 5 seconds. This condition was called “analytical head fixation.” 28% of cells were SAC tuned under this behavioral clamp, which was lesser than that in an equivalent subsample (31%, KS-test p=0.05). FIG. 47F shows tuning curves for head positions to the leftmost 20 percentile and rightmost 20 percentile were correlated (circular correlation r=0.67 p=1.3×10⁻¹¹, with 31% and 32% cells tuned in the two conditions (KS-test p=0.67). The preferred angles of tuning were highly correlated and did not have significantly different concentration (circular t-test p=0.86). FIG. 47G shows SAC tuning was recomputed in the head centric frame by accounting for the rat's head movements (obtained by tracking overhead LEDs attached to the cranial implant) and obtaining a relative stimulus angle, with respect to the body centric head angle. Overall tuning levels were comparable, between allocentric and this head centric estimation. First panel of FIG. 47G is the same as that in FIG. 47A since all SAC tuning reported earlier was in the allocentric or body centric frame. Using a subset of data when both overhead LEDs were reliably detected, 25% and 26% of cells were significantly tuned for the stimulus angle in the allocentric and egocentric frames (KS-test p=0.9). Preferred angle of SAC tuning for tuned cells was highly correlated (r=0.81 p=1.8×10⁻¹⁵) and not significantly different between the two frames (circular t-test p=0.82).

FIGS. 48A-48H show GLM estimate of SAC tuning. To estimate the independent contribution of stimulus angle to neural activity, while factoring out the contribution of head position and running speed, the generalized linear model (GLM) technique was used (see methods). FIG. 48A shows tuning curves obtained by binning methods were comparable with those from GLM estimation, for the same cells as used in FIG. 26. FIG. 48B shows sparsity levels were comparable (test p=0.07) and 40% of cells were found to be significantly tuned for stimulus angle using GLM based estimated, compared to 43% from binning in this subset of data (n=991). FIG. 48C shows that the preferred angle of firing between GLM and binning based estimates of SAC were highly correlated (circular correlation test r=0.86 p<10⁻¹⁵⁰). FIG. 48D shows the correlation between the SAC tuning curves from the two methods was significantly greater than that expected by chance, computed by randomly shuffling the pairing of cell ID across binning and GLM (KS-test p<10⁻¹⁵⁰). FIGS. 48E-48H show properties of SAC tuning responses based on GLM estimates were similar to those based on binning method, as shown in FIG. 26. FIG. 48E shows distribution of tuned cells as a function of the preferred angle (angle of maximal firing). There were more tuned cells at forward angles than behind. FIG. 48F shows median z-scored sparsity and its variability (SEM, shaded area, here and subsequently) of tuned cells as a function of their preferred angle. (Correlation coefficient r=−0.17 p=0.004). FIG. 48G shows median value of the full width at quarter maxima across the ensemble of tuned responses increased as a function of preferred angle of tuning. (r=+0.33 p<10⁻¹⁵⁰). FIG. 48H shows CDF of firing rate modulation index within versus outside the preferred zone (see methods) for tuned cells were significantly different (Two-sample KS test p=2.9×10⁻³⁷).

FIGS. 49A-49D show good performance but impaired spatial selectivity in a virtual navigation task (VNT). FIG. 49A shows an overhead schematic of virtual environment (left) and individual trial paths (thin lines) and mean paths (thick lines), colour-coded by start position (right). The white circle indicates the hidden reward zone. Scale bar, 50 cm. FIG. 49B shows a spike plot (grey, paths; red dots, spikes) and spatial rate map for a unit from the session in a, exhibiting low spatial selectivity (s, spatial sparsity). Firing rate spans 0 Hz to indicated value. FIG. 49C is the same as FIG. 49B but for a unit of higher spatial sparsity with fields near the start position of each trial. FIG. 49D shows the spatial sparsity in the VNT (0.34 (0.32, 0.36), n=384 units (median (95% confidence interval)) was slightly but significantly greater than in a two-dimensional random foraging task in the same virtual-reality system (VRF) (0.26 (0.24, 0.28), n=421 units, P=1.5×10⁻¹², two-sided Wilcoxon rank-sum test) and significantly less than in a real-world random foraging task (RWF) (0.7 (0.69, 0.71), n=626 units, P=1.2×10⁻¹⁰⁶, two-sided Wilcoxon rank-sum test) (left). FIG. 49D also shows group differences between VNT and RWF were significant after controlling for the number of spikes (P=4.7×10-6, two-way ANOVA; Methods) but not between VNT and VRF (P=0.39, two-way ANOVA) (right).

FIGS. 50A-50D shows that rats use a place navigation strategy to solve the task. FIG. 50A shows that performance, measured by rewards/meter, consistently improved across subsequent sessions in different session blocks (p=0.02, two-sided Wilcoxon sign-rank test on difference between % Improvement across consecutive days without a gap, n=27 differences). Thin gray lines indicate individual session blocks, with the thick black line indicating the mean (n=12 session blocks). FIG. 50B shows individual trials (thin colored lines) and mean path from each start position (thick black lines) for a single behavioral session with 4 start positions (top, left). Paths are color coded based on start position. All mean paths rotated to begin at the same point and heading, illustrating that rats take unique paths from each start position (right). Paths are color coded to match the colors in the left panel. The bottom is same as top but for a different behavioral session with 8 start positions. FIG. 50C shows the path correlation (see Methods) was significantly smaller (p=1.9×10⁻⁷, one-sided Wilcoxon sign-rank test) across start positions (0.58, [0.53, 0.63]) compared to within start positions (0.81, [0.77, 0.84], n=34 sessions for all statistics). Values are reported as median and 95% confidence interval of the median here and in FIG. 50D. FIG. 50D shows, as in FIG. 50C, the across start position correlation was smaller than the within start position correlation for each individual rat in the study. Rat 1: Across (0.58, [0.51, 0.68]) vs Within (0.88, [0.80, 0.89]), p=2.4×10⁻⁴, n=12 sessions). Rat 2: Across (0.53, [0.44, 0.63]) vs Within (0.82, [0.74, 0.85]), p=2.4×10⁻⁴, n=12 sessions). Rat 3: Across (0.56, [0.50, 0.69]) vs Within (0.76, [0.73, 0.84]), p=1.6×10⁻², n=6 sessions). Rat 4: Across (0.61, [0.47, 0.66]) vs Within (0.78, [0.73, 0.81]), p=0.06, n=4 sessions). One-sided Wilcoxon sign-rank test used throughout.

FIGS. 51A-51F show Further behavioral quantification. FIG. 51A shows the percentage of time rats spent in the goal-containing northeast (NE) quadrant (36, [33, 39]%) was significantly greater than chance (p=2.8×10⁴, two-sided Wilcoxon sign-rank test), and greater than all other quadrants (NW: 20, [19, 22]%; SE: 26, [23, 28]%; SW: 17, [16, 18]%). FIG. 51B shows that the median performance was 0.43, [0.38, 0.47] reward/meter (left); the median trial distance was 230, [210, 260] cm (middled); and the median trial time was 10, [9.5, 11] s of movement (right). FIG. 51C shows the quadrant occupancy as in FIG. 51A, split between 4-start sessions and 8-start sessions, exhibiting similar characteristics. FIG. 51D shows behavioral measures from FIG. 51B, split between 4-start sessions and 8-start sessions. No significant differences exist between the conditions in any measure. Rewards/meter: 4-start (0.46, [0.34, 0.49]), 8-start (0.40, [0.35, 0.46]), p=0.35. Trial Distance: 4-start (220, [205, 267]), 8-start (252, [217, 297]), p=0.30. Trial Time: 4-start (9.75, [8.47, 12.0]), 8-start (10.5, [9.9, 11.7]), p=0.27. FIG. 51E shows the occupancy index as a function of radial distance from the goal location (left). p=1.3×10-12, 34 sessions; one-way repeated-measures ANOVA. FIG. 51E also shows the population average, showing rats spend more time near the goal than expected by chance (right). Lines and shading indicate the median and 95% confidence interval of the median, color coded as in FIG. 51C. FIG. 51F shows speed index as a function of radial distance from the goal location (left). p=2.0×10⁻⁹, 34 sessions; one-way repeated-measures ANOVA. FIG. 51F also shows population average, showing rats run slower near the goal than expected by chance (right). Color conventions are as in FIG. 51E. n=34 sessions for all combined statistics; n=20 sessions for 4-start statistics; n=14 for 8-start statistics. Values are reported as median and 95% confidence interval of the median.

FIG. 52A-52G show an NMDAR antagonist impairs virtual navigation task performance. FIG. 52A shows trajectories from 6 rats injected with saline, on the first day in a new environment (top, black lines). The goal heading index (GHI) for each rat is indicated above. Full trajectories (bottom, green lines) during a probe trial (see Methods) immediately following the session above, demonstrating rats preferentially spent time near the learned reward site (open black circles). The large green dot indicates the starting position for the probe trial. Scale is as in FIG. 49. FIG. 52B shows trajectories from 6 rats injected with the NMDA antagonist (R)-CPPene (top, red lines) (see Methods). FIG. 52C shows trajectories (bottom, purple lines) from a probe trial immediately following the sessions in red. FIG. 52D shows that GHI is strongly positively correlated with rewards/meter (R=0.89, p=1.08×10⁻¹², two-sided t test, n=34 sessions). FIG. 52D shows that there was no significant difference (p=1, two-sided Wilcoxon sign-rank test) in rewards/meter between the saline (SAL, black, 0.19, [0.14, 0.24], n=6 rats) and CPP (red, 0.22, [0.15, 0.25], n=6 rats) conditions (top). Trial length (bottom) was not significantly different between the two conditions (p=0.41, two-sided Wilcoxon rank-sum test; SAL: 3.7 [3.3, 4.1] m, n=282 trials; CPP: 3.8, [3.1, 5.1] m, n=69 trials). FIG. 52E shows that rats traveled less distance overall in the CPP sessions (64, [15, 105] m, n=6 rats) compared to SAL sessions (260, [150, 300] m, n=6 rats; p=0.03, two-sided Wilcoxon sign-rank test) (top). Rats traveled less distance in the CPP probe trials (1.5, [0.12, 3.6] m, n=6 rats) compared to SAL probe trials (9.6, [4.9, 12] m, n=6 rats; p=0.03, two-sided Wilcoxon sign-rank test) (bottom). FIG. 52F shows that rats spent more time moving in the SAL sessions compared to CPP sessions (p=2.9×10-13, 2-way ANOVA with Saline/CPP group as a categorical variable and time (19 bins) as a continuous variable) (top). Rats spent less time moving in the CPP probe trials compared to the SAL probe trials (p=1.7×10-3, 2-way ANOVA with Saline/CPP group as a categorical variable and time (12 bins) as a continuous variable) (bottom). FIG. 52G shows that GHI was significantly greater than 0 in the SAL full session (0.11, [0.09, 0.28], n=6 rats, p=0.02, Right-tailed (one-sided) Wilcoxon sign-rank test throughout this panel) and SAL probe trials (0.08, [0.002, 0.14], n=6 rats, p=0.03), as well as the CPP full session (0.10, [0.05, 0.17], n=6 rats, p=0.02), indicating movement directed towards the reward zone. Goal heading index in the CPP probe trials was not significantly greater than 0 (−0.19, [−0.33, 0.09], n=4 rats, p=0.88), indicating equivalent time spent moving towards or away from the reward zone. 2 sessions were excluded due to insufficient movement.

FIGS. 53A and 53B show additional examples of spatial tuning in 4- and 8-start navigation tasks using the binning method. FIG. 53A shows example units as in FIGS. 49B and 49C. FIG. 54B shows example units as in a but for sessions with 8 start positions rather than 4.

FIGS. 54A-54E show differences between binning and GLM-derived maps; quantification of stability of GLM results for space, distance, and angle tuning. FIG. 54A shows 4 example units demonstrating the differences between binned (top) and GLM (bottom) maps. FIG. 54B shows sparsity of spatial, distance, and angular maps using the binning method versus the sparsity using the GLM. For allocentric space and episodic distance, but not allocentric angle, the binning method estimated larger sparsity on average than the GLM (Space: p=7.3×10⁻²⁹; Distance: p=7.4×10⁻³; Angle: p=0.06; n=384 units, two-sided Wilcoxon sign-rank test for all). FIG. 54C shows example rate maps for two units from the first (top row) and second (middle row) halves of a session (top). The stability of tuned spatial rate maps (0.25, [0.14, 0.40], n=111 units) was significantly higher than both the stability of untuned maps (0.14, [0.08, 0.23], n=273 units; p=0.02, two-sided Wilcoxon rank-sum test here and throughout the figure) and the stability expected from random shuffles of first and second half maps (−0.00, [−0.06, 0.08], n=384 units; p=3.7×10⁻⁷) (bottom). Untuned maps were also more stable than chance (p=5.2×10⁻⁴). FIG. 54D shows example path distance rate maps for two units from the first and second halves of a session. The stability of tuned path distance maps (0.38, [0.30, 0.44], n=181 units) was significantly higher than the stability of untuned maps (0.10, [0.03, 0.20], n=203 units; p=5.1×10⁻⁸) and of shuffled controls (−0.02, [−0.10, 0.03], n=384 units; p=1.8×10⁻¹⁹) (bottom). Untuned distance maps were also more stable than chance (p=3.2×10⁻³). FIG. 54E shows example angle rate maps for two units from the first and second halves of a session. The stability of tuned path angle maps (0.37, [0.28, 0.43], n=155 units) was significantly higher than the stability of untuned maps (0.09, [0.05, 0.17], n=229 units; p=1.9×10⁻⁸) and of shuffled controls (0.02, [−0.04, 0.05], n=384 units; p=3.4×10⁻¹⁷) (bottom). Untuned distance maps were also more stable than chance (p=1.7×10⁻³). No adjustments were made for multiple comparisons in FIGS. 54C-54E.

FIGS. 55A-55H show allocentric, path-centric and angular tuning. FIG. 55A shows spike plots and GLM-derived spatial rate maps for two units with significant spatial sparsity. p, mean rate; s, sparsity. Scale bar, 50 cm. FIG. 55B shows spike plots and GLM-derived distance rate maps (green traces, bottom) for two units with significant distance sparsity. Spikes are colour-coded according to path distance. In the bottom plots, elapsed time increases along the y axis. FIG. 55C shows spike plots and GLM-derived angular rate maps (red traces, bottom) for two units with significant angle sparsity. Spikes are colour-coded according to angle. In the bottom plots, session time increases radially outward. FIG. 55D shows percent units tuned for allocentric space (S): 29, (24, 34)%; path distance (D): 47, (42, 52)%; angle (A): 40, (35, 45)%; combinations of parameters SD: 19, (15, 23)%; SA: 18, (15, 23)%; DA; 23, (19, 28)%; and SDA: 12, (9, 15)%. n=384 total units. Numbers in the figure are unit counts, not percentages. FIG. 55E shows distribution of the spatial rate map maxima for spatially tuned cells (n=111). Density ranges from 0% to 6.1%. Scale bar, 50 cm. FIG. 55F shows distance rate maps for all path distance tuned cells (n=181), sorted by location of peak rate (median of 32, (24, 39) cm). FIG. 55G shows distribution of the angular rate map maxima for angle tuned cells (n=155). The mean vector (line) is at 70 (68, 72)⁰; maximum density is 3.0%. FIG. 55H shows the percentage of neurons tuned for space, distance and angle fluctuated as a function of path distance (Methods). Links between panels in FIGS. 55A and 55E, 55B and 55F, and 55C and 55G indicate the bottom panels are population summaries of the above panels.

FIGS. 56A-56C show that distance coding cells have similar selectivity across start positions. FIG. 56A shows spikes as a function of rat's position, for two different cells (top and bottom) are color coded based on the start position. FIG. 56B shows spikes as a function of the distance traveled, with trials from different start positions grouped together. The maps look qualitatively similar from all four start positions. The variations in firing rates could occur due to other variables, e.g., direction selectivity. FIG. 56C shows results from data from all the trials after using the GLM method. Spikes are shown as a function of the path distance and time elapsed. The GLM estimate of firing rate as a function of distance alone is shown by thick line.

FIGS. 57A-57J show examples of path distance tuning for longer distances in 4- and 8-start navigation tasks; additional properties of path distance tuning. FIG. 57A shows example units as in FIG. 55B. FIG. 57B shows example units as in FIG. 57A but for sessions with 8 start positions rather than 4. FIG. 57C shows the distance sparsity of units in 4-start sessions (0.13, [0.12, 0.14], n=183 units) was slightly but significantly greater (p=0.03, two-sided Wilcoxon rank-sum test) than the distance sparsity in 8-start sessions (0.11, [0.09, 0.13], n=181 units). FIG. 57D shows the effect in FIG. 57C was not present when controlling for the total number of spikes (p=0.43, two-way ANOVA, see Methods). FIG. 57E shows the distribution of occupancy times was skewed toward earlier distances, with a center of mass at 115 cm. FIG. 57F shows sample distance tuning curve (black) overlaid with the sum of two fitted Gaussians (green) (left). The individual Gaussians that were fitted are also shown (right). FIG. 57G shows the median goodness of fit (correlation coefficient between the original and fitted curve) was quite high (0.97, [0.96, 0.98], n=181 units), with no unit having a fit less than 0.89. FIG. 57H shows the distribution of the number of significant peaks in distance maps. 50% of units had more than one peaks, with a mean of 1.7, [1.5, 1.8] peaks. Error bars represent the 95% confidence interval of the mean obtained from a binomial distribution using the Matlab function binofit( ). FIG. 57I shows the peak index (peak amplitude of a fitted Gaussian divided by constant offset) of distance curves (2.2, [2.0, 2.4], n=300 peaks) was significantly higher (p=2.1×10⁻⁶⁹, two-sided Wilcoxon rank-sum test) than for shuffled data (0.63, [0.57, 0.68], n=463 peaks). FIG. 57J shows the width of fitted Gaussian components (width at half-max; 20, [18, 21] cm, n=300 peaks) was slightly but significantly smaller (p=0.03, two-sided Wilcoxon rank-sum test) than for shuffled data (22, [21, 23] cm, n=463 peaks).

FIGS. 58A-58F show path distance tuning is not easily explained by selectivity to time or distance to the goal. FIG. 58A shows the path distance (top row) and path time (bottom row) rate maps for three sample cells. sd and st represent the sparsity of rate maps for distance and time, respectively. Column 1 depicts a cell that is well-tuned in both the distance and time domains. Column 2 shows a cell that is better tuned in the distance domain. Column 3 shows a cell that is better tuned in the time domain. FIG. 58B shows rate maps in FIG. 58A are overlaid in the bottom row for ease of comparison. Distance between 0 and 200 cm and time between 0 and 10 s are normalized from 0 to 1 for visualization. FIG. 58C show sparsity of Path Time maps versus sparsity of Path Distance maps (left). Sparsity index (defined as (sd−st)/(sd+st)) was slightly but significantly greater than 0 (0.03, [0.02 0.05], n=384 cells; p=1.5×10-6, two-sided Wilcoxon sign-rank test) (right). FIG. 57D shows path distance (top row) and goal distance (bottom row) rate maps for three sample cells. sd and sg represent the sparsity of rate maps for path distance and goal distance, respectively. Column 1 depicts a cell that is well-tuned in both the frames of reference. Column 2 shows a cell that is better tuned in the path distance frame. Column 3 shows a cell that is better tuned in the goal distance domain. FIG. 57E shows rate maps in d are overlaid in the bottom row for ease of comparison. Distance between 0 and 200 cm and time between −200 and 0 cm are normalized from 0 to 1 for visualization. FIG. 58F shows sparsity of Goal Distance maps versus sparsity of Path Distance maps (left). Sparsity index (defined as (sd−sg)/(sd+sg)) was significantly greater than 0 (0.27, [0.23 0.31], n=384 cells; p=7.8×10⁻³⁷, two-sided Wilcoxon sign-rank test) (right)

FIGS. 59A-59J show examples of angular tuning in 4- and 8-start navigation tasks; additional properties of angular tuning. FIG. 59A shows example units as in FIG. 55C. FIG. 59B shows example units as in a but for sessions with 8 start positions rather than 4. FIG. 59C shows the angular sparsity of units in 4-start sessions (0.10, [0.09, 0.12], n=155 units) was not significantly different (p=0.77, two-sided Wilcoxon rank-sum test) than the angular sparsity in 8-start sessions (0.11, [0.09, 0.12], n=155 units). FIG. 59D shows that there was no significant difference when controlling for the total number of spikes (p=0.06, two-way ANOVA, see Methods). FIG. 59E shows that the distribution of occupancy times was skewed toward the north-east direction, with a mean vector pointing towards 56°. FIG. 59F shows sample angle tuning curve (black) overlaid with the sum of four fitted Von Mises curves (red) (left). The individual Von Mises curves that were fitted are also shown (right). FIG. 59G shows the median goodness of fit (correlation coefficient between the original and fitted curve) was quite high (0.98, [0.97, 0.98], n=155 units). FIG. 59H shows distribution of the number of significant peaks in angle maps. 83% of units had more than one peak, with a mean of 2.7, [2.5, 2.8] peaks. Error bars represent the 95% confidence interval of the mean obtained from the Matlab function binofit( ). FIG. 59I shows the peak index (peak amplitude of a fitted Von Mises curve divided by constant offset; 1.8, [1.7, 2.0], n=411 peaks) was significantly higher (p=2.1×10⁻³⁵, two-sided Wilcoxon rank-sum test) than for shuffled data (0.77 [0.72, 0.84], n=476 peaks). j, The width of fitted Von Mises curves (width at half-max; 47, [46, 49]°, n=411 peaks) was not significantly different (p=0.70, two-sided Wilcoxon rank-sum test) than for shuffled data (45, [44, 48]°, n=476 peaks).

FIGS. 60A-60C show episodic relationship between space, distance, and angle selectivity. FIG. 60A shows sparsity for rate maps in allocentric space (left), path distance (middle), and allocentric angle (right) versus the center distance coordinate (see Methods) for each cell. Significantly tuned cells are marked with large, colored dots and cells that are not significantly tuned are marked with small, black dots. FIG. 60B shows the percentage of cells significantly tuned as a function of their center distance coordinate for space (blue), distance (green), and angle (red). The combined plot at the far right is the same as FIG. 55H. FIG. 60C shows cross-correlations between the curves in FIG. 60B, overlaid with shuffled control cross-correlations, demonstrate that the relative ordering of parameter tuning—Distance, then Space, then Angle—is greater than expected by chance. Dotted black lines indicate the median and 95% range of the cross-correlation of the curves in FIG. 60B constructed from shuffled data (see Methods). Cross-correlation peaks above this range (Left, cyan, 11.25 cm indicating Distance leads Space; Middle, magenta, −150 cm indicating Space leads Angle; Right, orange, −161.3 cm indicating Distance leads Angle) indicate statistical significance at the p<0.05 level.

FIGS. 61A-61E show additional measures of performance correlate with neural tuning; speed does not correlate with performance. FIG. 61A shows the same values as in FIG. 70C plotted as a function of Trial Latency (see Methods). Correlation values and p-values are shown above each figure. Rw and pw represent the correlation value and p-value for the weighted best fit line. For unweighted fits, p-values are from a two-sided t test for each panel, with n=34 sessions. For weighted fits, p-values are calculated through a resampling procedure (see Methods). FIG. 61B is the same as FIG. 61A, but plotted as a function of Trial Distance (see Methods). FIG. 61C is the same as FIG. 61A, but plotted as a function of within-start path correlation (FIGS. 50B, 50C, see Methods). FIG. 61D is the same as FIG. 61A, but plotted as a function of goal heading index (see Methods). FIG. 61E shows that median speen in a session, including sessions from all rats, was not significantly correlated with behavioral performance as measured by rewards/meter, Trial Latency, Trial Distance, Path Correlation, or Goal Heading Index. Correlation values and p-values are shown above each figure. p-values are from a two-sided t test for each panel, with n=34 sessions.

FIGS. 62A-62G show experience-dependent changes in behavior, neural activation, and shifts in single unit path distance tuning. FIG. 62A shows trial latency (time spent running) decreased as a function of trial number (Effect of trial number on Trial Latency: p=5.0×10⁻⁵, 34 sessions; one-way repeated-measures ANOVA). The thick line is the median, and the thin lines are the 95% confidence interval of the median. FIG. 62B shows that mean speed did not significantly change as a function of trial number (Effect of trial number on Mean Speed: p=0.44, 34 sessions; one-way repeated-measures ANOVA). The thick line is the median, and the thin lines are the 95% confidence interval of the median.

FIG. 62C shows the fraction of total cells active (rate in a 3-trial boxcar average>0.2 Hz) increased with experience (correlation coefficient R=0.94, p=2.7×10⁻²⁵, n=52 trials, two-sided t test). FIG. 62D shows that averaged across the ensemble, the firing rates were higher in later trials than early trials (FIG. 73B). This was quantified on a cell-by-cell basis by computing the rate modulation index for each cell (see Methods), which was centered at 0.11, [0.08, 0.15], and significantly greater than 0 (p=1.8×10⁻¹³, two-sided Wilcoxon sign-rank test, n=384 units). FIG. 62E shows eight example cells with distance tuning curves exhibiting shifting with experience. Curves are estimated from the GLM using data only from trials 1-26 (light green) or 27-52 (dark green). The cells in the top three rows demonstrate backwards, or anticipatory, shifting. The cells in the bottom row demonstrate forwards shifting. FIG. 62F shows cross-correlation plots, sorted by the experiential shift (distance lag of the peak in the cross-correlation) of peak correlation, for all cells significantly tuned for distance but not angle, (n=88; 91 cells met this criteria, but 3 were excluded for having insufficient spiking in one of the two halves). FIG. 62G shows the median experiential shift (−7.5, [−15, 0] cm) was significantly different from 0 (p=0.014, two-sided Wilcoxon sign-rank test).

FIGS. 63A-63D show within-session clustering and forward movement of spatial, distance, and angle maps and their relationship with psychometric curves. FIG. 63A shows distributions of peaks of allocentric spatial rate maps (left, top, blue) and spatial occupancy (left, bottom, gray) in early trials, showing dispersed, fairly uniform distributions. Distributions of spatial peaks (right, top) and spatial occupancy (right, bottom) in later trials, showing clear clustering near the goal location. FIG. 63B shows the allocentric goal distance (see Methods) significantly decreased with increasing trial number (Neurons: R=−0.63, p=1.5×10⁻³, two-sided t test, n=22 trial blocks (every other trial from trial 5 to 47) here and for all other tests in this panel; Behavior: R=−0.64, p=1.2×10⁻³, two-sided t test, n=22 trial blocks), and the sparsity of these distributions increased with trial number (Neurons: R=0.67, p=5.7×10⁻⁴, two-sided t test, n=22 trial blocks; Behavior: R=0.63, p=1.8×10⁻³, two-sided t test, n=22 trial blocks). FIG. 63C shows the center of the distribution of path-distance tuning curve peaks and behavior both moved closer to the trial beginning with increasing trial number (Neurons: R=−0.91, p=2.8×10⁻⁹, two-sided t test; Behavior: R=−0.69, p=3.9×10⁻⁴), and the sparsity of these distributions increased with trial number (Neurons: R=0.89, p=2.6×10⁻⁸; Behavior: R=0.89, p=4.4×10⁻⁸). FIG. 63D shows the angle goal distance (see Methods) for angular tuning curve peaks and behavior decreased with increasing trial number (Neurons: R=−0.86, p=3.4×10⁻⁷, two-sided t test, n=22 trial blocks (every other trial from trial 5 to 47) here and for all other tests in this panel; Behavior: R=−0.55, p=7.6×10⁻³); the sparsity of these distributions increased with trial number (Neurons: R=0.67, p=7.1×10⁻⁴; Behavior: R=0.65, p=1.0×10⁻³).

FIGS. 64A-64D show temporal relationship between neural firing properties and behavior, split into high- and low-performing sessions. FIG. 64A shows that performance increased with trial number (top). This was true when including all cells (colored dots, same data as FIG. 73A, right), cells from sessions with high performance (top 50% of sessions, gray dots, “High”), or cells from sessions with low performance (bottom 50% of sessions, black dots, “Low’). Solid lines are exponential fits to the data. The firing rate of active cells increased with trial number (middle). Cross-correlation of the population firing rate with performance, for all sessions, high-performance sessions, and low-performance sessions is also shown (middle). For all data and high-performance sessions, the lag of the peak correlation is near 0, indicating a co-evolution of performance with firing rate. In low-performance sessions, there is a distinct asymmetry, indicating that neural changes precede behavioral changes. Dotted lines indicate the 99% range of shuffled cross-correlations. The marked point is the approximate center of this asymmetry, at −5 trials, and is above the chance line, indicating statistical significance at the p<0.01 level. FIG. 64B, as in FIG. 63C, shows the center of the distribution of path-distance occupancy shifted towards the trial beginning with experience within a session (top). The effect is more pronounced for sessions with high performance. The same as above but for the distribution of path-distance tuning curve peaks is shown in the middle panel. Cross-correlation of neural and behavioral experience plots are also shown (bottom). FIG. 64C is the same as FIG. 64B but for angle goal distance. FIG. 64D is the same as FIGS. 64B and 64C but for allocentric goal distance.

FIGS. 65A-65D show population vector decoding of path distance and allocentric angle. FIG. 65A shows decoded distance versus true distance for trials 1-15 (left) and trials 16-30 (right). FIG. 65B shows path-distance population vector overlap between entire session activity and activity in trials 1-15 (left) or between trials 1-15 and trials 16-30 (right). Lines and dots mark the smoothed peak correlation on the right-hand plot. Black lines indicate predictive shifts and gray lines indicate postdictive shifts, with a mean value of 15 cm. FIG. 65C shows decoded angle versus true angle for trials 1-15 (left) and trials 16-30 (right). FIG. 65D is the same as FIG. 65B but for angle. The best decoded angles span 0-90°. Right, experiential shift in angle representation was modest and varied as a function of angle (mean−3.2°), which could be due to different turning behavior at specific angles or different turning biases across sessions.

FIGS. 66A-66C show that tuning is correlated with behaviour. FIG. 66A shows a sample session with good performance (RPM), with two examples each of rate maps for allocentric space, path distance and angle, all with relatively high degrees of tuning. Scale bar, 50 cm. FIG. 66B shows a sample session with poor performance and poorly tuned rate maps. FIG. 66C shows that performance was positively correlated with mean firing rate (left). Each point represents a single session, with the size proportional to the number of units recorded in that session (minimum, four). The solid line is the unweighted best linear fit (R=0.36, P=0.04, two-sided t-test), and the dashed line is the best linear fit weighted by the number of cells (R=0.33, P=0.04) (Methods). From left to right, performance was also positively correlated with the percentage of cells tuned for allocentric space (unweighted: Ru=0.48, P=4.4×10⁻³; weighted: Rw=0.48, P=5×10⁻⁴); the percentage of cells tuned for path distance (Ru=0.40, P=0.02; Rw=0.41, P=5.4×10⁻³); and the percentage of cells tuned for angle (Ru=0.63, P=5.8×10-5; Rw=0.56, P<1×10-4). The orange and black circles represent the example sessions in a and b, respectively. n=34 sessions throughout FIG. 66C.

FIGS. 67A-67F show increased neural clustering correlated with improved behaviour within a session. FIG. 67A shows paths from early trials of a session were less efficient than later trials of the same session (left). Scale bar, 50 cm. Across all sessions, performance increased with trial number (P=1.9×10⁻⁴, one-way repeated-measures ANOVA, n=34 sessions) (right). Thick line: mean; thin lines: 95% confidence interval. FIG. 67B shows the firing rate of active cells increased with trial number (P=1.2×10⁻³, one-way repeated-measures ANOVA, n=384 units). FIG. 67C shows distribution of distance rate map peaks (green) and occupancy distribution (black) from an early trial (trial 5) across all rats with median distances of 148 cm and 118 cm, respectively (left). Distributions of peaks and occupancy from a later trial (trial 43) across all rats, with median distances of 114 cm and 107 cm, respectively (right). FIG. 67D shows median absolute decoding error as a function of trial number (effect of trial number on median error: P=0.01, 80 bins, one-way repeated-measures ANOVA; error on trials 1-15 compared to error on trials 16-30: P=1.1×10-10, n=1,200 (trial, bin) predictions, two-sided Wilcoxon rank-sum test) (left). Dotted lines indicate the 95% confidence interval (n=80 bins). Median error as a function of path distance for trials 1-15 (dark green) and trials 16-30 (light green) (right). Interaction effect between distance bin and trial group: P=3.1×10⁻¹¹, two-way ANOVA (Methods). FIG. 67E, as in FIG. 67C but for angle rate maps. Mean vector in early trials: 63° (mean vector length (MVL)=0.05) for neurons and 46° (MVL=0.15) for behaviour. MVL trials: 52° (MVL=0.18) for neurons and 54° (MVL=0.22) for behaviour. FIG. 67F is the same as FIG. 67D but for angle. Decoding error decreases as a function of trial number (effect of trial number on median error: P=1.7×10⁻⁷, 80 bins, one-way repeated-measures ANOVA; error on trials 1-15 compared to error on trials 16-30: P=5.6×10-14, n=1,200 (trial, bin) predictions, two-sided Wilcoxon rank-sum test) but not as considerably as path distance (left). Decoding error near 45° is smallest, even in earlier trials (interaction effect between angle bin and trial group: P=0.02) (right).

FIGS. 68A and 68B show the behavioral performance of individual rats. FIG. 68A is the same as FIG. 51A but plotting the median and 95% confidence interval of the median for individual rats. FIG. 58B is the same as FIG. 51B but for individual rats. For all measures, Rat 4's statistics (purple) are significantly different from Rat 1 (p=1.1×10⁻³) but not different from Rat 2 (p=0.06) or Rat 3 (p=0.11), using two-sided Wilcoxon rank-sum test for all comparisons.

FIGS. 69A and 69B show no difference in spatial tuning between 4- and 8-start sessions using binned maps. FIG. 69A shows the spatial sparsity of units in 4-start sessions (0.33, [0.30, 0.37], n=206 units) was not significantly different (p=0.56, two-sided Wilcoxon rank-sum test) than the spatial sparsity in 8-start sessions (0.35, [0.32, 0.37], n=178 units). FIG. 69B shows that there was no difference in spatial sparsity between 4-start and 8-start positions when controlling for the total number of spikes (p=0.18, two-way ANOVA, see Methods).

FIGS. 70A-70C show GLM-derived spatial sparsity and spatial occupancy. FIG. 70A shows that for GLM-derived spatial maps, the spatial sparsity of units in 4-start sessions (0.19, [0.16, 0.21], n=206 units) was not significantly different (p=0.36, two-sided Wilcoxon rank-sum test) than the spatial sparsity in 8-start sessions (0.20, [0.16, 0.22], n=178 units). FIG. 70B shows that there was no difference in spatial sparsity between 4-start and 8-start positions when controlling for the total number of spikes (p=0.11, two-way ANOVA, see Methods). FIG. 70C shows that the distribution of spatial occupancy averaged across all sessions was clustered towards the goal location, mirroring the pattern seen in the clustering of spatial field peaks (FIG. 55E).

FIGS. 71A-71C shows that path distance centers are aggregated towards short distances independent of trial length; path distance tuning is not easily explained by turning distance. FIG. 71A (Top, column 1) shows binned distance rate maps for all cells significantly tuned for path distance, sorted by location of peak rate, using only data from trials of length 0-75 cm (see Methods for cell inclusion criteria). FIG. 71A (Bottom) shows the distribution of the peak location of rate maps above (Median of 36, [32, 41] cm, n=94 peaks). Column 2, same analysis computed using only data from trials of length 75-150 cm (Median of 54, [43, 69] cm, n=111 peaks). FIG. 71A (Column 3) also shows the same analysis computed using only data from trials of length 150-225 cm. Note there is still aggregation of field peaks near the beginning of paths (Median of 71, [54, 81] cm, n=168 peaks), even though all paths in this data covered at least 150 cm. FIG. 71A (Column 4) shows the same analysis, but for the longest trials, of length 225-300 cm. The aggregation towards the beginning of trials (Median of 77, [69, 96] cm, n=155 peaks) is quite pronounced. FIG. 71A (Column 5) shows the same analysis but including data from all trials (Median of 71, [51, 84] cm, n=142 peaks). Note that these maps are computed using the binning method, and thus differ slightly from those in FIG. 55F which are computed using the GLM. FIG. 71B shows an example calculation of turning distance D. Path Distance versus angular speed shows the repeated movement trajectory across trials (black dots). The thick line is the median angular speed as a function of path distance (computed in bins of width 3.75 cm, smoothed with a Gaussian kernel with a sigma of 3.75 cm). The distance corresponding to the peak angular speed (red star) indicates the halfway point of the turning distance, or D/2, for that session. FIG. 71C shows that for each cell significantly tuned for path distance, the location of the peak is plotted against the turning distance D for the corresponding session. There is no correlation between the two (R=0.00, p=0.96, two-sided t test, n=176 cells), indicating that path distance peaks are not solely defined by turning. The dotted black line indicates the unity line. For many cells, the path distance peak was substantially larger than the turning distance, additionally indicating that distance selectivity was not entirely determined by the act of turning. For ease of viewing, random Gaussian jitter (sigma of 2 cm) is added to the turning distance for each cell. Statistics and the red best fit line are computed on the original data with no jitter. 5 cells were excluded from the original 181 distance-tuned cells for belonging to sessions with a turning radius>150 cm.

FIGS. 72A and 72B shows that Population tuning measures and distribution of distance fields for individual rats. FIG. 72A, as in FIG. 55B for individual rats, the Venn diagrams represent the number of cells significantly tuned for Allocentric Space (S, blue), Path Distance (D, green), or Allocentric Angle (A, red). The colored numbers represent the number of cells falling into each region of the Venn diagram (Blue, Space only; Green, Distance only; Red, Angle only; Cyan, Space and Distance; Magenta, Space and Angle; Yellow, Distance and Angle; Black, Space, Distance, and Angle). FIG. 72B, as in FIG. 55F for individual rats, shows qualitatively similar distributions of distance fields.

FIGS. 73A-73D shows experience-dependent changes in performance, firing rate, and distance clustering for individual rats. FIG. 73A, as in FIG. 67A, but for individual rats, performance increases as a function of trial number (fffect of trial number on performance −Rat 1: p=0.03, 12 sessions; Rat 2: p=0.09, 12 sessions; Rat 3: p=0.11, 6 sessions; Rat 4: p=0.03, 4 sessions; one-way repeated measures ANOVA). FIG. 73B, as in FIG. 67B, for individual rats, showing qualitatively similar patterns (effect of trial number on Population Rate—Rat 1: p=4.1×10⁻³, 113 units; Rat 2: p=0.28, 129 units; Rat 3: p=0.03, 60 units; Rat 4: p=0.04, 82 units; one-way repeated measures ANOVA). FIG. 73C and FIG. 73D, as in FIG. 63C, for individual rats, show qualitatively similar patterns. Green dots represent neural measures, and black circles represent behavioral measures. Correlation coefficients for FIG. 73C—Rat 1: Neurons: R=−0.92, p=1.8×10⁻⁹, two-sided t test, n=22 trial blocks (every other trial from trial 5 to 47) here and for all other tests in FIGS. 73C and 73D; Behavior: R=−0.64, p=1.2×10⁻; Rat 2: Neurons: R=−0.88, p=9.4×10⁻⁸; Behavior: R=−0.68, p=4.9×10⁻⁴; Rat 3: Neurons: R=−0.80, p=8.6×10⁻⁶; Behavior: R=−0.67, p=7.1×10⁻⁵; Rat 4: Neurons: R=−0.93, p=3.5×10⁻¹⁰; Behavior: R=−0.72, p=1.5×10⁻⁴. Correlation coefficients for FIG. 73D—Rat 1: Neurons: R=−0.68, p=5.7×10⁴; Behavior: R=0.68, p=4.5×10⁻⁴; Rat 2: Neurons: R=0.87, p=1.1×10⁻⁷; Behavior: R=0.67, p=7.0×10⁻⁴; Rat 3: Neurons: R=0.69, p=3.5×10⁻⁴; Behavior: R=0.74, p=8.2×10⁻⁵; Rat 4: Neurons: R=0.82, p=2.5×10⁻⁶; Behavior: R=0.70, p=3.1×10⁴.

### DETAILED DESCRIPTION

The hippocampus is a seahorse-shaped part of the brain, found in the inner folds of the bottom-middle section of the brain known as the temporal lobe. In particular, the hippocampus is a ridge of gray matter tissue elevating from the floor of each lateral ventricle in the region of the inferior or temporal horn. In humans, two hippocampi are present (one in each side of the brain). The hippocampus is a part of the limbic system and plays important roles in the consolidation of information from short-term memory to long-term memory, and in spatial memory that enables navigation. The hippocampus contains two main interlocking parts: the hippocampus proper (also called Ammon's horn) and the dentate gyrus.

Various theories of hippocampal function include the involvement of the hippocampus in response inhibition, episodic memory, and spatial memory/cognition. Thus, damage to the hippocampal region of the brain has effects on overall cognitive functioning, particularly memory such as spatial memory/cognition. Moreover, when the hippocampus is impaired, patients can't develop new long-term memories. Various studies have reinforced the impact that damage to the hippocampus has on memory processing, in particular the recall function of spatial memory. Moreover, damage to the hippocampus can occur from prolonged exposure to stress hormones such as glucocorticoids (GCs), which target the hippocampus and cause disruption in explicit memory.

The hippocampus is directly involved in a wide range of diseases of the brain, including Alzheimer's disease, Autism, epilepsy, depression, PTSD, and schizophrenia. For example, in various forms of dementia, including Alzheimer's, the hippocampus may be one of the first regions of the brain to suffer damage. Patients having Alzheimer's begin to lose their short-term memories, may find it difficult to follow directions, and often get lost of can't find their way. The hippocampus also loses volume as the disease continues, and patients lose their ability to function. When diseases of the brain damage the hippocampus, short-term memory loss and/or disorientation may be early symptoms. Damage to the hippocampus can also result from other injuries including oxygen starvation (hypoxia), encephalitis, and/or medial temporal lobe epilepsy. When a person has extensive, bilateral hippocampal damage, that person may experience anterograde amnesia: the inability to form and retain new memories. Alzheimer's disease is thought to reduce the size of the hippocampus. In Alzheimer's disease, this link is so well-established that watching the volume of the hippocampus can be used to diagnose the progress of the disease. At present there are no reliable treatments to cure Alzheimer's. The only way to help the patients is early diagnosis. In various embodiments, the proposed VR/AR system can be used for both diagnosis and treatment of Alzheimer's or other forms of dementia or hippocampal malfunctions, some of which are described above.

Additionally, there is a strong link between the hippocampus and epilepsy as the hippocampus is where many epileptic seizures begin. Between 50 and 75 percent of patients with epilepsy who have autopsies had damage to the hippocampus. The hippocampus is considered by many to be the generator of temporal lobe epilepsy (TLE) due to the frequent observation of the histopathology of sclerosis in the Sommer's sector and in the endfolium of the hippocampus of TLE patients. In addition, surgical removal of the sclerotic hippocampus often improves this epileptic condition.

Lastly, the hippocampus also appears to be affected (e.g., loses volume) in cases of severe depression as depression appears to reduce the size of the hippocampus. Some studies of depression have shown that the hippocampus wastes away and shrinks by up to 20 percent.

As outlined above, the hippocampus plays a key role in learning and memory, even in adults, and in a wide range of disorders including Autism, Alzheimer's, PTSD, depression, epilepsy, concussions, and stroke.

Artificial reality and virtual reality devices may be used to drive hippocampal activity. However, commercially available VR/AR devices lack several crucial features to achieve suitable results when driving hippocampal activity of a user—specifically, regarding immersion, embodiment, walls and edges, VOR delay removal, fatigue, and memory consolidation.

Immersion: Commercially-available VR headsets do not create complete immersion for a user. For example, the images are only in front of the eyes and do not extend to the periphery on the sides. Peripheral vision is crucial for immersion. Experiments have shown that motion of an object may be detected first on the periphery before we detect the object in the front (e.g., central) vision. The capability to detect a moving predator in the periphery and act quickly has been crucial for human survival such that specialized circuits have developed through evolution to allow for this fast reaction time. As explained above, commercially-available VR headsets do not have the capability to display images in the peripheral vision of a user and, thus, lack the ability to provide stimulation in the peripheral vision of the user. In various embodiments, a VR/AR system is provided where visual stimuli may be presented in the peripheral vision of a subject. In various embodiments, visual stimuli may be provided behind the subject (e.g., outside of the field of vision). In various embodiments, virtual stimuli may be provided that are configured to activate the peripheral vision. In various embodiments, activation of the peripheral vision may be performed by providing one or more naturalistic optic flow patterns. In various embodiments, a combination of hardware (e.g., tactile) and visual stimuli may allow for complete immersion and proper activation of neural circuits. In various embodiments, the systems described herein may be used to directly test the damaging effects of misfiring of neural circuits due to missing peripheral stimuli.

Embodiment: In commercially-available VR systems, a subject cannot see their hands or feet. This may create a sense of disembodiment and anxiety, as if the user has left their body and is hovering in a room. The effect caused by wearing a commercially-available VR system may be similar to experiments in sensory deprivation chambers and these experiments can create strong anxiety in users. In various embodiments, the issues of disembodiment and anxiety can be reduced (e.g., eliminated) through a unique set of hardware, software, and images. In various embodiments, the system may include a small overhead projector. In various embodiments, the system may include one or more reflecting mirrors. In various embodiments, the one or more reflecting mirrors may be positioned such that the virtual light source appears from overhead, just as in the natural world. In contrast, the light source in commercially-available VR systems is in the front of the eyes, not overhead. The disclosed system thus ensures that the users not only see their hands and feet in VR, but that they see their entire bodies and the shadow of their bodies in the VR. In various embodiments, the disclosed VR system may allow for a unique set of visual cues that have high spatial frequency on the floor. In various embodiments, specialized neural circuits in the visual cortex of the brain may respond strongly to this high spatial frequency and activate high frequency oscillations. In various embodiments, high spatial frequency may create a strong sense of embodiment and naturalistic movement signals.

Walls and edges: Commercial VR systems generally have an infinite plane with no edges, which is unnatural. Moreover, VR scenes in these systems may include walls where the subject is artificially stuck, without any sensory feedback of a wall in the natural world, which is also unnatural. These conflicting signals may interfere with the functioning of the hippocampal system, because the hippocampus requires a consistent integration of distal visual landmarks (cognitive mapping) and self-motion cues (path integration). In various embodiments, a VR system is provided where a virtual environment may be generated with high spatial frequency stimuli on a floor of the virtual environment and a virtual ground may be generated below the environment with low spatial frequency stimuli. For example, a virtual maze may be displayed about one meter above a virtual ground generated below. The virtual maze may include high spatial frequency stimuli on the floor, whereas the virtual ground may include low spatial frequency stimuli. In various embodiments, the display of a virtual environment (with high spatial frequency stimuli) and virtual ground (with low spatial frequency stimuli) may allow for the combination of locomotion and visual cues to generate a virtual edge by motion parallax, thus eliminating the unnatural problems of display using conventional VR that are either infinite or have walls without sensory feedback.

VOR delay removal: Commercially-available VR headsets use accelerometers to measure the head movement. The data from the accelerometer is then fed into a VR engine, which calculates the amount of change in the visual scene corresponding to the amount of head movement measured by the accelerometers. However, this process suffers from two major problems. First, the accelerometers are not accurate and, thus, the measurement of the exact head movement is not accurate. Second, the above computations are very resource intensive and slow. Even the fastest VR system requires more than 20 milliseconds to compute this quantity and render the appropriate VR scene. The human brain is capable of detecting discrepancies between head movements and the movement of the world, because a discrepancy suggests that something else is moving in the world while we are scanning the world by head movement, e.g., the movement of a large predator. When the brain recognizes a discrepancy, this causes stress on the body. Prolonged use may cause dizziness, similar to and/or worse than seasickness while sailing on rough seas, due to a mismatch between the head movement and the surrounding visual scene movement. While being dizzy or sea-sick, it is very difficult to learn new information. Seasickness and/or dizziness has profound adverse effects on the brain circuits called VOR reflex and, in some cases, can cause epileptic seizures. In various embodiments, the disclosed VR system eliminates this problem entirely using several neurobiological principles. First, in various embodiments, instead of measuring the acceleration of a head, the system allows the user to move their head naturally while a VR/AR screen surrounds them. Thus, the user is able to see exactly what they should see, without any delay. Second, in various embodiments, the VR/AR is immersive so that when the user moves their head, the user will still see the same VR scene and not a blank patch and/or other artifacts. Third, in various embodiments, the disclosed VR/AR system displays visual stimuli on the walls with low spatial frequency, so that small differences in the leg movement and the VR scene update are not noticeable by the brain.

Fatigue: Commercially-available VR headsets may be mentally exhausting because the brain has to perform new calculations at every moment, far more complex calculations than in the real world. Users may complain of VR fatigue and stress, which is especially a problem for patients who suffer from neurological problems. The disclosed VRAR system has been designed in such a way that it is relaxing to use in ways that commercially-available VR systems cannot be used. For example, a user may take a nap in the disclosed VR/AR system. This activity of taking a nap is difficult to do in commercially-available VR systems because the smallest head movements during napping causes changes in the accelerometers, which change in the visual scenery that wakes up the subject. In various embodiments, the disclosed VR/AR systems do not require accelerometers, thus reducing the spatial frequency of visual cues on the wall and making the VR/AR environment very more comfortable.

Memory consolidation: Extensive research shows that the hippocampus generates specific brain waves, called sharp wave ripples, during napping. These brain waves are crucial for turning temporary memory into long-term, stable memory via a process called memory consolidation. By eliminating any change in VR/AR when the user is stationary (by eliminating accelerometers), the disclosed VR/AR system is able to ensure that users can perform certain activities (e.g., take naps) in VR such that the hippocampus may generate sharp wave ripples. The sharp wave ripples ensure that memories are learned and consolidated into long-term memories.

In various embodiments, the present disclosure provides VR/AR based therapy for treating epilepsy. Epilepsy is a major disease where the hippocampal neurons are overactive. As set out herein, VR/AR systems according to the present disclosure may be used to reduce Hippocampal activity and thereby prevent or treat epilepsy.

Pharmaceutical therapies have serious side effects and for many patients are ineffective. Alternative approaches often involve brain surgery. In contrast, VR/AR is completely noninvasive.

It will be appreciated that there are various forms of epilepsy, each of which may require a different degree of Hippocampal inhibition for effective treatment. In exemplary embodiments, VR devices according to the present disclosure cause 60% of neurons in the hippocampus to shut down. In various embodiments, systems according to the present disclosure are optimized interactively with a given patient in order to treat their given form of epilepsy. In various embodiments, a learning system is used to determine the VR/AR parameters.

In various embodiments, patient data collected from a VR/AR device and/or sensors may be stored in a datastore. In various embodiments, data are provided from sensors, AR or VR device, and/or the datastore to a machine learning system. In various embodiments, data may be provided to the learning system in real time. In various embodiments, by receiving data live from the user, learning system provides high level analysis that provides adjustment and adaptation of a VR/AR environment, through changes in the various parameters according to the recorded data.

In some embodiments, a feature vector is provided to the learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

In various embodiments, the present disclosure provides VR/AR based systems and methods for manipulating brain rhythms, thereby treating neurological disorders and/or improving learning and memory.

Brain rhythms are known to be crucial for learning. The theta rhythm in the hippocampus is known to be crucial for learning. Loss of theta rhythm results in loss of learning and memory. A treatment for Alzheimer's disease may target theta rhythm. However, there has not previously been any reliable way to increase the amplitude or rhythmicity of theta oscillations. Use of VR/AR based systems according to the present disclosure for even a short time dramatically enhances theta rhythm.

Each patient has slightly different theta rhythm. Even a small difference in theta rhythm can have a significant impact on learning. Accordingly, systems and methods provided herein may be employed to adjust (e.g. retune) brain rhythms (including theta rhythm) in a patient-specific fashion to treat memory problems. In various embodiments, systems according to the present disclosure are optimized interactively with a given patient in order to enhance theta rhythm. In various embodiments, a learning system is used to determine the VR/AR parameters, for example by monitoring a user via EEG.

In various embodiments, the present disclosure provides VR/AR based systems and methods for increasing neuroplasticity and for diagnosing neuroplasticity disorders.

A major reason for learning and memory disorders is loss of neuroplasticity. One test of Neuroplasticity in memory is the Morris Water Maze task, used by pharmaceutical companies for testing drugs that target neuroplasticity. However, many drugs that work in mice in the water maze do not work for humans. Further, there is no reliable way to boost neuroplasticity in specific brain regions, e.g., the hippocampus, in a noninvasive fashion, without adverse side effects. Data using the systems described herein show that neuroplasticity is substantially boosted in VR/AR (see attached manuscripts). Thus, VR/AR can be used for boosting neuroplasticity on demand in specific brain regions, without evident side effects.

When the mice are swimming in the water maze, to escape drowning, they are using an entirely different memory system (based in the amygdala, the fear center) than a patient that sits in a doctor's office or at home. Recollection of the name of a loved one, for example, is a pleasant memory that are is controlled by different brain structures. Entirely different brain regions are involved in swimming (motor cortex) and fear (amygdala) versus happy recollection of past events (hippocampus). This difference helps explain why pharmaceuticals that work in the context of fearful memories in mice do not work for happy memories in humans.

Accordingly, the present disclosure provides for virtual reality learning tests that are more analogous to the happy recollection tests applied in the human context. Rats may be unstressed as they explore a VR environment to obtain sugared water. They can terminate the task exactly when they want. The same virtual reality used for rats can be used for human patients. Therefore, the VR/AR devices described here can be used for early diagnosis of memory impairments and hippocampal malfunction in patients as well as laboratory animals, thereby greatly enhancing the success of therapies tested in rodents to work in humans.

As set out herein, the effect of virtual reality experience on plasticity and memory formation. Neuroplasticity signals are detected in the hippocampus, which are directly related to behavioral performance. Accordingly, VR testing provides a reliable tool for diagnosing neuroplasticity disorders. By applying a substantially similar test to a mouse and a human, the failure rate of a pharmaceutical may be minimized when transitioning from mouse to human testing.

Augment reality (AR) and virtual reality (VR) typically reproduce real world environments where users perform tasks in a way similar to real world experiences. AR/VR experiences allow users to climb virtual mountains, play virtual sports games, jump out of an airplane, shoot targets, and engage in other physically demanding real-world behavior.

It will be appreciated that a variety of virtual and augmented reality devices are known in the art. For example, various head-mounted displays providing either immersive video or video overlay are provided by various vendors. Some such devices integrate a smart phone, the smart phone providing computing and wireless communication resources for each virtual or augmented reality application. Some such devices connect via wired or wireless connection to an external computing node such as a personal computer. Yet other devices may include an integrated computing node, providing some or all of the computing and connectivity required for a given application.

Virtual or augmented reality displays may be coupled with a variety of motion sensors in order to track a user's motion within a virtual environment. Such motion tracking may be used to navigate within a virtual environment, to manipulate a user's avatar in the virtual environment, or to interact with other objects in the virtual environment. In some devices that integrate a smartphone, head tracking may be provided by sensors integrated in the smartphone, such as an orientation sensor, gyroscope, accelerometer, or geomagnetic field sensor. Sensors may be integrated in a headset, or may be held by a user, or attached to various body parts to provide detailed information on user positioning.

In various embodiments, a mobile phone may be attached to the body of a user to thereby record motion data using components such as, for example, an internal gyroscope, internal accelerometer, etc.

It will also be appreciated that various embodiments are applicable to virtual and augmented reality environments in general, including those that are presented without a headset. For example, a magic window implementation of VR or AR uses the display on a handheld device such as a phone as a window into a virtual space. By moving the handheld device, by swiping, or by otherwise interacting with the handheld device, the user shifts the field of view of the screen within the virtual environment. A center of a user's field of view can be determined based on the orientation of the virtual window within the virtual space without the need for eye-tracking. However, in devices including eye-tracking, more precision may be obtained.

Because VR/AR technology can provide detailed data about position and motion for a user via various sensors in a head-mounted display and/or at other body parts, a VR/AR system may provide a broad understanding of user behavior. In various embodiments, data recorded by the VR/AR system may include positional and/or motion data for a head mounted display, positional and/or motion data for one or more handheld sensors, positional and/or motion data for a torso sensor, and positional and/or motion data for one or more foot-mounted sensors or leg mounted sensors. In various embodiments, data recorded by the VR/AR system may include what was in the field of view of the user, whether the user began an action, whether the user stopped before completing the action, etc.

In various embodiments, the VR/AR system may determine the position of one or more body part (e.g., hand, foot, head, etc.) and/or record the position over time. In various embodiments, one or more sensors may be attached to or otherwise associated with a body part to track a three-dimensional position and motion of the body part with up to six degrees of freedom, as described above. In various embodiments, the VR/AR system may determine a plurality of positions of one or more body parts. In various embodiments, the plurality of positions may correspond to points along a three-dimensional path taken by the sensor associated with (e.g., attached to) the body part.

In various embodiments, the VR/AR system may track the position and/or motion of the head. In various embodiments, the system may utilize sensors in a head-mounted display to determine the position and motion of the head with six degrees of freedom as described above. In various embodiments, for more nuanced motions, one or more additional sensors may provide position/motion data of various body parts.

In various embodiments, positional data may be recorded with infrared sensors. In various embodiments, a gyroscope and/or accelerometer may be used to record positional information of a user and/or forces experienced by the user, either separately or concurrently with other sensors, such as the infrared sensors. In various embodiments, the gyroscope and/or accelerometer may be housed within a mobile electronic device, such as, for example, a mobile phone that may be attached to the user.

In various embodiments sensors are provided that track various attributes of a user while performing an activity in a virtual environment. Such sensors can include, but is not limited to, Heart rate variability (HRV), Electrothermal activity (EDA), Galvanic skin response (GSR), Electroencephalography (EEG), Electromyography (EMG), Eye tracking, Electrooculography (EOG), Patient's range of motion (ROM), Patient's velocity performance, Patient's acceleration performance, and Patient's smoothness performance.

In various embodiments, additional sensors are included to measure characteristics of a subject in addition to motion. For example, cameras and microphones may be included to track speech, eye movement, blinking rate, breathing rate, and facial features. Biometric sensors may be included to measure features such as heart rate (pulse), inhalation and/or exhalation volume, perspiration, eye blinking rate, electrical activity of muscles, electrical activity of the brain or other parts of the central and/or peripheral nervous system, blood pressure, glucose, temperature, galvanic skin response, or any other suitable biometric measurement as is known in the art.

In various embodiments, an electrocardiogram (EKG) may be used to measure heart rate. In various embodiments, an optical sensor may be used to measure heart rate, for example, in a commercially-available wearable heart rate monitor device. In various embodiments, a wearable device may be used to measure blood pressure separately from or in addition to heart rate. In various embodiments, a spirometer may be used to measure inhalation and/or exhalation volume. In various embodiments, a humidity sensor may be used to measure perspiration. In various embodiments, a camera system may be used to measure the blinking rate of one or both eyes. In various embodiments, a camera system may be used to measure pupil dilation. In various embodiments, an electromyogram (EMG) may be used to measure electrical activity of one or more muscles. The EMG may use one or more electrodes to measure electrical signals of the one or more muscles. In various embodiments, an electroencephalogram (EEG) may be used to measure electrical activity of the brain. The EEG may use one or more electrodes to measure electrical signals of the brain. Any of the exemplary devices listed above may be connected (via wired or wireless connection) to the VR/AR systems described herein to thereby provide biometric data/measurements for analysis. In various embodiments, breathing rate may be measured using a microphone.

In various embodiments, a VR system is provided for delivering a VR experience to a user using a treadmill (e.g., omnidirectional) and one or more projectors within a chamber. In various embodiments, the VR systems described herein may be applied to humans and animals (e.g., mammals) alike. For example, the system may be constructed such that a human subject fits within the chamber and the treadmill supports the weight of the human subject. In another example, the system may be scaled down for testing with a rodent model such that a rodent (e.g., rat, mouse) fits within the chamber and the treadmill supports the weight of the rodent.

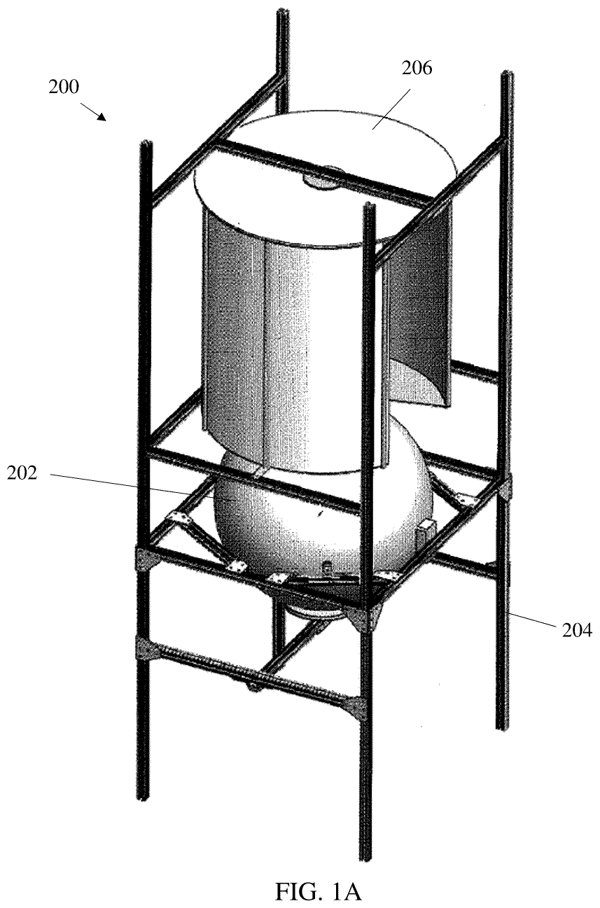

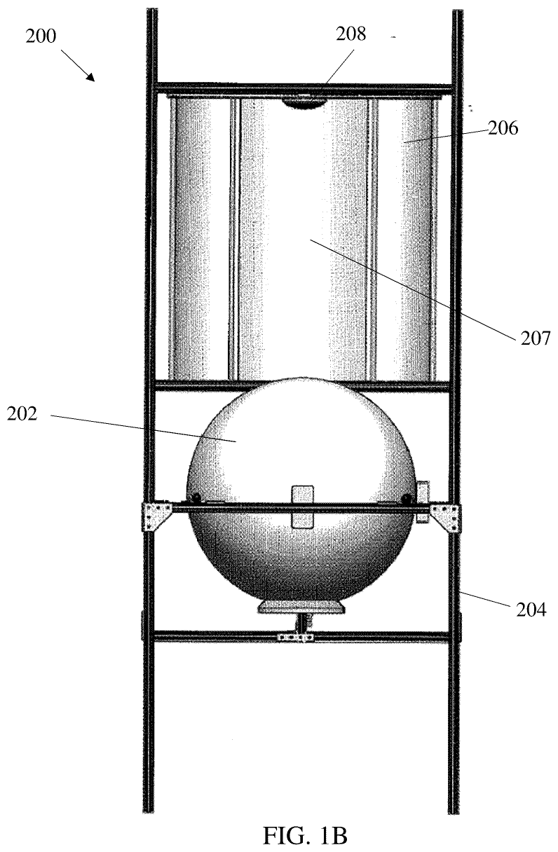

FIG. 1A illustrates a perspective view of an exemplary VR system 200 according to embodiments of the present disclosure. FIG. 1B illustrates a front view of the exemplary VR system 200 according to embodiments of the present disclosure. As shown in FIGS. 1A-1B, the VR system 200 includes a treadmill 202 coupled to a frame 204. In various embodiments, the treadmill 202 is an omnidirectional treadmill. In various embodiments, the treadmill 202 includes an outer housing, an inner sphere within the outer housing, and a fluid disposed between the inner sphere and the outer housing. In various embodiments, the outer housing may cover a portion of the surface area of the inner sphere such that a user may walk on an exposed portion of the inner sphere. In various embodiments, the outer housing may cover up to (and including) 99% of the surface area of the inner sphere. In various embodiments, the user interacts with the remaining, exposed portion of the inner sphere, for example, by directly contacting the inner sphere while walking in a particular direction. As the user walks on the inner sphere, the user may remain stationary while experiencing the sensation a natural walking experience. In various embodiments, the treadmill 202 may be configured to be acoustically quiet so as not to cause acoustic stress on the subject using the VR system 200.

In various embodiments, the inner sphere may include a metal (e.g., aluminum, steel, stainless steel, etc.). In various embodiments, the inner sphere may include a polymer (e.g., polyethylene, polyurethane, polyethylene terephthalate, polycarbonate, polystyrene, poly(methyl methacrylate), polytetrafluoroethylene, etc.). In various embodiments, the outer housing may include a metal (e.g., aluminum, steel, stainless steel, etc.). In various embodiments, the outer housing may include a polymer (e.g., polyethylene, polyurethane, polyethylene terephthalate, polycarbonate, polystyrene, poly(methyl methacrylate), polytetrafluoroethylene, etc.). In various embodiments, the material of the inner sphere and/or the outer housing may be a low-friction material configured to minimize friction between the surfaces of the inner sphere and outer housing as the inner sphere rotates within the outer housing. In various embodiments, an inflatable cushion may be provided between the inner sphere and the outer housing.

In various embodiments, the treadmill 202 may have any suitable size for the particular subject for which the VR system 200 will be used. For example, for a rodent model, the treadmill 202 may be 4 mm to 10 mm in diameter.

In various embodiments, the fluid may be air (e.g., at standard temperature and pressure). In various embodiments, the fluid may be a compressed gas (e.g., compressed air). In various embodiments, the fluid may be a liquid (e.g., water). In various embodiments, the fluid may be supplied via a tube 210.

In various embodiments, the treadmill 202 includes one or more sensor(s) for determining the motion of the treadmill 202 as the user operates (e.g., walks on) the treadmill 202. In various embodiments, the one or more sensors include one or more laser CMOS sensor(s). In various embodiments, two laser CMOS sensors are used to track three rotational axes of the treadmill 202.

In various embodiments, the treadmill 202 operates in a linear direction, allowing a user to move only in a particular direction or the reverse direction while remaining stationary relative to the VR system 200.

As shown in FIGS. 1A-1B, the VR system 200 further includes a VR chamber 206 coupled to the frame 204 and disposed above the treadmill 202. In various embodiments, the treadmill 202 extends into the bottom of the VR chamber 206 such that a user may interact with the treadmill 202 while inside the VR chamber 206. In various embodiments, an inner surface 207 of the VR chamber 200 may be a display configured to display a VR environment to a user. In various embodiments, the inner surface 207 of the VR chamber 200 may be configured to receive a projection. In various embodiments, the inner surface 207 may include a screen material.

In various embodiments, the VR chamber 206 includes one or more projector 208 (e.g., a picoprojector) configured to project a VR environment on the inner surface 207 of the VR chamber 206. In various embodiments, the VR chamber 206 includes one or more mirrors 212 configured to reflect the projected image(s) from the one or more projector 208 onto the inner surface 207 of the VR chamber 206. In various embodiments, the VR chamber 206 includes one or more speakers for transmitting sound. In various embodiments, the VR chamber 206 includes a reward delivery system configured to controllably deliver a reward to the subject (e.g., a rodent).

In various embodiments, the one or more mirror 212 has a curved surface. In various embodiments, the one or more mirror 212 is polished using special software such that the image that is formed on the inner surface 207 of the chamber 206 (all around the user) is undistorted. In various embodiments, the inner surface 207 around the user (on which the projected image falls) is made of thin light reflective material. In various embodiments, the material is sound insulating to thereby muffle any echo of the user's footsteps. In various embodiments, the one or more mirror 212 have surface curvatures according to Snell's law to thereby project a suitable image onto the inner surface 207 of the VR chamber 206.

In various embodiments, an exemplary implementation of the VR system 200 includes positioning an animal (e.g., a rodent) within the VR chamber 206 and on top of the exposed portion of the inner sphere of the treadmill 202. In various embodiments, the animal 220 may be secured in place, for example, by head fixation and/or a body harness. In various embodiments, a VR environment may be presented to the animal 220 via one or more projectors and one or more speakers within the VR chamber 206. As the animal 220 walks in place, the treadmill 202 rotates in the intended direction of the animal 220 and the rotation of the inner sphere of the treadmill 202 is recorded by laser sensors. The recorded motion data is transmitted in real time to a computer, which updates the perceived visual and auditory environment (provided by the projector and/or speakers inside the VR chamber 206). In various embodiments, the animal 220 may be rewarded based on behavior/actions performed or not performed. In various embodiments, the treadmill 202 may be any suitable size (e.g., diameter) to allow a user to walk in any direction. For an animal 220, the diameter may be 50 cm. For a human, the diameter may be larger, such as, for example, up to six feet.

One example of a tactile stimulus is a puff of air. For example, air may be blown on the face when a subject is moving fast, or reduce the airflow to lower speed when they are walking slowly, to complete the sensory feedback.

In various embodiments, the offset is determined by the range of offsets to which neurons are sensitive. More particularly, in the Hebbian model of associative learning, the proximate firing of neurons builds an association. Neurons are sensitive to an offset of just 5 ms between two stimuli, and in some circumstances are sensitive to offsets of 1 ms. Neurons are also sensitive to an offset of about 10 ms. Offsets of 100 ms create a consciously perceptible effect, such as when an old film has unsynchronized audio and video leading to a feeling wrongness or unpleasantness.

In some embodiments, the offset is static, and a second stimulus is presented with the same offset. In some embodiments, the offset is selected on the basis of a disease condition of the user. For example, each of a plurality of disease conditions may have their own associated delay. In some embodiments, the offset is selected on the basis of various characteristics of a user, such as for example age, sex, or disease condition.

In some embodiments, the learning system is provided with additional information about the user, such as dynamical brain state, age, sex, or presence of factors such as caffeine or alcohol. In some embodiments, characteristics of the stimulus, such as the stimulus contrast or sound frequency, and their precise timing, are also provided to the learning system. For example, the type of stimulus and its frequency may be provided.

In some embodiments, in addition to providing an updated offset, the learning system provides characteristics for the second pair of stimuli. For example, the learning system may determine type of stimulus and its frequency.

It will be appreciated additional stimuli may be presented. For example, a trio of stimuli may be provided instead of a pair.

The hippocampus gets inputs from dozens of neocortical sensory areas. The hippocampal function depends on the exact timing and correlations between these inputs. When a person experiences the real world, all stimuli are sent to the hippocampus at a synchronized time. This allows the hippocampus to function in a routine manner, for example as described in Hebbian theory. However, in virtual reality, the latency or timing between these inputs is not synchronized as in nature. There are very precise mechanisms in the neurons and synapses that are sensitive to the change in timing by just 10 milliseconds, let alone many seconds. This causes the neural circuits to disconnect (or form wrong connections), resulting in reduced inputs to the hippocampus and hence neural shut down. This mechanism may be characterized as a form of Hebbian plasticity.

Accordingly, any VR environment that has such mismatch between different sensory systems' signals, or where some expected sensory stimulus is missing, reduction in neural activity, particularly in the hippocampus is experienced.

As outlined above, in epilepsy, the strength of neural connections leads to highly synchronized electrical events. The above techniques employ VR to naturally disconnect this circuit and thus disrupt the abnormal electrical events.

In Alzheimer's disease, different sensory inputs are pathologically shut down (e.g., one test of Alzheimer's is patient's ability to smell different things). So, one can use VR to provide an early diagnosis of missing or mistimed inputs. Missing inputs in Alzheimer's can cause imbalance of excitation-inhibition, and hyperactivity in the hippocampus as reported in several human studies. The VR/AR systems described herein can be used to reduce hippocampal hyperactivity in Alzheimer's and other cortico-hippocampal impairments.

Enhanced rhythmicity in VR can be used to enhance the connection between different inputs, resulting in better learning. This provides a method of treating the memory deficits in conditions such as Alzheimer's disease and other forms of learning deficits.

As described above, different sensory stimuli are modulated in a VR environment, and their precise timing is varied. This can be used to encourage or discourage activity in different brain regions in order to treat disorders like epilepsy and/or PTSD.

In various embodiments, vestibular cues are manipulated using the platforms provided herein. For example, by either allowing a patient to rotate their heads and bodies by 360 degrees, as one can do when standing freely. In another example, head movement range is restricted, for example to +/−30 degrees, as one does when sitting in a chair or driving a car.

It will be appreciated that a variety of motions can be accommodates by VR/AR apparatus such as those described herein. For example, a subject may walk on a treadmill rather than just sitting. An exemplary embodiment of a spherical treadmill suitable for people and animals of various sizes is described above.

It will be appreciated that while various embodiments are described in terms of the effects on hippocampal activity, the present disclosure is also applicable to manipulation of neuroplasticity. Neuroplasticity in the hippocampus can occur very quickly, within just a few seconds of exploration. The theta rhythm plays a crucial role in determining how much plasticity.

Similarly, while various embodiments are described in terms of theta rhythm, the present disclosure is applicable to other rhythms as well. For example, the present disclosure may be used to affect the SWS or slow wave sleep rhythm (that occurs also during immobility) and gamma rhythm, that increases with running speed in the hippocampus. The gamma rhythm impaired in Schizophrenia. These rhythms are also altered in VR environments described herein, and after an experience in VR.

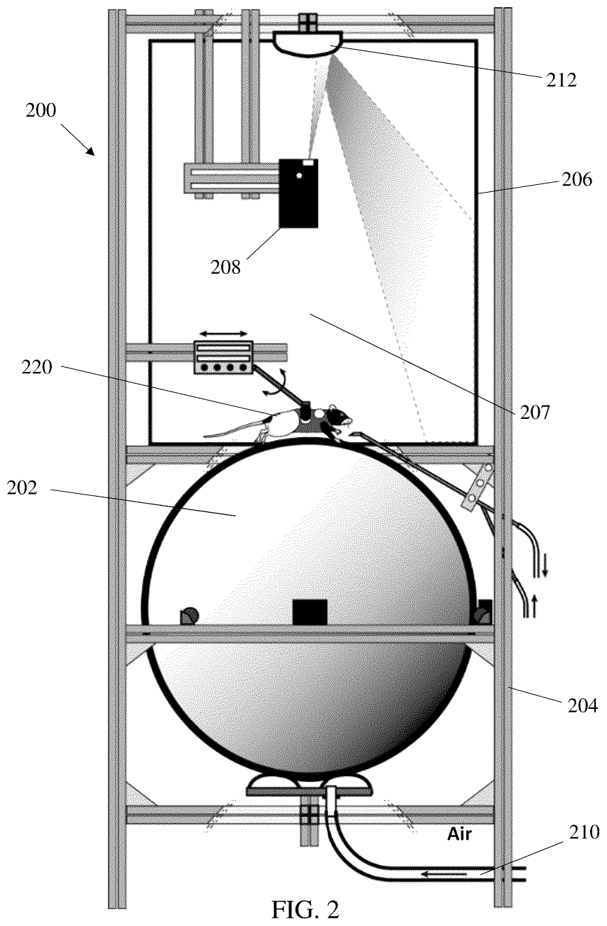

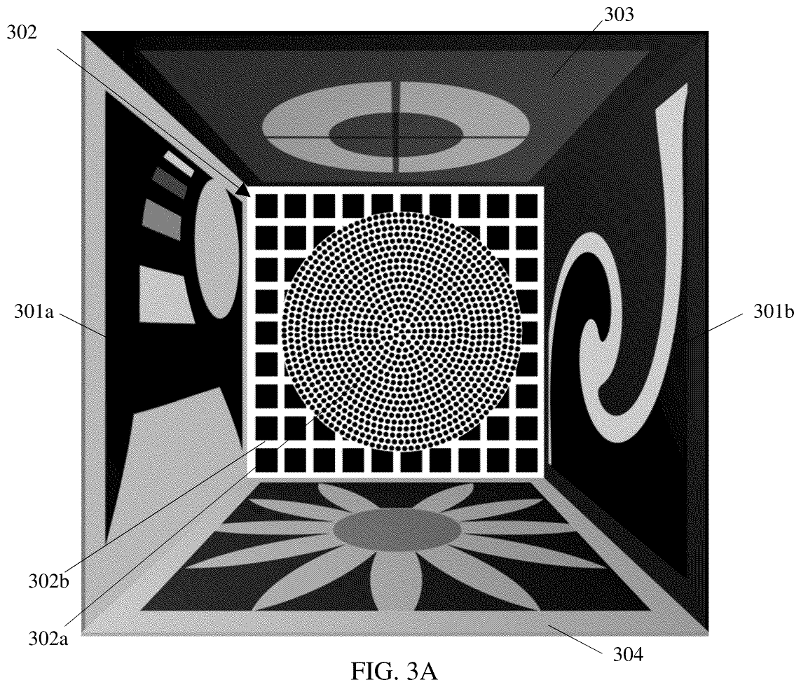

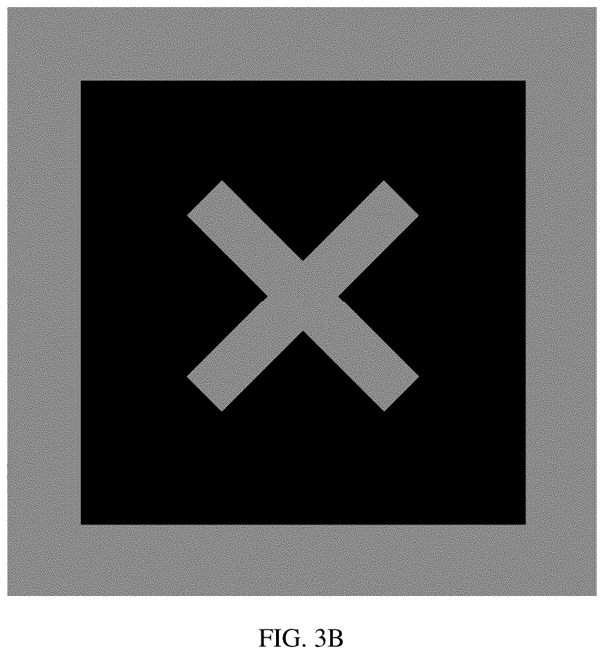

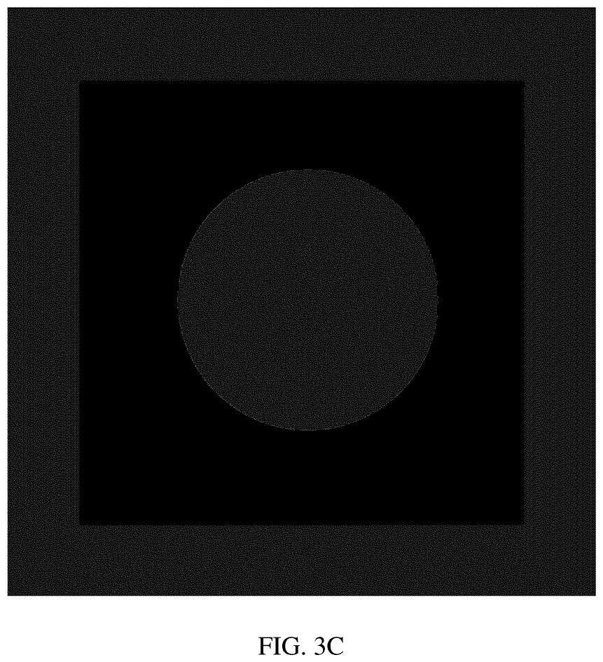

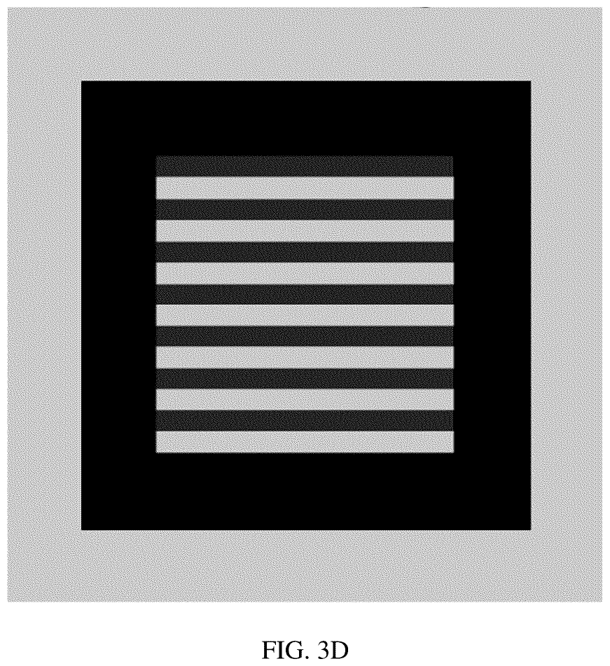

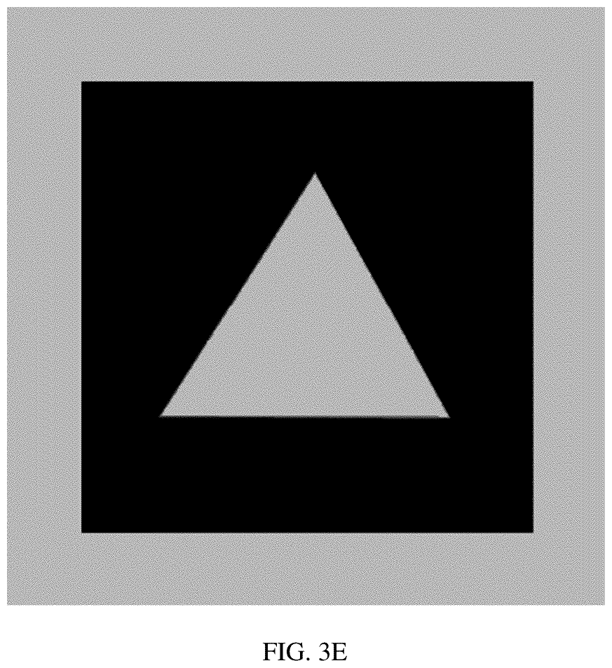

FIG. 2 illustrates a cross-sectional view of the exemplary VR system 200. The VR system 200 is substantially similar to the VR systems 200 illustrated in FIGS. 1A-1B. In various embodiments, the VR system 200 may be used to drive hippocampal activity without movement by the user. For example, one or more visual stimulus may be presented on the internal wall(s) of the VR system 200. Exemplary visual stimuli are provided in FIGS. 3A-3I. In various embodiments, a virtual floor may be presented on a floor of the VR system 200. In various embodiments, the visual stimuli may have low spatial frequency. In various embodiments, the virtual floor may include high spatial frequency. In various embodiments, presenting the one or more visual stimulus to a user may cause changes in the user's hippocampal activity without motion by the user. As demonstrated in the attached manuscripts, a hippocampus can be driven reliably using an autonomously moving stimulus, without any movement from the subject. This is important because, even if a user is comfortable, using VR may require active participation of the subject, which is not sustainable over longer periods of time. Because movement of the user throughout a treatment session may become uncomfortable over longer periods of time, the disclosed systems and methods allow for a user to be comfortable and receive treatment (e.g., hippocampal stimulation) over any suitable time frame. In various embodiments, the autonomously moving stimulus (e.g., in AR) can be used to “fix” the wiring diagram of hippocampus even without the active participation of subject, even when the subject is resting passively. In various embodiments, the disclosed therapy is useful for elderly patients, who are more likely to have memory deficits and hence are unable to sustain attention for long periods.

FIGS. 3A-3I illustrate various visual stimuli. In various embodiments, the visual stimuli may include one or more colors. In various embodiments, the visual stimuli may include one or more of: blue, green, white and black. In various embodiments, the visual stimuli may not include red. In various embodiments, the visual stimuli may be generated using a mathematical algorithm. In various embodiments, the algorithm may generate low spatial frequency stimuli on the inner surface 207 (e.g., walls) of the chamber 206. In various embodiments, the algorithm may be prevented from generating high spatial frequency stimuli on the walls of the chamber 206. In various embodiments, the algorithm may generate high spatial frequency stimuli on a floor of the chamber 206.

In various embodiments, as shown in FIG. 3A, a user may be presented with a different visual stimulus on each side of the inner surface 207 of the chamber 206. In various embodiments, each peripheral side may include a peripheral visual stimulus 301a, 301b. In various embodiments, each peripheral visual stimulus 301a, 301b may be the same. In various embodiments, each peripheral visual stimulus 301a, 301b may be different (as shown in FIG. 3A). In various embodiments, the user may be presented with a floor visual stimulus 302 on a floor of the chamber 206. In various embodiments, the floor visual stimulus 302 includes a platform 302a suspended over a virtual ground 302b. In various embodiments, the floor visual stimulus 302 may include a high spatial frequency stimulus that is configured to reduce sea-sickness and/or dizziness of the user. In various embodiments, the high spatial frequency stimulus may include a plurality of closely-packed shapes (e.g., small circles) that make up a larger shape (the circular platform 302a). In various embodiments, the virtual ground 302b may include a grid pattern (e.g., square cross-hatching). In various embodiments, the pattern and/or shape of the virtual ground 302b may be selected to contrast against the pattern and/or shape of the platform 302a. In various embodiments, the VR chamber 206 may have any suitable size to provide a VR/AR environment to a user (e.g., a human).

In various embodiments, the user may be presented with a forward visual stimulus 303. In various embodiments, the forward visual stimulus 303 may include one or more shapes and/or patterns. For example, the forward visual stimulus may include a target (e.g., a toroid with cross-hair). In various embodiments, the user may be presented with a top visual stimulus on a top surface (e.g., a ceiling) of the chamber 206. In various embodiments, the user may be presented with a rear visual stimulus 304 on a rear surface (e.g., a wall) of the chamber 206. In various embodiments, each peripheral visual stimulus 301a, 301b, forward visual stimulus 303, top visual stimulus, and/or rear visual stimulus 304 may include a low spatial frequency visual stimulus that is configured to reduce sea-sickness and/or dizziness of the user when used in conjunction with the high spatial frequency visual stimulus as the floor visual stimulus 302.

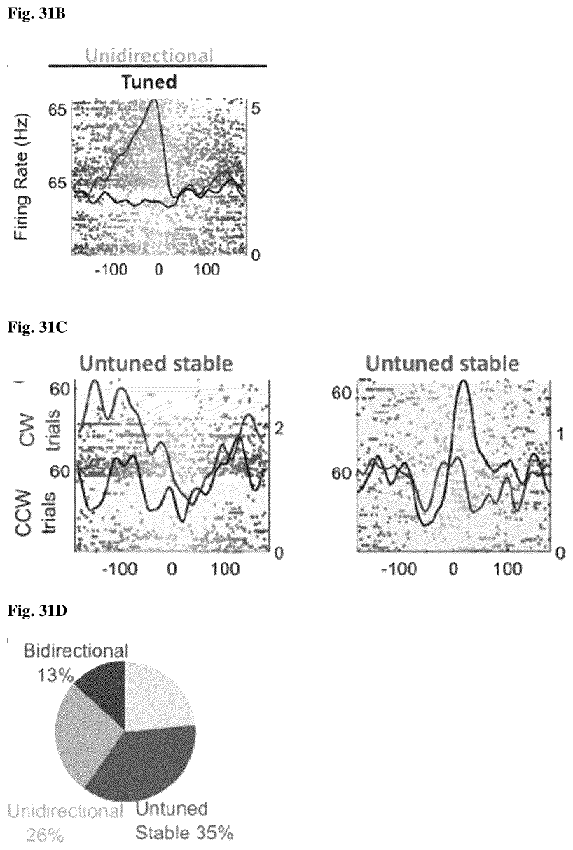

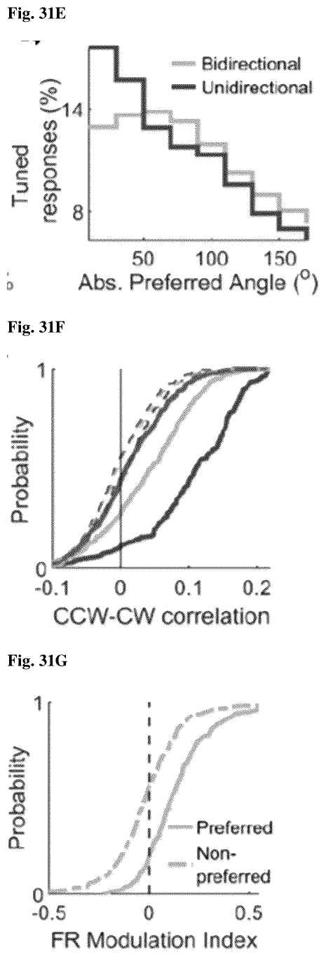

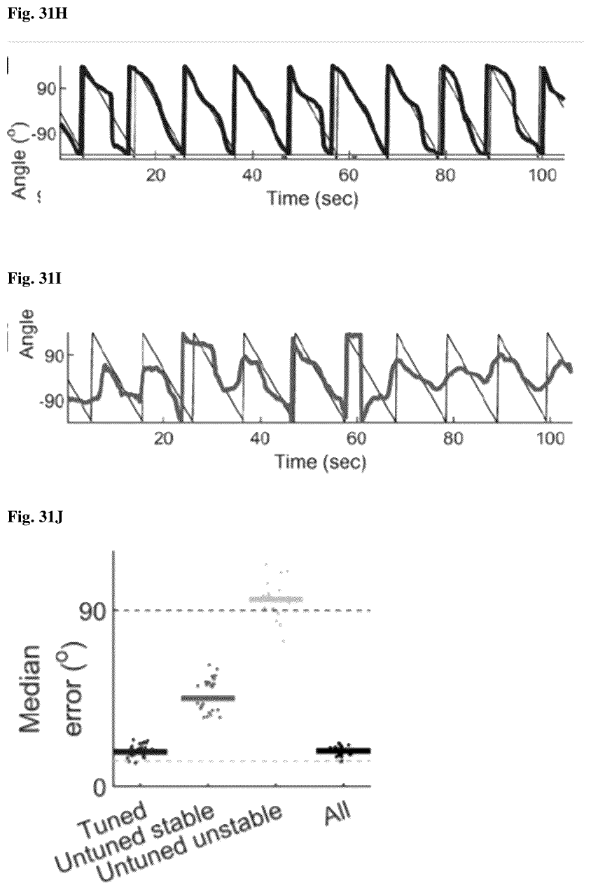

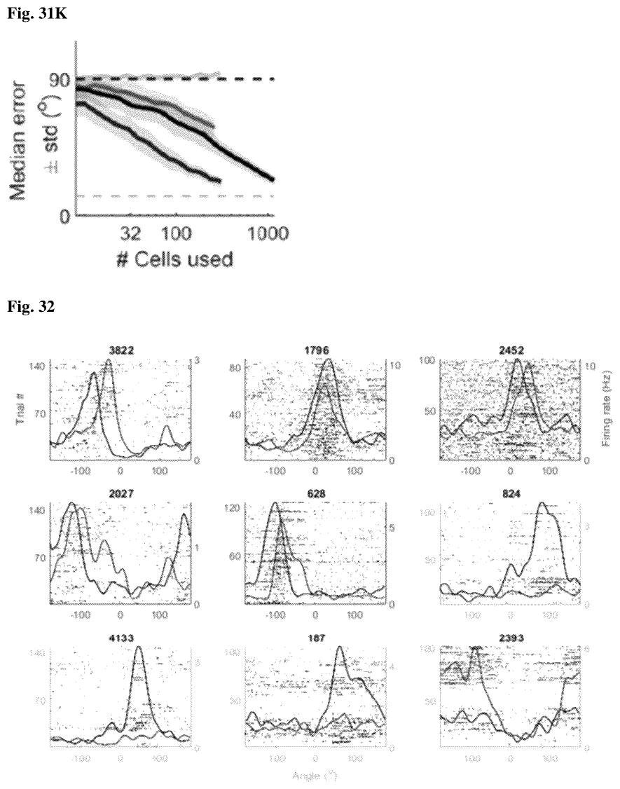

In various embodiments, the visual stimuli may include one or more shapes and/or patterns. For example, as shown in FIG. 3B, the visual stimuli may include an ‘X’. In another example, as shown in FIG. 3C, the visual stimuli may include a circle. In another example, as shown in FIG. 3D, may include a series of parallel lines (e.g., a grating). In another example, as shown in FIG. 3E, the visual stimuli may include a triangle. In another example, as shown in FIG. 3F, the visual stimuli may include a swirled shape. In another example, as shown in FIG. 3G, the visual stimuli may include a target (e.g., toroid with cross-hairs). In another example, as shown in FIG. 3H, the visual stimuli may include a flower shape (e.g., central circle with petal-like extensions extending radially therefrom). In another example, as shown in FIG. 31, the visual stimuli may include a plurality of shapes, such as, for example, one or more circles, one or more quadrilaterals, etc. In various embodiments, each of the plurality of shapes may have similar sizes or different sizes.

In various embodiments, the visual stimuli may be responsive. In various embodiments, the visual stimuli may change in size as the user moves towards or away from the particular visual stimuli. For example, the forward visual stimulus 302 may became bigger when the user walks towards the forward direction. In another example, the forward stimulus may be rotated when the user moves their head. This is a top-down view of a 4×4 meter virtual room with a 2 m diameter virtual platform suspended 0.5 over the virtual ground. In various embodiments, a relative size of the visual stimuli may be adjusted in real time. In various embodiments, the relative size of each visual stimulus may be adjusted based on a ratio of the walking/running speed of the user and the size of the visual stimulus. For example, the relative size may be determined as the speed of the user divided by the size of the particular visual stimulus. In various embodiments, because the floor visual stimulus has a small size, the resulting spatial frequency is high.

In various embodiments, spatial frequency may be defined relative to the visual acuity of a user. For example, where visual acuity is about 1 degree, any visual stimuli having a size of around 1 degree will have high spatial frequency. In another example, using a visual acuity of about 1 degree, any stimuli that is larger than the visual acuity (e.g., 100 degrees) will have low spatial frequency.

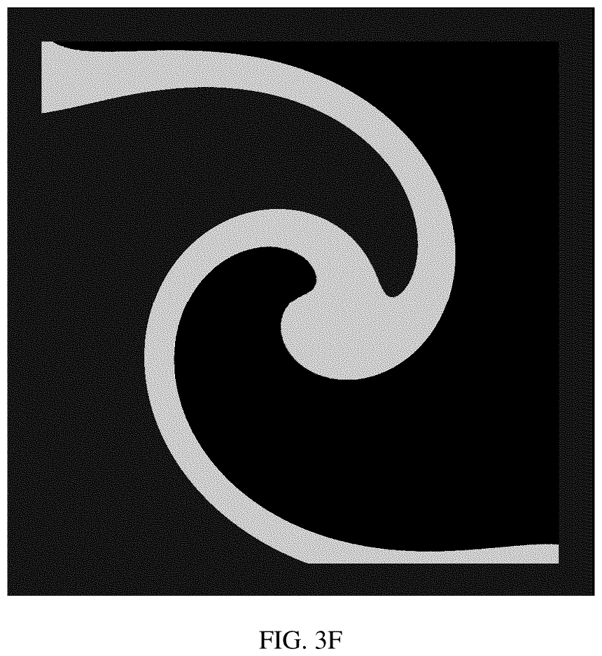

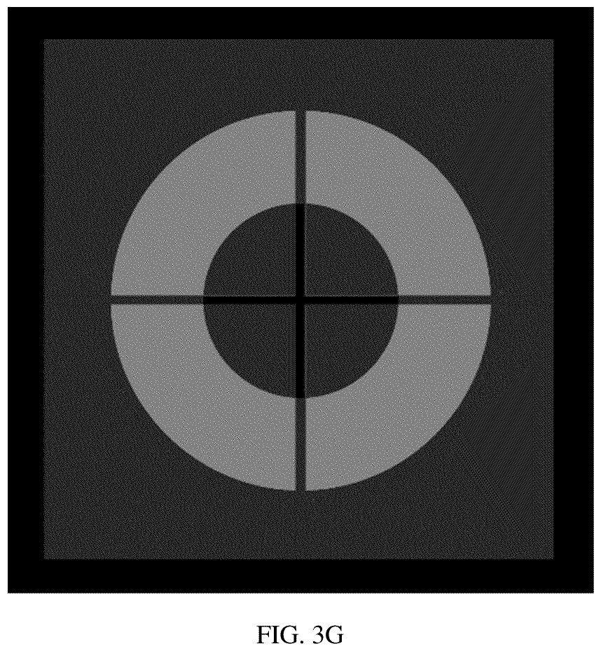

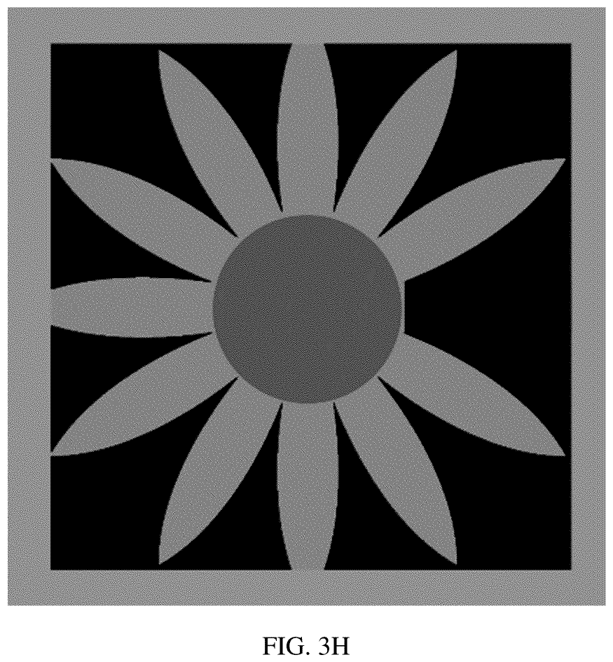

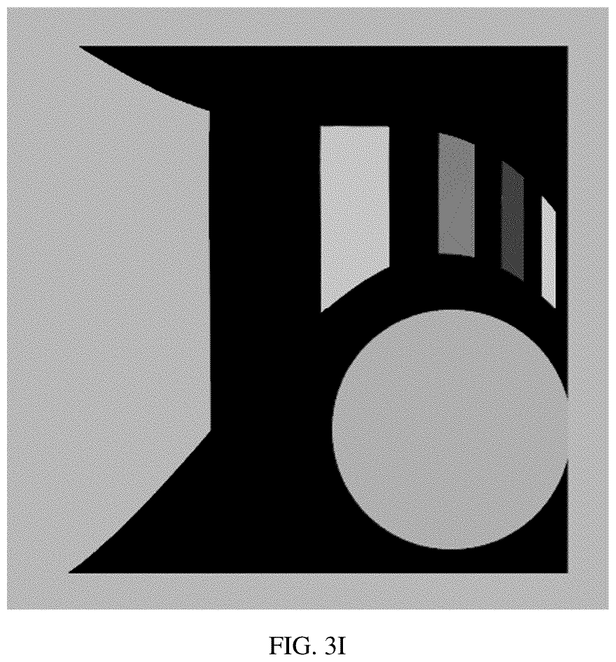

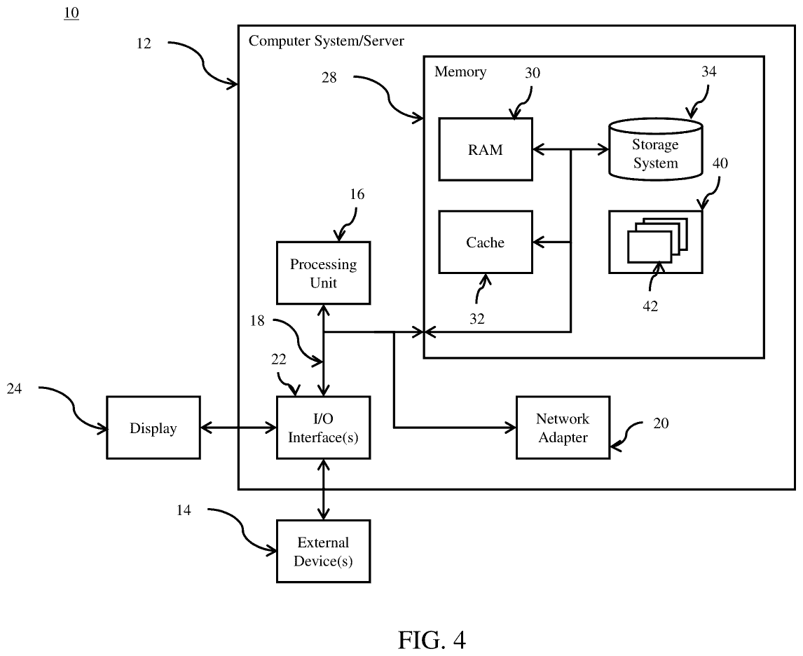

Referring now to FIG. 4, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Unity, OpenGL, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Example 1: Enhanced Hippocampal Theta Rhythmicity and Emergence of Eta Oscillation in Virtual Reality

Hippocampal theta rhythm is a therapeutic target because of its vital role in neuroplasticity, learning and memory. But theta rhythmicity curiously differs across species, and is shown herein to be greatly amplified when rats run in virtual reality. A novel, eta rhythm emerges in CA1 cell layer, primarily in interneurons. Thus, multisensory experience governs hippocampal rhythm. VR can be used to control brain rhythms, to alter neural dynamics and plasticity.

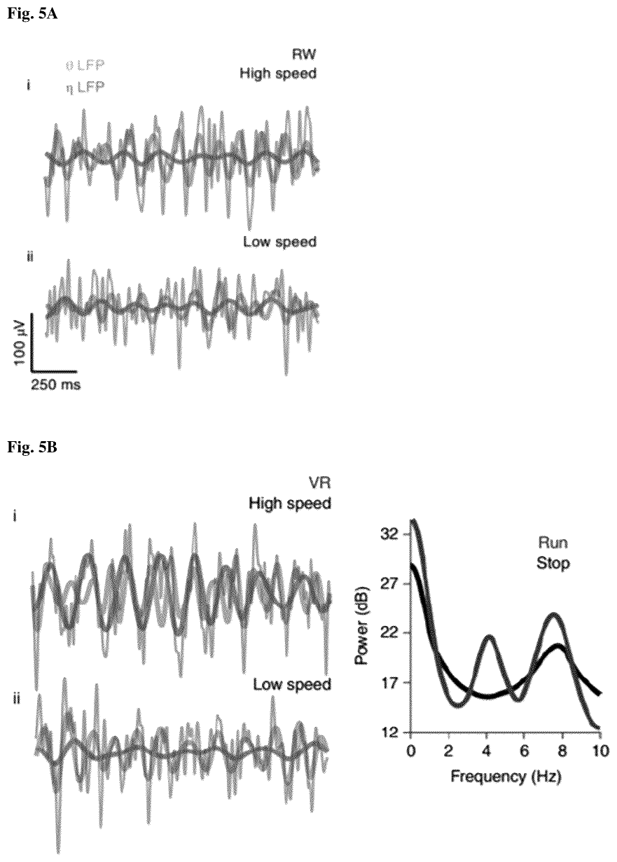

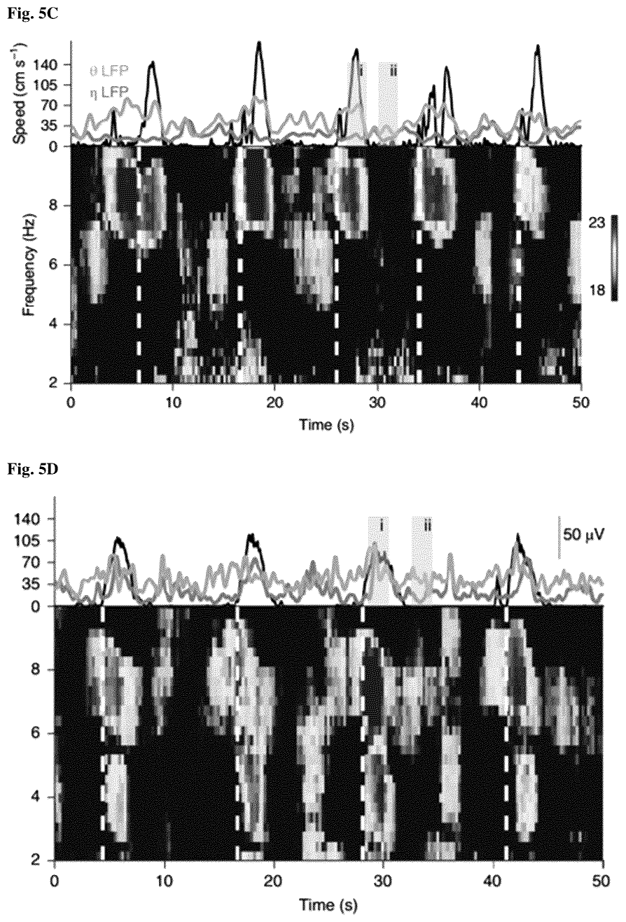

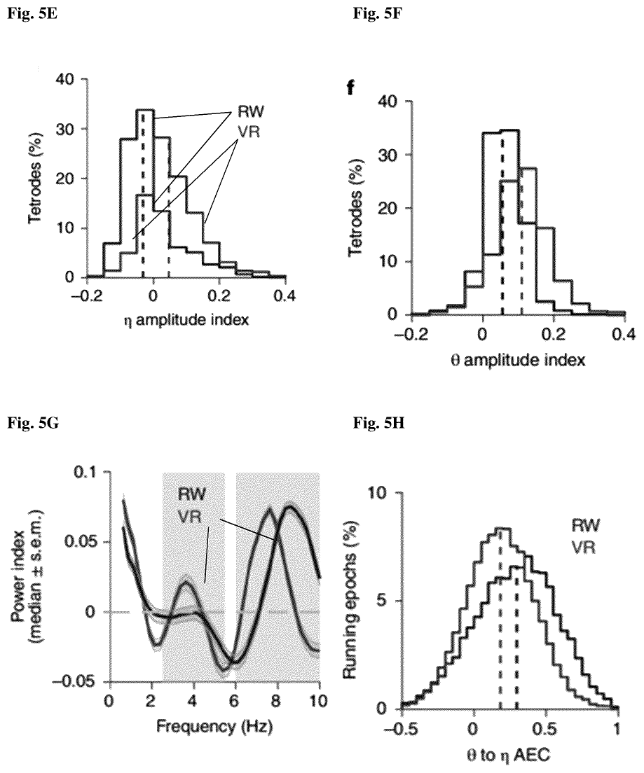

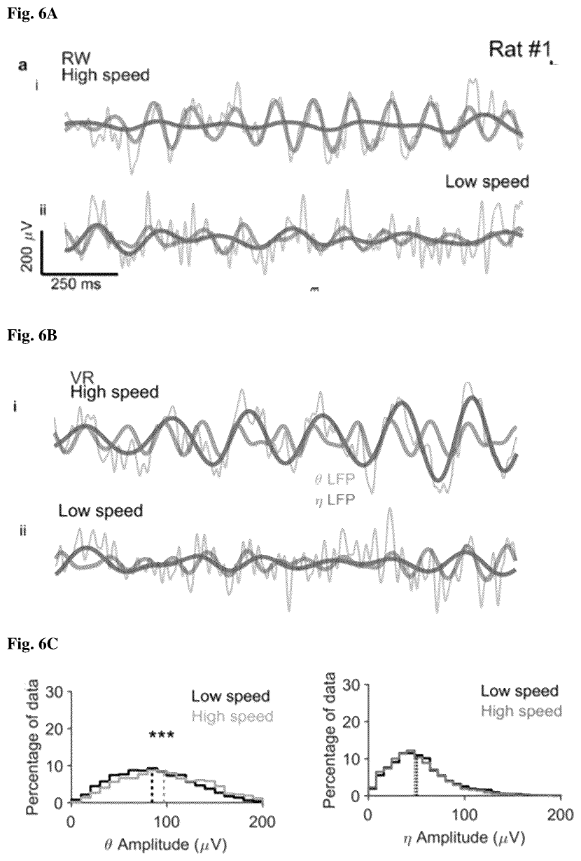

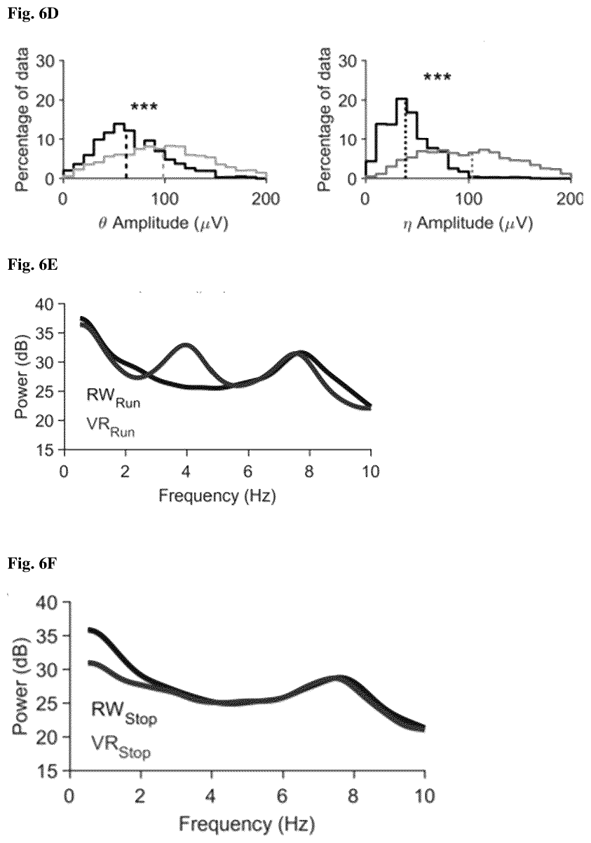

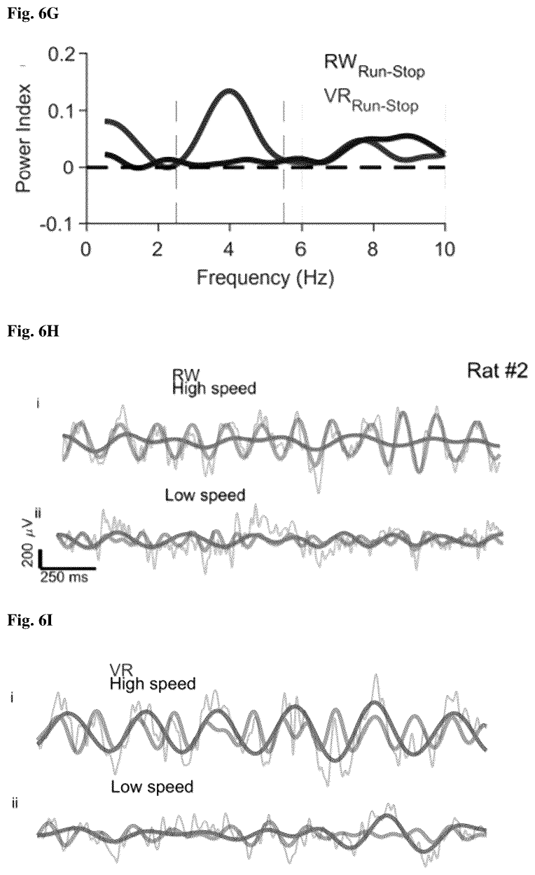

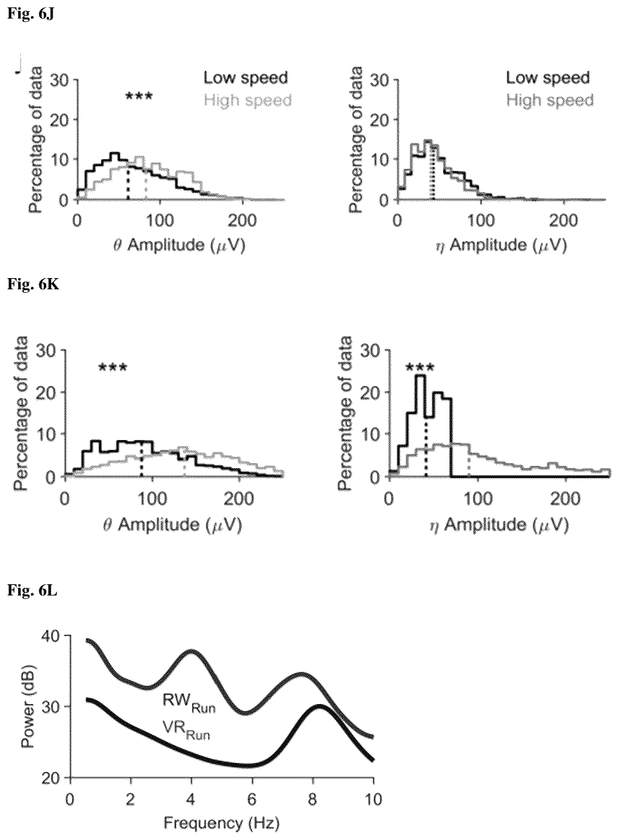

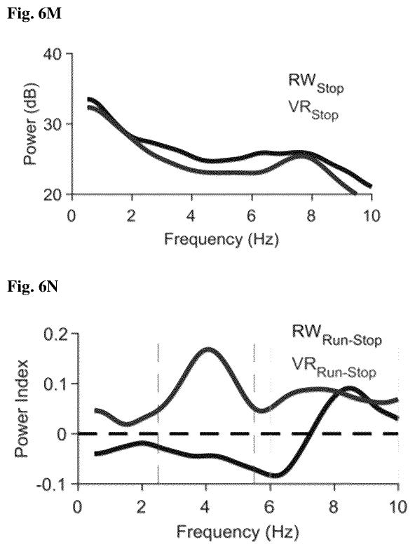

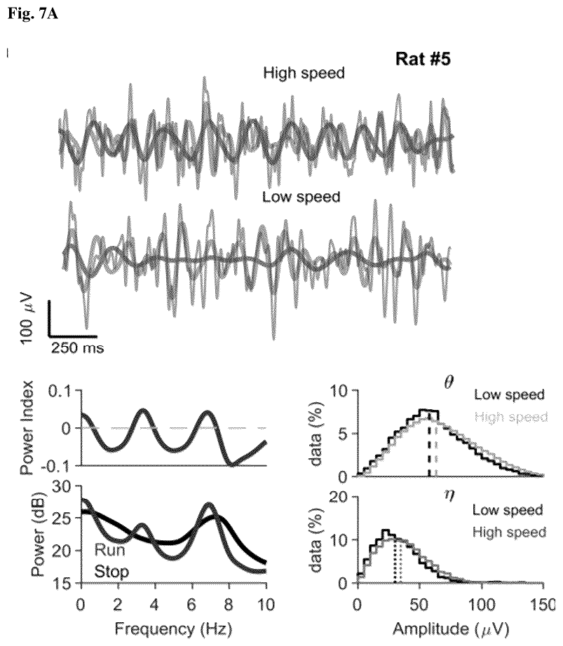

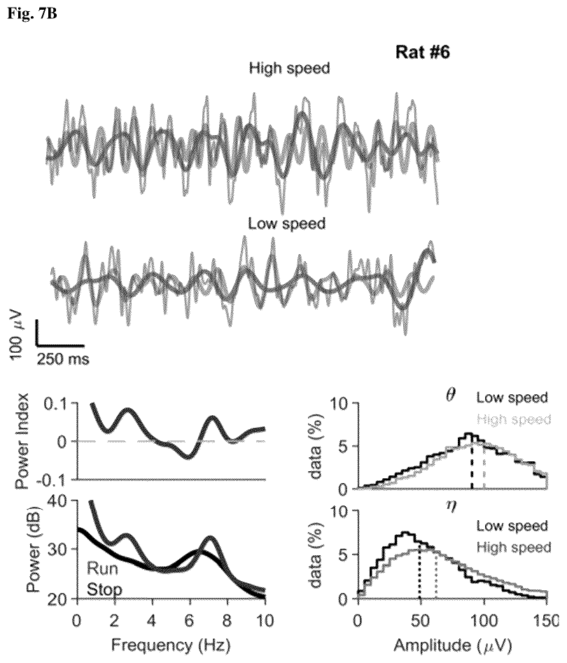

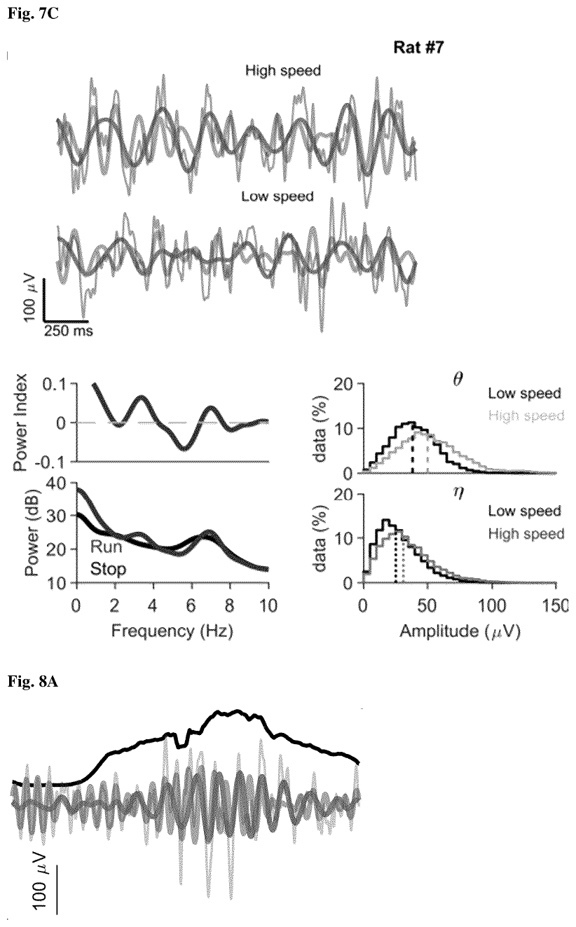

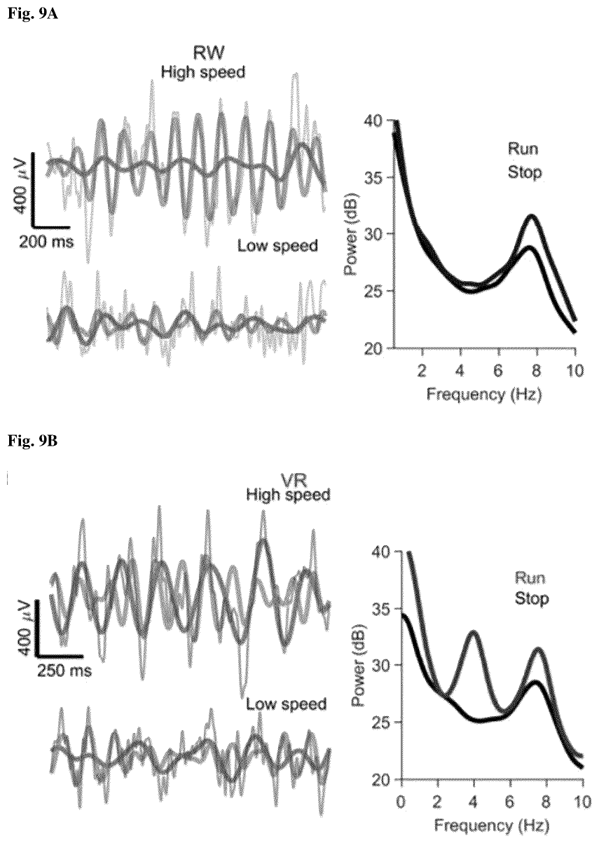

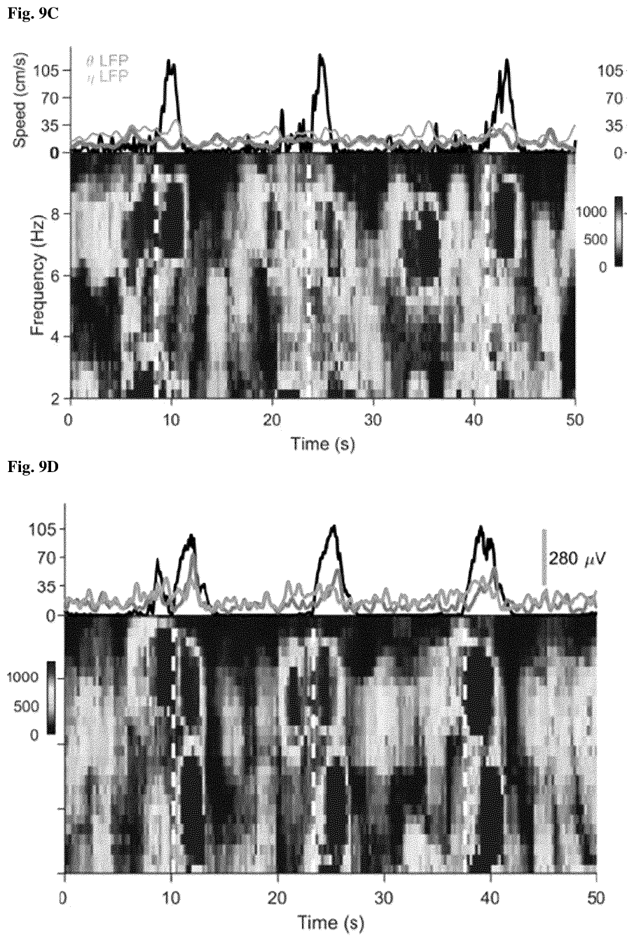

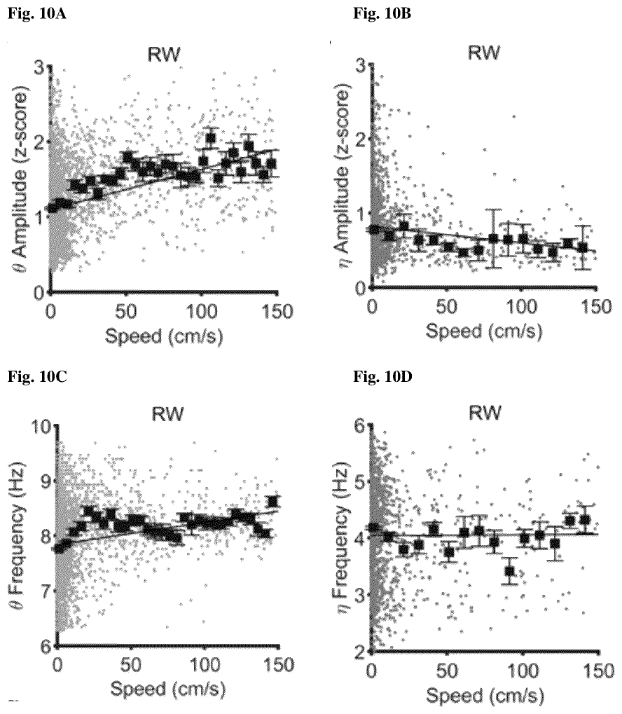

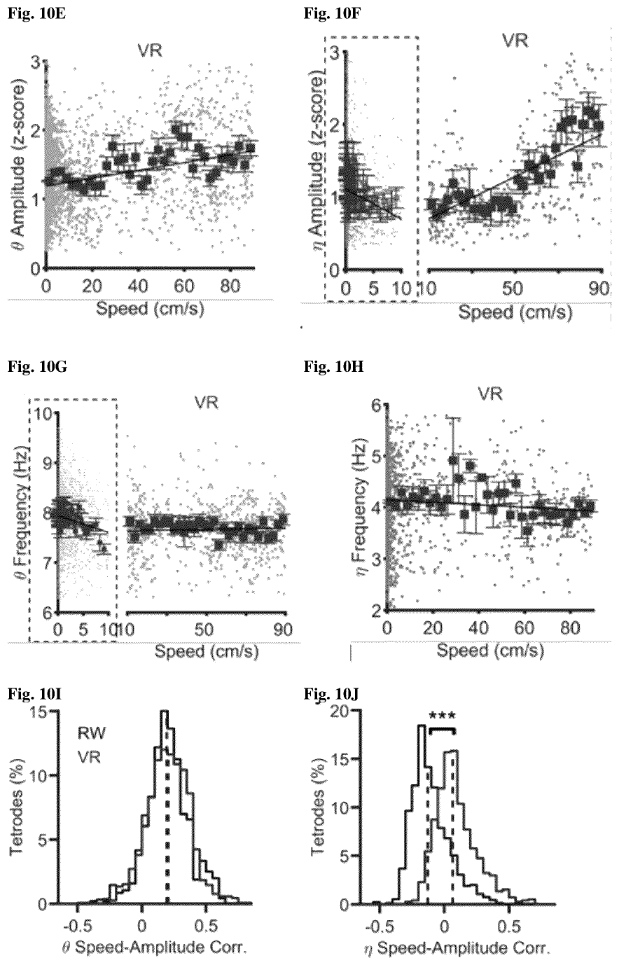

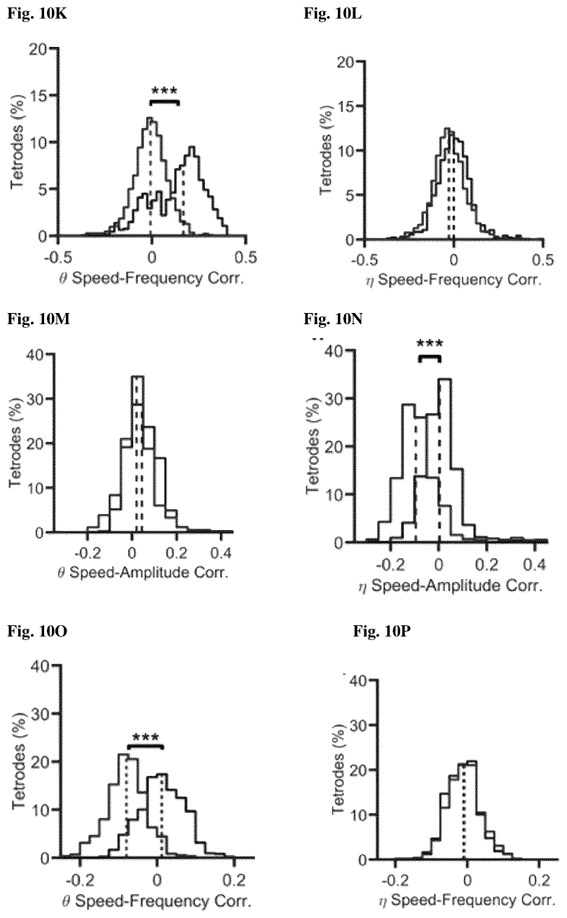

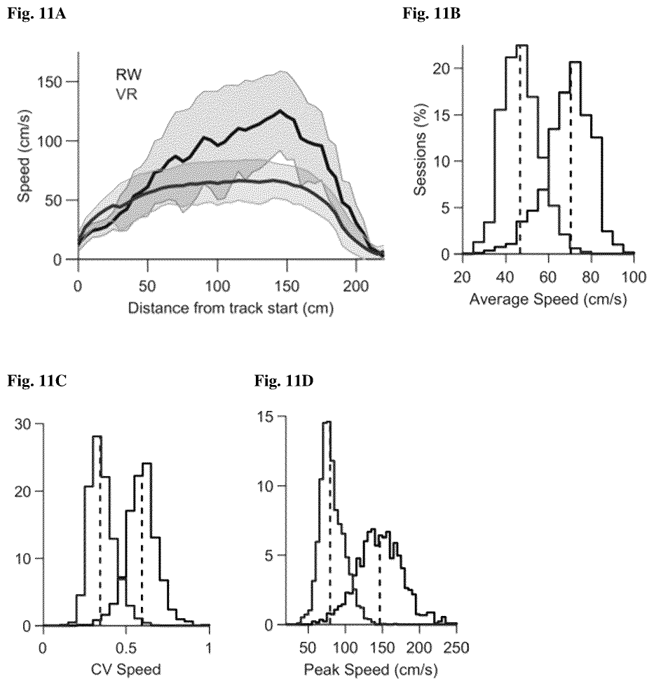

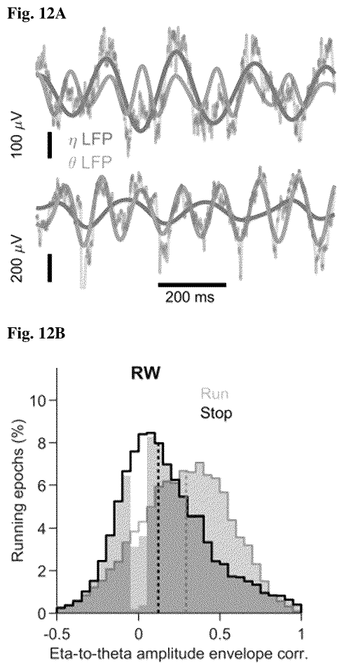

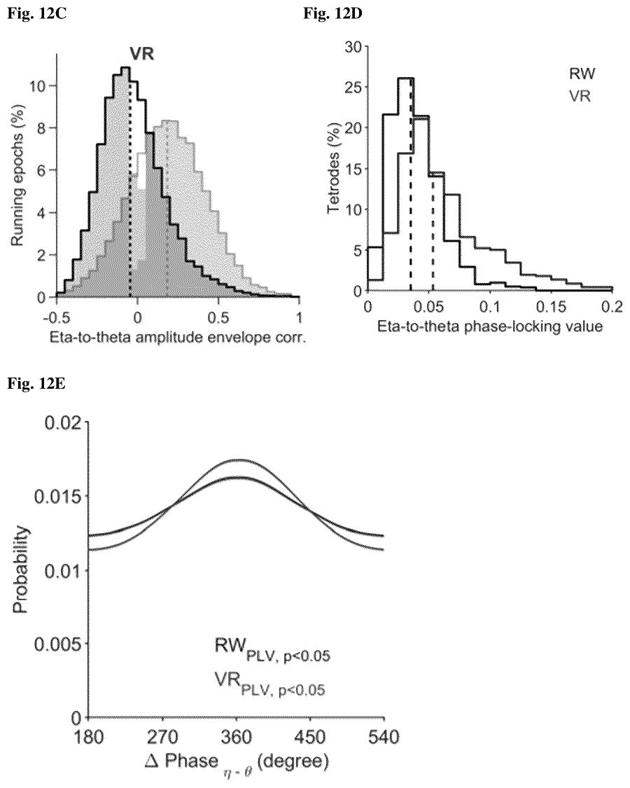

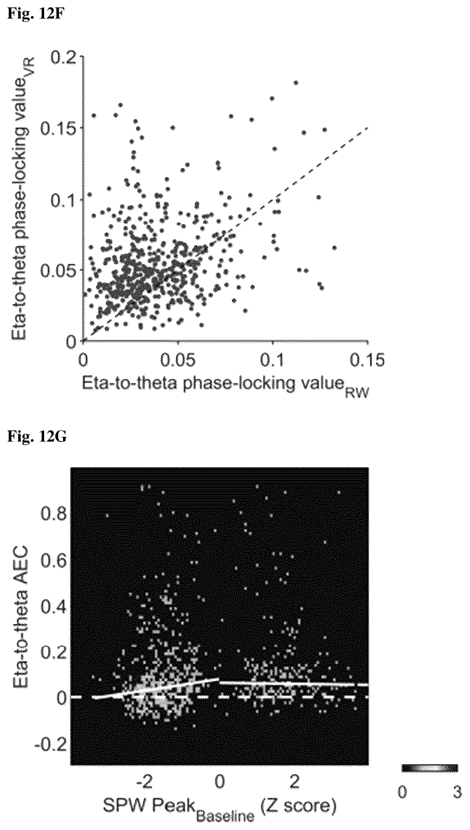

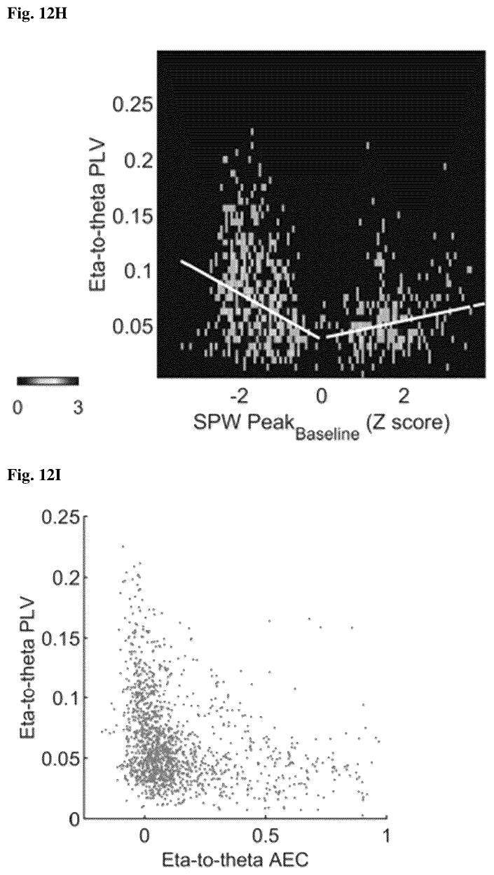

Rats were trained to run on a 2.2 m track, either in real world (RW) or visually identical virtual reality (VR)1. Local field potential (LFP) was measured from 991 and 1637 dorsal CA1 tetrodes of 4 and 7 rats across 60 RW and 121 VR sessions, respectively. Consistent with previous studies1, LFP showed 6-10 Hz theta (θ) oscillations when the rats run in either RW or VR (FIGS. 5A, 5B, and 6-10), that were diminished at lower speeds. However, during runs at higher speeds in VR, but not in RW, novel 2-5 Hz oscillations were also detected on several tetrodes (FIGS. 5A, 5B, and 6-10); termed hippocampal eta (η) oscillations. Like theta, eta was enhanced at high (>15 cm/s) compared to low speeds (FIG. 5B). Thus, the power spectra of the LFP from many tetrodes during runs in VR revealed a peak not only in the theta (˜7.5 Hz), but also in the eta (˜4 Hz) band (FIG. 1b). The latter was absent during immobility. In contrast, the power spectra in RW exhibited a single peak at ˜8 Hz during run, as commonly seen¹² (FIG. 5A). This is clearer in the spectrograms (FIGS. 5C,5D). Theta frequency is slightly reduced in VR (FIG. 5D), and there is another peak in power in the eta band during run in only VR. This is different from the type 2 theta (around 6 Hz) that appears only during periods of immobility. The LFP spectral power could be influenced by several nonspecific factors, e.g., the electrode impedance, anatomical localization, and behavior. Hence, the LFP amplitude difference was computed between periods of high (30-60 cm/s) and low (5-15 cm/s) speed runs in RW and VR and called amplitude index (difference divided by the sum). Remarkably, 71% (29.9%) of all tetrodes showed significantly greater eta amplitude during high speed running epochs in VR (RW), indicating small but significant eta in the RW on some tetrodes. Indeed, eta amplitude index was 600% greater in VR than in RW (FIG. 5E), while theta amplitude index was only 100% greater (FIG. 5F). The latter is slightly different from previous reports¹, because those used a wider frequency range to compute theta, which included eta contributions. To confirm these findings, a more restrictive analysis examined LFP power spectra separately during run and immobility. The power index, similar to the amplitude index, was then computed as the power difference during run and stop, at each frequency, and detected tetrodes with significant, prominent peaks in the eta or theta bands (see methods). This more restrictive analysis showed that 18.6% of tetrodes in VR had significantly prominent eta power index peaks compared to only 1.1% tetrodes in RW. Similar analysis of the theta band revealed comparable power index in RW and VR (84.1% and 80.4%, respectively). As a further confirmation, the analysis was restricted to the LFP data from only those tetrodes that recorded both RW and VR experiments on the same day without any intervening tetrode adjustments. This too showed two distinct peaks in eta and theta bands in VR, but only one peak at theta in RW (FIG. 5G). These results showed significant and sustained increase in eta oscillations during run, compared to stop, in VR but not in RW. In general, rats tended to run a bit slower and had greater periods of immobility in VR than in RW (FIGS. 11A-11D), which could explain the reduction in eta power index compared to eta amplitude index. Eta may be present in the RW, even though a clear peak may not be visible in the power spectra at low frequencies, which are more vulnerable to noise and signal variability (see below). Hence, the correlation between the instantaneous amplitudes of theta and eta band LFP was computed regardless of the presence of a clear peak in the power spectrum (FIGS. 5H and 12A-12I). A majority of electrodes in both RW (69.93%) and VR (84.1%) showed significant correlation between theta and eta amplitudes, even when the contribution of running speed (see below) was factored out.

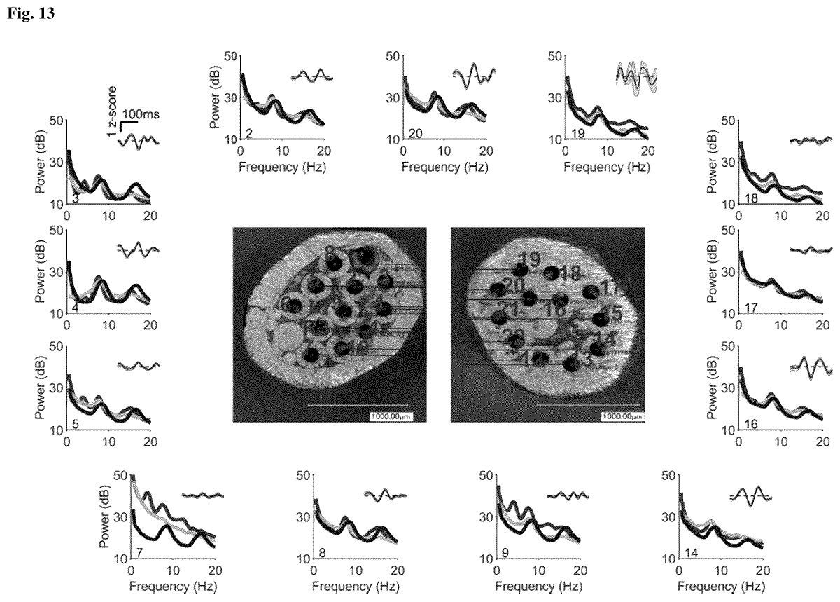

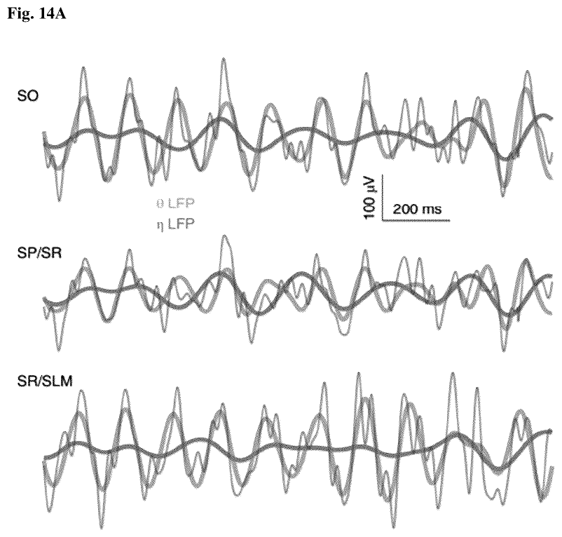

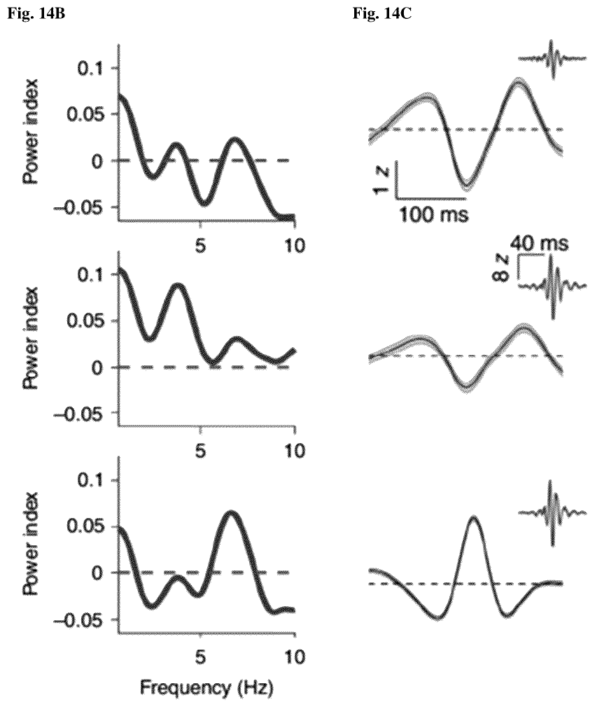

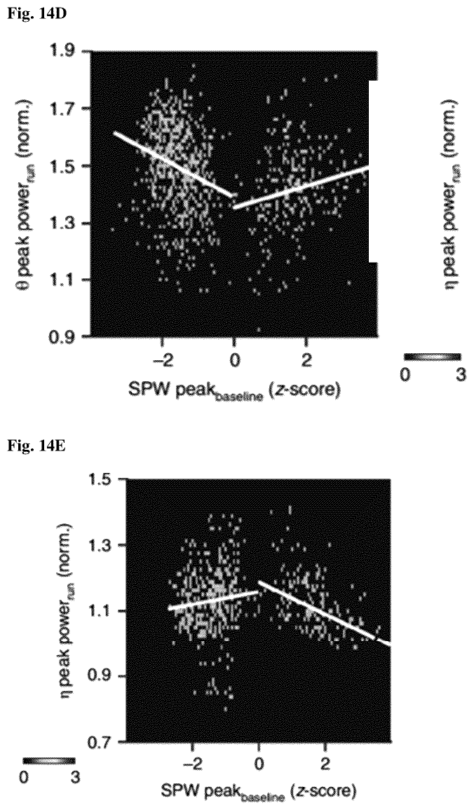

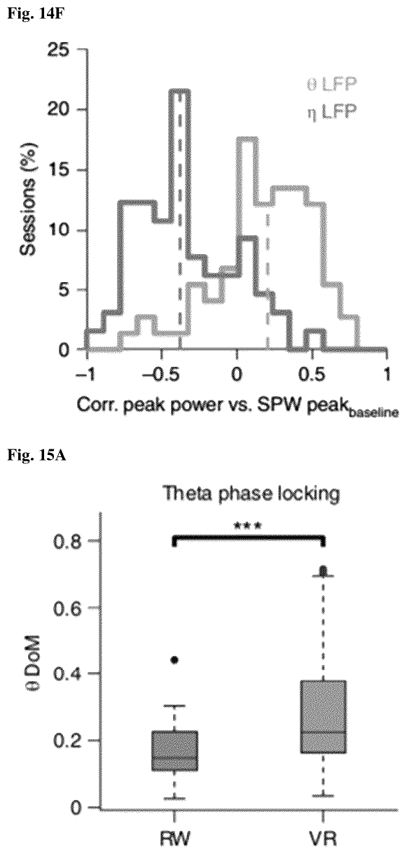

Why was significant and prominent eta, as defined by the strict definition of the power spectrum, often seen in only a subset of simultaneously recorded electrodes (FIG. 13)? The anatomical depth of the electrodes in CA1 could be a key determining factor. In the RW, the lowest theta and sharp wave (SPW) amplitudes occur near the CA1 pyramidal cell layer³. Both increase away from the cell layer into the dendritic region, and the SPW polarity reverses at the cell layer. Thus, SPW amplitude and polarity provide an accurate estimate of the anatomical location of an electrode with respect to the CA1 cell layer. Hence, the amplitude and polarity of SPWs were measured during the baseline sessions preceding the tasks and compared to the theta or eta power on the same electrodes during run in VR (FIGS. 14A-14F, see methods). The SPW amplitude was significantly correlated with the theta power for both the positive and negative polarity SPWs, such that the smallest theta occurred on tetrodes with the smallest SPW (FIGS. 14A-14D), similar to RW findings³. In contrast, eta power during run was significantly anti-correlated with the SPW amplitude during immobility for both the positive and negative polarity SPWs, with the highest eta power coinciding with the lowest SPW amplitude (FIGS. 14A-14C, 14E).

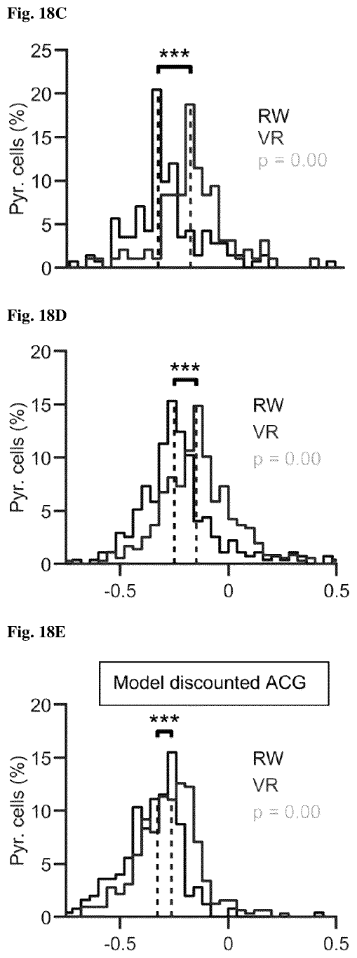

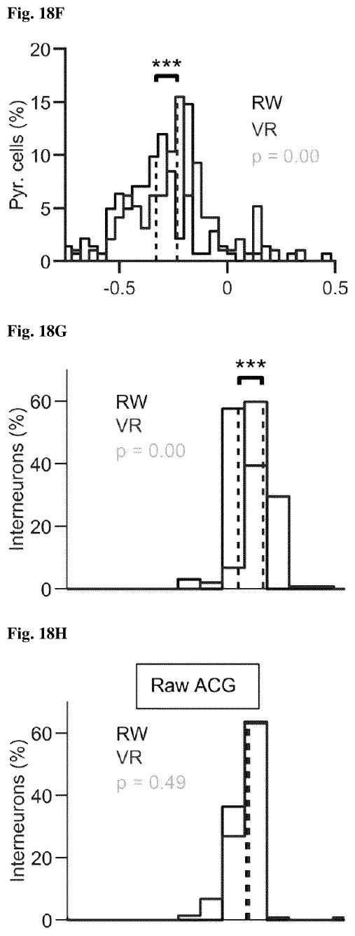

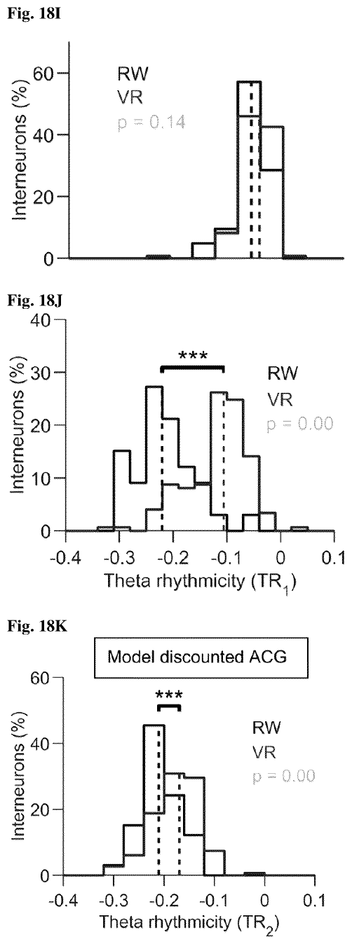

This ensemble analysis could be influenced by differences in behavior across sessions. Therefore, the correlation was computed between the SPW amplitude and theta or eta power across only those electrodes that were recorded simultaneously within a session. As expected, the SPW and theta amplitudes were significantly positively correlated for the majority of sessions (FIG. 14F). But the correlation was significantly negative between SPW and eta amplitude (FIG. 14F). Thus, while theta magnitude is smallest in the CA1 cell layer and larger in the dendrites, eta amplitude shows the opposite pattern, with highest amplitude near CA1 cell layer in VR. Eta was distinct from the type-II theta that appears during immobility for several reasons. Similar to type-I theta, whose amplitude increases with running speed, eta amplitude too increased with running speed. Further, the seed-dependence of theta and eta amplitude was non-monotonic in VR and speed-dependence of theta frequency differed between VR and RW (FIGS. 18A-18J).

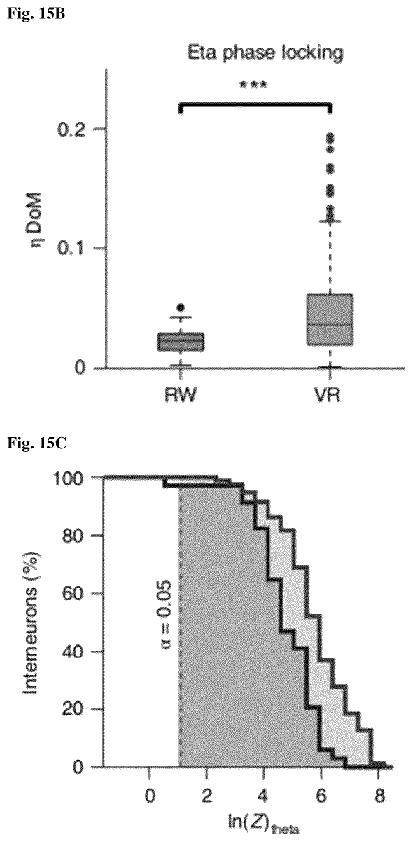

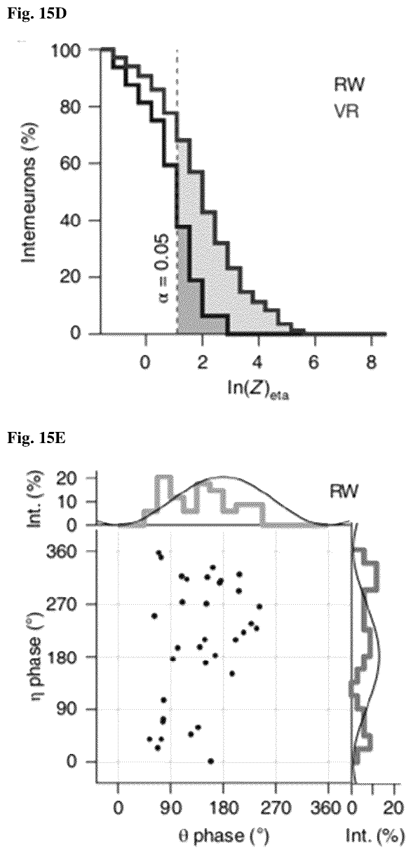

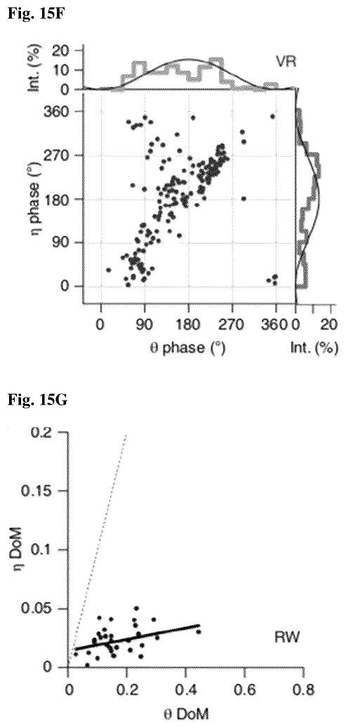

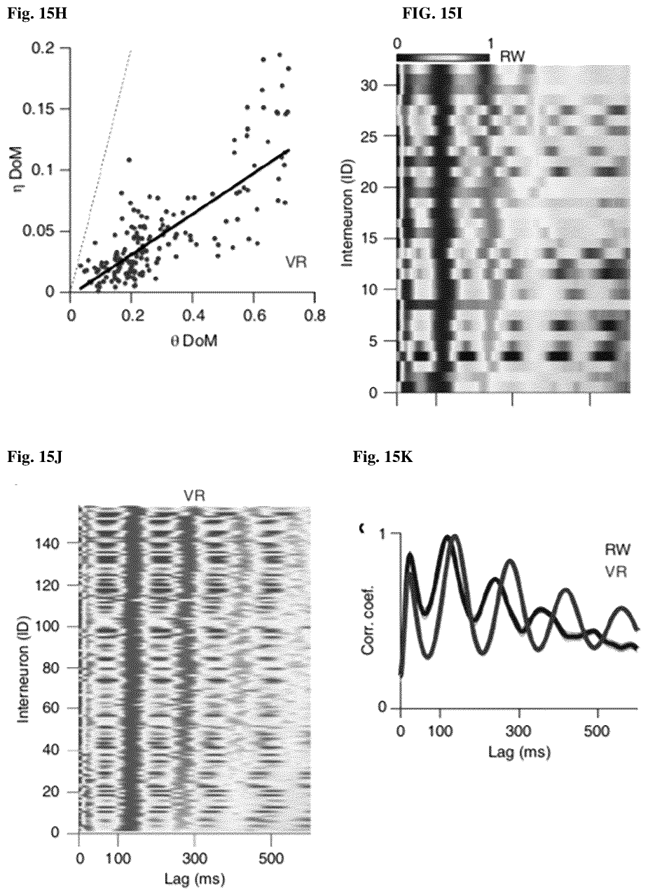

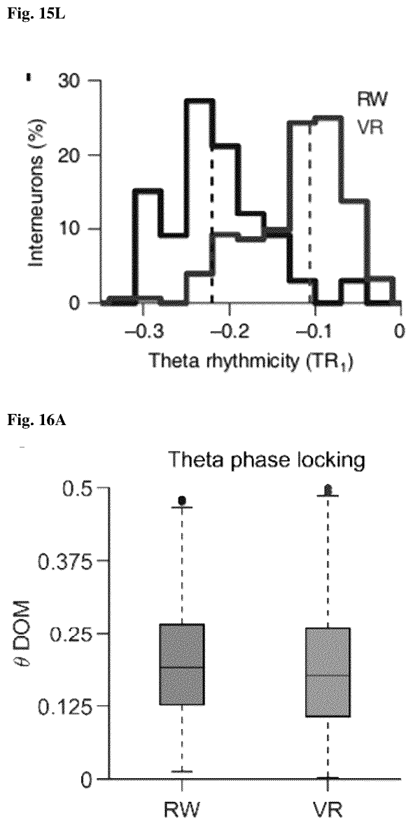

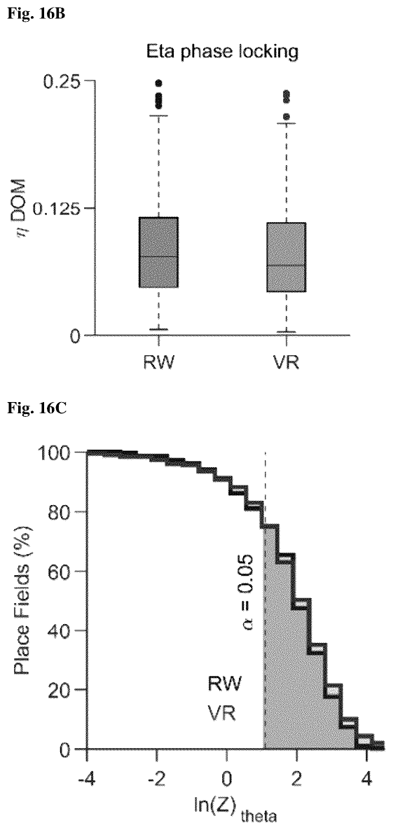

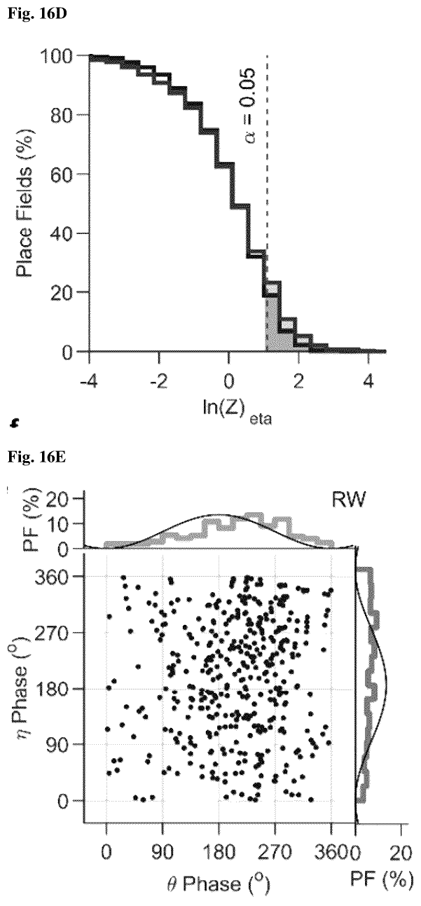

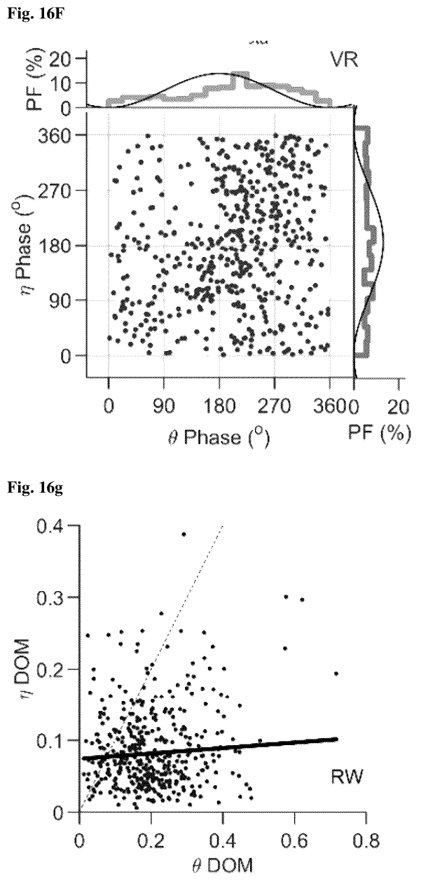

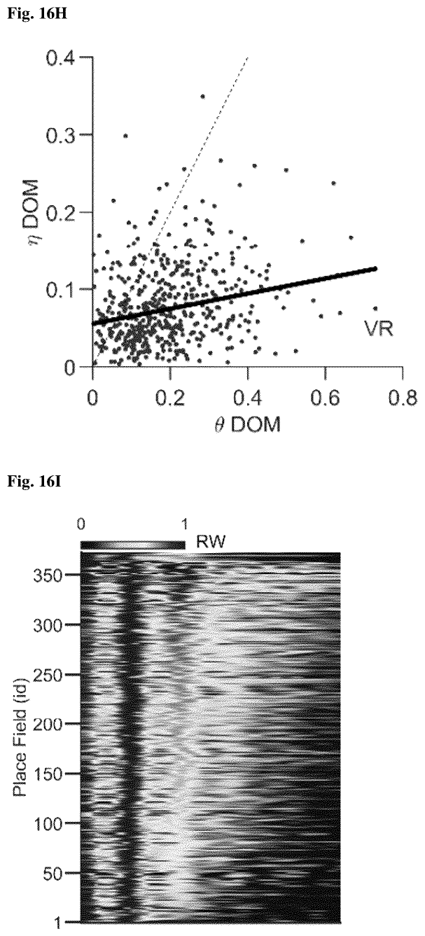

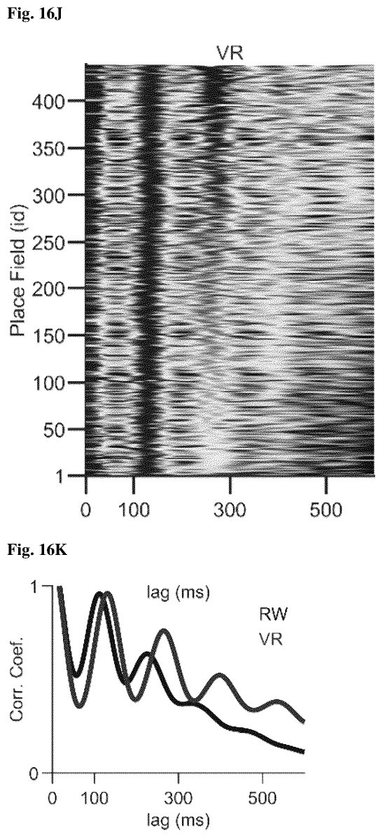

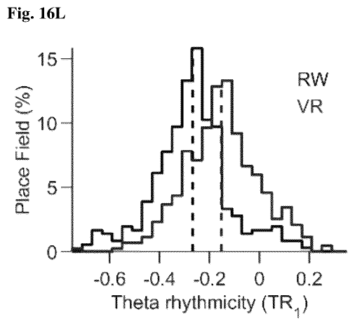

Hippocampal theta is influenced by the medial septal inputs^{4,5}, which target hippocampal inhibitory neurons. Hence, the rhythmicity was examined for 34 and 174 putative inhibitory interneurons in RW and VR, respectively. The number of interneurons in VR are far greater than in RW, which is not the case for pyramidal neurons, because of previously reported large shut down of CA1 pyramidal cells in VR1. The magnitudes of both theta (FIG. 15A) and eta (FIG. 15B) phase locking of the interneurons were nearly twice as large in VR than RW. All interneurons showed significant theta phase locking in both RW and VR (FIG. 15C). But, significantly eta phase locked interneurons in VR (66.6%) was far greater than in RW (35.3%) (FIG. 15D). The interneuron's preferred theta phase was similar in both worlds (FIGS. 15E,15F). But, the population of interneurons, and not pyramidal neurons (FIGS. 16A-16L), showed greater eta phase preference in VR by preferentially firing near the eta peak (FIG. 15E, 15F). As a result, far greater (circular) correlation was seen between eta and theta phase preferences of interneurons in VR (FIG. 15F) than in RW (FIG. 15E). Similarly, eta to theta co-modulation of interneurons was stronger in VR (FIG. 15H) than in RW (FIG. 15G).

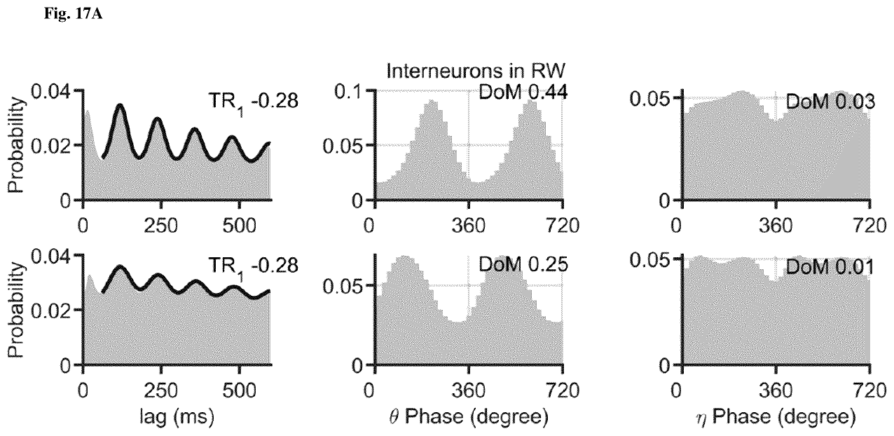

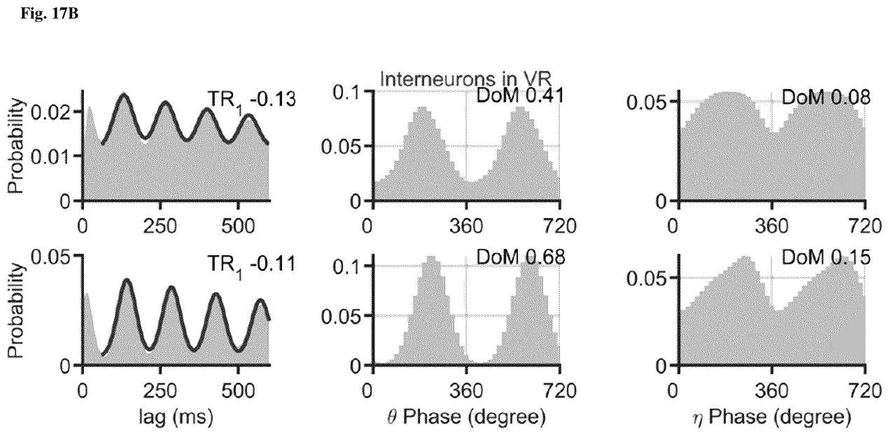

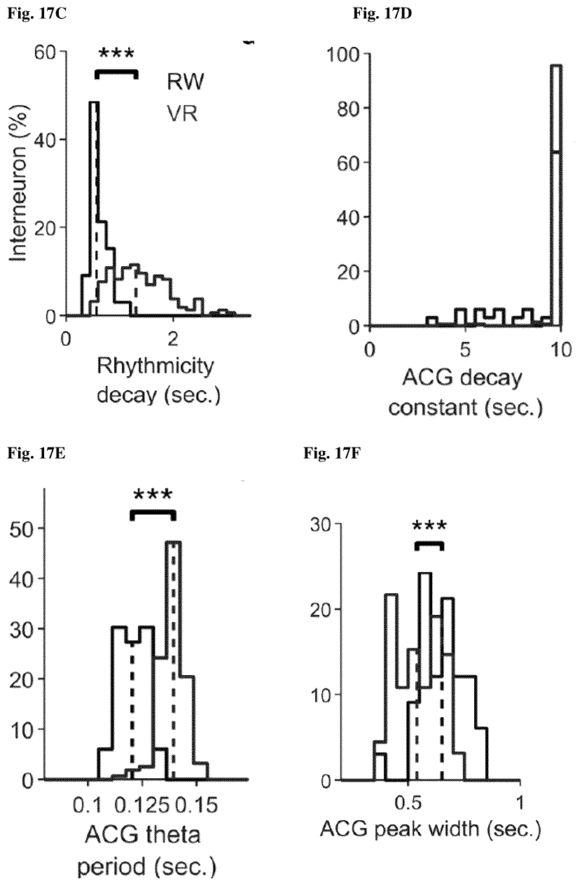

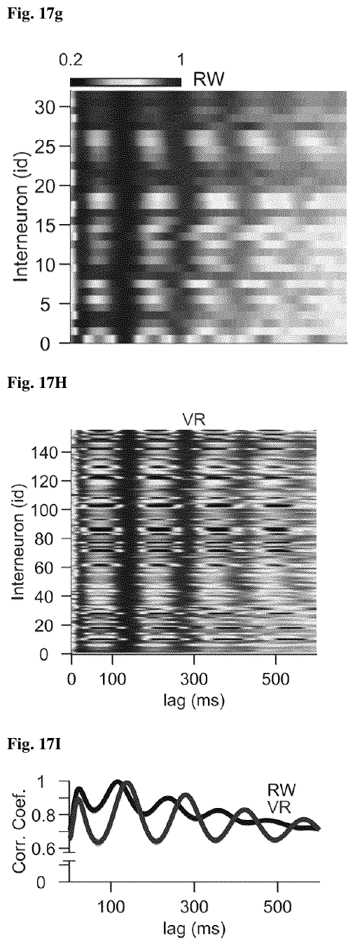

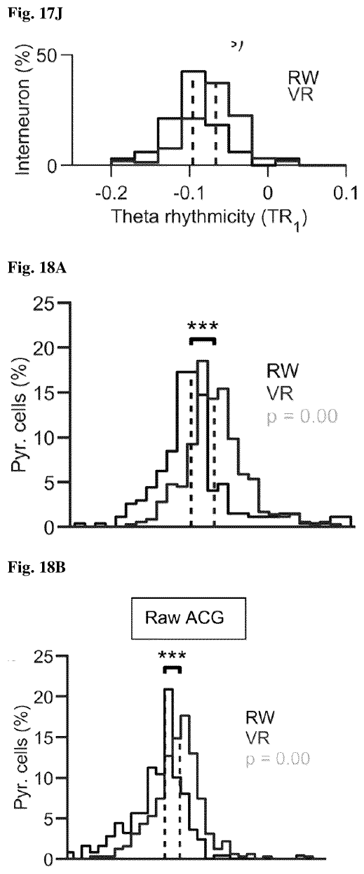

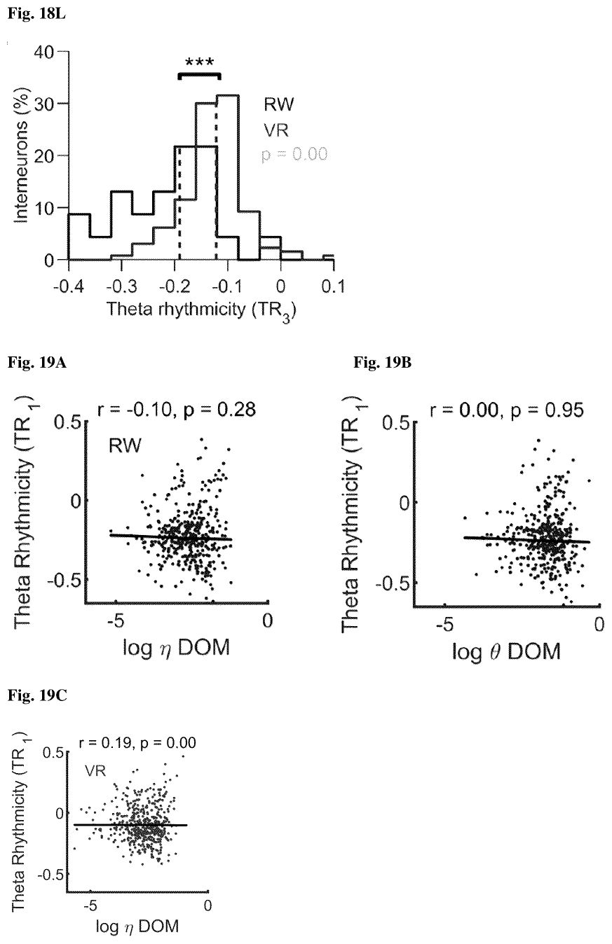

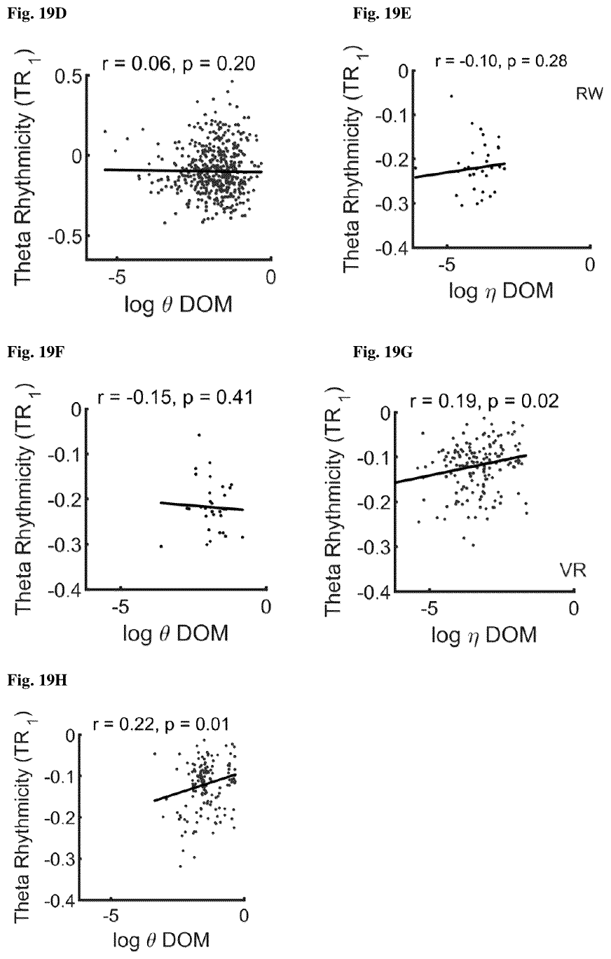

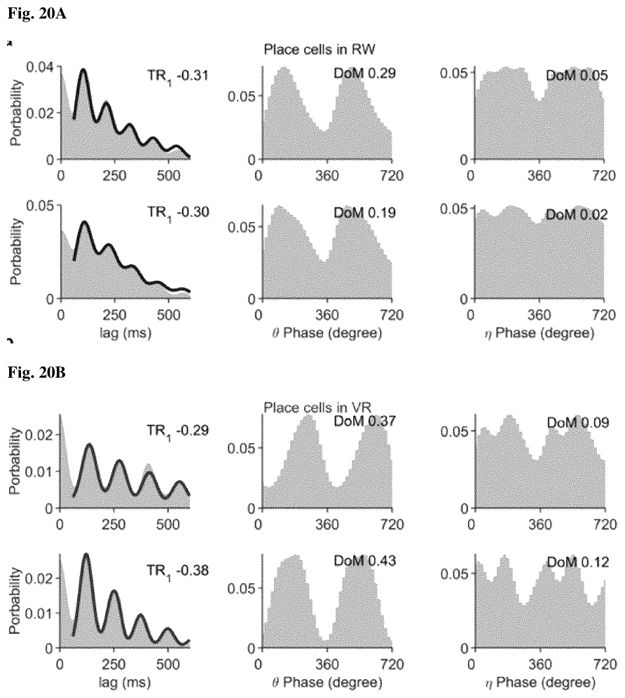

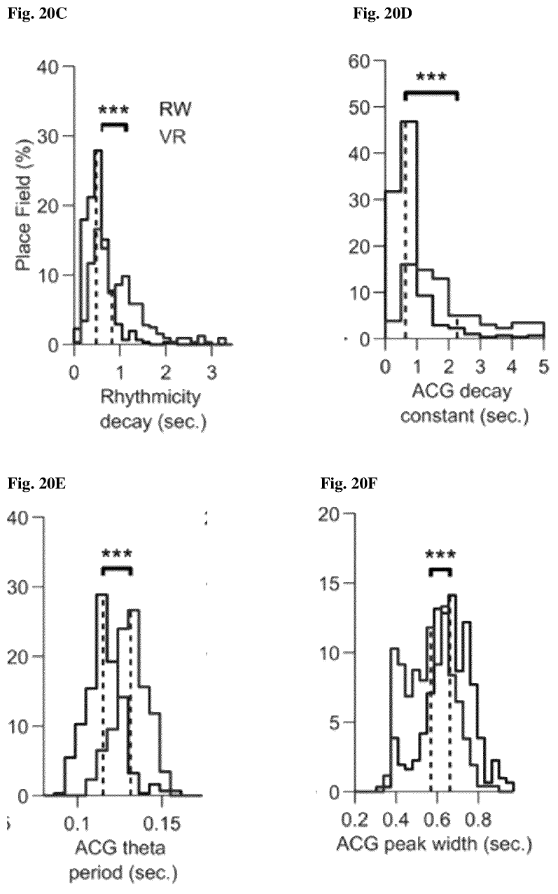

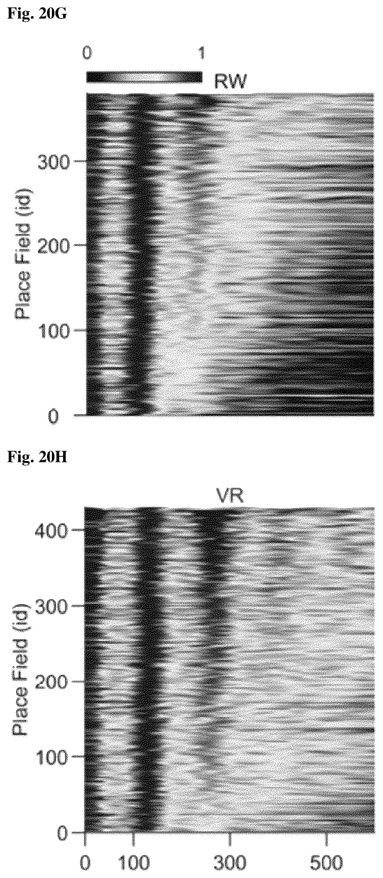

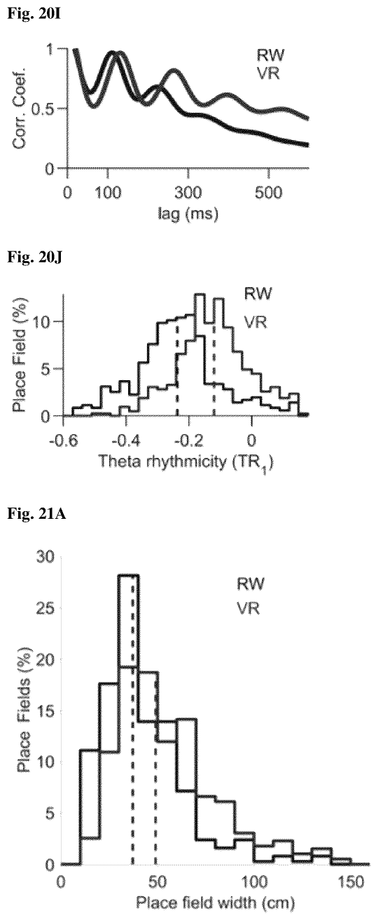

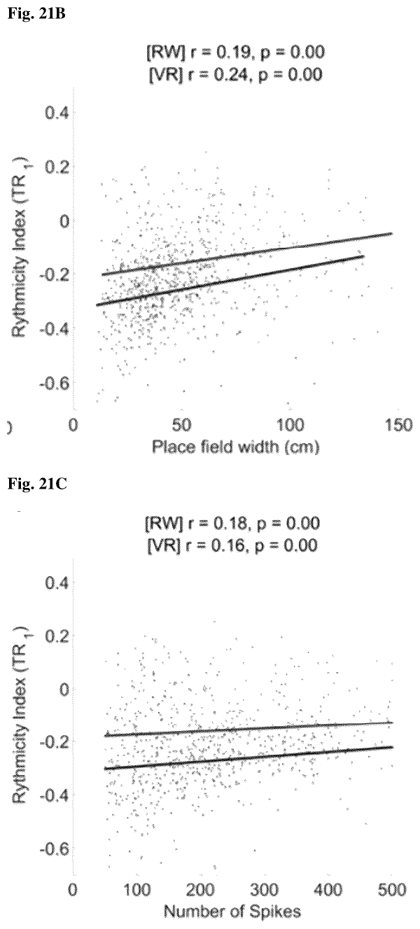

Finally, the interneurons' autocorrelations showed greater theta rhythmicity in VR than in RW (FIG. 15I-15k), evidenced by larger amplitudes of second, third and fourth peaks (FIGS. 15I, 17A-17J, 18A-18L). This suggests that increased theta rhythmicity of interneurons maybe related to the emergence of eta rhythm in VR. Indeed, interneurons with higher theta rhythmicity showed greater theta and eta phase locking in VR (FIGS. 19A-19H), but not in RW. The CA1 pyramidal neurons too showed enhanced theta rhythmicity in VR (FIGS. 20-22). But, unlike the interneurons, the CA1 pyramidal neurons showed very little eta modulation in both RW and VR.

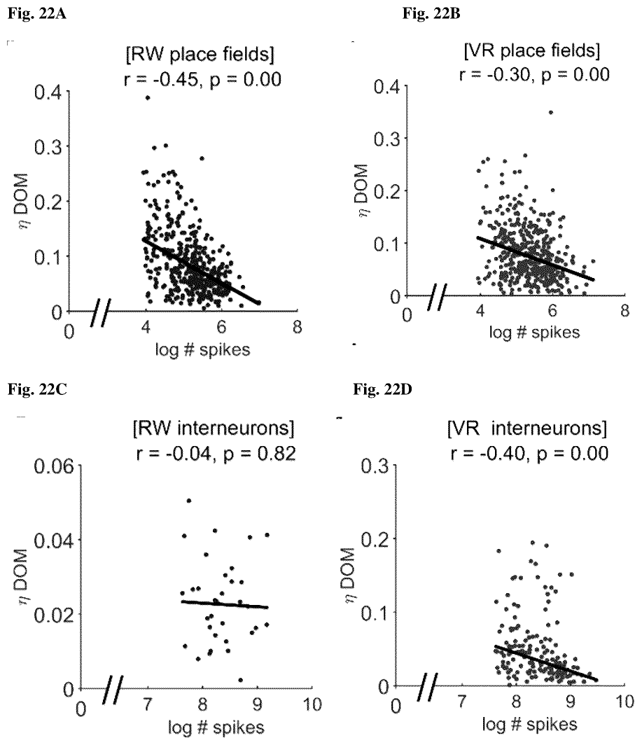

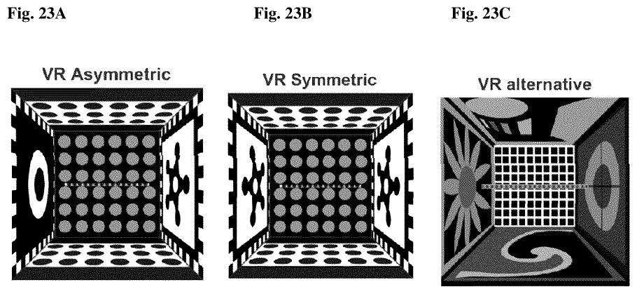

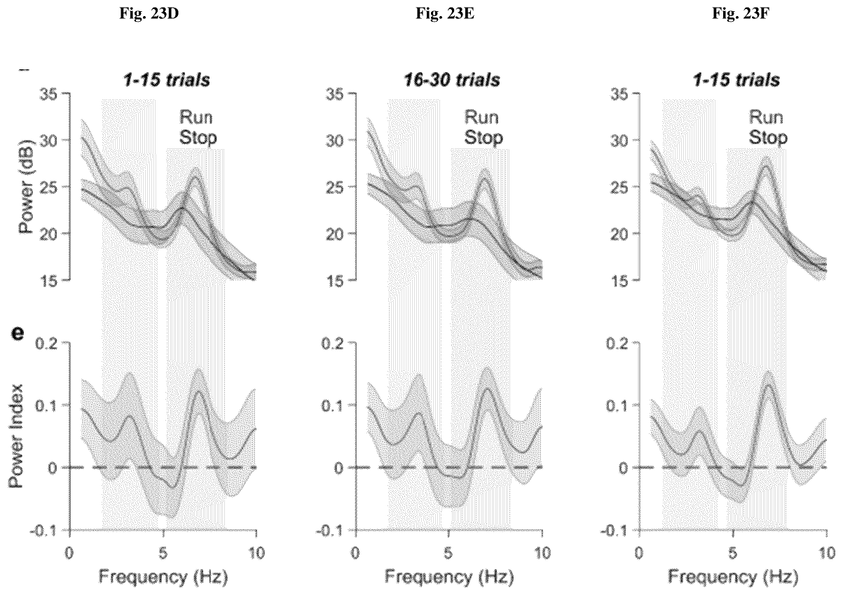

These results revealed the crucial role of multisensory inputs in hippocampal rhythmogenesis. Rodents' dorsal hippocampal CA1 can simultaneously exhibit two distinct slow oscillations, eta and theta while running in the RW, and both are substantially enhanced while running in a visually similar VR (FIGS. 23A-23F). Notably a third of electrodes showed significant eta modulation in the RW and this fraction doubled in VR.

While theta was strongest on dendrite rich regions of CA1, eta was weaker in those areas and strongest in the cell CA1 layer. This suggested that eta may arise locally within CA1 while theta may come from other sources, such as medial septum. Consistently, a third of CA1 interneurons showed significant eta modulation in the RW and this fraction doubled in VR, reflecting similar changes in the LFP. In fact, the eta and theta amplitudes were highly correlated for the majority of electrodes in both VR and RW during run, and not immobility. In contrast, very few place cells, which have more extensive dendrites, showed eta modulation but most showed strong theta modulation in both RW and VR. The rodent eta rhythm in VR may be related to the irregular bouts of 1-5 Hz oscillations reported in humans and nonhuman primates while they are immobile, and performing tasks in VR⁶⁻⁹. On the other hand, eta amplitude in our studies is greater during locomotion than immobility. When humans walk, a higher, ˜8 Hz theta oscillation appears in some hippocampal LFP, which is either absent or substantially reduced in VR^{6,10}.

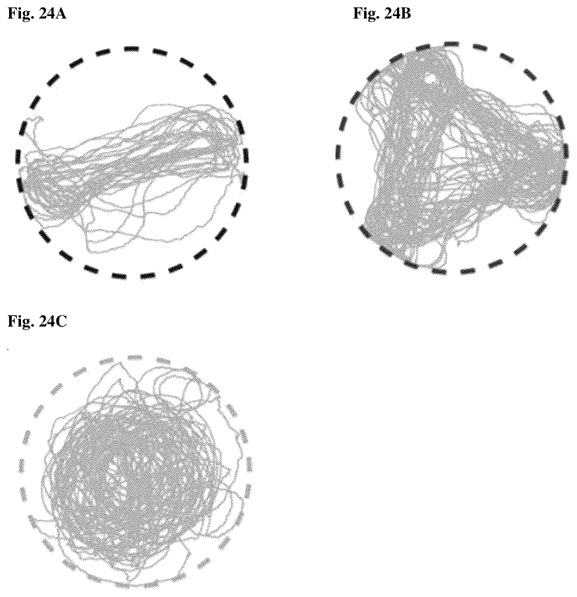

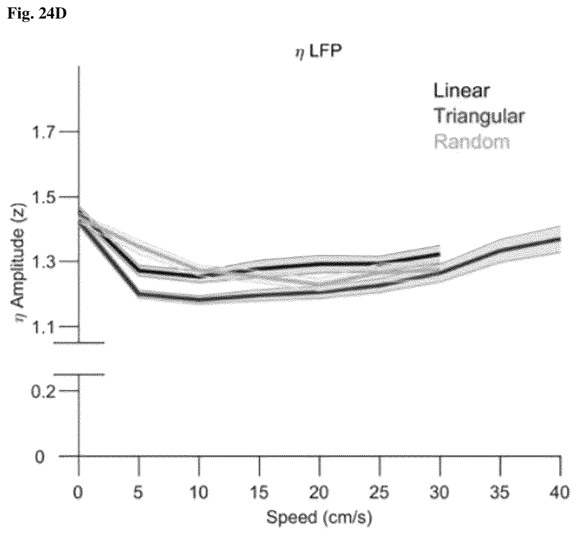

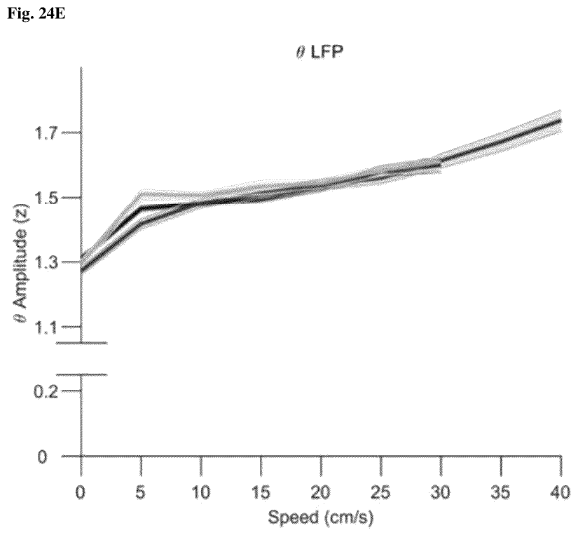

One possible reason for these differences could be that humans were immobile in these VR studies and could make only restricted eye and hand movements, while rats in our studies ran similarly in RW and VR making the full sets of running movements. However, because of the body-fixed condition in VR, the linear acceleration is minimized. Hence, it was hypothesized that running movements of the body, without significant linear acceleration, is sufficient to enhance eta and theta rhythms (FIGS. 24A-24E) and that the presence of linear acceleration in RW makes theta frequency speed- or acceleration-dependent^{2.5}, thereby reducing the overall theta rhythmicity. The enhanced theta rhythmicity in VR, when coupled with low frequency signals, especially via phase-phase coupling could generate stronger eta rhythm in VR. Acceleration-dependence of theta frequency in RW would therefore not only reduce theta rhythmicity but also reduce eta rhythm. This was further supported by our findings that eta-theta phase-phase coupling was much greater in VR than RW, but the eta-theta amplitude coherence was comparably large in both worlds. This can explain why a clear power spectral peak in eta band was seen only in VR, but increased eta band power during run was also seen in a third of electrodes in the RW. Other studies have reported theta skipping in excitatory neurons in certain tasks¹¹. This is probably different than the findings herein because a major difference was not observed in the eta modulation of pyramidal neurons, and eta rhythm was seen at the level of field potential.

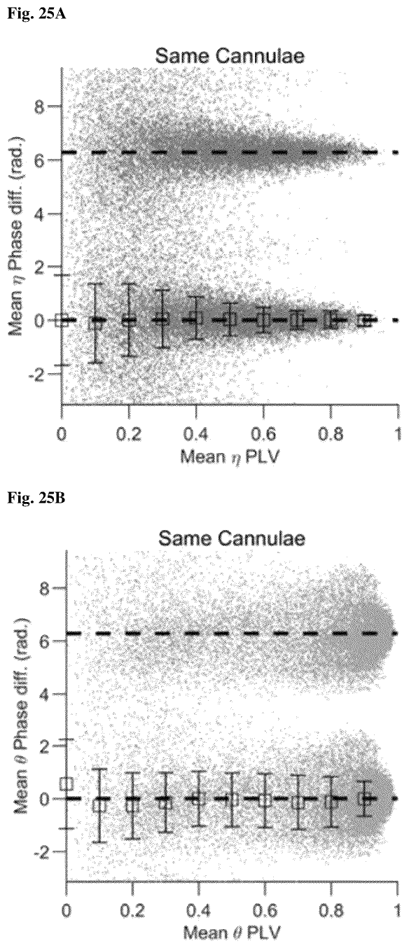

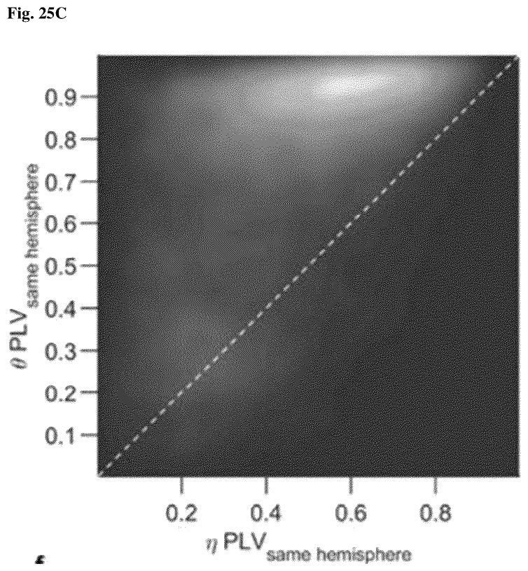

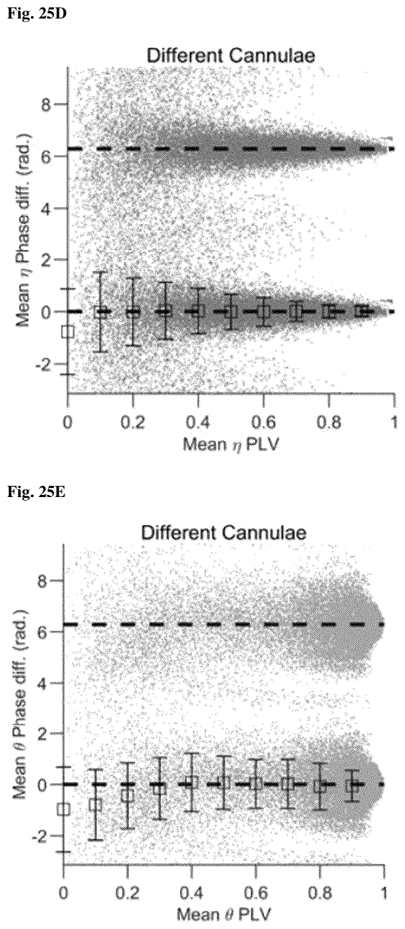

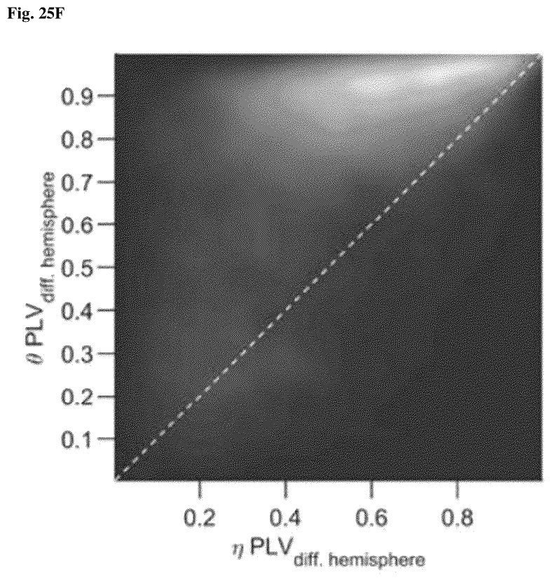

Analysis of eta-theta coherence in these tasks (FIGS. 25a-25f), and investigation of interneurons, similar to the RW data, could be useful. The eta rhythm is unlikely to be related to the respiration related rhythm¹², because it is weaker in the cell layer and stronger below the cell layer, unlike eta rhythm. Eta is not volume conducted signal from other brain areas since it is highest in CA1 cell layer and lower above and below.

It is plausible that eta is generated within the CA1 cell layer by a local network of excitatory-inhibitory neurons. CA1 slices show eta band signals. Accordingly, it is not the pyramidal neurons but the inhibitory interneurons' activity that was differentially modulated by eta in VR compared to RW. This is further supported by several studies demonstrating the role of CA1 interneurons in hippocampal slow oscillations¹³. The reduced theta frequency in VR could arise due to a slowdown of CA1 excitatory-inhibitory network due to the shutdown of a large number of pyramidal neurons¹. Coupled with theta, eta can enhance the rhythmicity and alter the speed dependence of the theta rhythm in VR. This mechanism can be most prominent at the large dendritic branches of the pyramidal cells, where theta is largest. Recent theories suggest that memories are encoded on segments of dendritic branches in pyramidal neurons flanked by inhibitory synapses^{14,15}. This would result in decoupling of the dendritic activity from the soma, as observed recently¹⁶.

The eta oscillations, nearly half as slow as theta, could segregate activity of hippocampal neural populations into parallel streams of information processing throughout theta cycles^{11,17} Eta can enhance hippocampus to cortex interaction, since the 4 Hz rhythm is dominant in neocortex¹⁸.

Eta rhythm and enhanced theta rhythmicity in VR would influence neural synchrony and via NMDAR-dependent synaptic plasticity^{19,20} in dendritic branch specific fashion, to alter hippocampal circuit and learning^{14,15,16}. Impaired hippocampal slow oscillations have been implicated in several cognitive impairments. Virtual reality could be used to enhance hippocampal slow oscillations and neuroplasticity to treat learning and memory impairments.

### REFERENCES

1 Ravassard, P. et al. Multisensory control of hippocampal spatiotemporal selectivity. Science 340, 1342-1346 (2013).
2 Kropff, E., Carmichael, J. E., Moser, E. I. & Moser, M. B. Frequency of theta rhythm is controlled by acceleration, but not speed, in running rats. Neuron 109, 1029-1039 (2021).
3 Buzsiki, G. Hippocampal sharp waves: their origin and significance. Brain Res. 398, 242-252 (1986).
4 Winson, J. Loss of hippocampal theta rhythm results in spatial memory deficit in the rat. Science. 201, 160-163. (1978).
5 Fuhrmann, F. et al. Locomotion, Theta Oscillations, and the Speed-Correlated Firing of Hippocampal Neurons Are Controlled by a Medial Septal Glutamatergic Circuit. Neuron 86, 1253-1264 (2015).
6 Bohbot, V. D., Copara, M. S., Gotman, J. & Ekstrom, A. D. Low-frequency theta oscillations in the human hippocampus during real-world and virtual navigation. Nat Commun. 14415 (2017).
7 Ekstrom, A. D. et al. Human hippocampal theta activity during virtual navigation. Hippocampus. 15, 881 (2005).
8 Jutras, M. J., Fries, P. & Buffalo, E. A. Oscillatory activity in the monkey hippocampus during visual exploration and memory formation. Proc Natl Acad Sci USA. 110, 13144-13149 (2013).
9 Goyal, A. et al. Functionally distinct high and low theta oscillations in the human hippocampus. Nature Communications. 11, 2469 (2020).
10 Aghajan, Z. M. et al. Theta Oscillations in the Human Medial Temporal Lobe during Real-World Ambulatory Movement. Curr Biol. 27, 3743-3751 (2017).
11 Brandon, M. P., Bogaard, A. R., Schultheiss, N. W. & Hasselmo, M. E. Segregation of cortical head direction cell assemblies on alternating θ cycles. Nat Neurosci. 16, 739-748 (2013).
12 Yanovsky, Y., Ciatipis, M., Draguhn, A., Tort, A. B. & Brankack, J. Slow oscillations in the mouse hippocampus entrained by nasal respiration. J Neurosci. 34, 5949-5964. (2014).
13 Jackson, J. et al. Reversal of theta rhythm flow through intact hippocampal circuits. Nat Neurosci 17, 1362-1370. (2014).
14 Mehta, M. R. Cooperative LTP can map memory sequences on dendritic branches. Trends Neurosci. 27, 69-72. (2004).
15 Mehta, M. R. From synaptic plasticity to spatial maps and sequence learning. Hippocampus. 25, 756-762 (2015).
16 Moore, J. J. et al. Dynamics of cortical dendritic membrane potential and spikes in freely behaving rats. Science 355 (2017).
17 Deshmukh, S. S., Yoganarasimha, D., Voicu, H. & Knierim, J. J. Theta modulation in the medial and the lateral entorhinal cortices. J Neurophysiol. 104, 994-1006 (2010).
18 Karalis, N. et al. 4-Hz oscillations synchronize prefrontal-amygdala circuits during fear behavior. Nat Neurosci. 19, 605-612 (2016).
19 Kumar, A. & Mehta, M. R. Frequency-Dependent Changes in NMDAR-Dependent Synaptic Plasticity. Front Comput Neurosci. 5 (2011).
20 Narayanan, R. & Johnston, D. Long-term potentiation in rat hippocampal neurons is accompanied by spatially widespread changes in intrinsic oscillatory dynamics and excitability. Neuron 56, 1061-1075. (2007).

### Materials and Methods

Subjects and surgery: Detailed methods have been described previously¹. Briefly, seven, adult, male, Long-Evans rats (approximately 3.5 months old at the start of training) were implanted with 25-30 g custom-built hyperdrives containing up to 22 independently adjustable tetrodes (13 μm nichrome wires) positioned over both dorsal CA1 areas (−4.0 mm A. P., 2.4 mm M. L. relative to bregma). Surgery was performed under isoflurane. Analgesia was achieved by using Lidocaine (0.5 mg/kg, sc) and Buprenorphine (0.03 mg/kg, ip). Dura mater was removed and the hyperdrive was lowered until the cannulae were 100 μm above the surface of the neocortex. The implant was anchored to the skull with 7-9 skull screws and dental cement. The occipital skull screw was used as ground for electrophysiology. Electrodes were adjusted each day until stable single units were obtained. Positioning of electrodes in CA1 was confirmed through the presence of SPW ripples during immobility.

Virtual reality and real world tasks: The virtual environment consisted of a 220×10 cm linear track floating 1 m above the virtual floor and centered in a 3×3×3 m room^{1,21}. Alternating 5 cm-wide green and blue stripes on the surface of the track provided optic flow. A 30×30 cm white grid on the black floor provided parallax-based depth perception. Distinct distal visual cues covered all 4 walls and provided the only spatially informative stimuli in the VR. In RW, rats ran back and forth on a 220×6 cm linear track that was placed 80 cm above the floor. The track was surrounded by four 3×3 m curtains that extended from floor to ceiling. The same stimuli on the walls in the virtual room were printed on the curtains, thus, the distal visual cues were similar in RW and VR.

Data acquisition, LFP processing, spike detection, sorting and cell classification: Spike and LFP data were collected by 22 independently adjustable tetrodes. Signals from each tetrode were digitized at 32 kHz and wide band pass-filtered between 0.1 Hz and 9 kHz (DigiLynX System, Neuralynx, MT). This was down-sampled to 1.25 kHz to obtain the LFPs, or filtered between 600-6000 Hz for spike detection. LFP positive polarity was downward¹. Unless otherwise stated, the bandpass LFP filtering was done by using a zero-lag forth order Butterworth filter. Spikes were detected offline using a nonlinear energy operator threshold₁. After detection, spike waveforms were extracted, up-sampled fourfold using cubic spline, aligned to their peaks and down-sampled back to 32 data points. PyClust software (a modified version of redishlab.neuroscience.umn.edu/mclust/MClust.html) was used to perform spike sorting²². These were then classified into putative pyramidal neurons and interneurons based on spike waveforms, complex spike index and rates¹. Offline analyses were performed using custom code written in MATLAB (MathWorks).

Analysis of LFP and spike data during behavior: Running epochs were defined as continuous periods of running (>10 cm/s) for 2 sec. or more. Immobility was defined as periods of low speed (<2.5 cm/s) for 2 sec. or more. Hence, the low speed range, which excludes periods of immobility, was taken as a range from 5 to 15 cm/sec. In addition, correlation coefficients of the amplitudes of the different frequency bands with speeds were computed below and above 10 cm/sec to capture dynamics during transition periods from the rest to run and running epochs. Spectral analysis of oscillatory activity was computed using a multitaper method^{23,24} by Chronux toolbox (chronux.org). The window size of 4 sec. (average running time during the task) and 3-5 tapers were used with a 75% overlap over frequencies ranging from 0.5 to 30 Hz. The spectral power was computed separately during running epochs and immobility states.

The power spectral index was computed as the difference of power between running epochs and immobility at each frequency over their sum (FIGS. 5H, 14B, 6A-6N, and 7A-7C). Significance of the power difference between running epochs and immobility in the theta and eta bands was determined using Kruskal-Wallis nonparametric test (u=0.01). To reduce nonspecific effects, the power spectra of each tetrode were normalized by average power in 0.5-30 Hz range on that tetrode separately for the running epochs and immobility states. Theta and eta power peaks were detected using peak prominence of 0.01 or more within the respective frequency bands (findpeaks.m from signal toolbox in MATLAB). The prominence was defined as the height of the peaks at the levels of highest troughs (Mathworks). With few exceptions this led to the detection of eta peaks predominantly during running epochs in VR. The prominence of eta index peaks greater than 5 percentile of the theta index peaks was considered as significant. Peak power was computed as an average power within 1 Hz at the detected peak.

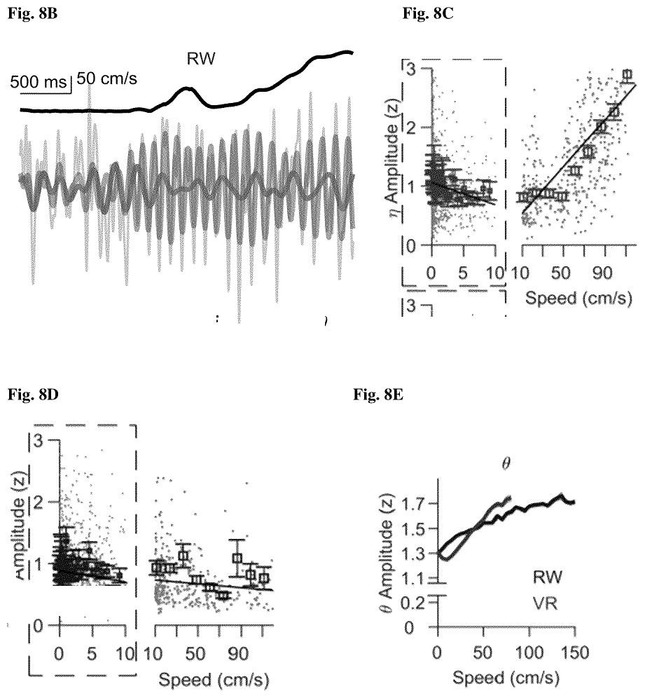

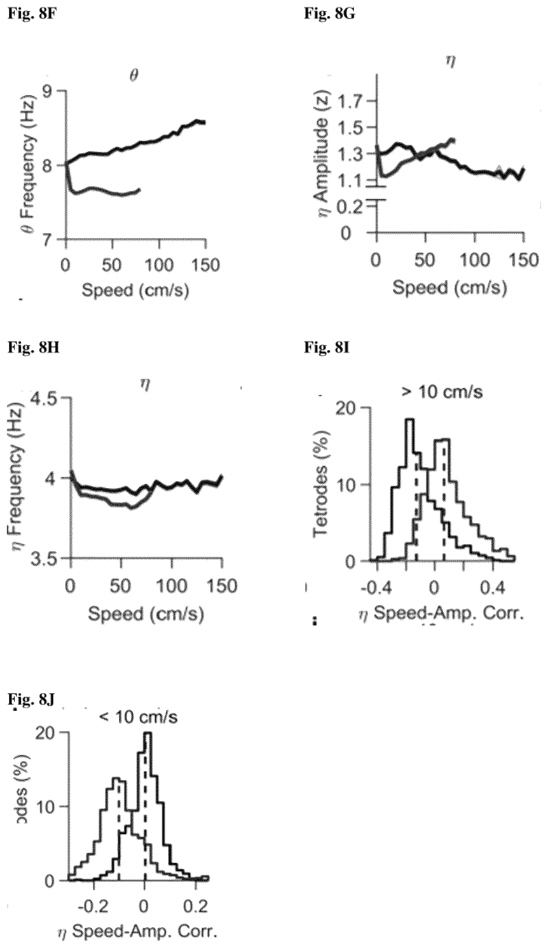

This power spectrum based method requires comparatively long periods of unitary behavior (e.g. run or stop) over which the power spectra are computed. To obtain an estimate of the instantaneous values of eta and theta bands, the LFP data was filtered in either eta (2.5-5.5 Hz) or theta (6-10 Hz) ranges and computed its Hilbert transform. Amplitude difference index was computed as the difference of the mean amplitude in theta (or eta) band during high (30-60 cm/s) and low (5-15 cm/s) speed runs, divided by their sum (FIGS. 5C, 5D). Significance level of theta (or eta) modulation of LFP was determined by comparing the distributions of the LFP amplitude in theta (or eta) band during high (30-60 cm/s) versus low (5-15 cm/s) speed runs, and using a nonparametric Kruskal-Wallis test. Alternative, non-parametric estimate was also done by computing robust regression fits between amplitude envelope and speed. Theta frequency was computed using three methods: cycle detection using Hilbert transformed phase jumps, the derivative of Hilbert transform phase, and the short time Fourier transform. The cycle method results are reported (FIGS. 8A-8I).

Sharp wave and ripple detection: To estimate the electrode depth an SPW ripple analysis was performed during periods of immobility in baseline sessions preceding the tasks. LFP data was filtered in ripple (80-250 Hz) band. This signal was z-scored by subtracting the mean value and dividing by the standard deviation of the ripple band LFP to obtain the z-scored ripple band signal. Double-threshold crossing method was applied to the LFP z-scores^{25,26} to detect ripple events. All time points with larger than a first threshold (z>3) were identified as part of a ripple event. Only events with a peak value larger than a second threshold (z>10) and duration larger than 30 ms were retained (FIG. 14C). Ripple events separated by less than 50 ms were stitched together. To detect the concomitant SPW, LFP were filtered in 6-25 Hz range. These signals were z-scored and amplitude detected at the times of each associated ripple peak. These SPW were averaged across all the ripples in a session to obtain the average SPW (FIG. 14C).

Place field detection: A unit was considered track (goal) active if its mean firing rate on track (goal) was at least 1 Hz. Opposite directions of the track were treated as independent and linearized. A place field was defined as a region where the firing rate exceeded 5 Hz for at least 5 cm. The boundaries of a place field were defined as the point where the firing rate first drops below 5% of the peak rate (within the place field) for at least 5 cm, and exhibits significant activity on at least five trials¹.

Phase locking detection and characterization: Instantaneous amplitudes and phases were estimated by Hilbert transform of the filtered signals as below:

LFP(t)=ρ(t)e^{-iφ(t)}

where ρ(t) is instantaneous amplitude and φ(t) is instantaneous phase.

Rayleigh circular uniformity test was computed to test significance of phase locking. The first circular moment was given as:

$$m ′ = ( \frac{1}{n} ) {\sum }_{j = 1}^{n} {e}^{i {φ}_{j}} = \overline{R} {e}^{i μ} ,$$

where φⱼ are phases of n spikes. The preferred LFP phase of spikes is thus given by μ and the magnitude of phase locking was given by R—. The magnitude of phase locking was defined as depth of modulation (DoM)²⁷. The Rayleigh statistics Z=R⁻²n (n>50, only neurons with at least 50 spikes were used), and the probability of the null hypothesis of sample uniformity (P=e^{-Z}) was applied^{28,29}.

Spike autocorrelogram (ACG), Gaussian mixture model (GMM) fit and theta rhythmicity index (TR) calculation: Spike-time autocorrelograms were computed using accuracy of 1 ms, smoothed by 20 ms Gaussian function, and normalized by the number of spikes to obtain probability at lags. Autocorrelograms Y(t) were fit using the following Gaussian mixture mode1^{11,30,31}:

$$Y ( t ) = [ ( a \times {e}^{\frac{- t}{{τ}_{i}}} ) {\sum }_{n = 1}^{N} ( \frac{1}{\sqrt{2 π} σ} {e}^{\frac{- {( t - n \times w )}^{2}}{2 \times {a}^{2}}} ) + b ] {e}^{\frac{- t}{{τ}_{2}}}$$

where t is the autocorrelation lag time (ranging from 60-600 ms) and a, τ₁, w, σ, b, τ₂ are the fit parameters. The Gaussian terms are used to fit theta peak and its harmonics (w is a first theta lag). τ₁ and τ₂ are the exponential decay constants for the magnitude of rhythmicity and overall ACG falloff rate due to finite amount of data, respectively. a is the rhythmicity factor and b is constant background or non-rhythmic component. N=5 five Gaussians n were used to fit the ACG. The amplitude of the first Gaussian (n=1) provides an estimate of theta modulation, while removing nonspecific effects arising from the duration of the place field and the duration of recording. Theta rhythmicity was defined as TR(n)=(amplitude (n+1)−amplitude (n))/max(amplitude (n), amplitude (n+1)) where n is a ACG peak amplitude at theta or its harmonics and varies from 1 to 3. Theta skipping^{11,17} was defined as TR with n=1.

Statistics: Unless otherwise stated, statistical significance between two distributions of linear variables was evaluated using nonparametric Kruskal-Wallis test. Tests for populations significantly different from zero were also performed using the nonparametric Kruskal-Wallis test. Average values are reported in the form mean±s.e.m. unless otherwise stated. Median values of histograms are depicted as a dashed line in all main figures. Circular statistics were computed using the CircStat toolbox. Binomial confidence interval was computed using the Clopper-Pearson method from statistics toolbox in MATLAB (binofit.m). Data distributions were assumed to be normal, but this was not formally tested. To reduce the contribution of outliers, unless otherwise stated we used nonparametric Spearman's rank correlation to compute all correlation coefficients including partial correlations. No statistical methods were used to pre-determine sample size in these exploratory studies, but our sample sizes are similar to those reported in previous publications,^{1,11,13,21}. Neural and behavioral data analyses were conducted in an identical way regardless of the identity of the experimental condition from which the data were collected, with the investigator blinded to group allocation during data collection and/or analysis. Hippocampal units were isolated and cluster by three different lab members blindly.

The experiments were conducted by three different lab members. Similar sessions in real world and virtual reality were run overall rats, which were selected randomly. Covariates were controlled across sessions and within rats. No rat was excluded. The presence of the sharp wave ripples was used to identify hippocampal tetrodes. In addition, classified putative pyramidal cells were used to verify selection. Both methods were widely used to verify hippocampal tetrodes.

The software used for data acquisition and analysis are available using the web links mentioned in the Methods. PyClust is a modified version of redishlab.neuroscience.umn.edu/mclust/MClust.html.

### REFERENCES

21 Cushman, J. D. et al. Multisensory Control of Multimodal Behavior: Do the Legs Know What the Tongue Is Doing? PLoS One 8 (2013).
22 Willers, B. Multimodal sensory contributions to hippocampal spatiotemporal selectivity. Doctoral Dissertation, University of California Los Angeles (2013).
23 Mitra, P. P. & Pesaran, B. Analysis of dynamic brain imaging data. Biophys J. 76, 691-708 (1999).
24 Bokil, H., Andrews, P., Kulkarni, J. E., Mehta, S. & Mitra, P. P. Chronux: a platform for analyzing neural signals. J Neurosci Methods. 192, 146-151 (2010).
25 Ego-Stengel, V. & Wilson, M. A. Disruption of ripple-associated hippocampal activity during rest impairs spatial learning in the rat. Hippocampus 20, 1-10 (2010).
26 Ji, D. & Wilson, M. A. Coordinated memory replay in the visual cortex and hippocampus during sleep. Nat Neurosci. 10, 100 (2007).
27 Skaggs, W. E., McNaughton, B. L., Wilson, M. A. & Barnes, C. A. Theta phase precession in hippocampal neuronal populations and the compression of temporal sequences. Hippocampus 6, 149-172 (1996).
28 Sirota, A. et al. Entrainment of neocortical neurons and gamma oscillations by hippocampal theta rhythm. Neuron. 60, 683-697 (2008).
29 Siapas, A. G., Lubenov, E. V. & Wilson, M. A. Prefrontal phase locking to hippocampal theta oscillations. Neuron. 46, 141-151 (2005).
30 Royer, S., Sirota, A., Patel, J. & Buzsiki, G. Distinct representations and theta dynamics in dorsal and ventral hippocampus. J Neurosci. 30, 1777-1787 (2010).
31 Climer, J. R., DiTullio, R., Newman, E. L., Hasselmo, M. E. & Eden, U. T. Examination of rhythmicity of extracellularly recorded neurons in the entorhinal cortex. Hippocampus. 25, 460-473 (2015).

### Example 2: Moving Bar of Light Generates Angle, Distance and Direction Selectivity in Place Cells

Primary visual cortical neurons selectively respond to the position and motion direction of specific stimuli retrospectively, without any locomotion or task demand. At the other end of the visual circuit is the hippocampus, where in addition to visual cues, self-motion cues and task demand are thought to be crucial to generate selectivity to allocentric space in rodents that is abstract and prospective. In primates, however, hippocampal neurons encode object-place association without any locomotion requirement. To bridge these disparities, rodent hippocampal responses to a vertical bar of light were measured in a body-fixed rat, independent of behavior and rewards. When the bar revolved around the rat at a fixed distance, more than 70% of dorsal CA1 neurons showed stable modulation of activity as a function of the bar's angular position, while nearly 40% showed canonical angular tuning, in a body-centric coordinate frame, termed Stimulus Angle Cells or Coding (SAC). The angular position of the oriented bar could be decoded from only a few hundred neurons' activity. Nearly a third of SAC were also tuned to the direction of revolution of the bar and most of these responses were retrospective. SAC were invariant with respect to the pattern, color, speed and predictability of movement of the bar. When the bar moved towards and away from the rat at a fixed angle, neurons encoded its distance and direction of movement, with more neurons preferring approaching motion. Thus, a majority of neurons in the hippocampus, a multisensory region several synapses away from the primary visual cortex, encode non-abstract information about stimulus-angle, distance and direction of movement, in a manner similar to the visual cortex, without any locomotion, reward or memory demand. These responses may influence the cortico-hippocampal circuit and form the basis for generating abstract and prospective representations.

Sensory cortical neurons generate selective responses to specific stimuli, in the egocentric (e.g. retinotopic) coordinate frame, without any locomotion, memory or rewards¹. In contrast, the hippocampus is thought to contain an abstract, allocentric cognitive map, supported by spatially selective place cells², grid cells³ and head direction cells⁴. Such robust hippocampal responses are thought to require both distal visual cues⁵ and self-motion cues^{6,7}, e.g. via path integration⁸, which requires specific sets of self-movements. In addition, the angular and linear optic flow generated by locomotion could contribute to hippocampal activity, but this has not been directly tested. Recent studies have shown significant modulation of hippocampal activity by an auditory⁹⁻¹² or a social stimulus^{13,14}. These tasks required either specific actions, rewards or memory to generate selectivity. The stimulus related hippocampal activity modulation reduced to chance level when task demand and stimulus locked rewards were omitted^{9-11,15} In particular, no study has investigated if hippocampal neurons can encode the angular position and direction of movement of a visual stimulus without bodily movements; it is commonly thought that such compass information requires locomotion^{8,16,17}. To understand hippocampal function, it is necessary to know if place cells encode information about the angular position and motion direction of a specific moving visual stimulus, like sensory cortices, regardless of movement, memory or reward.

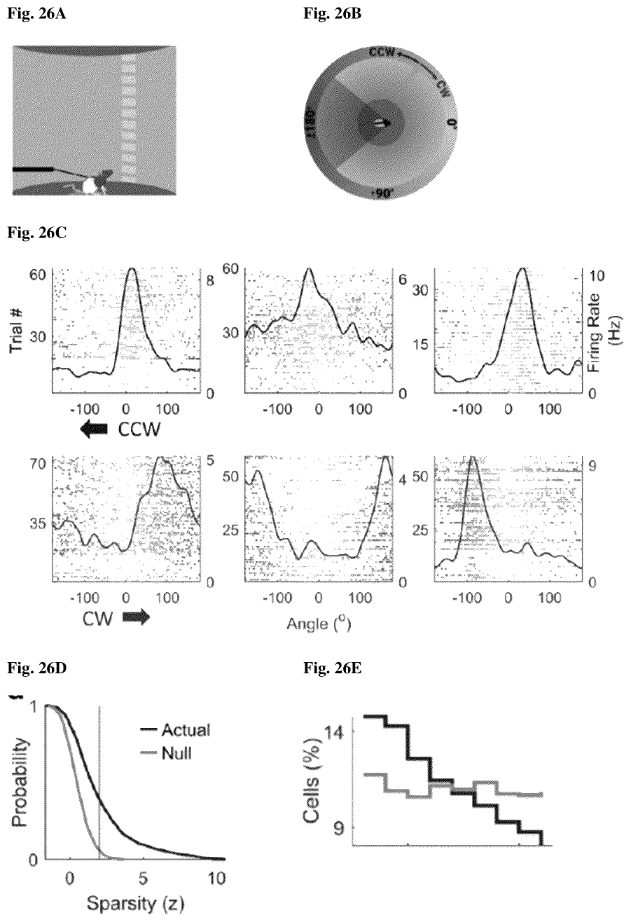

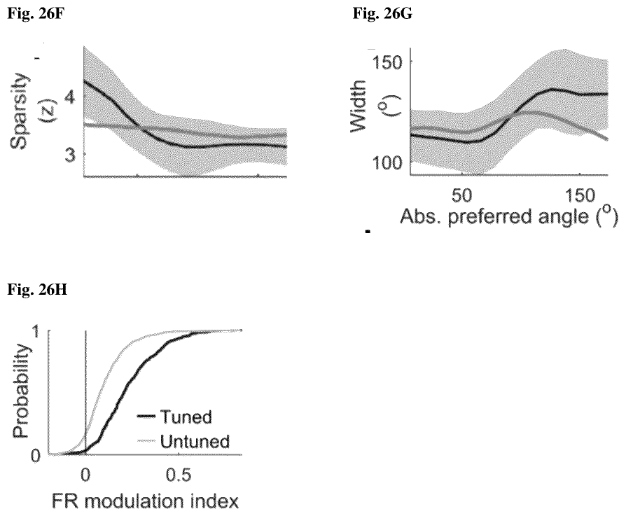

To address these questions, rats were gently held in place on a large spherical treadmill, surrounded by a cylindrical screen¹⁸. They were free to move their heads around the body, but not fully turn their body. They were given random rewards to keep them motivated, similar to typical place cell (e.g. random foraging) experiments. The only salient visual stimulus was a vertical bar of light, 74 cm tall, 7.5 cm wide, 33 cm away from the rat, thus subtending a 13° solid angle. In the first set of experiments, the bar revolved around the rat at a constant speed (36°/s), without any change in shape or size (FIGS. 26A, 26B), independent of rat's behavior or reward delivery. The bar's revolution direction switched between CW (clockwise) and CCW (counter-clockwise) every four revolutions. In subsequent experiments, when the identity, movement and trajectory of the bar was varied, selective responses were found in all cases.

Stimulus Angle Coding (SAC) in Large Fraction of CA1 Neurons

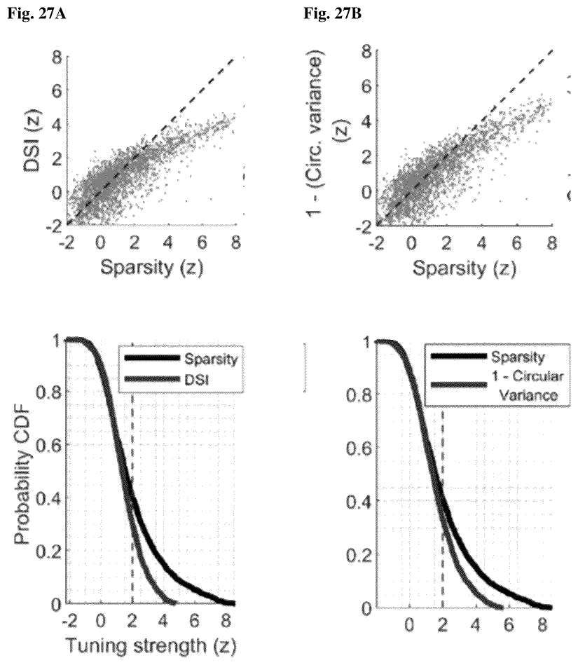

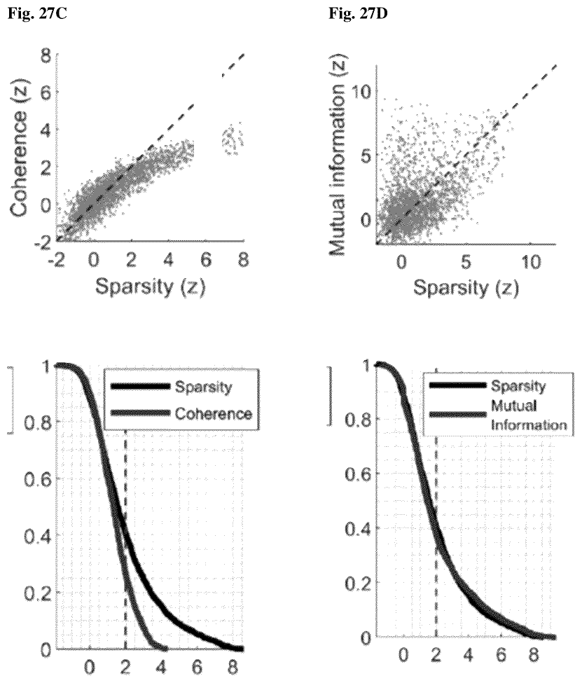

The activity was measured for 1191 putative pyramidal neurons (with firing rate above 0.2 Hz during the experiment) from the dorsal CA1 of 8 Long Evans rats in 149 sessions using tetrodes (see methods¹⁹). Many neurons showed clear modulation of firing rate as a function of the bar position (FIG. 26C), with substantial increase in firing rates in a limited region of visual angles, referred to herein as stimulus angle coding (SAC) or stimulus angle cells. Across the ensemble of neurons, 464 (39%) showed significant (sparsity (z)>2, corresponding to p<0.023, see methods, see FIGS. 27A-27D for other metrics) stimulus angle tuning in either the CW or CCW direction (FIG. 26D).

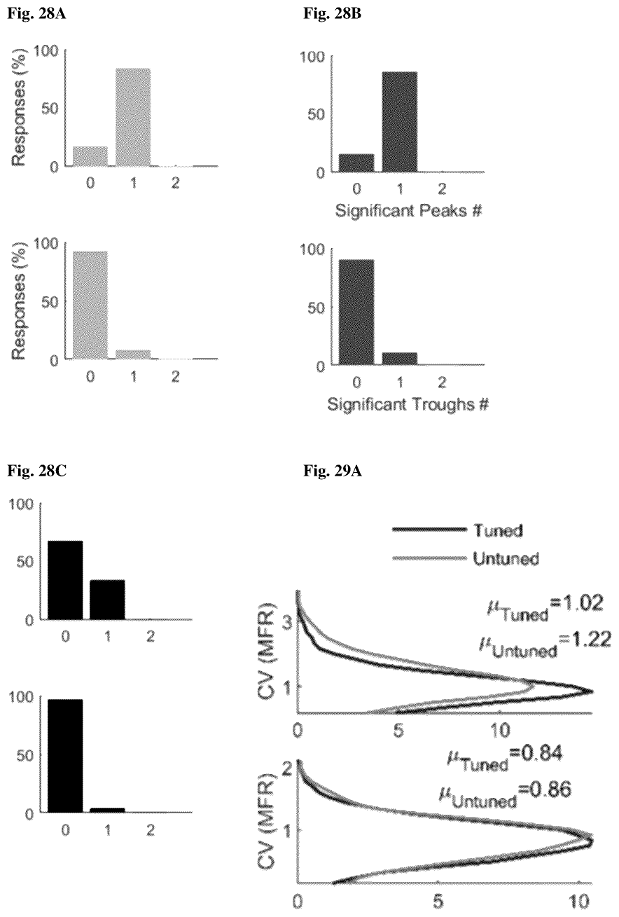

Like the primary visual cortical responses and hippocampal place cells, most tuning curves were unimodal (FIGS. 28A-28C) with a single preferred angle where the firing rate was the highest. But, virtually no neurons showed an off response (a significant decrease in firing rate). The preferred angles spanned the entire range, including angles behind the rat (FIG. 26E). These responses resembled striate cortical neurons in many ways^{1,20}. More neurons encoded the positions in front of the rat (0°) and there was a gradual, two-fold decline in the number of tuned cells for angles behind (±180°). The strength of SAC (FIG. 26F, see methods) was much larger near 0° compared to 180°. The width of the tuning curves also increased gradually as a function of the absolute preferred angle from 0° to 180° (114° vs 144° FIG. 26G), and was quite variable at every angle, spanning on average about a third of the visual field, similar to place cells on linear tracks^{21,22}.

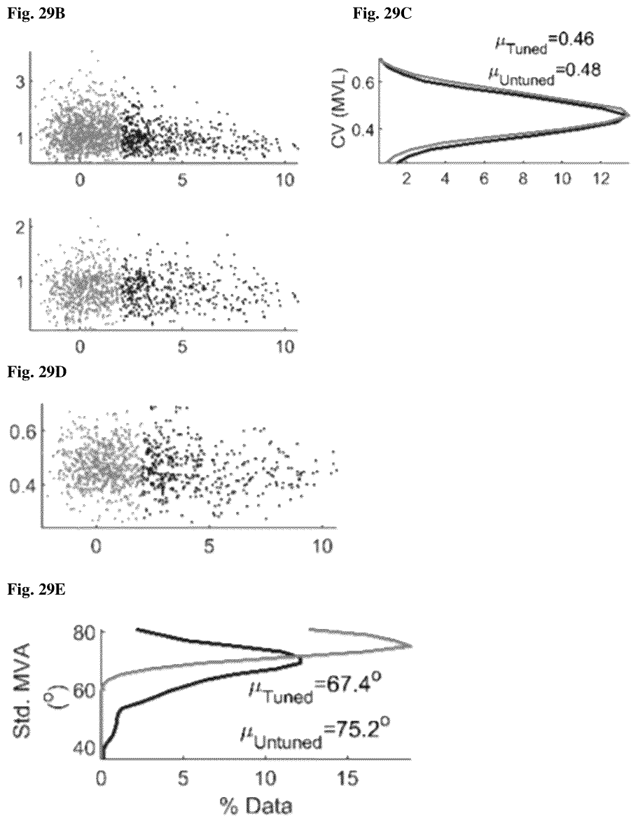

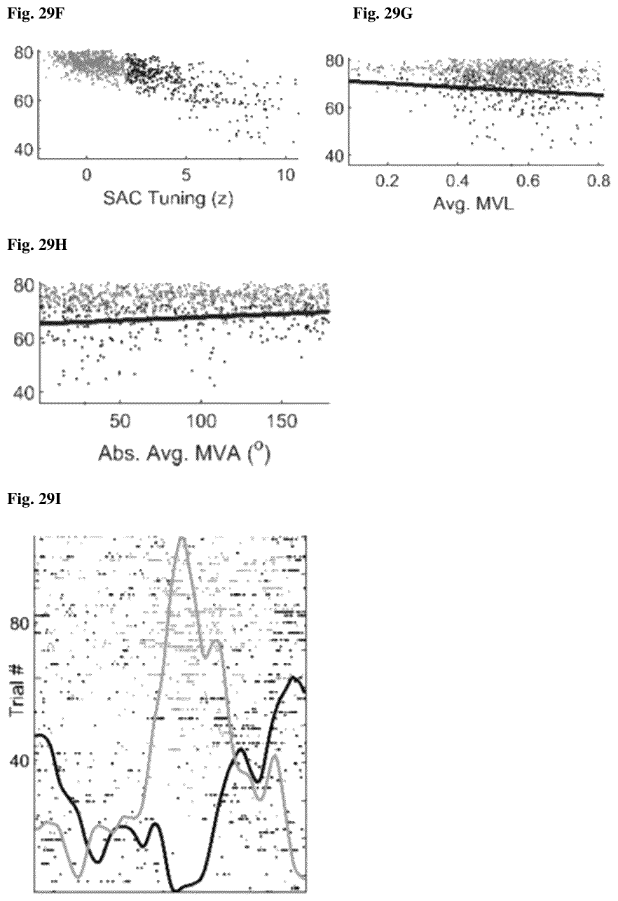

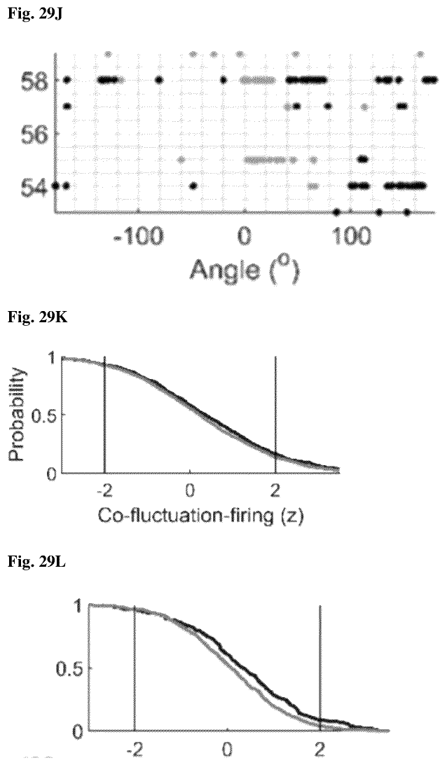

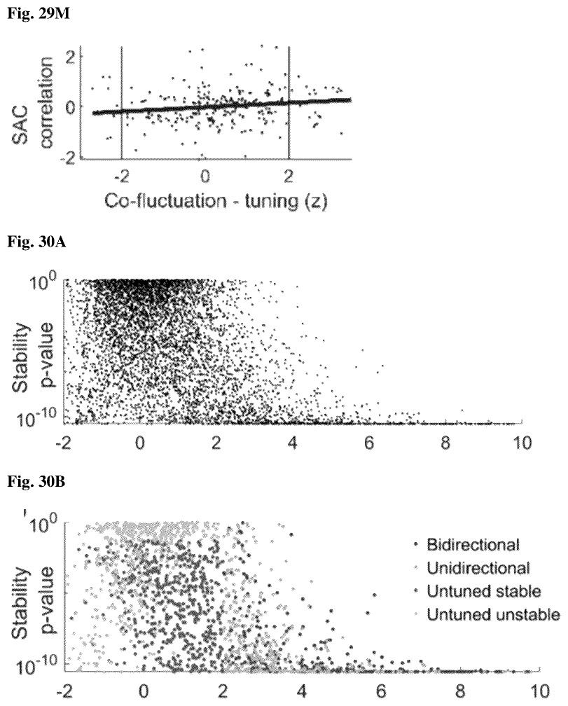

Hippocampal place cells on 1D tracks have high firing rates within the field and virtually no spiking outside²¹. In contrast, the firing rates of SAC were often nonzero outside the preferred angle of SAC, as evidenced by modest values of the firing rate modulation index (FIG. 26H, see methods). On the other hand, these broad SAC tuning curves resembled the directional tuning of CA1 neurons recently reported in the real world and virtual reality¹⁷, with comparable fraction of neurons showing significant angular tuning. SAC trial to trial variability was quite large, but comparable to recent experiments in visual cortex of mice under similar conditions²³. Notably, the variability in the mean firing rate across trials was small and unrelated to the degree of angular tuning. However, the trial-trial variability of the preferred angle was quite large and predictive of the degree of SAC of a neuron (FIGS. 29A-29H).

Revolution Direction Selectivity of SAC

In the primary visual cortex, majority of neurons respond selectively to the angular position of the oriented bar, regardless of its movement direction, and a minority of neurons are sensitive to the movement direction of the barn. But, a majority of hippocampal neurons on linear tracks are highly directional^{19,21}. Further, in both areas, if a neuron is active in both directions, then it shows significant and stable modulation in both directions.

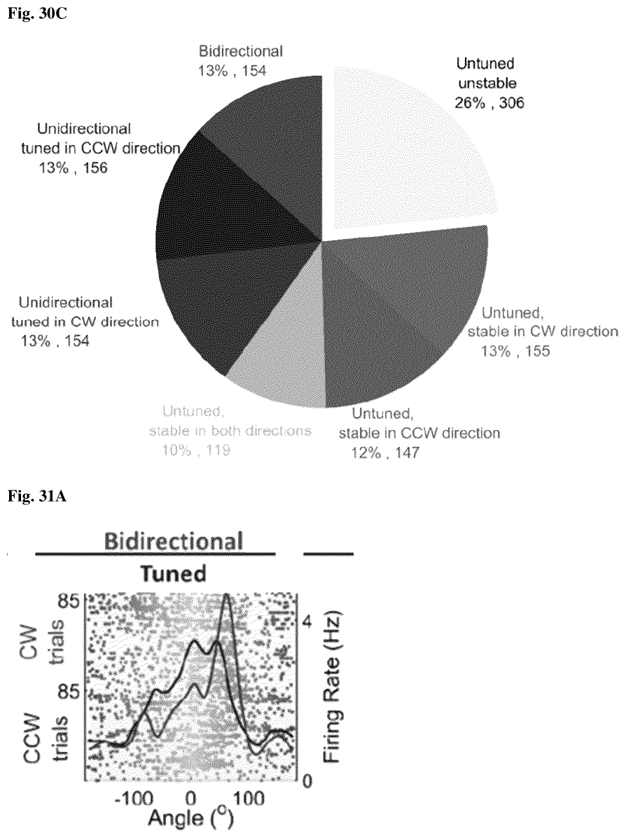

To bridge these discrepancies, the selectivity, directionality and stability (see methods) of the SAC were inspected. The degree of tuning varied continuously across neurons with no clear boundary between tuned and untuned neurons (FIGS. 30A-30C).

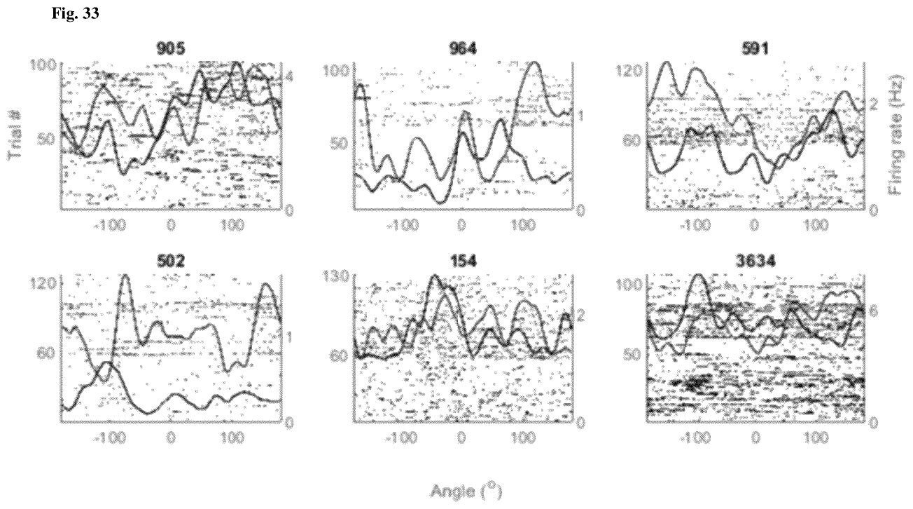

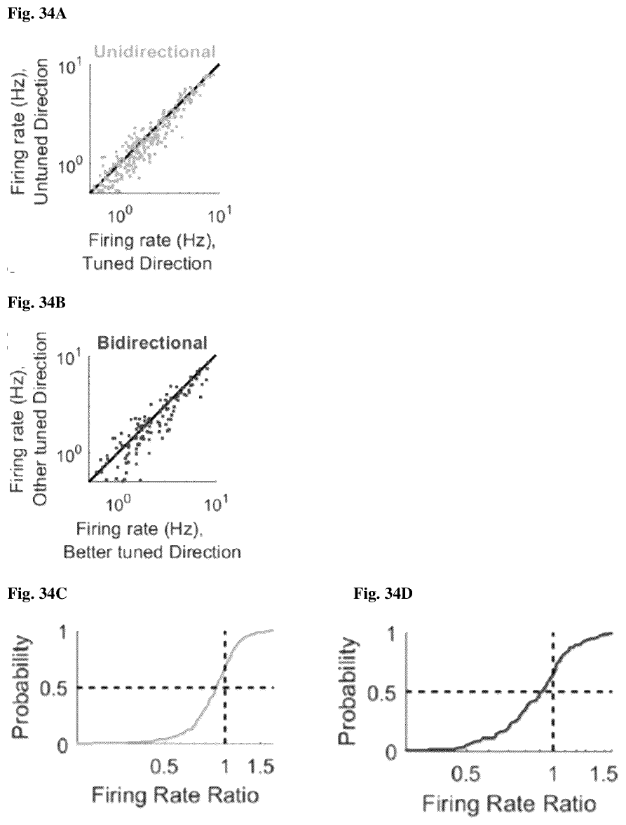

To examine the tuning properties across this population the neurons were separated according to their degree of tuning in the two movement directions, as commonly done₁₅. Some neurons were bidirectional, with significant (z>2) SAC in both movement directions (FIGS. 31A and 32). However, a larger subset of neurons was unidirectional, with significant (z>2) angle selectivity in only one movement direction (FIGS. 32B and 32). For the tuned direction, SAC were stable, showing consistent firing rate modulation as a function of angle across trials. Surprisingly, there were many untuned-stable neurons (see methods), which showed consistent, significantly stable spiking across trials, but the SAC, quantified by z-scored sparsity, was not significantly different than chance (FIGS. 31C and 33). Across the ensemble, about 13% (154) of neurons were bidirectional, 26% (310) were unidirectional, and 35% (421) were untuned-stable (FIGS. 31D and 33). Thus, the vast majority (74%, 885) of hippocampal pyramidal neurons were consistently influenced by the angular position and direction of the revolving bar. However, unlike visual cortex, far more SAC neurons were unidirectional, and unlike hippocampal place cells and visual cortex, far greater number of neurons showed untuned but stable responses. The majority of tuned neurons had their preferred angle around 0°, i.e. in front of the rat (FIG. 31E) and this bias was greater for the bidirectional cells.

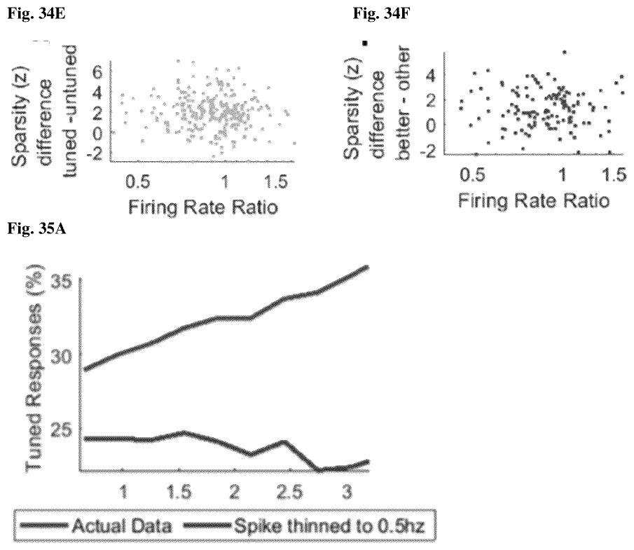

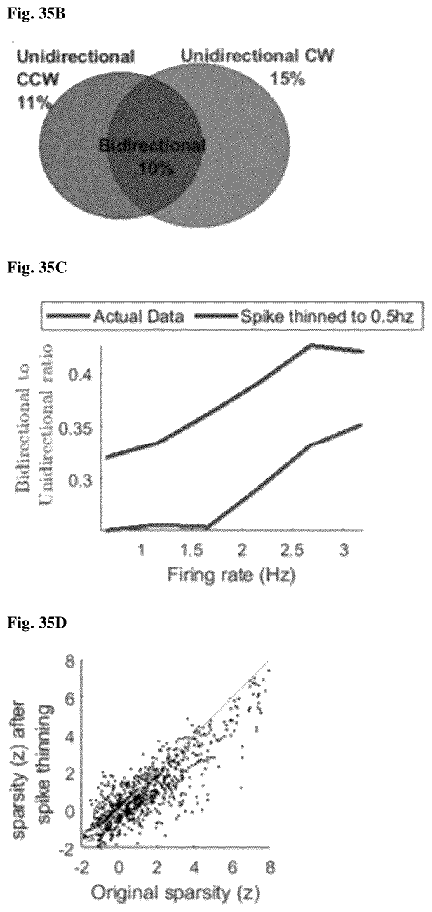

The differences in firing rates and tuning properties were then examined between the two movement directions. For both the unidirectional and bidirectional cells, the firing rate was substantially different between the two directions of movement (FIGS. 34A-34F). Further, the mean firing rates of neurons was invariably larger in the direction in which the stimulus angle tuning was greater, compared to the less tuned, or untuned, direction (FIGS. 34A-34F). This disparity in firing rates between the tuned and untuned directions arose largely from the increase in firing rate within the preferred zone (±90° around the preferred angle) in the tuned direction (FIG. 31G). Higher rate cells were more likely to be bidirectional than unidirectional, even when the contribution of firing rates differences to strength of tuning were factored out (FIGS. 35A-35D). Finally, the tuning curves in the CW and CCW directions were significantly correlated for bidirectional cells (FIG. 31F). This was true, although to a smaller extent, for unidirectional cells and untuned-stable cells, but not for the untuned unstable cells.

Population Vector Decoding of SAC

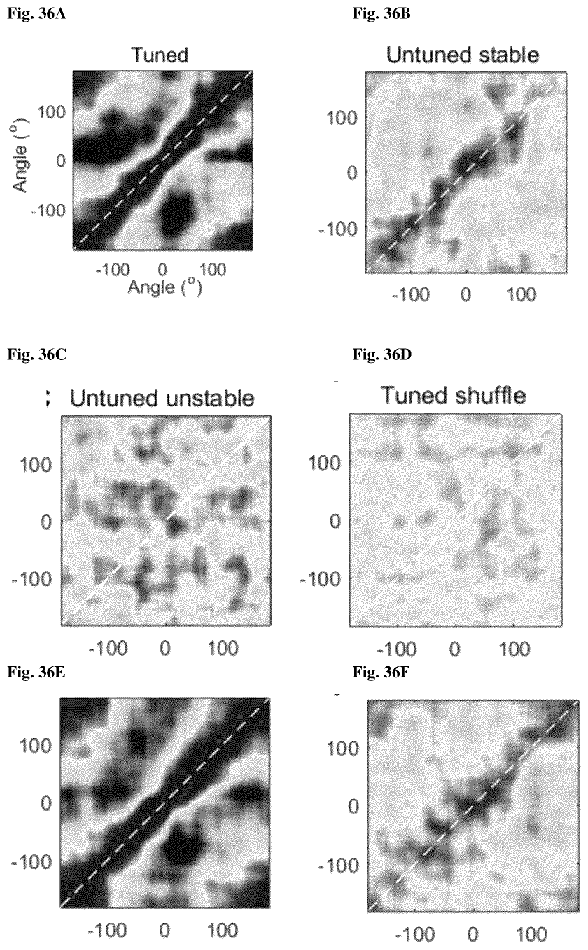

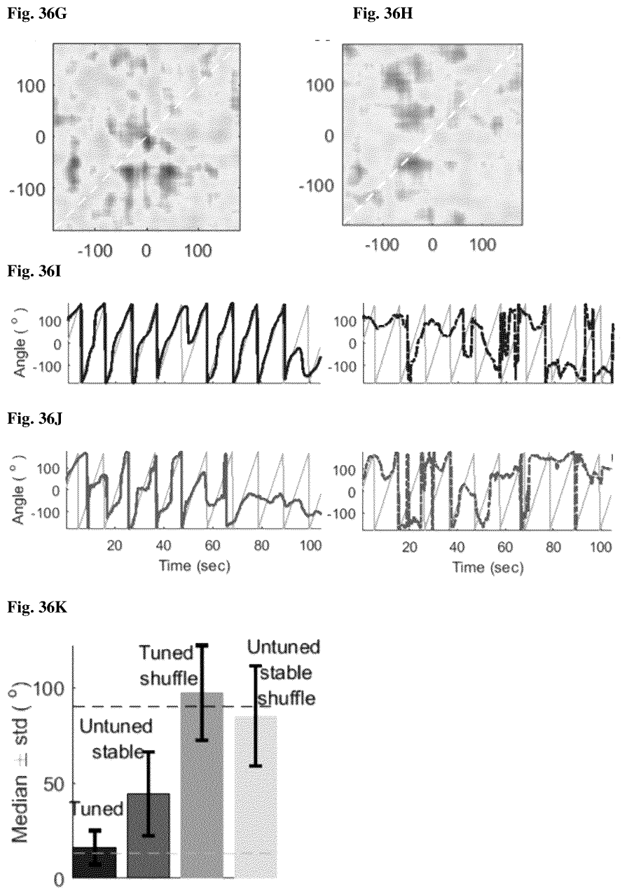

In addition to individual cells showing stable stimulus angle encoding, the population responses were also coherent for tuned and untuned-stable populations (FIGS. 36A-36K, see methods). Ensemble of a few hundred place cells is sufficient to decode the rat's position using population vector decoding²⁴. Using similar methods, the position of the bar was decoded using different ensembles of SAC (see methods).

The ensemble of 310 tuned cells (CCW), with a short temporal window of 250 ms, could decode the position of the oriented bar with a median accuracy of 17.6° (FIGS. 31H, 31J) comparable to the bar width (13°). This is qualitatively similar to the spatial decoding accuracy of place cells^{24,25}. Additionally, the 266 untuned but stable cells could also decode the position of the bar significantly better than chance, but the median error was 45.2° (FIGS. 31I, 31J) which is larger than that for the tuned cells. The unstable cells did not contain significant information about the bar position. Decoding performance improved when using a larger number of tuned or untuned stable cells, but not when using more unstable responses (FIG. 31K). Thus the ensemble of untuned stable cells contained significant SAC information, even though these individual cells did not²⁶. This was not the case for the untuned unstable cells.

Most Neurons Show Retrospective SAC

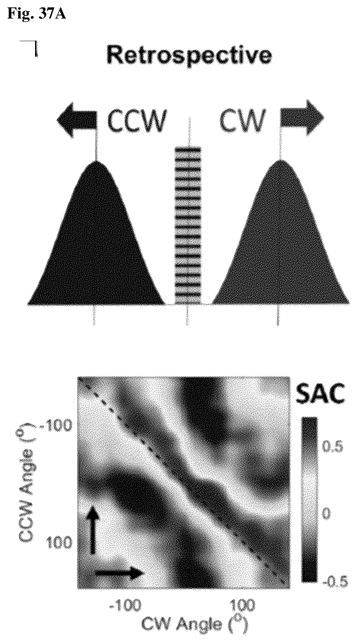

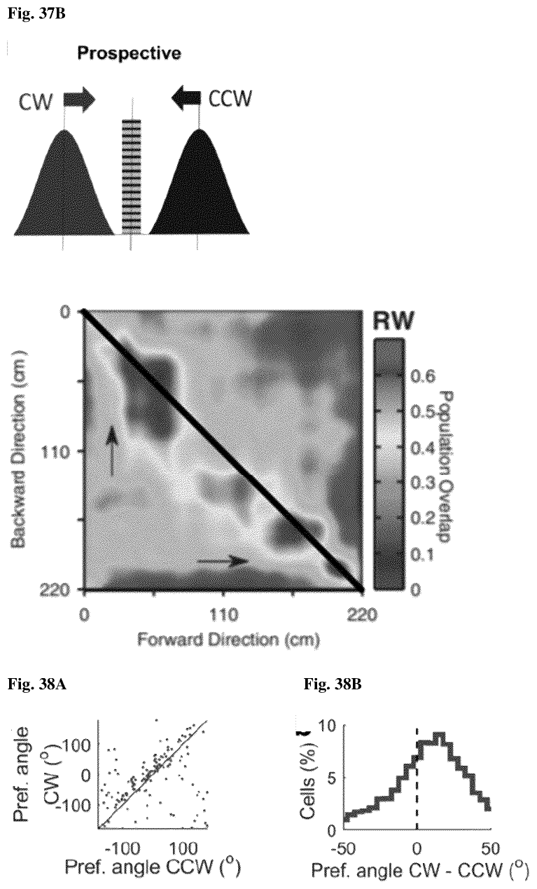

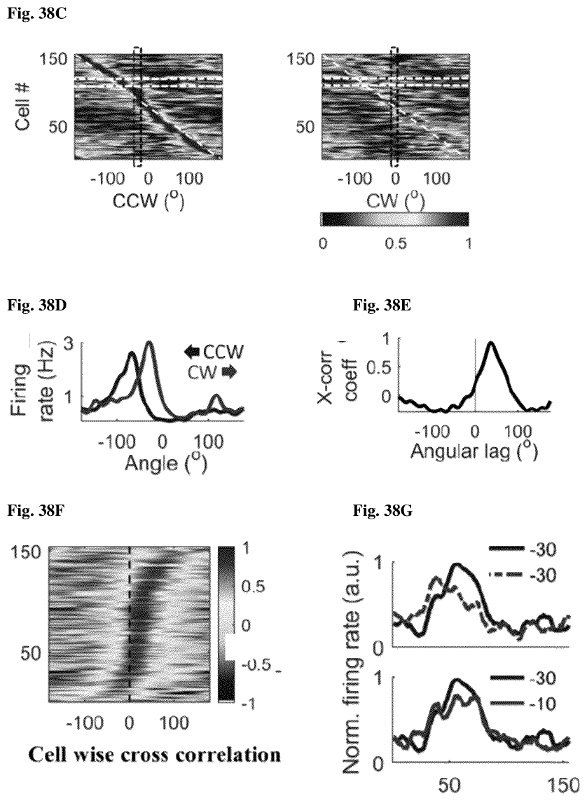

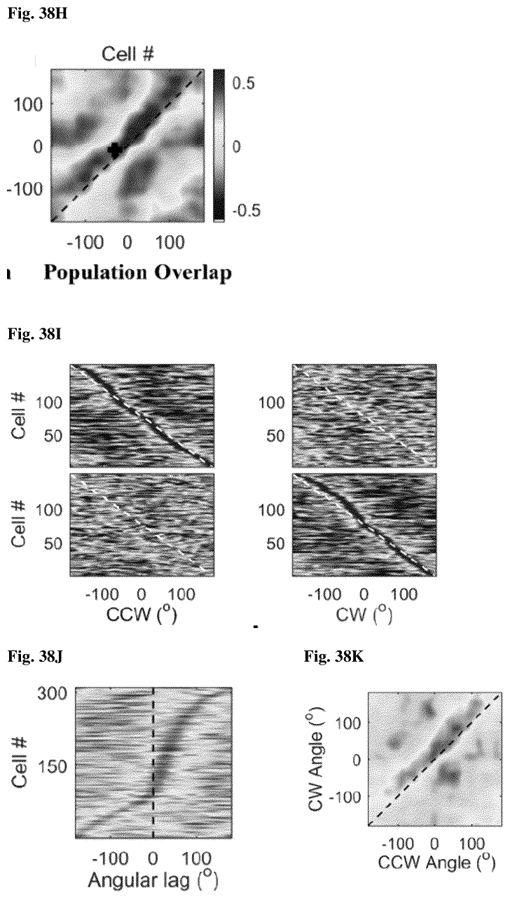

Under most conditions, visual cortical neurons respond to the stimulus with a short latency, i.e., retrospectively, whereas most hippocampal bidirectional cells on linear tracks are prospective, i.e., they fire before the rat approaches the place field from the opposite movement directions^{19,25,27} However, the converse was observed for these hippocampal bidirectional SAC (FIGS. 37A, 37B). Here (example cell, FIG. 38D), the preferred angle in the CCW direction lagged behind that in the CW direction, i.e., in both directions the neuron responded to the bar after it had gone past a specific angle, which is a retrospective response. Hence, the circular difference was computed between the preferred angle between the CW and CCW directions (bidirectional population response, FIGS. 38A, 38B), which were predominantly positive. Are only the peaks of SAC retrospective or do the entire tuning curves have lagged responses? To address this, the cross correlation was computed between the entire tuning curves between the CW and CCW directions. A majority (80%) of neurons showed maximum correlation at positive latency. Thus, most neurons responded to the oriented bar retrospectively, i.e., with a lag. The median latency to response was 276.2 ms (leading to 19.9° median shift in cross correlation FIG. 38F). This retrospective coding was evident across the entire ensemble of bidirectional cells, such that the population vector overlap between the CW and CCW directions was highest at values slightly shifted from the diagonal (FIG. 38H, see methods).

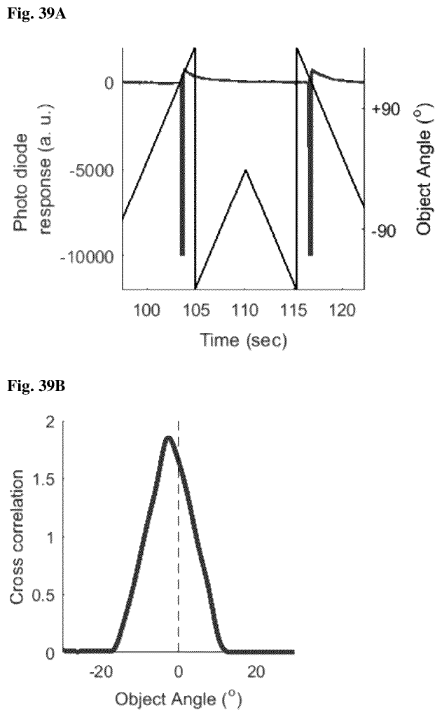

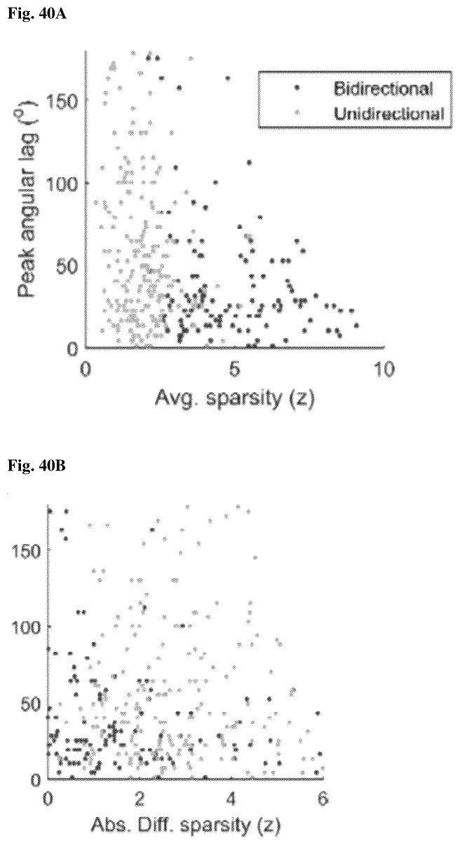

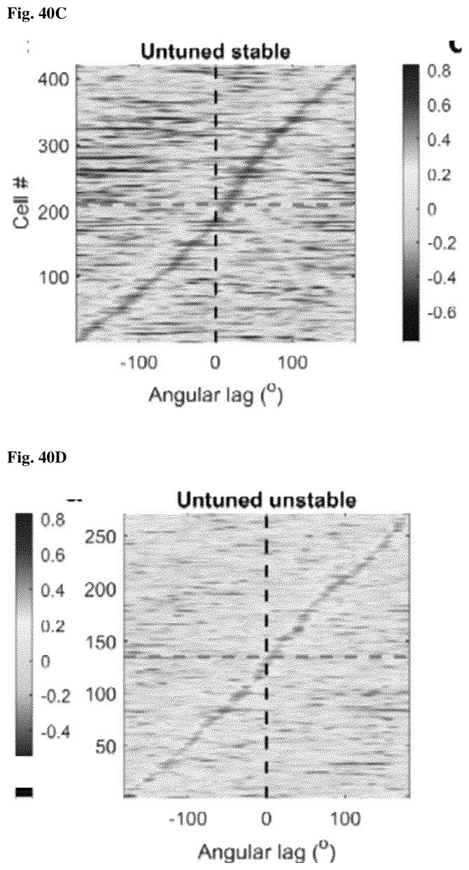

Additional experiments using a photodiode showed that this lag could not be explained by the latencies in the recording equipment (FIGS. 39A, 39B, equipment latency of 38.9 ms was removed from all numbers reported herein). In fact, retrospective tuning was found even for the unidirectional cells, even though the tuning was not significant in one of the revolution directions, resulting in weaker correlations (FIGS. 38I-38K). The range of latencies was larger for the unidirectional cells than bidirectional cells (FIGS. 38F, 38J), but median latency in cross correlations (19.9°, or 276.2 ms temporal latency of response) was comparable to bidirectional cells. Thus, the retrospective coding does not arise due to difference in tuning strengths. The larger range of latencies and weaker correlations for unidirectional cells could arise because significant tuning is present in only one direction. Small but significant temporal bias was observed in the untuned-stable cells but not for the unstable cells (FIGS. 40A-40D).

Invariance of SAC Tuning

When the distal visual cues are changed by even a small amount, hippocampal CA1 neurons show remapping, i.e., large changes in place cells' firing rate, degree of spatial selectivity, and the preferred location or receptive field^{28,29}. On the other hand, primate hippocampal neurons show selectivity to a combination of object identity and its retinotopic position³⁰.

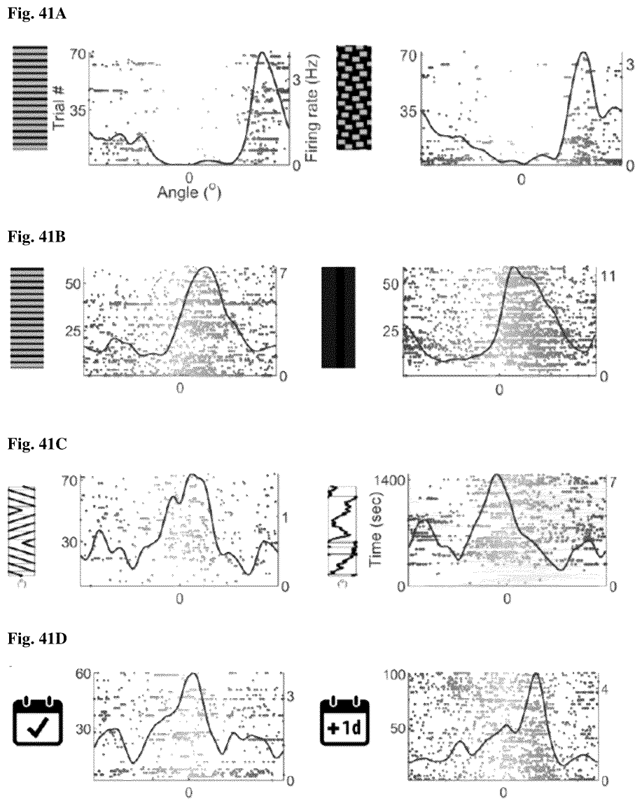

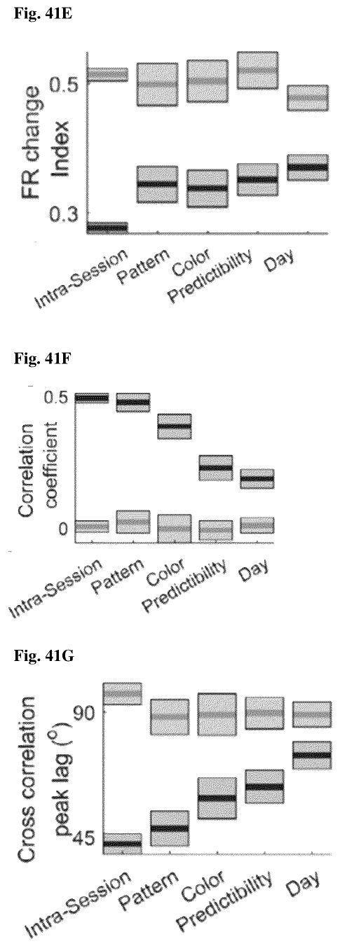

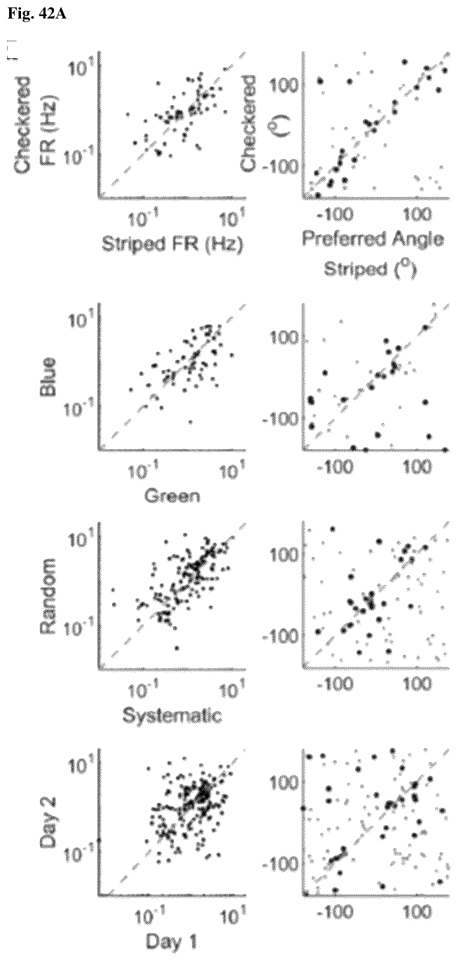

To address this in a systematic fashion, the responses of the same set of neurons were recorded, on the same day, to bars of light with different visual features (see methods) without any other changes in stimuli or behavior. In one experiment, the stimulus was changed minimally (green-black stripes vs green-black checkered pattern, FIGS. 4a, e-g). Neural firing rates, strength of SAC, preferred tuning location and tuning curve profiles were largely invariant and comparable to spontaneous fluctuations (FIG. 41A, see methods). To further test this invariance, we changed the vertical bar substantially by changing both color and pattern (green-black horizontal stripes vs blue with one vertical black line, FIG. 41B). This resulted in significantly more changes in all measures of SAC, though this too was far less than expected by chance. Thus, unlike complete remapping with change in visual cues, SAC was invariant to substantial changes in visual cues.

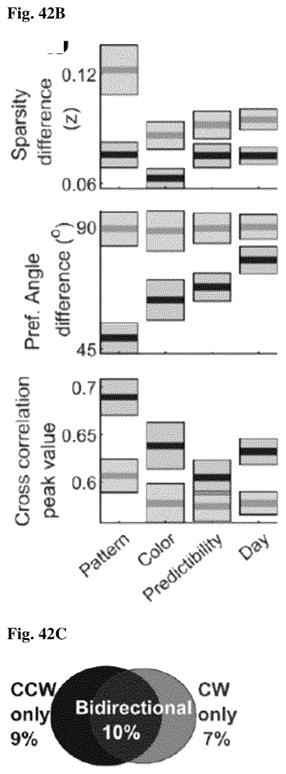

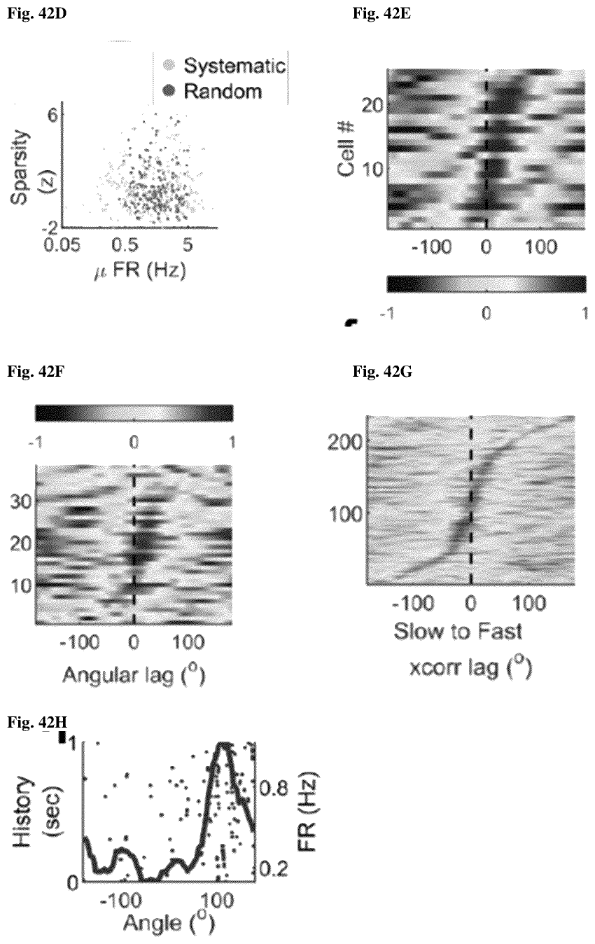

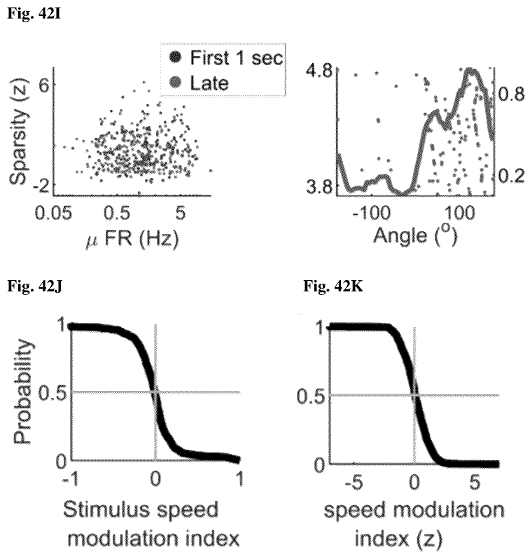

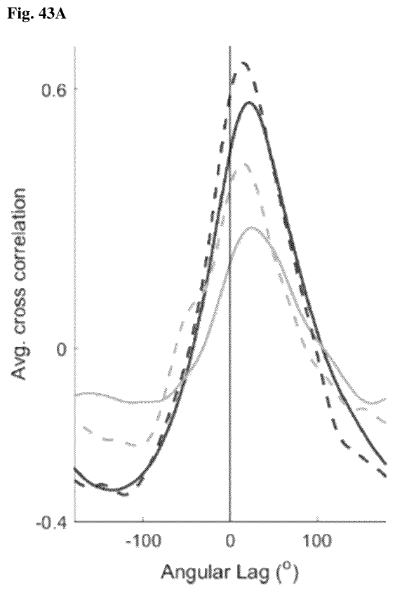

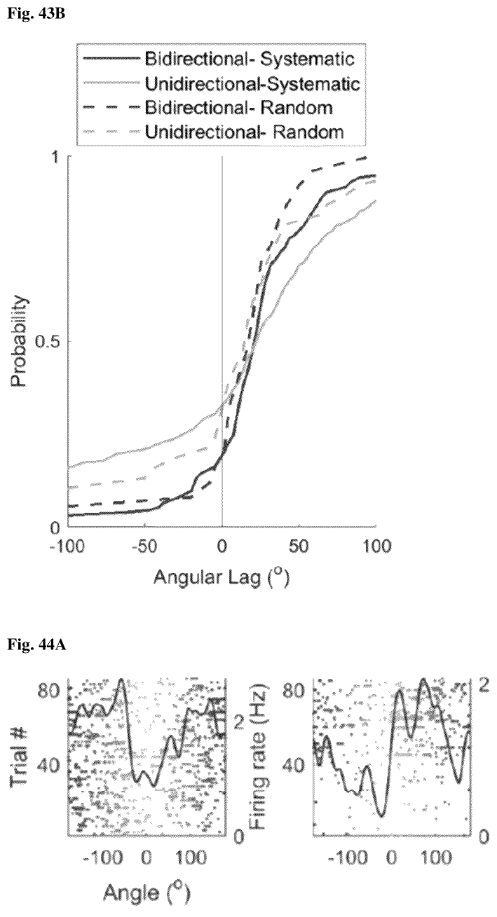

Sequential tasks can influence neural selectivity in the hippocampus^{7,31} and visual cortex³². Hippocampal neurons also show selectivity in sequential, non-spatial tasks^{11,13,14} and sequential versus random goal-directed paths induce place field remapping³³. Hence, the above experiments did not include any systematic behavior or rewards related to the moving bar. To compute the contribution of the sequential movement of the bar of light to SAC, experiments were performed where the movement of the vertical bar was less predictable. The bar moved only 56.7° in one direction on average, and then abruptly changed speed and direction, referred to herein as the randomly moving bar paradigm (FIG. 41C). 26% of neurons showed significant SAC, which was far greater than chance, though lesser than the systematic condition (FIGS. 42A-42K). The other results were qualitatively similar to systematically moving bar of light, including the percentage of unidirectional, bidirectional and untuned-stable cells. (FIGS. 42A-42K). Thus, the SAC cannot arise entirely from sequential movement of the bar, and the retrospective latencies were unaffected (FIGS. 43A, 43B) by systematic or random motion of the bar. To directly ascertain the effect of predictability on SAC, the randomly moving bar data in the first 1-second after stimulus direction flip was separately analyzed from an equivalent subsample of data from later when the stimulus had moved in the current direction for at least 3 seconds (FIGS. 42A-42K, see methods). SAC was similar in these two conditions. Further, SAC were not systematically biased by the angular movement speed of stimulus, nor did hippocampal firing encode stimulus speed beyond chance (FIGS. 42A-42K).

Recent studies have reported spontaneous, slow remapping of place cells over several days³⁴. The activity of the same cells was measured for more than one day, and measured changes in SAC without any changes in stimuli or their predictability. There was substantial remapping across two days, evidenced by very low correlation between the tuning curves of the same neuron across two days (FIG. 41D). This was not due to difference in novelty, because rats had experienced this stimulus for at least one week.

There was a consistent pattern of remapping across these experiments, as measured by the correlation coefficient of the tuning curves (FIG. 41F). The smallest change in tuning curves occurred with the smallest change in stimulus, i.e., pattern change. Greater change with change in color, even greater change with alteration in stimulus predictability and the largest change occurred spontaneously across two days. This occurred due two mechanisms. First, the preferred tuning angle rotated across different conditions, with the lowest amount of change for pattern change, larger for color, followed by predictability and time. Second, even when this change in the preferred tuning angle was factored out, similar pattern of changes in correlations persisted (FIGS. 42A-42K).

Overlapping Neural Populations Encode Stimulus Angle, Distance and Spatial Position

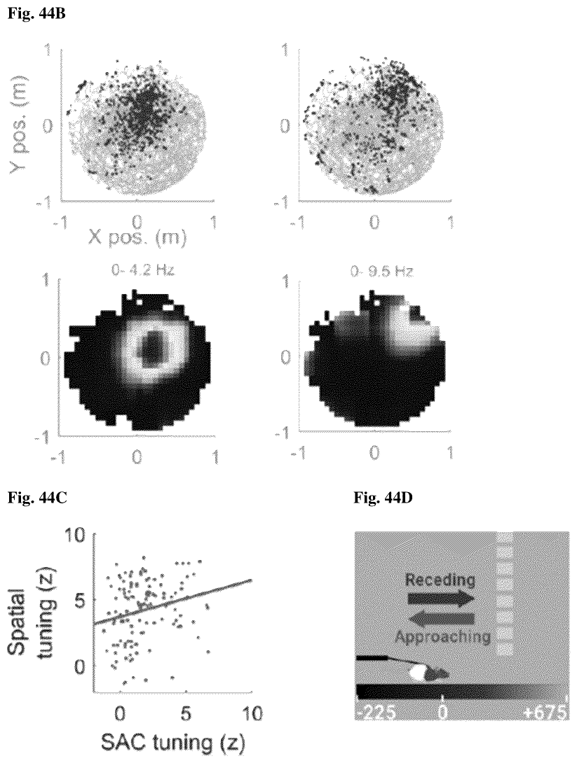

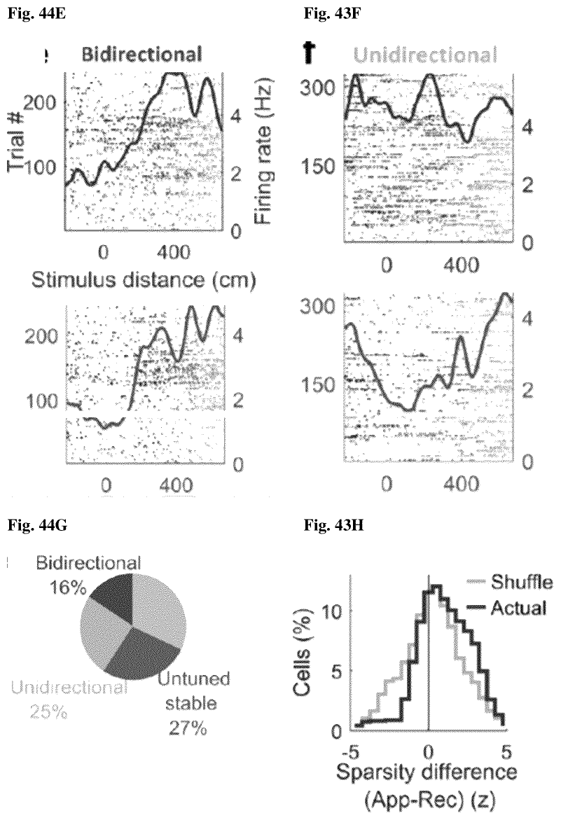

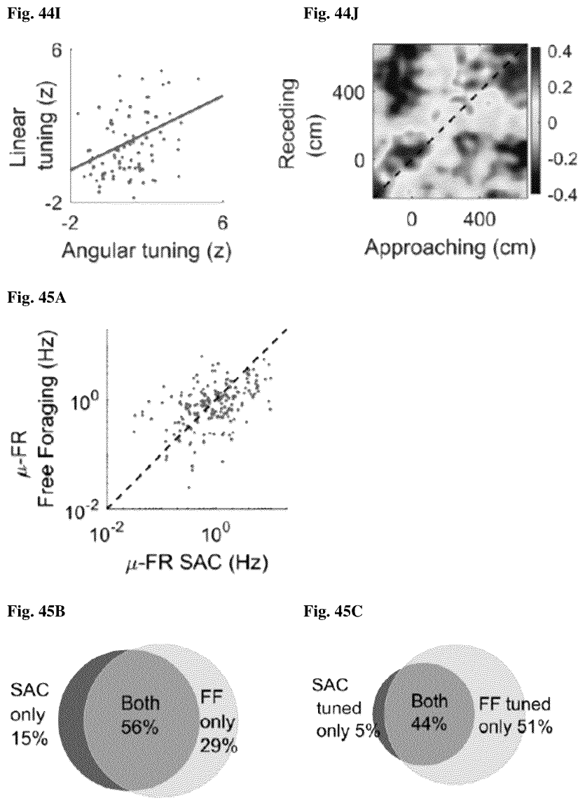

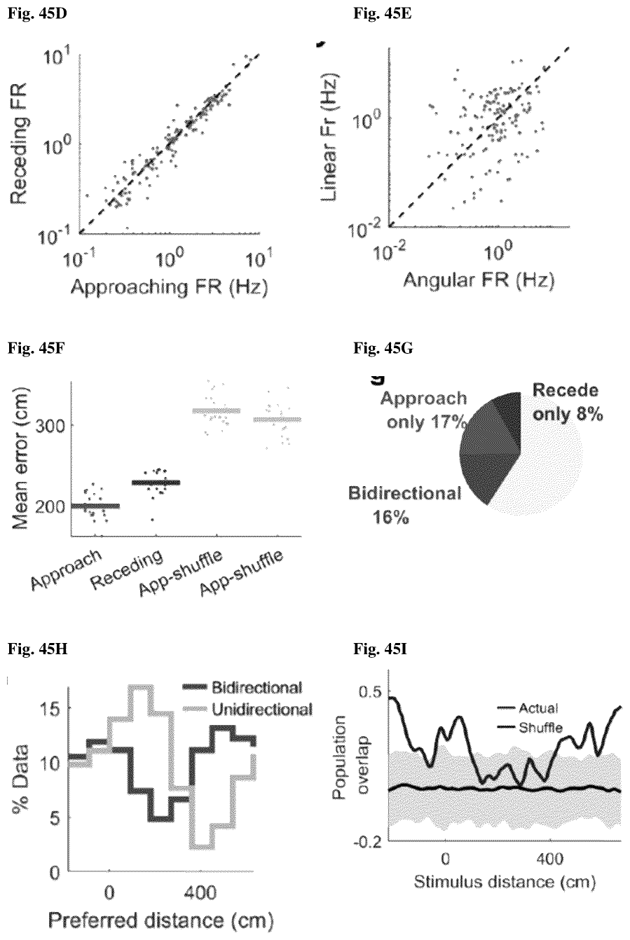

During spatial exploration majority of rodent hippocampal neurons show spatially selective responses, aka “place cells.” What is the relationship between SAC vs spatial selectivity of neurons? In additional experiments, the activity of the same set of CA1 neurons was measured, on the same day, during the SAC protocol and while rats freely foraged for randomly scattered rewards in two-dimensional environments (FIG. 44A, see methods). Out of 341 pyramidal cells, 56% were active in both experiments, whereas 29% and 15% were active only during exploration or during SAC respectively. Firing rates during exploration and SAC experiments were strongly correlated (FIGS. 45A-45I). Of the population of cells active in both experiments, 44% showed significant tuning to both spatial position and stimulus angle, whereas 51% showed significant tuning to only space. The strength of tuning was significantly correlated between these two experiments (FIG. 44C). Thus, the majority of SAC cells were also place cells.

Spatial exploration involves not only angular optic flow but looming signals too. Hence, 147 place cells were measured where the stimulus moved towards or away from a body-fixed rat, completing one lap in 10 seconds, without any change in angular position (illustration—FIG. 44D, example cells—FIGS. 44E, 44F). The firing rates of 41% of neurons showed significant modulation as a function of the stimulus distance (FIG. 44G) and 27% of cells had untuned but stable responses. Neurons not only encoded distance but also direction of movement, with 17% and 8% of neurons showing significant tuning to only the approaching (coming closer) or receding (moving away) bar of light, respectively. Neural firing rates were very similar for approaching and receding stimuli, but stimulus distance coding was much stronger for approaching movements (FIG. 44H). For matched cells recorded in both stimulus distance and angular experiments (see methods), firing rates (FIGS. 45A-45I) as well as the strength of tuning were correlated, suggesting that the same population of neurons can encode both distance and angle (FIG. 44I). The preferred distance (i.e., the position of maximal firing) for the bidirectional cells, was not uniform but bimodal, with majority of neurons active near the rat (0 cm) or farthest away (500 cm), and very few neurons representing the intermediate distances (FIGS. 45A-45I). Retrospective response was also seen in these experiments, with the population overlap between approaching and receding responses shifted to values above the diagonal (FIGS. 44J, FIGS. 45A-45I) corresponding to a retrospective shift of 70.6 cm or 196.1 ms.

### Discussion

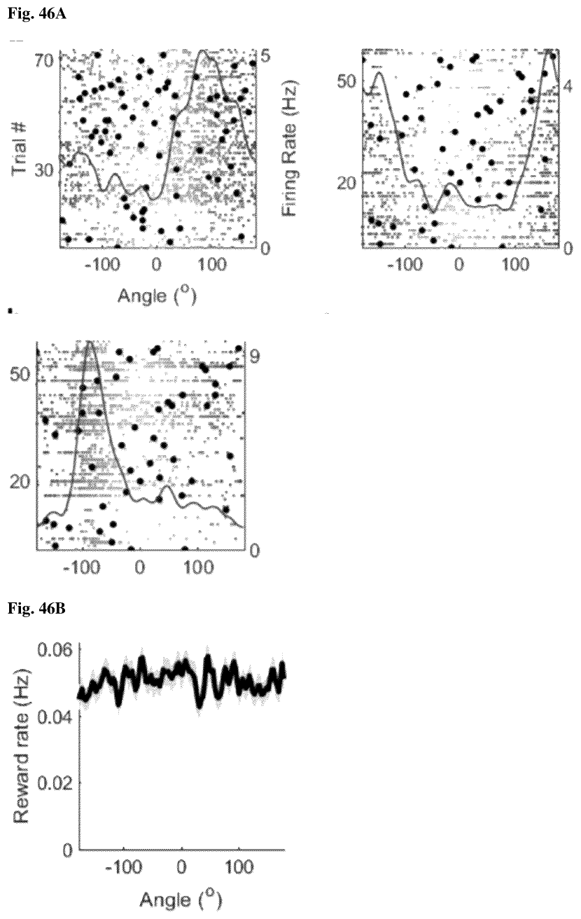

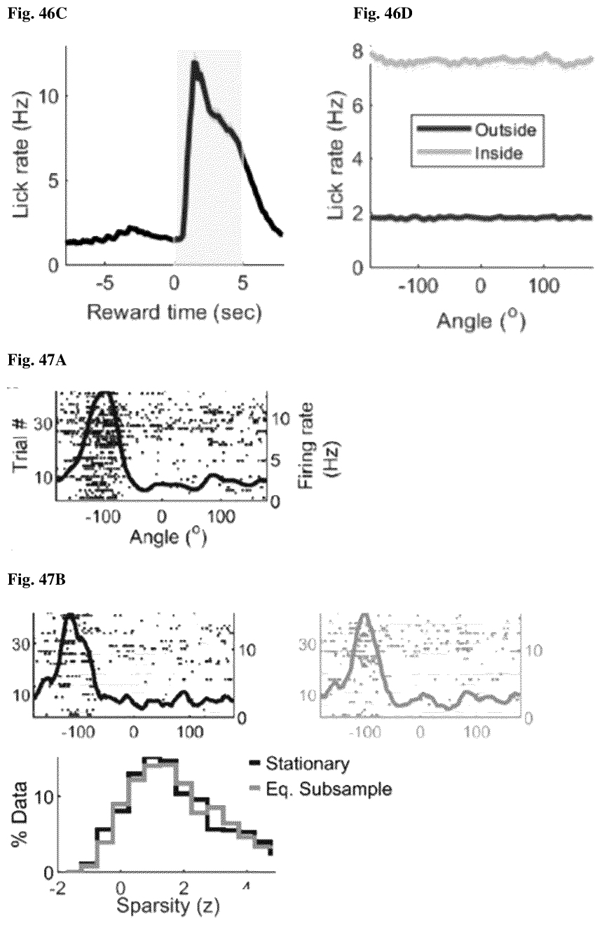

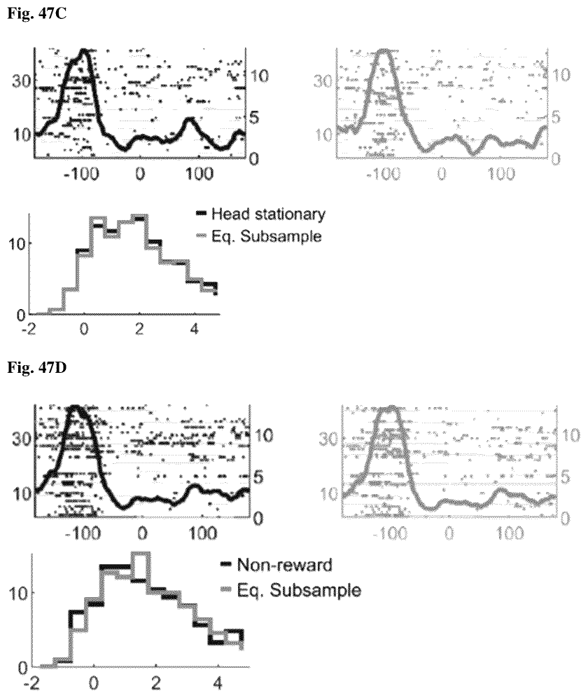

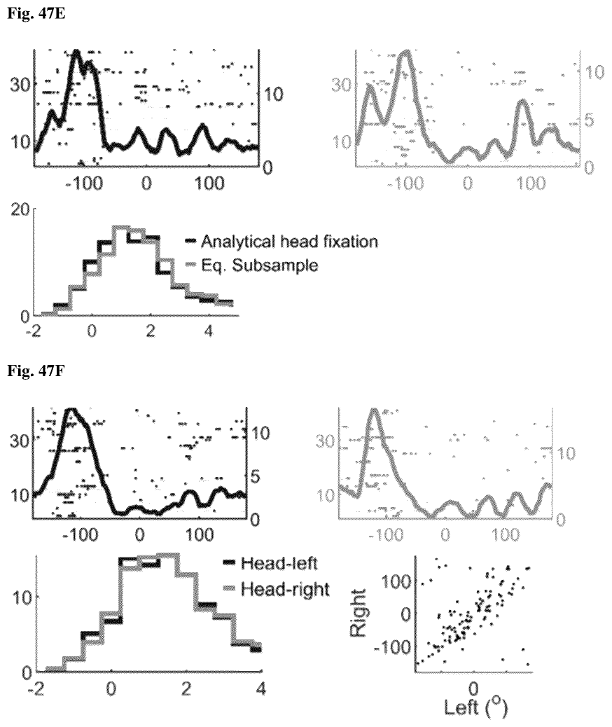

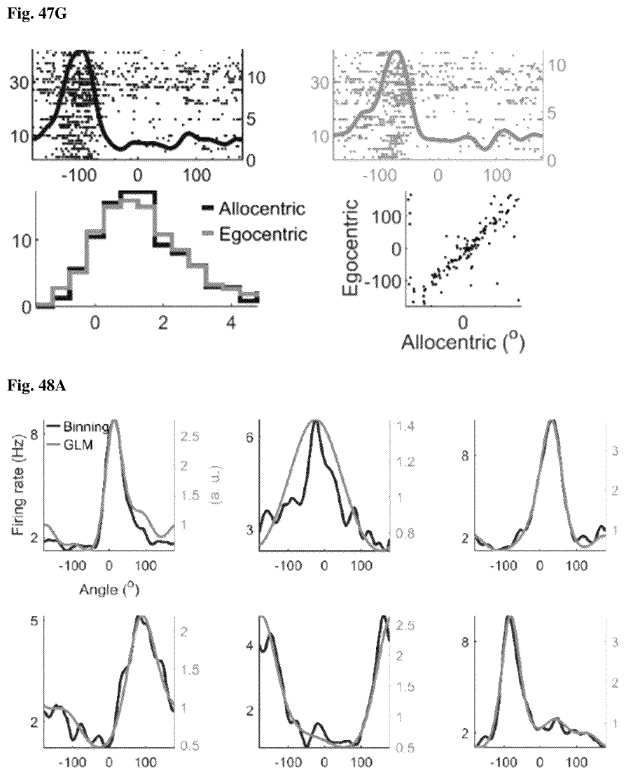

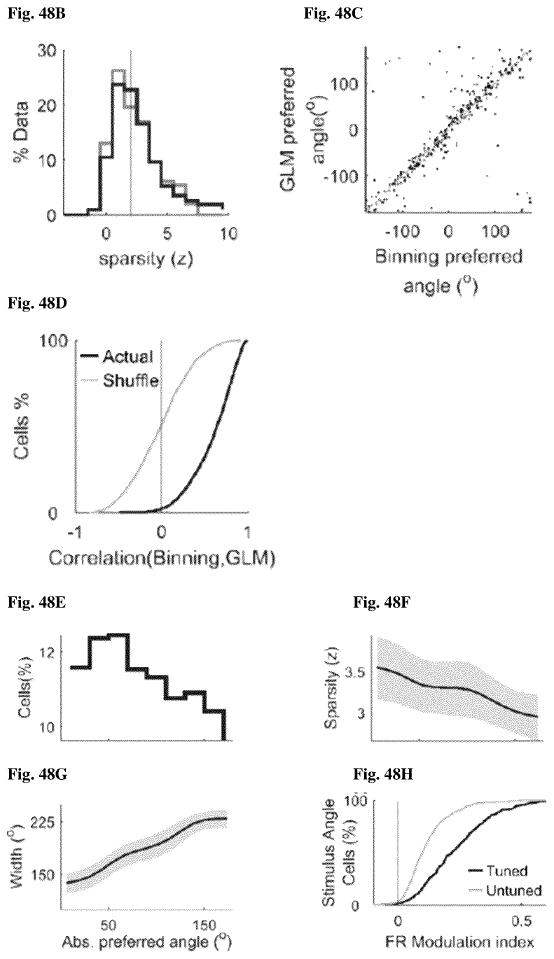

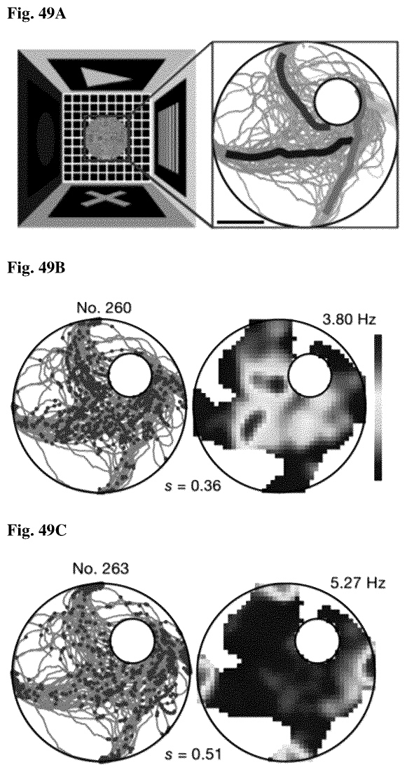

These results demonstrate that a moving bar of light can reliably modulate the activity of majority of hippocampal place cells, without any task demand, memory, reward contingency or locomotion requirements (see FIGS. 46A-46D for reward related controls, FIGS. 47A-47G for behavior related controls, and FIGS. 48A-48H for GLM estimates). Neurons encoded both the angular position and linear distance of the bar of light, with respect to the rat. In addition, neurons were selective to the direction of angular or linear movement. Thus, these responses provided a vectorial representation of the stimulus positions around the rat. Only a few hundred neurons were sufficient to accurately decode the angular position of visual stimulus. Positions in front of the rat and near him were overrepresented. A majority of neurons that encoded the bar position were also spatially selective during real world exploration and the strength of SAC and spatial tunings were correlated across neurons. However, unlike place cells that remap when the behavior is sequential vs random³³, the stimulus angle tuning was relatively unchanged when the predictability or sequential nature of stimuli was altered. Even more striking, while place cells are predictive or prospective^{25,27,35}, including in a virtual reality setup similar to that used here¹⁹, the stimulus angle tuning was retrospective in nature (FIGS. 37A, 37B).

These results have similarities and important differences compared to recent findings of social neurons in the hippocampus, where a small subset of neurons encoded the linear position of a demonstrator animal^{13,14}. However, those experiments required strong training, tasks, or rewards. Without these behavioral requirements there was no significant modulation of hippocampal activity^{9-11,15}. Other experiments showed that a small subset of hippocampal neurons could respond to sensory cues during auditory discrimination task, but robust responses required stimulus locked rewards and behavior¹⁰⁻¹². Thus, hippocampal selectivity in those experiments cannot be attributed solely to the stimulus position. In contrast, the experiments described herein show that the neural responses can be attributed solely to the stimulus angle. Indeed, the stimulus angular tuning was relatively invariant to changes in the pattern or color of the bar of light or the randomness of stimulus movement. Further, a majority of neurons showed significant modulation in our experiments, enough to decode the bar position from a few hundred neurons. The differences between the prior results and those presented herein could be because the hippocampus is involved in creating spatial representations from the visual cues and the experiments described herein created stimulus movement while eliminating nonspecific cues. This is supported by the strong correlation between the degree of visual stimulus angle position tuning and allocentric spatial tuning across neurons.

These results show that during passive viewing, rodent hippocampal activity patterns fit the visual hierarchy³⁶. For example, the SAC show similar angular dependence as visual cortex, e.g., larger tuning curve width for more peripheral stimuli and over-representation of the nasal compared to temporal positions²⁰. This nasal-temporal magnification increases with increasing processing stages from the retina to thalamus and striate cortex²⁰, but the hippocampal magnification reported herein is much smaller. Further, like the visual cortex, hippocampal neurons too showed retrospective responses but with larger response latency, suggesting that visual cortical inputs reached the hippocampus to generate SAC. The larger latency is consistent with the response latencies in the human hippocampus³⁷ and the progressive increase in response latencies in the cortico-entorhinal-hippocampal circuit during Up-Down states³⁸⁻⁴⁰. However, there were no off responses in the SAC and the tuning curves were broader and more unidirectional than in the primary visual cortex. This could arise due to processing in the cortico-hippocampal circuit, especially the entorhinal cortex⁴⁰, or due to the contribution of alternate pathways from the retina to the hippocampus⁴¹.

Hippocampal spatial maps are thought to rely on the distal visual cues⁵. Rats can not only navigate using only vision in virtual reality, but they preferentially rely on vision¹⁸. Robust hippocampal coding for visual cue position, angle, and movement direction reported herein without any movements further supports these findings. But these findings cannot be explained by path integration. Instead, they can be explained by a refinement of the multisensory-pairing hypothesis^{7,17}. In the absence of any correlation between physical stimuli, rewards and internally generated self-motion, hippocampal neurons can generate robust, invariant, non-abstract responses to the visual stimulus angle, distance, and direction, akin to cortical regions. Consistently, these responses are retrospective in nature, similar to cortical responses, with additional latency. However, these responses are less robust than place cells. Visual cues combined with uncorrelated locomotion cues can generate direction head-selectivity but not spatial selectivity¹⁷, whereas consistency between locomotion, reward and visual cues generates spatial selectivity in the hippocampus^{7,46} and primary visual cortex³². Place cells robustly respond to not only visual^{42,43} but also multisensory cues on the track^{27,35,44} and to self-motion cues^{7,19,45}. It was hypothesize that the greatly enhanced correlations between all the cues could be encoded more robustly via synaptic plasticity to generate anticipatory or prospective coding of absolute position^{21,47}. This is further supported by the finding that robust responses and prospective coding were also seen in purely visual virtual reality, but for relative distance, not absolute position, since only the optic flow and locomotion cues were correlated at identical distance¹⁹. Thus, the retrospective coding of moving stimulus angle, position and direction could form the basis for generating a wide range of invariant, anticipatory spatial maps via multisensory associations.

### REFERENCES

1. Hubel, D. H. & Wiesel, T. N. Receptive fields, binocular interaction and functional architecture in the cat's visual cortex. J. Physiol. 160, 106 (1962).
2. O'Keefe, J. & Dostrovsky, J. The hippocampus as a spatial map. Preliminary evidence from unit activity in the freely-moving rat. Brain Res 34, 171-5. (1971).
3. Fyhn, M., Molden, S., Witter, M. P., Moser, E. I. & Moser, M. B. Spatial representation in the entorhinal cortex. Science (80-.). 305, 1258-1264 (2004).
4. Taube, J. S., Muller, R. U. & Ranck Jr., J. B. Head-direction cells recorded from the postsubiculum in freely moving rats. II. Effects of environmental manipulations. J Neurosci 10, 436-47. (1990).
5. O'Keefe, J. & Nadel, L. The hippocampus as a cognitive map. (Clarendon Press, 1978).
6. Foster, T. C., Castro, C. A. & McNaughton, B. L. Spatial selectivity of rat hippocampal neurons: dependence on preparedness for movement. Science (80-.). 244, 1580-2. (1989).
7. Aghajan, Z. M. et al. Impaired spatial selectivity and intact phase precession in two-dimensional virtual reality. Nat. Neurosci. 18, 121-128 (2015).
8. McNaughton, B. L. et al. Deciphering the hippocampal polyglot: the hippocampus as a path integration system. J Exp Biol 199, 173-85. (1996).
9. Sakurai, Y. Involvement of auditory cortical and hippocampal neurons in auditory working memory and reference memory in the rat. J. Neurosci. 14, 2606-2623 (1994).
10. Sakurai, Y. Coding of auditory temporal and pitch information by hippocampal individual cells and cell assemblies in the rat. Neuroscience 115, 1153-1163 (2002).
11. Aronov, D., Nevers, R. & Tank, D. W. Mapping of a non-spatial dimension by the hippocampal-entorhinal circuit. Nature 543, 719-722 (2017).
12. Itskov, P. M. et al. Sound sensitivity of neurons in rat hippocampus during performance of a sound-guided task Sound sensitivity of neurons in rat hippocampus during performance of a sound-guided task. 1822-1834 (2012). doi:10.1152/jn.00404.2011
13. Omer, D. B., Maimon, S. R., Las, L. & Ulanovsky, N. Social place-cells in the bat hippocampus. Science (80-.). 359, 218-224 (2018).
14. Danjo, T., Toyoizumi, T. & Fujisawa, S. Spatial representations of self and other in the hippocampus. Science (80-.). 359, (2018).
15. Mou, X. & Ji, D. Social observation enhances cross-environment activation of hippocampal place cell patterns. Elife 5, 1-26 (2016).
Taube, J. S., Muller, R. U. & Ranck Jr., J. B. Head-direction cells recorded from the postsubiculum in freely moving rats. I. Description and quantitative analysis. J Neurosci 10, 420-35. (1990).
16. Acharya, L., Aghajan, Z. M., Vuong, C., Moore, J. J. & Mehta, M. R. Causal Influence of Visual Cues on Hippocampal Directional Selectivity. Cell 164, 197-207 (2016).
17. Cushman, J. D. et al. Multisensory Control of Multimodal Behavior: Do the Legs Know What the Tongue Is Doing? PLoS One 8, e80465 (2013).
18. Ravassard, P. et al. Multisensory control of hippocampal spatiotemporal selectivity. Science 340, 1342-6 (2013).
19. Malpeli, J. G. & Baker, F. H. The representation of the visual field in the lateral geniculate nucleus of Macaca mulatta. J. Comp. Neurol. 161, 569-594 (1975).
20. Mehta, M. R., Quirk, M. C. & Wilson, M. A. Experience-dependent asymmetric shape of hippocampal receptive fields. Neuron 25, 707-15. (2000).
21. Ahmed, O. J. & Mehta, M. R. The hippocampal rate code: anatomy, physiology and theory. Trends Neurosci 32, 329-338 (2009).
22. de Vries, S. E. J. et al. A large-scale standardized physiological survey reveals functional organization of the mouse visual cortex. Nat. Neurosci. 23, 138-151 (2020).
23. Wilson, M. A. & McNaughton, B. L. Dynamics of the hippocampal ensemble code for space. Science (80-.). 261, 1055-8. (1993).
24. Resnik, E., McFarland, J. M., Sprengel, R., Sakmann, B. & Mehta, M. R. The Effects of GluA1 Deletion on the Hippocampal Population Code for Position. J. Neurosci. 32, 8952-68 (2012).
25. Stefanini, F. et al. A distributed neural code in the dentate gyrus and in CA1. Neuron (2020).
26. Battaglia, F. P., Sutherland, G. R. & McNaughton, B. L. Local sensory cues and place cell directionality: additional evidence of prospective coding in the hippocampus. J Neurosci 24, 4541-4550 (2004).
27. Muller, R. U., Kubie, J. L., Bostock, E. M., Taube, J. S. & Quirk, G. J. Spatial firing correlates of neurons in the hippocampal formation of freely mfile:///C:/Users/mayank/Downloads/scholar (6).risoving rats. (1991).
28. Colgin, L. L., Moser, E. I. & Moser, M. B. Understanding memory through hippocampal remapping. Trends Neurosci 31, 469-477 (2008).
29. Suzuki, W. A., Miller, E. K. & Desimone, R. Object and place memory in the macaque entorhinal cortex. J Neurophysiol 78, 1062-1081 (1997).
30. Pastalkova, E., Itskov, V., Amarasingham, A., Buzsaki, G. & Buzsiki, G. Internally generated cell assembly sequences in the rat hippocampus. Science (80-.). 321, 1322-1327 (2008).
31. Saleem, A. B., Diamanti, E. M., Fournier, J., Harris, K. D. & Carandini, M. Coherent encoding of subjective spatial position in visual cortex and hippocampus. Nature 562, 124-127 (2018).
A. Markus, E. J. et al. Interactions between location and task affect the spatial and directional firing of hippocampal neurons. J Neurosci 15, 7079-94. (1995).
32. Ziv, Y. et al. Long-term dynamics of CA1 hippocampal place codes. 2013, (2013).
33. Geiller, T., Fattahi, M., Choi, J.-S. S. & Royer, S. Place cells are more strongly tied to landmarks in deep than in superficial CA1. Nat. Commun. 8, 14531 (2017).
34. Felleman, D. J. & Van, D. C. E. Distributed hierarchical processing in the primate cerebral cortex. Cereb. cortex (New York, NY 1991) 1, 1-47 (1991).
35. Quiroga, R. Q., Reddy, L., Kreiman, G., Koch, C. & Fried, I. Invariant visual representation by single neurons in the human brain. 435, 1102-1107 (2005).
36. Hahn, T. T., Sakmann, B. & Mehta, M. R. Phase-locking of hippocampal interneurons' membrane potential to neocortical up-down states. Nat Neurosci 9, 1359-1361 (2006).
37. Hahn, T. T., Sakmann, B. & Mehta, M. R. Differential responses of hippocampal subfields to cortical up-down states. Proc Natl Acad Sci USA 104, 5169-5174 (2007).
38. Hahn, T. T. G., McFarland, J. M., Berberich, S., Sakmann, B. & Mehta, M. R. Spontaneous persistent activity in entorhinal cortex modulates cortico-hippocampal interaction in vivo. Nat. Neurosci. advance on, 1531-1538 (2012).
39. Beltramo, R. & Scanziani, M. A collicular visual cortex: Neocortical space for an ancient midbrain visual structure. Science (80-.). 363, 64-69 (2019).
40. Ji, D. & Wilson, M. A. Coordinated replay of awake experience in the cortex and hippocampus during sleep. Nat Neurosci 10, 100-107 (2007).
41. Haggerty, D. C. & Ji, D. Activities of visual cortical and hippocampal neurons co-fluctuate in freely moving rats during spatial behavior. Elife 4, e08902 (2015).
42. Royer, S. et al. Control of timing, rate and bursts of hippocampal place cells by dendritic and somatic inhibition. Nat. Neurosci. 15, 769 (2012).
43. Villette, V., Malvache, A., Tressard, T., Dupuy, N. & Cossart, R. Internally Recurring Hippocampal Sequences as a Population Template of Spatiotemporal Information. Neuron 88, 357-366 (2015).
44. Aronov, D. & Tank, D. W. Engagement of Neural Circuits Underlying 2D Spatial Navigation in a Rodent Virtual Reality System. Neuron 84, 442-456 (2014).
45. Mishkin, M., Health, M., Mehta, M. R., Barnes, C. A. & McNaughton, B. L. Experience-dependent, asymmetric expansion of hippocampal place fields. Proc Natl Acad Sci USA 94, 8918-21. (1997).
46. Ringach, D. L., Shapley, R. M. & Hawken, M. J. Orientation selectivity in macaque V1: Diversity and laminar dependence. J. Neurosci. 22, 5639-5651 (2002).
47. Ghodrati, M., Zavitz, E., Rosa, M. G. P. & Price, N. S. C. Contrast and luminance adaptation alter neuronal coding and perception of stimulus orientation. Nat. Commun. 10, (2019).
48. Berens, P. CircStat: A MATLAB Toolbox for Circular Statistics. J. Stat. Softw. 31, 1-21 (2009).
48. Berens, P. CircStat: A MATLAB Toolbox for Circular Statistics. J. Stat. Softw. 31, 1-21 (2009).

Methods/Subjects

Eight adult male Long-Evans rats (3 months old at the start of experiments) were individually housed on a 12-hour light/dark cycle. Their total food intake (15-20 g of food per day) and water intake (25-35 ml of water per day) were controlled and monitored to maintain body weight. Rats received 10-12 ml of water in a 20-minute experiment. All experimental procedures were approved by the UCLA Chancellor's Animal Research Committee and were conducted in accordance with USA federal guidelines.

Experimental Apparatus

Rats were body restricted with a fabric harnesses as they ran on an air-levitated spherical treadmill of 30 cm radius. The rat was placed at the center of a cylindrical screen of radius 33 cm and 74 cm high. Visual cues were projected on the screen. Although the rat was free to run and stop voluntarily, his running activity was decoupled from the projector and hence had no effect on the visual cues. Body restriction allowed the rat to scan his surroundings with neck movements. Running speed was measured by optical mice recording rotations of the spherical treadmill at 60 Hz. Head movement with respect to the harnessed and fixed body was recorded at 60 Hz using an overhead camera tracking two red LEDs attached to the cranial implant using the methods described before₁₉. Rewards were delivered at random intervals (16.2 s±7.5 s, 2 rewards, 200 ms apart) to keep the rats motivated and the experimental conditions similar to typical place cell experiments.

Behavioral Pre-Training

All experiments were conducted in acoustically- and EMF-shielded rooms. The rats were conditioned to associate a tone with sugar-water reward. They were gently body-fixed in the apparatus that allowed them to move their heads with respect to the body but the body could not turn around. In order for the rats to remain calm in the apparatus for long periods, they were trained to navigate in a visually rich virtual maze where a suspended, striped pillar indicated rewarded position. After surgery, rats were exposed to the revolving bar environment for the first time, where the movement of the rat had no impact on the movement of the revolving bar. Six out of eight rats never experienced virtual reality after the revolving bar experiments began.

Experiment Design

The salient visual stimulus was a 13 degrees wide vertical bar of light which revolved around the rat at a constant speed (10 s per revolution) without any change in shape or size (FIG. 26A). Three different textures of visual cues were used as shown in FIGS. 41A-41G. The results were qualitatively similar for all of them hence the data were combined. Each block of trials consisted of four clockwise (CW) or four counterclockwise (CCW) revolutions of the bar of light. There were 13-15 blocks of trials in each session. During the random bar of light experiment, the bar revolved at one of the six speeds: ±36°, ±72°, or ±108° per second and spanning angles ranging 30° to 70° at any given speed, before changing the speed at random. Reward dispensing was similar to the systematic bar of light experiment, with no relation to the angular position or speed of the stimulus. Manipulations of stimulus color, pattern, movement predictability, and linearly moving stimulus were performed in a pseudo-random order in the same VR apparatus. Real world two-dimensional random foraging experiments and stimulus angle experiments were performed in a pseudo-random order, with an intermittent baseline of 25-40 minutes.

Surgery

All rats were implanted with 25-30 g custom-built hyperdrives containing up to 22 independently adjustable tetrodes (13 μm nichrome wires) positioned bilaterally over dorsal CA1 (−3.2 to −4.0 mm A. P., ±1.75 to ±3.1 mm M. L. relative to Bregma). Surgery was performed under isoflurane anesthesia and heart rate, breathing rate, and body temperature were continuously monitored. Two−2 mm-diameter craniotomies were drilled using custom software and a CNC device with a precision of 25 μm in all 3 dimensions. Dura mater was manually removed and the hyperdrive was lowered until the cannulas were about 100 μm above the surface of the neocortex. The implant was anchored to the skull with 7-9 skull screws and dental cement. The occipital skull screws were used as ground for recording. Rats were administered about 5 mg/kg carprofen (Rimadyl bacon-flavored pellets) one day prior to surgery and for at least 10 days during recovery.

Electrophysiology

The tetrodes were lowered gradually after surgery into the CA1 hippocampal sub region. Positioning of the electrodes in CA1 was confirmed through the presence of sharp-wave ripples during recordings. Signals from each tetrode were acquired by one of three 36-channel head stages, digitized at 40 kHz, band pass-filtered between 0.1 Hz and 9 k Hz, and recorded continuously.

Spike Sorting

Spikes were detected offline using a nonlinear energy operator threshold, after application of a non-causal fourth order Butterworth band pass filter (600-6000 Hz). After detection, 1.5 ms spike waveforms were extracted. Spike sorting was performed manually using an in-house clustering algorithm written in Python.

Tuning Curves and z-Score Calculation

Procedures similar to that described previously were used¹⁹. The angular occupancy of the vertical bar and spikes were binned in N=120 bins of width 3° each and smoothed with a Gaussian of σ=12°. Clockwise and counter-clockwise movement directions were treated separately. To quantify the degree of modulation, sparsity s of an angular rate map was computed, where rₙ is the firing rate in the nnₜₜₕ angular bin:

$$s = 1 - \frac{1}{N} \frac{{( {Σ}_{n} {r}_{n} )}^{2}}{( {Σ}_{n} {r}_{n}^{2} )}$$

To assess the statistical significance of sparsity, a bootstrapping procedure was used that does not assume a normal distribution. Briefly, for each cell, in each movement direction, spike trains as a function of the vertical bar from each block of trials were circularly shifted by different angles and the sparsity of the randomized data computed. This procedure was repeated 250 times with different sets of random value shifts. The mean value and standard deviation of the sparsity of randomized data was used to compute the z-scored sparsity of actual data using the function zscore in MATLAB. The observed sparsity was considered statistically significant if the z-scored sparsity of the observed spike train was greater 2, which corresponds top<0.0228 in a one tailed t-test.

Similar procedure was employed for testing the significance angular tuning in the random bar of light condition. To keep the analysis comparable to systematic condition, spike trains were circularly shifted with respect to behavioral data by different random amounts for each block of 40 seconds, which is comparable to the time taken by the systematic visual cue to undergo four revolutions.

In addition to sparsity, SAC was quantified using several other measures.

Angle Selectivity index ASI=A₂/A₂+A₀,

where AA2 is the second harmonic_{z} component from the Fourier transform of the binned SAC response and AA0 is the DC level. This formulation of ASI is analogous to Orientation selectivity index (OSI), which is widely used in visual cortical selectivity quantification.⁴⁸⁻⁵⁰

Mean resultant length (MVL)=Σₙrₙ·(e^{iθ}^{ₙ}⁾

Where rn is the firing rate in the n^{th} angular bin θₙ is the angular position corresponding to this bin and n is summed over 120 bins.

Coherence (CH)=correlation coefficient ({r_{n,raw}},{r_{n,smoothed}})

$$Mutual Information ( MI ) = {Σ}_{C} p ( C \vert {θ}_{n} ) \cdot {log}_{2} \frac{p ( C \vert {θ}_{n} )}{p ( C )}$$

Where p(CC)=Σₙ p(θn)·pp(C|θₙ)
and C is the average spike count in 0.083 second window which corresponds to 1 angular bin that is 3° wide. Statistical significance of these alternative measures of selectivity was computed similar to that for sparsity and is detailed in FIGS. 27A-27D.

### Tuning Curve Width Quantification

Full width at quarter maxima of the SAC rate map was computed around the maxima of the firing rate, i.e., the preferred angle, as the width at which the tuning curve first crossed 0.25 times the peak value. 0.25 of maximum and not 0.5, i.e., FWHM as commonly done, was chosen because the tuning curves are often very broad with nonzero activity at nearly all angles, which is missed by FWHM.

Modulation Index calculation

Firing rate modulation index of stimulus angle tuning (used in FIG. 26G) was quantified as (FR_{within}−FR_{outside})/(FR_{within}+FR_{outside}), where FR_{within} and FR_{outside} are average firing rates in their respective zones. Similar definition of FR modulation index was used in FIG. 31G, to quantify the effect of uni-directional tuning inside and outside of the preferred zone, as (Fr_{tuned}−FR_{untuned})/(FR_{tuned}+FR_{untuned}), where FR_{tuned} and FR_{unturned} are the average firing rates in the respective directions. Similarly in FIG. 4k, to quantify the effect of stimulus speed, as (FR_{fast}−FR_{slow})/(FR_{fast}+FR_{slow}), where FR_{fast} and FR_{slow} are the average firing rates during stationary epochs of respective stimulus movement speeds.

Spike Train Thinning

Neurons with larger number of spikes, e.g., due to longer experiments, have greater sparsity than when the number of spikes is less. To remove this artifact and compare the degree of SAC across all neurons and conditions, a spike thinning procedure was used. Randomly chosen spikes were removed such that that the effective firing rate became 0.5 Hz for all neurons and then the sparsity of this thinned spike train was computed (FIGS. 35A-35D). This procedure was used separately for CW and CCW directions to allow comparison of the degree of tuning in both directions, independent of the firing rate changes.

Stability Analysis

Stability of neural angular tuning was quantified for CW and CCW directions separately. All the trials were split into two randomly chosen, equal and non-overlapping groups (˜30 trials each) and separate tuning curves computed for each half, with 120 equally spaced, non-overlapping angular bins. The correlation coefficient was computed between these two groups (C_{actual}), which is a measure of stability. To compute the significance of stability, this procedure was repeated 30 times, with different random grouping of trials, and correlation coefficient computed between the two groups computed each time. This provided a distribution of thirty values of stability C_{actual}. Same procedure was used for rate maps computed using random data (see z-score methods above) and correlation computed between two groups to obtain thirty different values of C_{random}. A cell's SAC was considered significantly stable if the following conditions were met: the nonparametric rank-sum test comparing the thirty C_{actual} with thirty C_{random} was significant at p<0.05 and C_{actual}>C_{random} Untuned-stable responses were identified as responses with significant stability, but non-significant tuning (sparsity (z)<2) and treated as a separate population in FIG. 31A-31K.

Population Vector Overlap

To evaluate the properties of a population of cells, sessions were divided into trials in the CCW and CW movement directions of the visual bar. Population vector overlap between CCW and CW movement direction at angles (θᵣ, θₘ) for N single units was defined as the Pearson correlation coefficient between vectors rate of the i^{th} (μ_{1,r}, μ_{2,r}, . . . μ_{N,r}) & (μ_{1,m}, μ_{2,m}, . . . μ_{N,m}) where μ_{i,p} is the normalized firing neuron at p^{th} angular bin. Correlation coefficient of these sub-populations taken across angles indicates the existence of retrospective coding (FIGS. 38H, 38K, and 44J.). Similarly, for computing coherence in either direction, population vector overlap between two groups of trials of the same bar movement direction (as defined above, stability analysis methods) was computed separately for CCW and CW trials (FIGS. 36A-36K). Populations of tuned, untuned but stable, and untuned-unstable cells were treated separately.

Decoding Analysis

Using the stability labels as obtained from above, recorded cells were divided into three populations: tuned (sparsity z>2), untuned (sparsity z<2) and stable, and untuned and unstable. All the trials across all the cells within each population were separated into two groups: ten randomly chosen trials were treated as the “observed trials,” and these data were decoded using the firing rate maps obtained from the remaining trials, or the “lookup trials.” The commonly used population vector overlap method was used between the lookup and observed trials using a window of 250 ms. Briefly, at each 250 ms time point in the “observed data,” the correlation was computed between the observed population vector and the lookup population vectors at all angles. The circularly weighted average of angles, weighted by the (non-negative) correlations provided the decoded angle. The entire procedure was repeated 30 times for different sets of 10 trials. The error was computed as the circular difference between the decoded and actual angle at the observed time. Decoding of the stimulus distance (FIGS. 45A-45K) was done similarly but by finding the distance corresponding maximum correlation between “looking” and “observed” data, since circular averaging is unavailable for linear distance close and away from the rat.

Same Cell Identification

Spike sorting was performed separately for each session using custom software¹⁹. Identified single units were algorithmically matched between sessions to enable same cell analysis (FIGS. 41A-41G and 44A-44J). All the isolated cells in one session were compared with all the isolated cells in another session under investigation. Each putative unit pair was assigned a dissimilarity metric based on the Mahalanobis distance between their spike amplitudes, normalized by their mean amplitude. Dissimilarity numbers ranged from 2.5×10⁻⁵ to 17.2 across all combinations of units between two sessions. Putative matches were iteratively identified in an increasing order of dis-similarity, until this metric exceeded 0.04. These putative matches were further vetted, using an error index defined on their average spike waveforms.

Estimating the Independent Contribution of Head Position, Running Speed and Stimulus Angle Using GLM

To compute the independent contributions of head position, running speed, and stimulus angle, a GLM-based estimation of firing was employed, using the glmfit function in MATLAB, as described recently¹⁷. Head position and running speed were decoded in GLM using basis functions consisting of sinusoids. The log of running speed was used to ensure similar amount of data in each bin, and bins with zero speed were assigned an arbitrary, small value, which was on average was equal to half the minimum non-zero running speed. Spike train and behavior data were downsampled to 100 ms bins. The extreme one percentile of head position data and top one percentile of running speed data was excluded to remove the effects of outliers and ensure a good fit. CCW and CW tuning curves for stimulus angle were computed seperately. The statistical significance of the resulting tuning curves was estimated by computing sparsity and a bootstrapping method described above and used recently¹⁷.

Quantification of Population Remapping

To compute the amount of remapping of firing rate, strength of tuning, preferred angle of firing and similarity between CCW and CW SAC, we used the responses of the same cells recorded from different experimental conditions and defined remapping metrices as firing rate modulation index, difference between z-scored sparsity, circular distance between the angles corresponding to maximal firing, correlation coefficient between the firing rate profiles and the peak value and angular latency corresponding to the cross correlation between their tuning curves in the two conditions. This calculation was repeated 100 times using a random permutation to break the same cell pairing, to obtain a null distribution. The mean and standard deviation of this distribution was plotted in FIGS. 41E-41G and FIGS. 42A-42K, and compared with the actual value of the corresponding remapping metric.

Quantification of Trial to Trial Variability of SAC

Angular movement of the visual stimulus was separated into different trials starting and ending at 0°, which is the angular position in front of the rat. Mean firing rate in each trial was obtained by binning the spikes in that trial into 120 angular bins (3 degrees wide), and finding the average value of firing rates in each bin. Similarly, mean vector angle and mean vector length were obtained using circ_r and circ_mean functions of the Circular Statistics toolbox in MATLAB⁵⁰ either by using all trials or only those trials when at least 5 spikes were recorded (each trial was 10 s long, yielding 0.5 Hz lower bound on mean firing rate).

To determine if the variability was correlated across simultaneously recorded tuned cells, a co-fluctuation index for firing rate was defined for all cell pairs as the Spearman correlation between the trial-wise firing rate vectors of both cells. Co-Fluctuation FR=spearman({F_{1,k}},{F_{2,k}}), where F_{i,k} denotes the mean firing rate of i^{th} cell on the k^{th} trial. Bootstrapping procedure to access significance of this index was employed by obtaining 100 shuffled indices when the order of trials was randomly reassigned. Similarly, to estimate the co-fluctation of SAC, a similarity metric was defined for each trial as S_{i,k}=crcf(r_{n,k}, Rₙ) where n denotes the angular bins, Rₙ overall tuning curve, and r_{n, k} is the firing rate in the n^{th} bin for the k^{th} trial and crcf is the correlation coefficient function. Co-fluctuation of tuning was defined analogously as Co-Fluctuation SAC=spearman ({S_{1,k}},{S_{2,k}}), and bootstrapped similarly as the firing rate co-fluctuation index.

Three major pillars of hippocampal function are spatial navigation¹, Hebbian synaptic plasticity² and spatial selectivity³. The hippocampus is also implicated in episodic memory⁴, but the precise link between these four functions is missing. Here we report the multiplexed selectivity of dorsal CA1 neurons while rats performed a virtual navigation task using only distal visual cues⁵, similar to the standard water maze test of spatial memory¹. Neural responses primarily encoded path distance from the start point and the head angle of rats, with a weak allocentric spatial component similar to that in primates but substantially weaker than in rodents in the real world. Often, the same cells multiplexed and encoded path distance, angle and allocentric position in a sequence, thus encoding a journey-specific episode. The strength of neural activity and tuning strongly correlated with performance, with a temporal relationship indicating neural responses influencing behaviour and vice versa. Consistent with computational models of associative and causal Hebbian learning^{6,7}, neural responses showed increasing clustering⁸ and became better predictors of behaviourally relevant variables, with the average neurometric curves exceeding and converging to psychometric curves. Thus, hippocampal neurons multiplex and exhibit highly plastic, task- and experience-dependent tuning to path-centric and allocentric variables to form episodic sequences supporting navigation.

The hippocampus is thought to mediate spatial navigation1 by cognitive mapping³ or path integration^{9,10}, represented by the allocentric selectivity of place cells and built using distal visual cues and Hebbian synaptic plasticity¹¹⁻¹³. However, a precise link is lacking between N-methyl-d-aspartate receptor (NMDAR)-dependent synaptic plasticity^{6,7}, emergent place field plasticity^{7,14-16} and navigational performance1. Additionally, episodic-like responses are seen in primate, human^{4,17,18} and rodent¹⁹⁻²³ hippocampi in certain tasks, but their relevance to spatial navigation is unclear. Computational models of learning by the associative component of Hebbian plasticity² predict clustering of neural responses, whereas the temporally asymmetric form, or the spike-timing-dependent plasticity (STDP)^{24,25}, predicts increased activity and anticipatory shift^{6,7,14}. The latter has been observed on narrow, one-dimensional paths^{7,14-16}, but neither has been observed in two dimensions or during navigation. To address these issues, we trained four adult rats to execute a virtual navigation task (VNT) similar to the Morris water maze⁵ and measured hippocampal neural responses and their experience-dependent plasticity. Virtual reality (VR) entirely removes non-specific cues and human intervention and ensures navigation using only distal visual cues, which is a fundamental feature of cognitive mapping. The appetitive reinforcement, similar to most place cell experiments, allows rats to run many trials and removes stress experienced in the water maze that could impair synaptic plasticity. The trial-based structure of the task allows us to explore the neural encoding of sequences of behaviorally relevant events and measures, such as initiation and direction of movement, distance travelled and the expected reward position.

Performance in the VNT

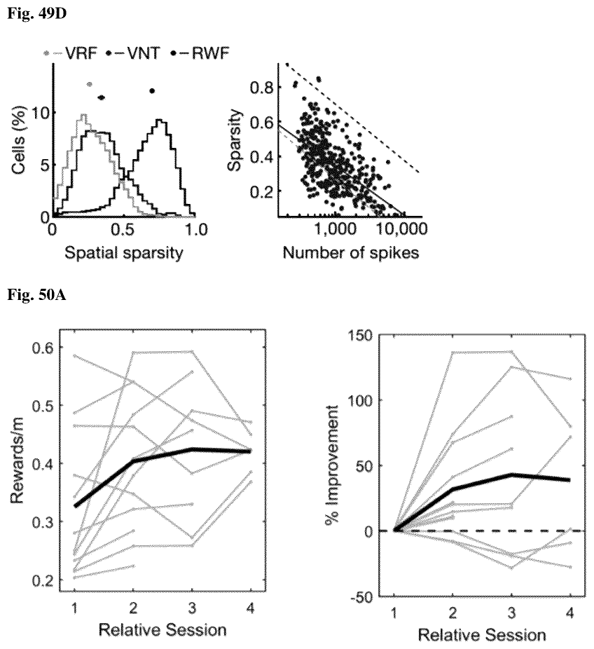

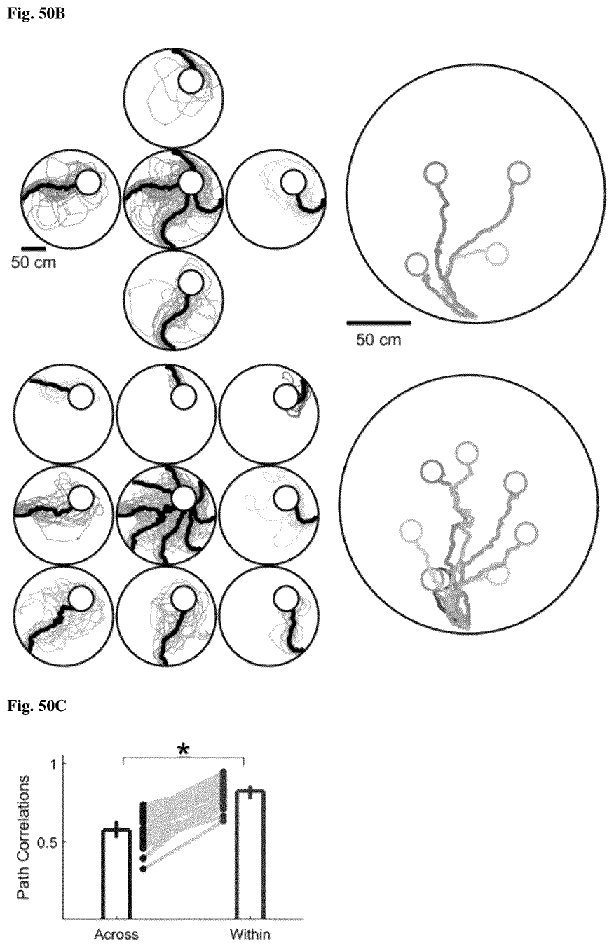

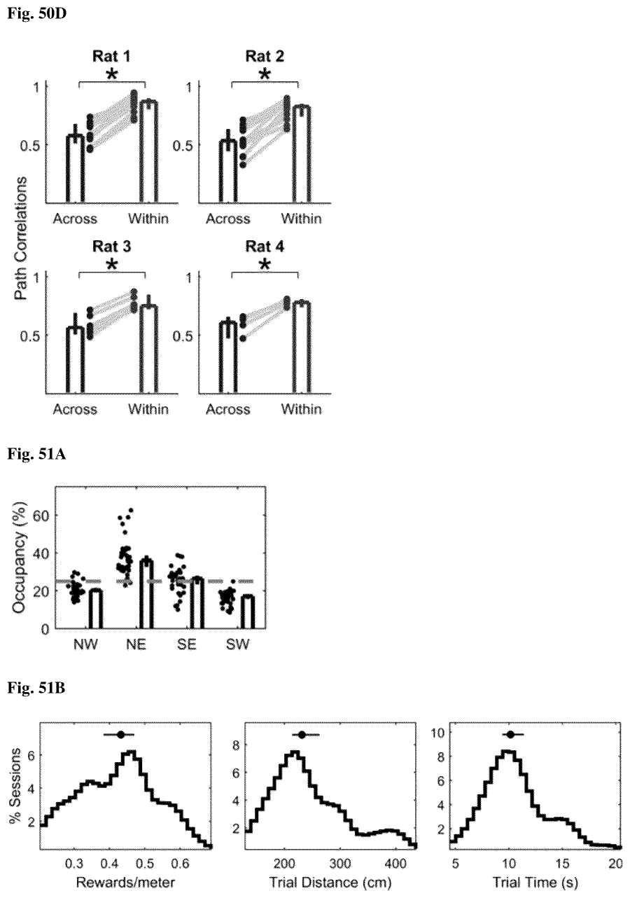

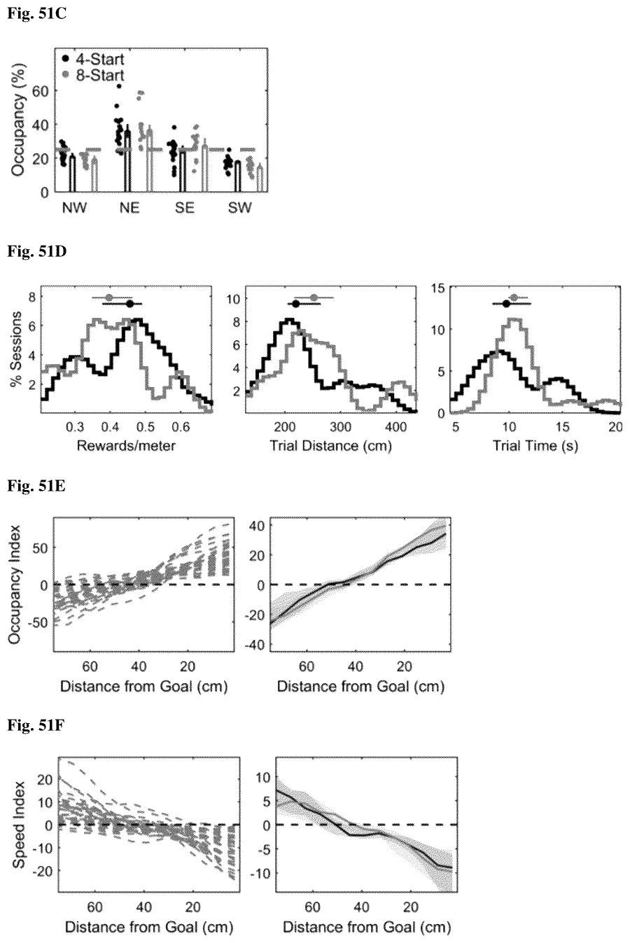

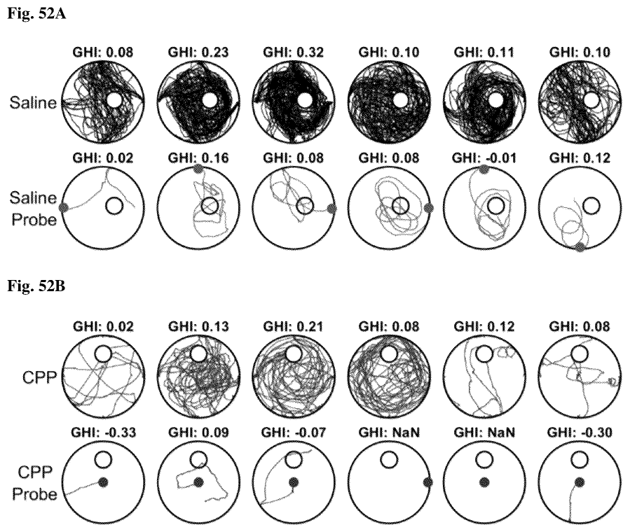

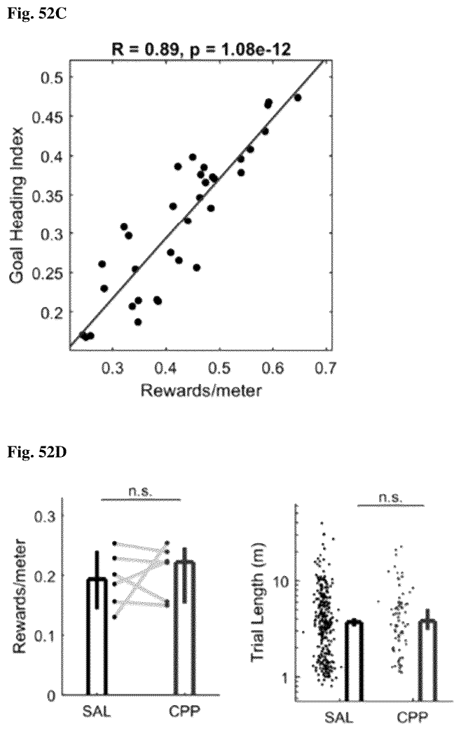

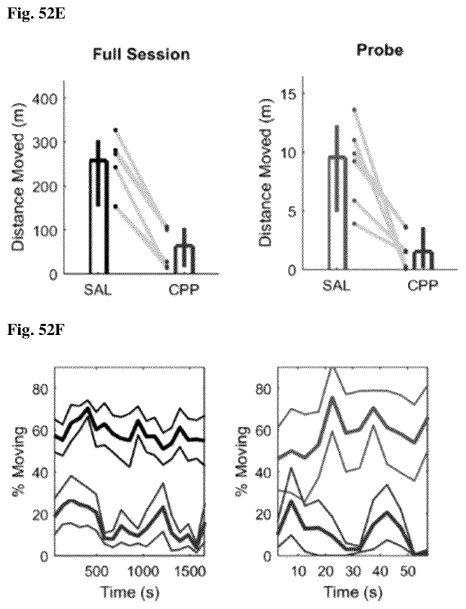

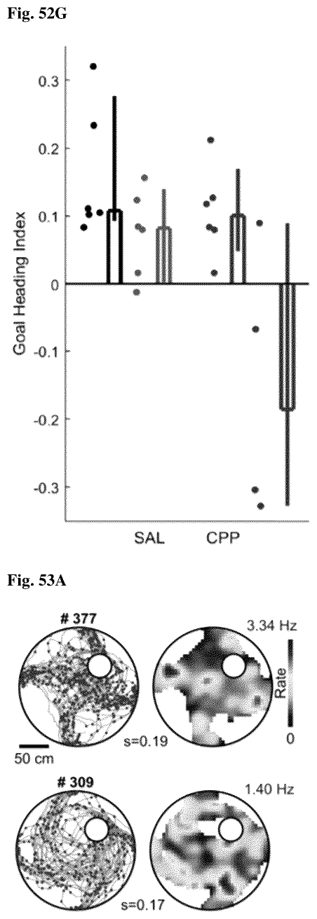

Rats readily learned to navigate to the hidden goal location using only distal visual cues in VR⁵, as measured by rewards per metre (RPM) of distance travelled. The task required different paths from the four start positions (FIG. 49A), which cannot be achieved using stereotyped paths. To further ensure the use of a navigational strategy, experimental conditions—such as the number of start positions, reward zone size and cues on the walls—were changed every 2-4 d. Well-trained rats continued to improve within these ‘session blocks’ across days (FIGS. 50A-50D). Furthermore, paths were distinct from each other in a more difficult task with eight start positions (FIGS. 50A-50D). Time spent in the target quadrant, or near the reward zone, and running speed near the reward zone further demonstrated efficient navigational behaviour (FIGS. 51A-51F, Supplementary Information). To verify that this task involved NMDA-dependent plasticity^{1,7,14,16,26}, six additional rats were injected with either saline or the competitive NMDAR antagonist (R)-CPPene^{27,28} (3.5 mg kg⁻¹; Methods). Upon task completion, a single probe trial was given. (R)-CPPene did not affect average trial length but greatly reduced overall locomotion and number of trials (FIGS. 52A-52G). Hence, the goal heading index (GHI) (Supplementary Information) was developed, which does not require many trials to compute (FIGS. 52A-52G). GHI was significantly greater than zero in probe sessions after saline, but not (R)-CPPene, injections (FIGS. 52A-52G), indicating the NMDAR dependence of the VNT, consistent with findings in one-dimensional virtual navigation in mice²⁹ and humans³⁰.

### Limited Allocentric Selectivity

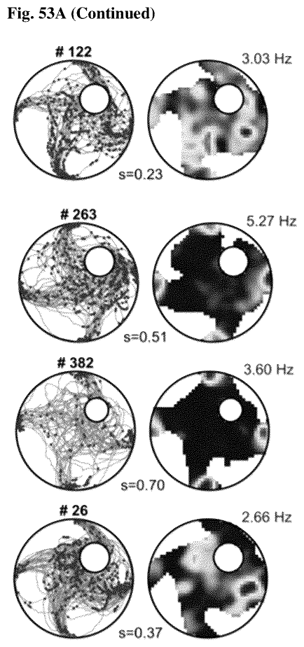

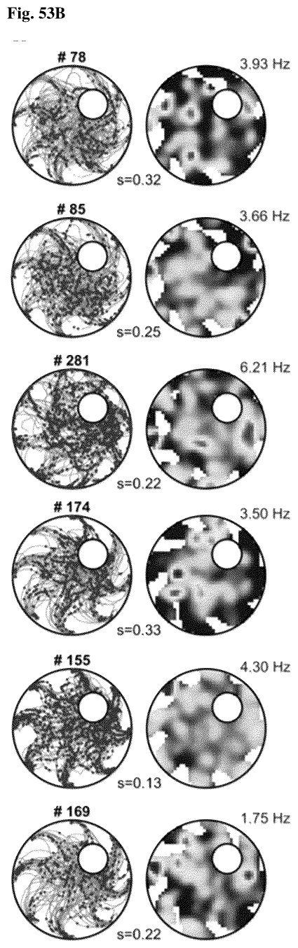

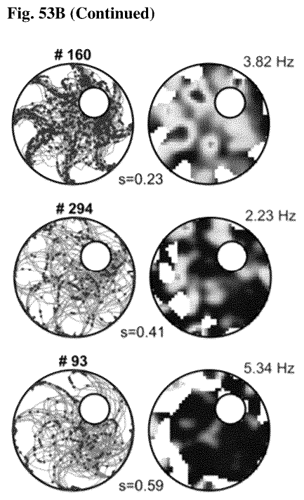

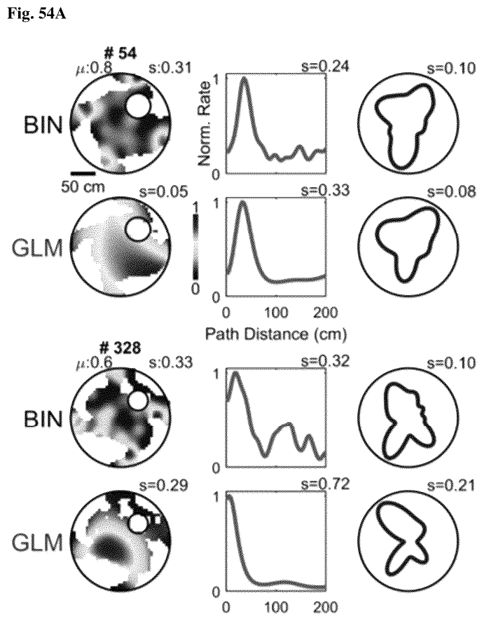

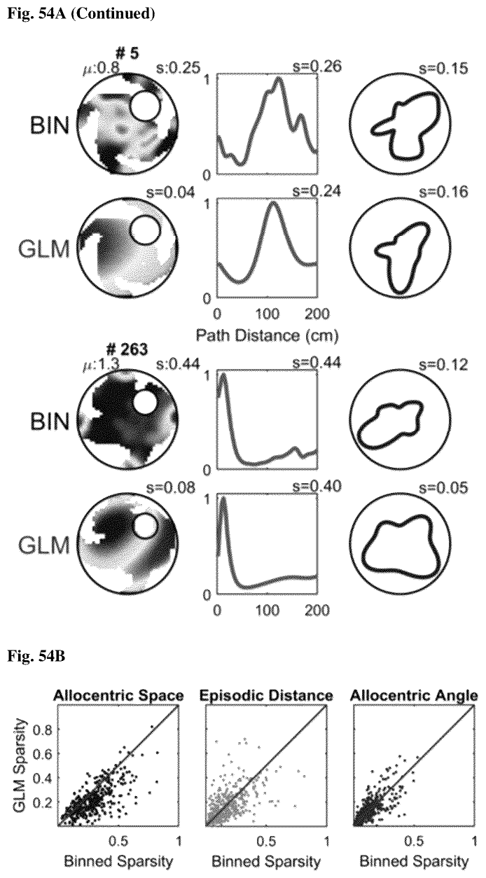

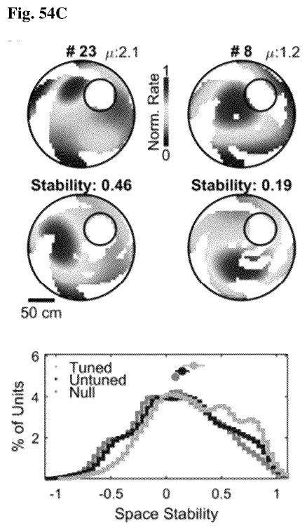

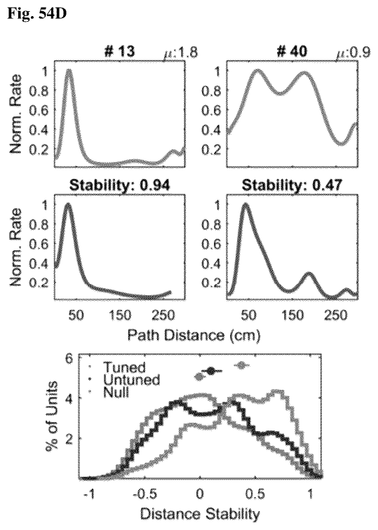

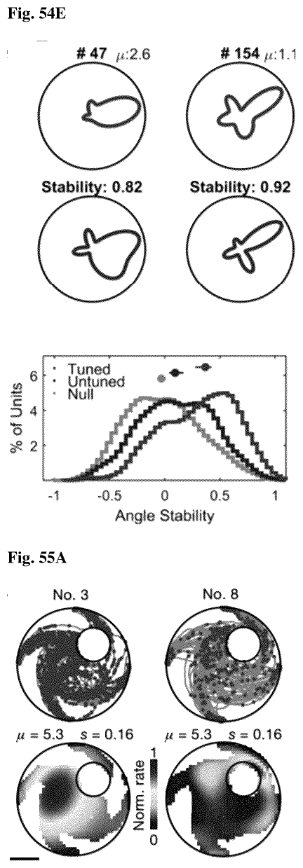

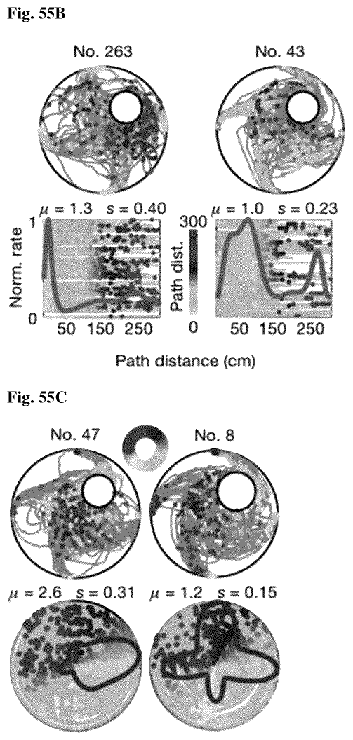

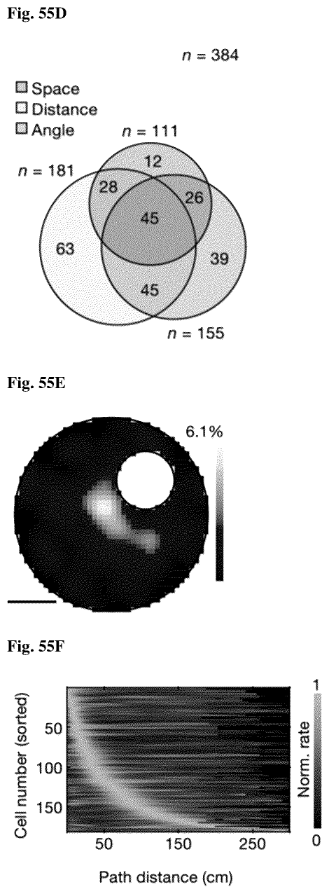

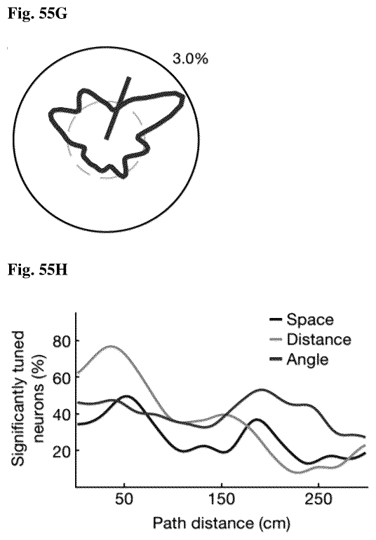

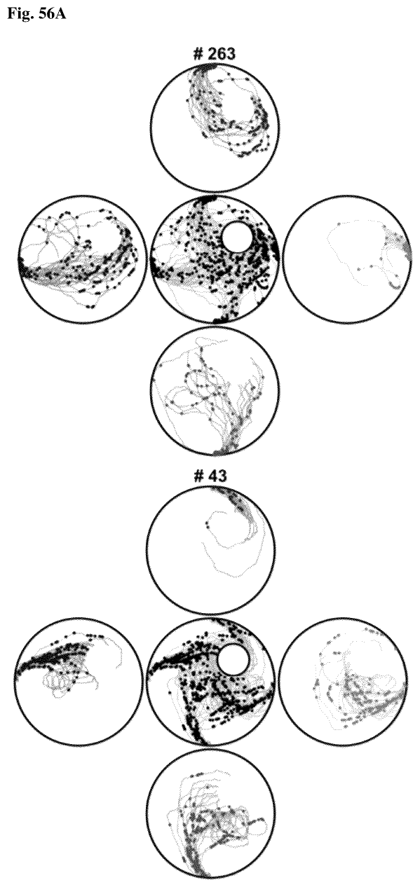

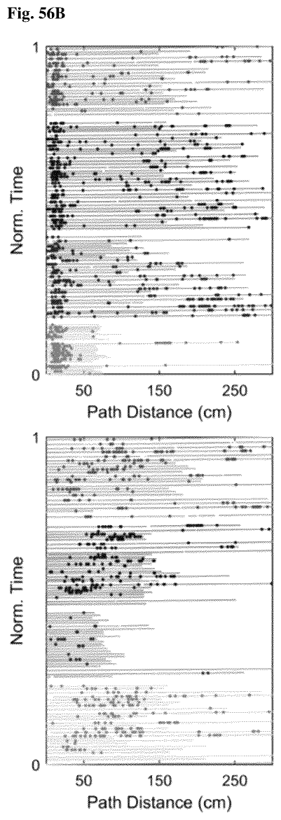

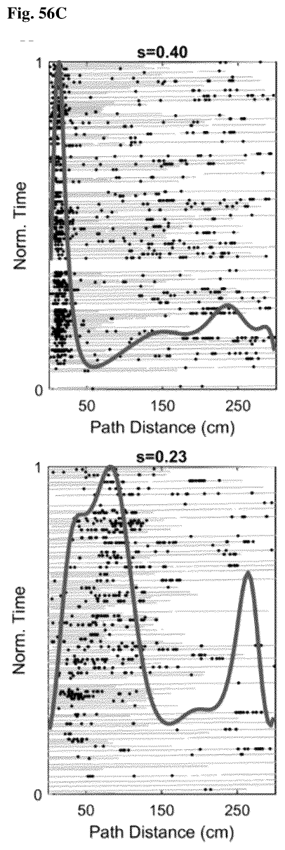

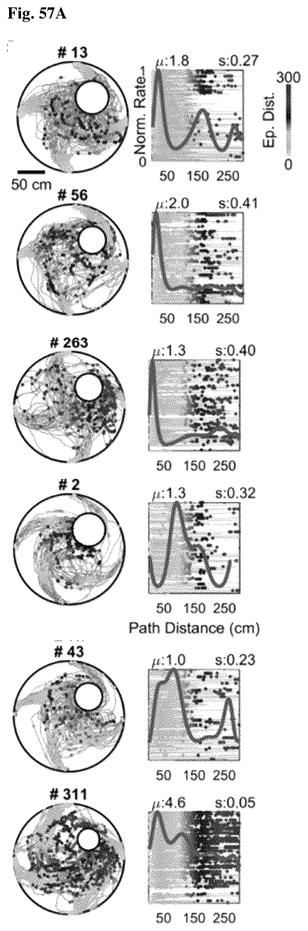

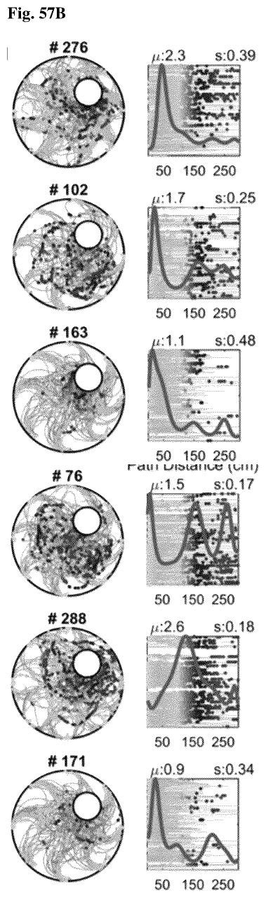

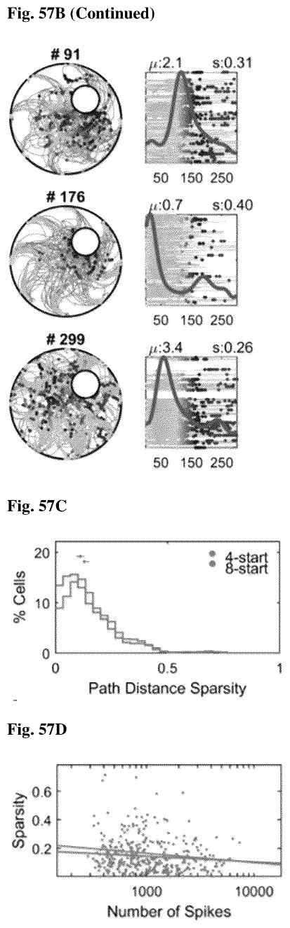

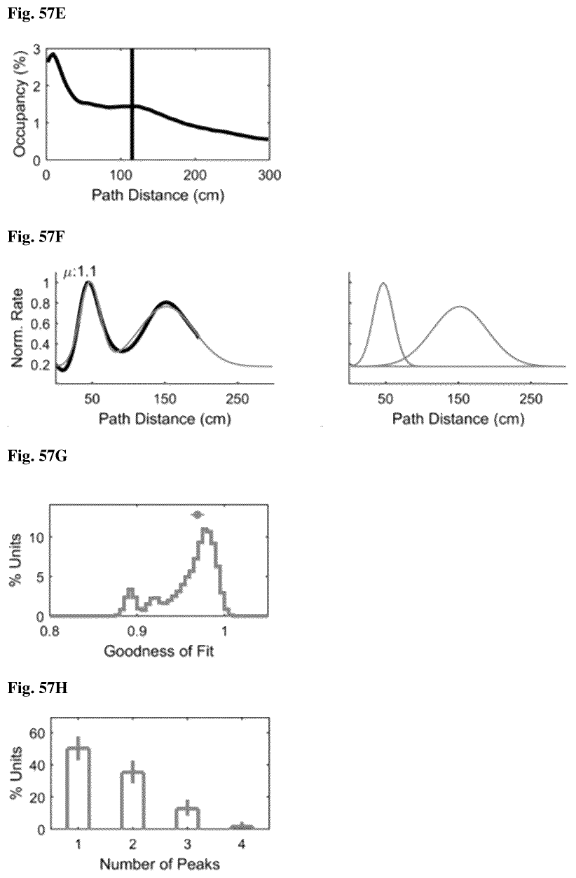

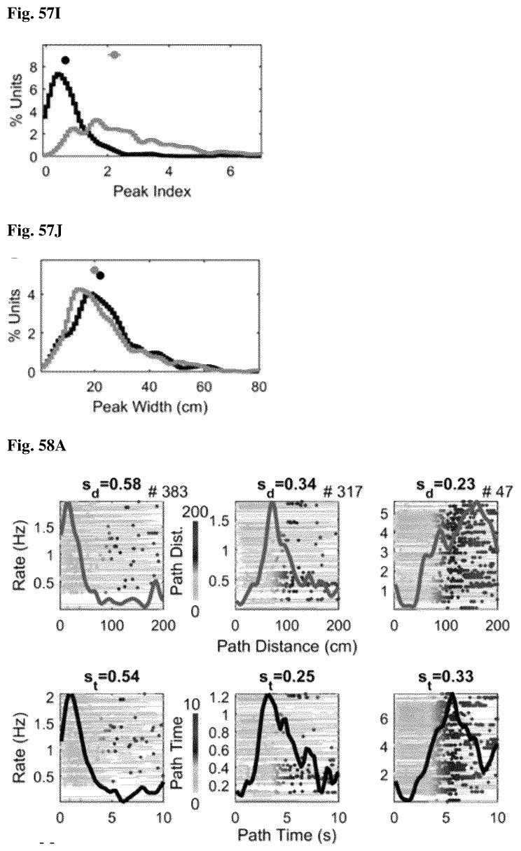

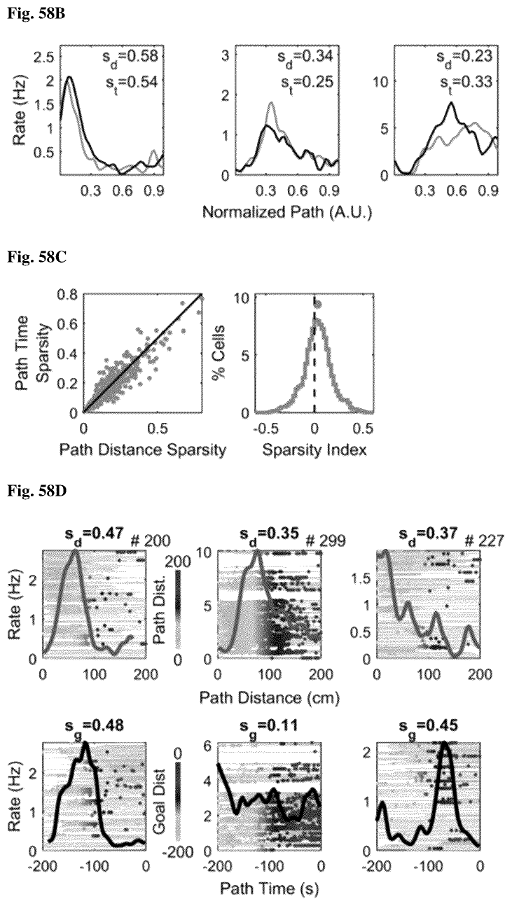

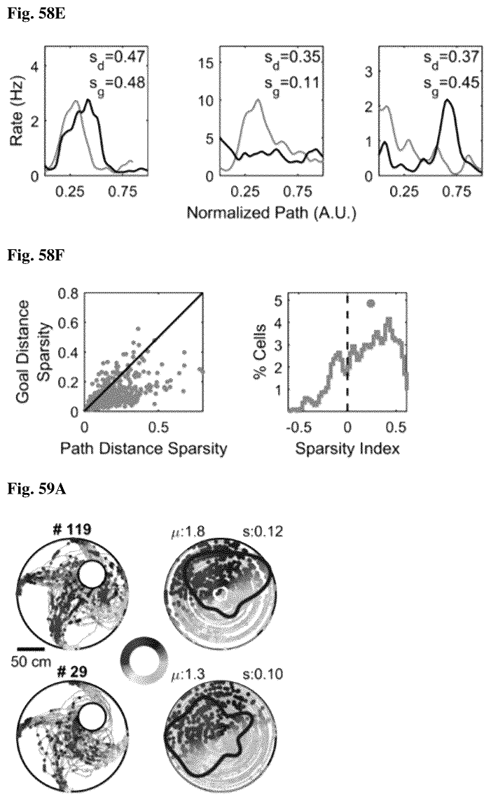

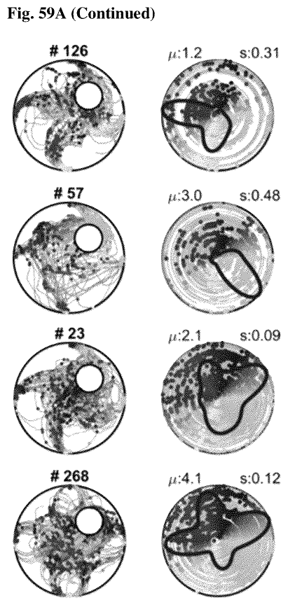

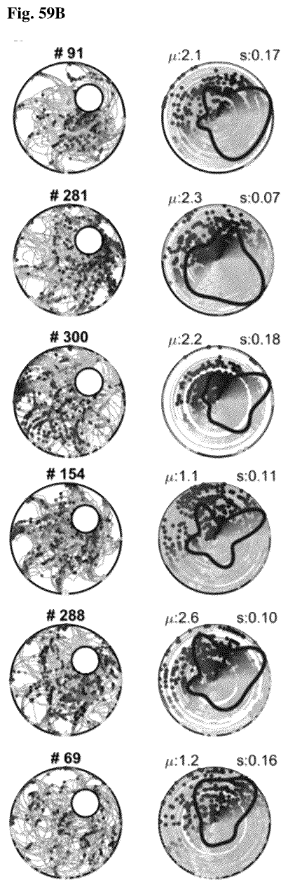

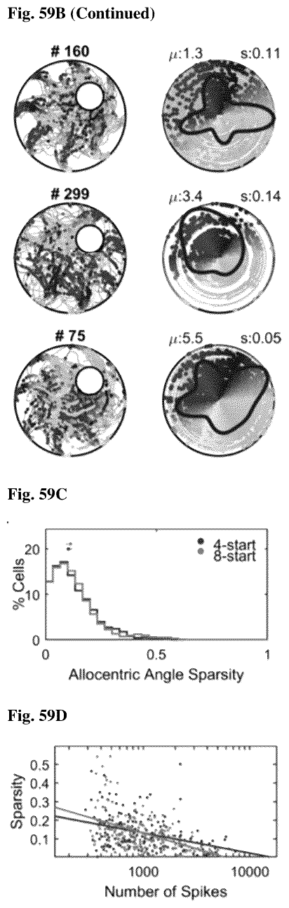

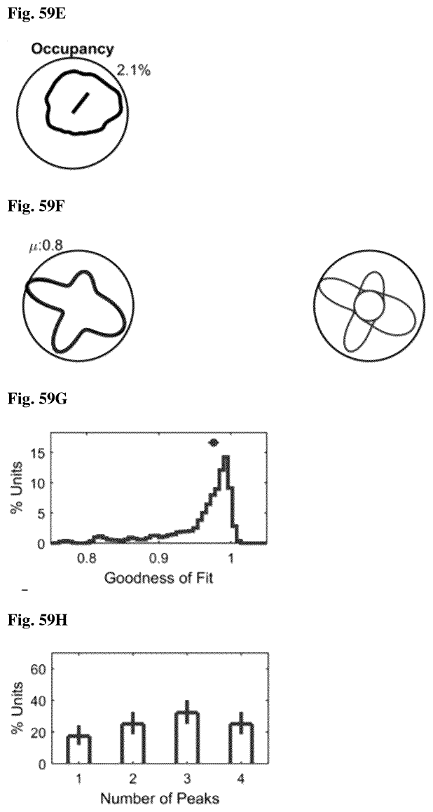

The activities of 384 putative CA1 pyramidal neurons were meausred from four rats in 34 sessions using tetrodes (Methods). In contrast to typical random foraging tasks in a two-dimensional real world (RW) environment, CA1 neurons showed relatively little allocentric spatial selectivity in virtual navigation (FIGS. 49C-49E, 53A, and 53B), similarly to that reported in a random foraging task in the same VR system²¹. Despite such low allocentric spatial selectivity, rats executed the navigation task exceedingly well. To explain this, it was hypothesized that hippocampal neurons could contain information about distance travelled and the direction of the reward^{12,23,31,32}, which could be sufficient for navigation. Because these variables are collinear, we further developed a generalized linear model (GLM)³³ to include these parameters as covariates and estimate their independent contribution to neural activity. Using this more sensitive analysis, few cells (˜30%) were significantly modulated by allocentric space (FIGS. 55A, 55D), and they were significantly less stable than place fields in an RW foraging task³³ (FIGS. 54A-54E, Supplementary Information). Allocentric place field peaks were neither uniformly distributed across the maze nor clustered near the start position34 but were significantly clustered near the reward zone (FIG. 55E), where the occupancy was the highest (Supplementary Information). Most neurons encode distance and angle By contrast, ˜50% of neurons were significantly modulated by path distance—that is, the distance travelled from the start of a trial, regardless of the allocentric start position (FIGS. 55B, 55D, 56A-56C, and 57A-57J)—similarly to place fields in real and virtual world one-dimensional mazes²⁰. Path distance field centers spanned ˜200 cm but clustered towards short distances, with a median distance of 32 cm (FIGS. 55B, 55F). This mirrors the behavioural oversampling of early distances (FIGS. 57A-57J) and might be related to navigation in an open-field event arena³⁵. This overrepresentation was not because all trials contained short distances, as distance fields were still aggregated when computed only for long trials (Supplementary Information). Path distance fields often were multi-peaked (FIGS. 57A-57J) and were more unstable than typical RW place fields but more stable than allocentric spatial maps in our experiments (FIGS. 57A-57J). Similar selectivity was observed when trial progression was measured as time elapsed³⁶ rather than distance travelled, with a small but significant preference for distance over time^{20,36} (FIGS. 58A-58F). Very few cells showed clear tuning to the path distance measured from the goal (FIGS. 58A-58F), and distance tuning was not explained by motor signals from turning alone (Supplementary Information). Similarly to two-dimensional random foraging tasks in both RW and VR33, ˜40% of neurons were significantly modulated by angle with respect to the distal visual cues (FIGS. 55B, 55D, 55G, and 59A-59J). Many angular tuning curves were multi-peaked, similar to those in the RW and VR33 (FIGS. 59A-59J). The peak angles spanned all directions (FIGS. 55C, 55G, and 59A-59J) but clustered towards the northeast direction, which, for most maze locations, is towards the hidden reward zone²³. In line with the other variables, angular field clustering matched the behavioural distribution (FIGS. 59A-59J). The angular tuning curves were slightly less stable than the distance fields (FIGS. 54A-54E) but similar to those during random foraging in VR³³. To determine whether neurons with different codes were distinct, we quantified the overlap among the populations of cells significantly modulated by space, distance and angle (FIGS. 55D, Supplementary Information). The proportion of cells that were modulated by two or more variables was approximately equal to chance levels, indicating that individual CA1 neurons can multiplex and simultaneously encode angular, allocentric and path-centric information, rather than being segregated populations^{19,37,38}.

### Neural Selectivity and Navigational Accuracy

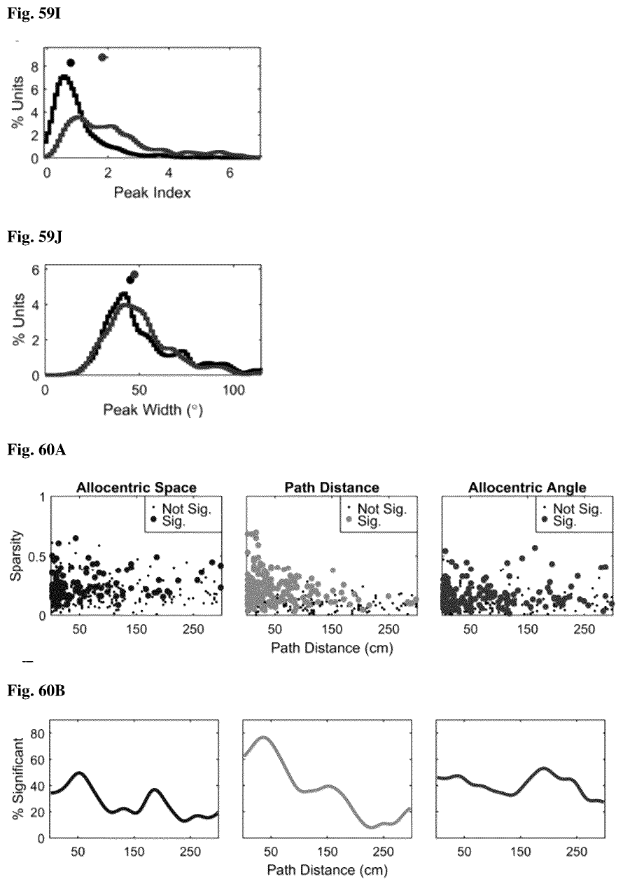

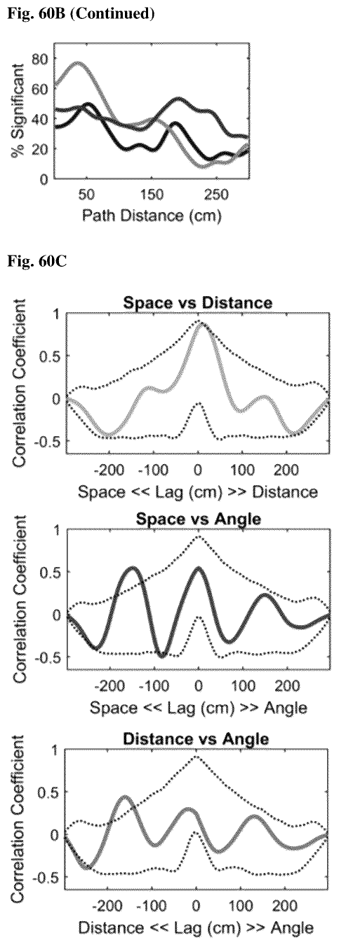

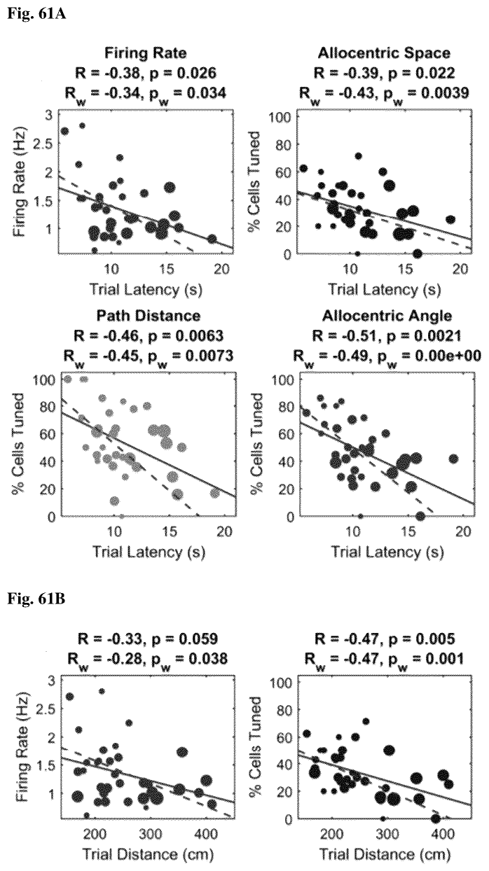

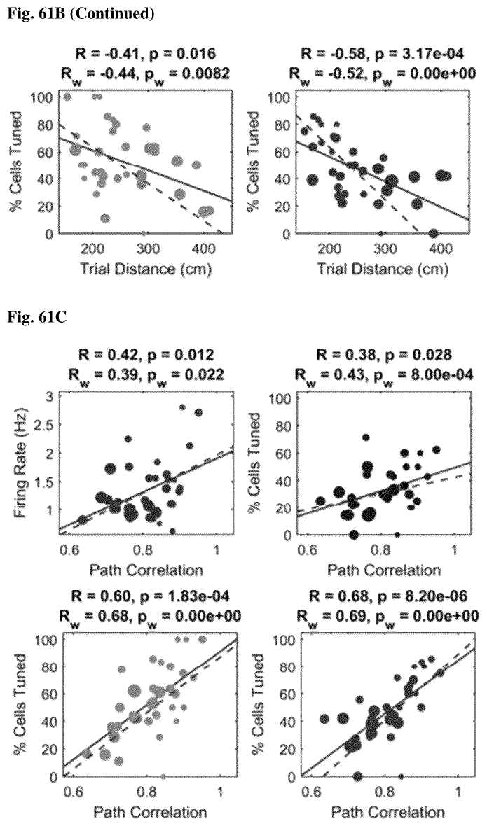

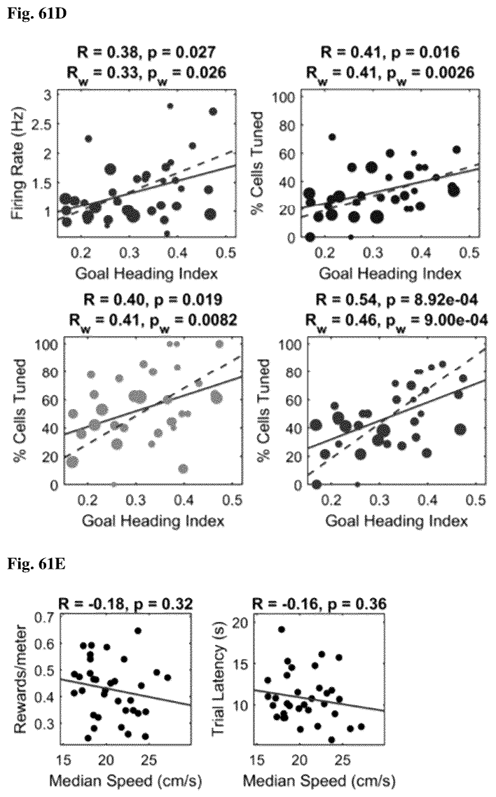

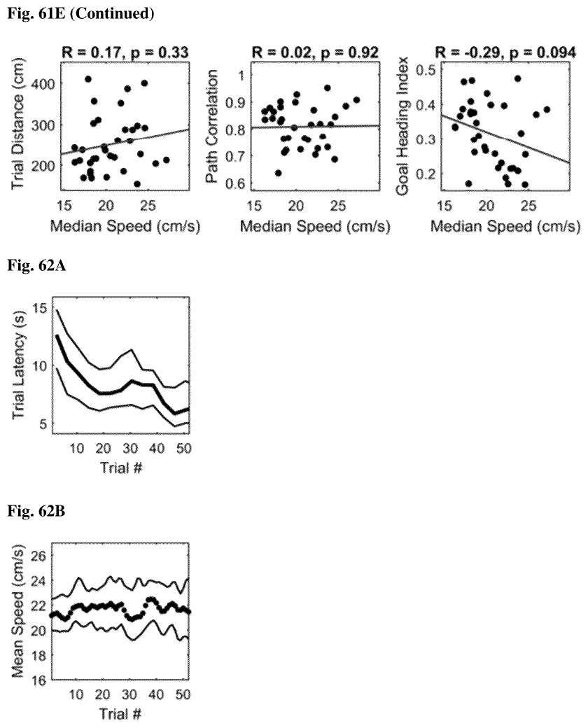

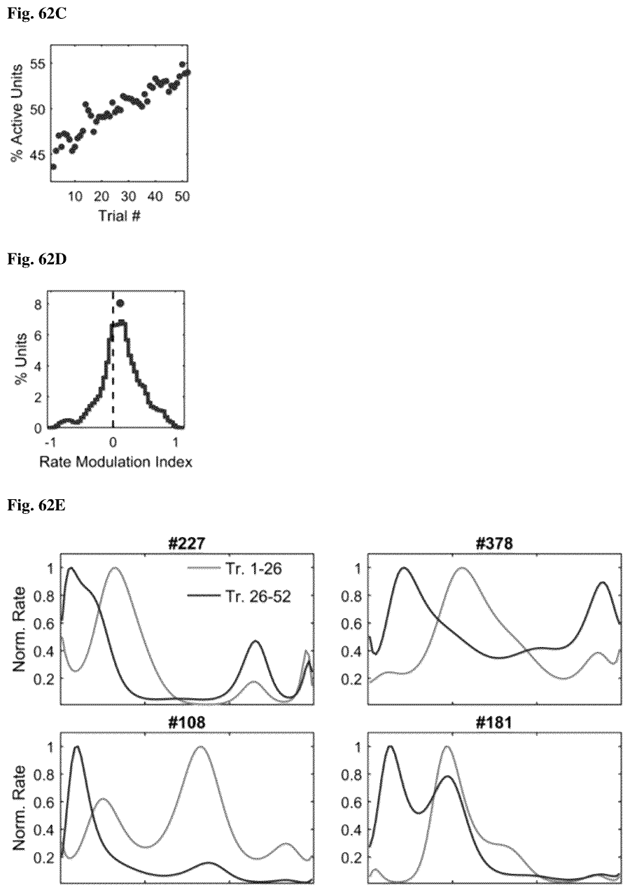

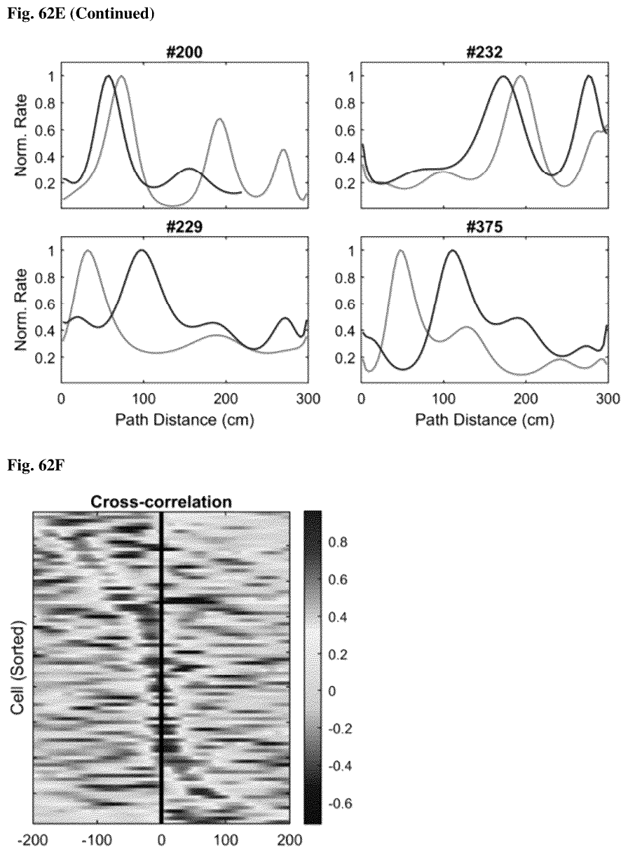

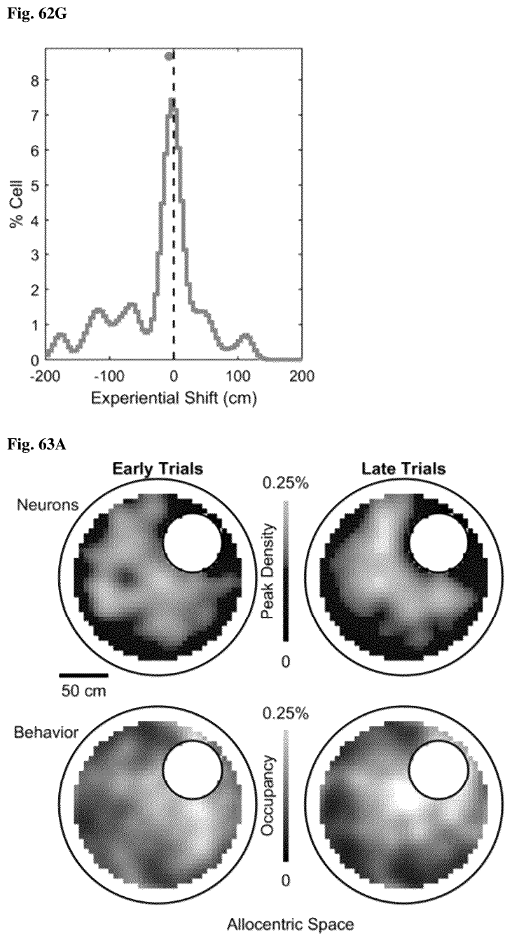

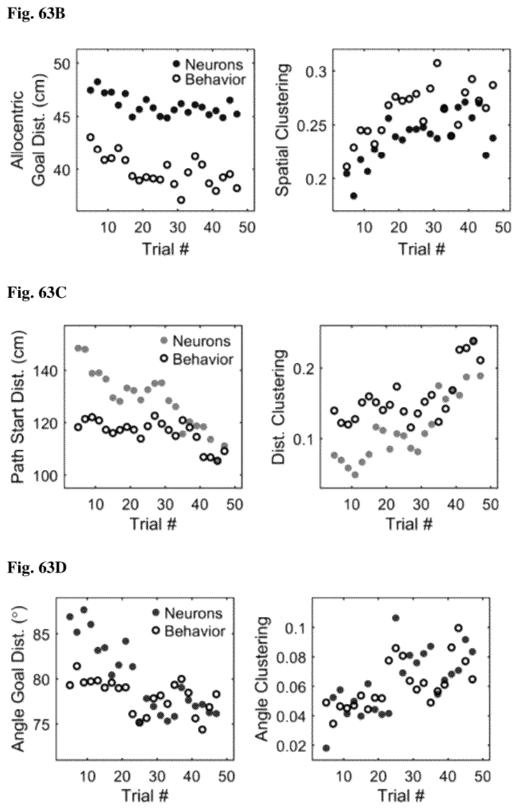

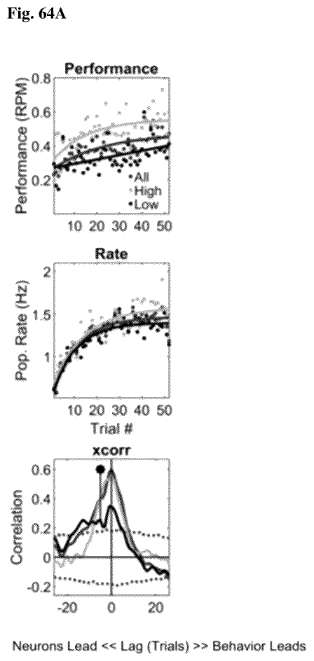

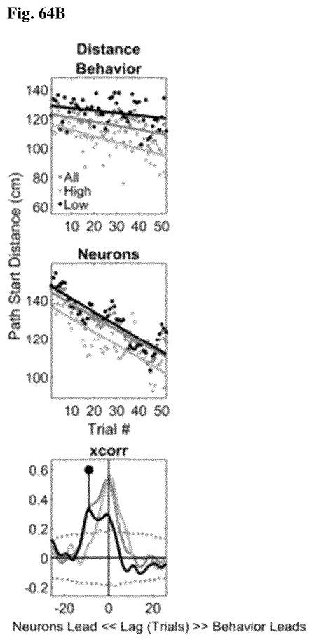

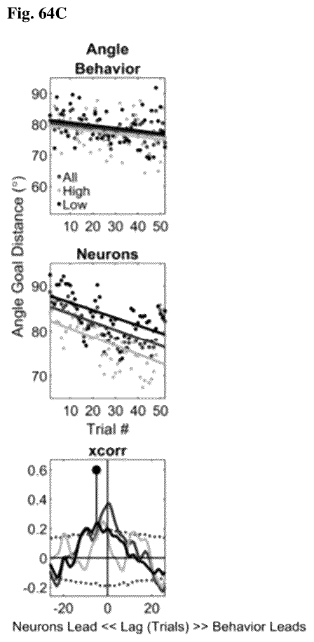

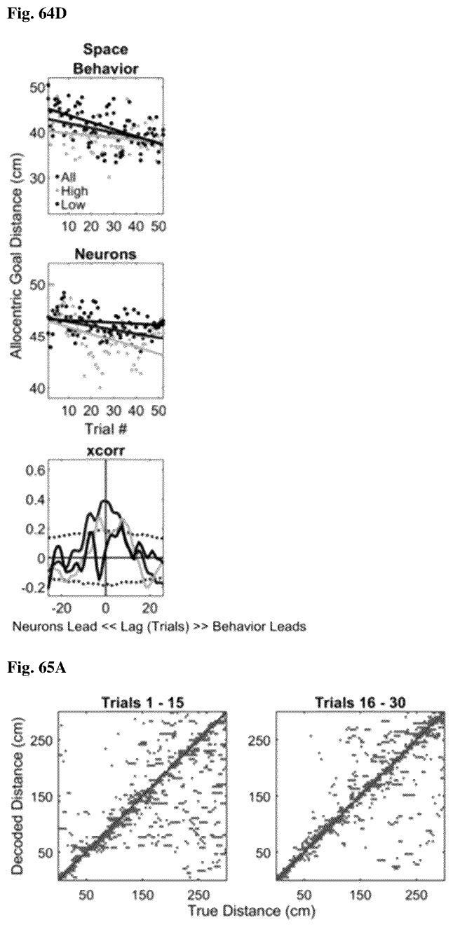

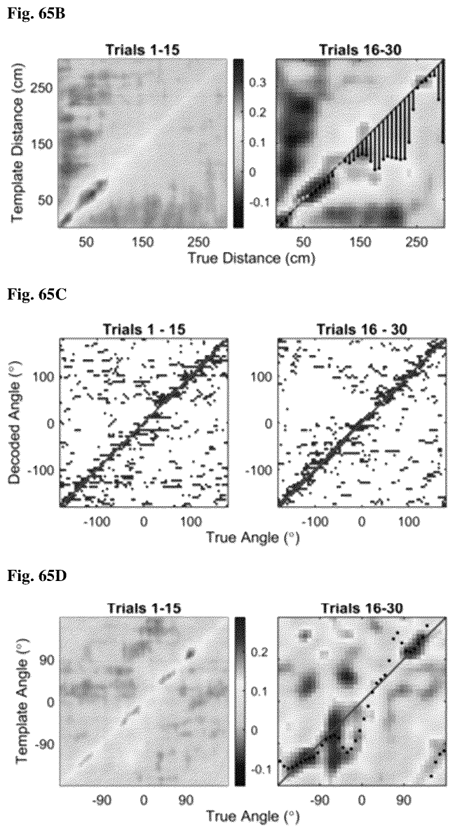

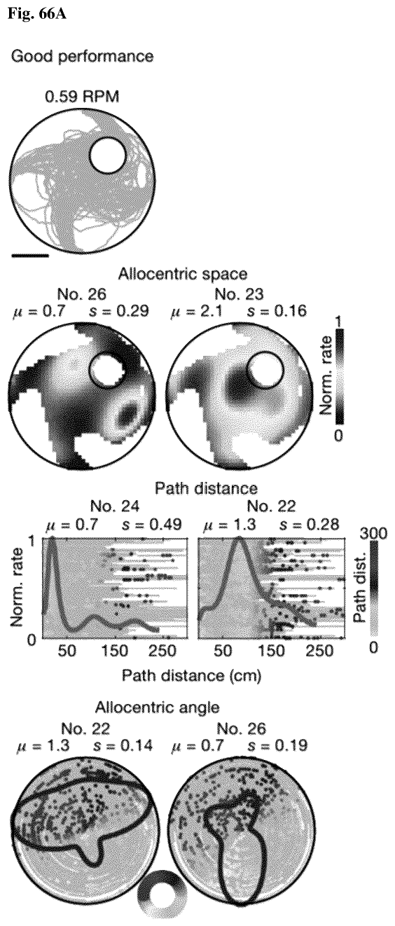

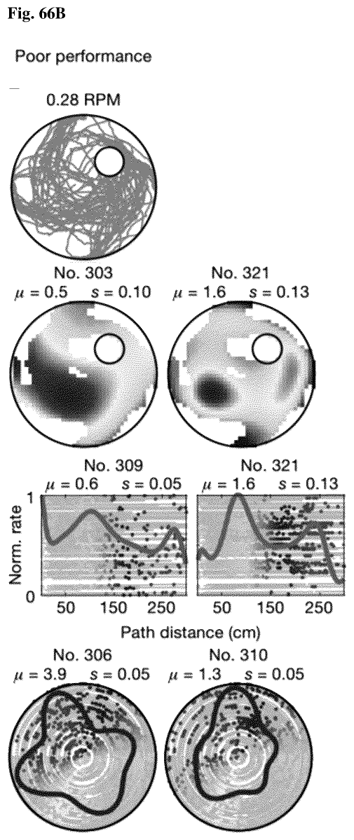

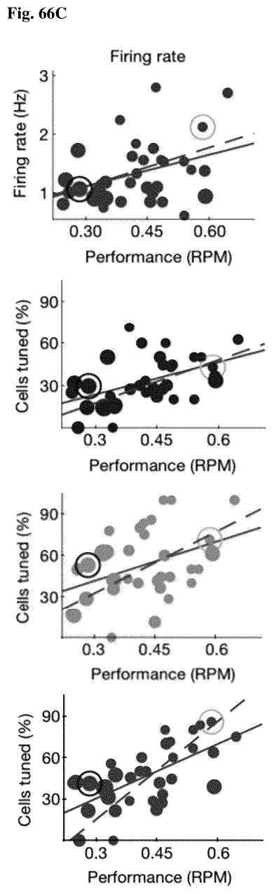

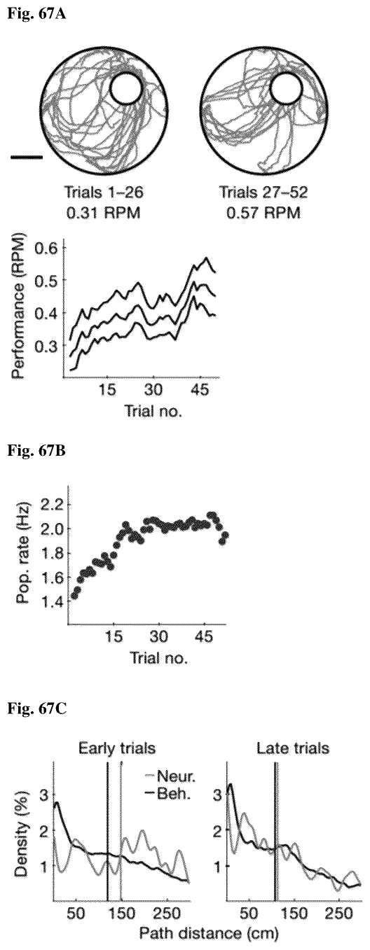

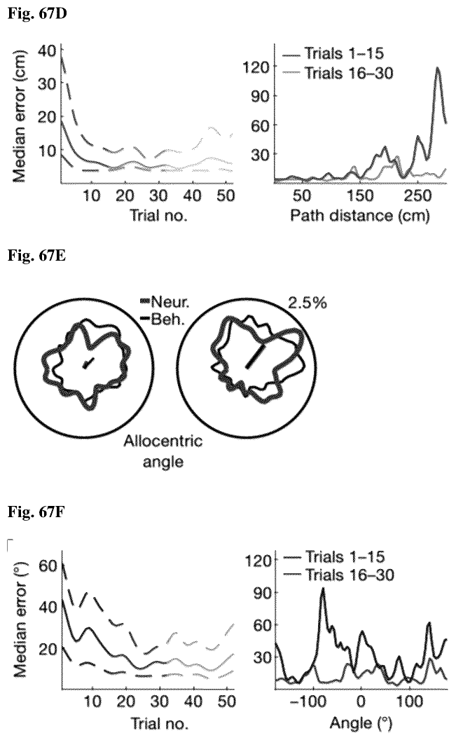

To determine whether these three neural codes were organized in an episodic and need-dependent way, the percentage of cells were quantified that were significantly tuned for space, distance or angle as a function of the distance at which the firing rate of the cell was maximal (FIGS. 55H and 60A-60C). Path distance tuning was highest at short distances (<50 cm). Allocentric space tuning showed two peaks, near 50 cm and 200 cm, congruent with the distribution of path lengths from nearby (50 cm) and distant (200 cm) start locations. Angle selectivity was relatively high throughout, with a peak at 200 cm—that is, when the rat is near the reward. This ordering of distance, space and then angle tuning would not arise simply by chance (FIG. 60C), suggesting that each journey is encoded as a continuous episode, made of the three codes, each becoming more prominent when it is needed the most. Although rats were well trained, the performance varied significantly across days (FIGS. 51A-51F, Supplementary Information). This was leveraged to investigate the relationship between behaviour and neural tuning (FIGS. 61A-61E and 66A-66C). The mean firing rate of cells in a session was significantly positively correlated with performance (FIGS. 61A-61E and 66C), independent of running speed (FIGS. 61A-61E and 66A). Notably, performance was positively correlated with the percentage of cells significantly tuned to angle, allocentric space or path distance (FIGS. 61A-61E and 66C). Neurometric and psychometric plasticity Performance improved significantly (up to 50%) within behavioural sessions each day, without increased running speed (FIGS. 62A-62G and 67A). Consistent with computational theories of navigational learning^{6,7,14,24,25}, ˜45% of cells were active in the initial trials within a session, increasing to 55% by trial 50 (FIGS. 62A-62G). The mean firing rate of active cells also increased with experience^{7,14-16}, from 1.5 Hz to 2.1 Hz (FIG. 67B). Next, experience-dependent changes in the path distance fields as a function of experience were examined, focusing on 88 cells with significant tuning to path distance but not angle (FIGS. 62A-62G). The cross-correlation among the GLM-derived path distance maps between first and second halves of a session (Supplementary Information) had significantly negative peak lags (median of −7.5 cm), indicating a net backward shift with experience^{7,14-16}, although some cells shifted forward. The plasticity of neural ensemble responses was then measured, particularly their clustering (FIGS. 55E-55G, Supplementary Information). With experience, allocentric spatial rate map peaks clustered towards the unmarked reward zone (FIGS. 63A, 63B); path distance peaks clustered towards the beginning of trials (FIGS. 63C and 67C); and angular tuning peaks clustered towards the quadrant containing the reward zone (FIGS. 63D and 67E). These changes in neuromeric curves tracked corresponding changes in psychometric curves, measured by occupancy. For all parameters, clustering was not necessarily near regions of reward but, rather, near regions of high occupancy⁸. We computed the temporal relation between changes in neural coding and behaviour using a cross-correlation analysis (FIGS. 64A-64D). The neural-behavioural correlation was strongest in high-performing sessions, but neural responses significantly preceded behavior in low-performing sessions. This might indicate a differential relationship between neural responses and behaviour at different stages of learning: a weak but causal relationship when performance is low, allowing neural responses to drive subsequent behaviour, and a strong and rapid relationship when performance is high, allowing efficient behaviour to be encoded in neural networks. Finally, neural selectivity and its experience dependence was evaluated using population vector decoding (FIGS. 65A-65D, 67D, and 65F). Decoding accuracy was high for path distance even in early trials at short distances (FIG. 67D), with large errors occurring after 150 cm. Decoding error decreased substantially within the first approximately ten trials, particularly at great distances. After experience within a session, even large distances had relatively small decoding errors, less than expected from error accumulation of distance by path integration. The population vector overlap among rate maps generated from all cells in late trials showed significant anticipatory shift compared to early trials (FIGS. 65A-65A-65D). Similarly, for angle, decoding near 45° was near asymptotic levels in early trials, with the improvement coming from angles that were less represented in the neural population (FIG. 67F). Angle decoding at all directions also considerably improved with experience, with subtle and varied anticipatory shifts (FIG. 67F, right), probably owing to systematic differences in angular behaviour within and across sessions.

### Discussion

The nature of hippocampal responses and their potential contribution to navigation were measured in a purely visually guided navigation task where all other cues, including olfactory and vestibular cues, were uninformative. This is similar to the vast majority of primate and human neurophysiology studies of hippocampal function where only visual cues are spatially informative^{18,30,39,40}. Thus, these experiments in rodents help to bridge the gap between rodent and human studies and reveal several similarities^{16,41}. During random-foraging-like tasks in VR, hippocampal spatial selectivity is weak in primates³⁹ and humans⁴¹, instead showing schema-like responses¹⁸. Analogously, hippocampal neural codes were found to be markedly different, and more complex, than place cells in the RW, where multiple sensory modalities contribute. Allocentric spatial selectivity is far less than in the RW in rodents and similar to that during random foraging in the same VR²¹. Thus, navigational task demand did not improve allocentric spatial selectivity. These results could depend on the nature of multisensory cues involved^{42,43}. On the other hand, this is consistent with the finding that mice lacking robust allocentric place cells⁴⁴ and primates without clear place cells³⁹ can navigate reliably. In contrast to the weak spatial selectivity, very strong angle and path distance tuning was observed. This could be because, in this task, only visual and podokinetic (step-counting) cues are spatially informative, and these are fairly dissociated owing to the multiple start positions. This could facilitate the dominance of path distance and angular selectivity, as opposed to allocentric selectivity, which might require other stimuli, especially olfactory or tactile²¹. This does not imply that allocentric spatial selectivity is never used for navigation but, rather, that it is not strictly necessary.

Remarkably, the same cell could multiplex and represent all three variables: allocentric space, angle and path distance. This supports the hypothesis that the hippocampus learns a schema, involving both spatial and non-spatial components, for accurate navigation⁴⁵. Contemporary studies have shown that most place cells are directionally selective in one and two^{33,46-48} dimensions in RW^{23,49} and VR^{20,21}, demonstrating multiplexed representation. In these experiments, distance coding independent of allocentric position could be interpreted as egocentric, because path distance is purely defined by the beginning of a trial.

### Clustering of Neural Responses

A large clustering of path distance fields was observed exclusively at the beginning of the paths, where navigational demands are highest³⁵. This is different from the nearly homogeneous distributions in rats and mice in RW or VR linear tracks^{20,34}, linear treadmill tasks²², clustering near goal locations on a treadmill³⁷ or annular water maze task¹¹ or bats approaching a visible goal²³. Unlike random foraging in RW^{33,46} and VR³³, where angle field peaks of were uniformly distributed, these were clustered towards the hidden reward zone. Thus, hippocampal angular tuning can be influenced by an un-cued, remembered location²³. Hebbian plasticity could explain both path distance and directional clustering⁸. Consistent with navigational strategies in the wild²³, both clustered towards regions of high behavioural occupancy: near the start for path distance and towards the goal for angle. Indeed, performance was correlated with the degree of spatial, distance and angular tuning, suggesting their key role in navigation³¹. Emergent properties of Hebbian synaptic plasticity Distance field clustering at the start positions and angle tuning towards the goal in a task-dependent manner are supported by models of navigational learning^{6-8,14,16,24,25} by STDP^{8,24,25}, as verified on linear tracks^{7,14-16}. Accumulation of this shift across days could contribute to the observed clustering at the start position³⁴. Transient NMDAR blockade caused significant impairment in the VNT performance, strengthening the link among Hebbian plasticity, neural plasticity and behavioural plasticity. In our study, rats chose to run very few trials with NMDA antagonists, which precluded a direct measurement of their effect on neural activity²⁸. Models of STDP also predict changes in the shape of the receptive fields, making them negatively skewed^{7,16,50}, which has been observed in subthreshold membrane potential of place cells⁵¹. Direct comparisons with these studies are difficult owing to the multi-peaked path distance fields and more subtle experiential effect on extracellular spikes compared to subthreshold membrane potential^{7,16,50,51}. The anticipatory shift in path distance fields is larger compared to that on linear tracks in the RW^{7,14-16}, which could arise owing to differences in task demand, and the absence of RW proximal cues that might anchor neural responses. Enhanced theta rhythmicity and slower eta rhythm observed in VR⁵² could further boost NMDAR-dependent plasticity⁵³. The time course of plasticity here is slower than that in one dimension^{7,14,16}, perhaps because each start position is experienced in an interleaved manner, and paths are more variable here. This experiential plasticity occurred every day, which is suggestive of reconsolidation⁵⁴. This is consistent with models^{16,55} and experiments suggesting partial pruning of hippocampal memory trace during sleep, resulting in improved signal-to-noise ratio of memories and improved performance the next day. There was also a significant increase in the number of cells that were active in the maze with experience¹⁴. This cannot be explained directly by STDP, which requires spiking. The activation of CA1 might have been inherited by STDP-related changes in the pre-synaptic structure, such as CA3 or entorhinal cortex^{16,56-58}, although this is difficult⁵⁷. Alternatively, dendritic spike⁵⁹ or plateau potential-induced, NMDAR-mediated plasticity within CA1 could activate more cells^{16,53,60} with experience. This resonates with the correlation between increased hippocampal activation and path integration performance in humans⁶¹. Improved receptive field selectivity would improve behaviour. Conversely, better behaviour—that is, more direct, systematic paths—would result in greater receptive field selectivity and plasticity. Thus, the two should be correlated, with receptive field improvements slightly preceding behavioural improvements. This prediction was strongly supported by the data: fluctuations between performance and neural firing rates, as well as various measures of neural selectivity, were strongly and significantly correlated, with neural responses preceding behaviour in many cases. Path integration and generalization The visual cues were entirely different among each start position, and rats had to behave differently to reach the hidden goal. Thus, the distance selectivity could not arise owing to specific movements or vestibular or non-specific cues. It must be computed de novo from each start position while overcoming the differences in visual cues, implicating path integration. The distance and direction tuning resemble path integration in various RW^{22,32,49} and VR^{20,21} tasks. However, path integration is thought to crucially depend on vestibular cues, which are missing in VR. Additionally, error builds up rapidly with path integration, whereas we found very little error buildup despite the absence of vestibular cues, highly variable behaviour and visual cues providing contrasting information from the four start arms. We previously hypothesized^{21,26} that 1-5-s motifs of activity from the medial entorhinal cortex⁶² drive hippocampal activity. When combined with multisensory inputs by somatic and dendritic-spike-mediated Hebbian plasticity1^{6,59}, it might generate selectivity to abstract quantities, such as distance, space and angle, to generate path integration9 and multisensory association^{16,20,21}. Thus, distance coding could be an abstraction or generalization that factors out visual, turning or other sensory cues that differ across start positions yet can integrate podokinetic cues to generate invariant, behaviorally relevant representations. This might be related to the relationship between path distance and entorhinal-hippocampal activation in humans³⁵. The angular tuning, which could be allocentric or egocentric^{23,63}, might be influenced by the specific set of distinct visual cues on the walls, as observed during random foraging³³, supporting cognitive mapping³ or spatial view responses in primates³⁹. However, the experience-dependent clustering of preferred angle towards the invisible reward zone would require additional computations, such as associative plasticity or reinforcement^{11,37}.

### Episodic Responses

These neural responses could form the basis of flexible, episodic spatial memory¹⁷, which is commonly thought to require information about what, when and where. Here, the ‘where’ information could be provided by the spatial and angular selectivity. The clustering of allocentric place cells near the hidden reward zone^{11,37} supports this hypothesis, along with the experiential forward movement¹³ and increased clustering. The ‘when’ information could be provided, in part, by the distance selectivity, triggered, but not determined, by self-motion. Indeed, most distance-selective cells were also selective for time elapsed³⁶. Notably, there was a significant temporal relationship among these variables: first distance and then angle and space, indicative of an episodic representation. Experience dependence of these representations could provide distinct information across trials and start positions. A significant portion of neurons multiplexed and encoded all three codes. Thus, not only the ensemble of neurons but also individual neurons could provide information about ‘what’ happened during the entire experience. These results provide evidence for the sequential, episodic arrangement of simultaneously existing allocentric and path-centric information in hippocampal neurons that is correlated with navigational performance. These responses show substantial neuroplasticity, greater than in the RW but consistent with computational models of spatial learning by Hebbian synaptic plasticity. Thus, the results help to bridge the gap in understanding about the cellular mechanisms of Hebbian plasticity, hippocampal episodic and allocentric selectivity and navigational performance. These results open up the possibility of testing rodents and humans under nearly identical, non-invasive and non-aversive conditions using VR to diagnose learning and memory disorders and achieve effective translation of treatments across species.

### REFERENCES

1. Morris, R. G. M. Synaptic plasticity and learning: selective impairment of learning in rats and blockade of long-term potentiation in vivo by the N-methyl-d-aspartate receptor antagonist AP5. J. Neurosci. 9, 3040-3057 (1989).
2. Bliss, T. V. P. & Lsmo, T. Long-lasting potentiation of synaptic transmission in the dentate area of the anesthetized rabbit following stimulation of the perforant path. J. Physiol. 232, 331-356 (1973).
3. O'Keefe, J. & Dostrovsky, J. The hippocampus as a spatial map. Preliminary evidence from unit activity in the freely-moving rat. Brain Res. 34, 171-175 (1971).
4. Scoville, W. B. & Milner, B. Loss of recent memory after bilateral hippocampal lesions. J. Neurol. Neurosurg. Psychiatry 20, 11-21 (1957).
5. Cushman, J. D. et al. Multisensory control of multimodal behavior: do the legs know what the tongue is doing? PLoS ONE 8, e80465 (2013).
6. Blum, K. I. & Abbott, L. F. A model of spatial map formation in the hippocampus of the rat. Neural Comp. 8, 85-93 (1996).
7. Mehta, M. R., Quirk, M. C. & Wilson, M. A. Experience-dependent asymmetric shape of hippocampal receptive fields. Neuron 25, 707-715 (2000).
8. Tsodyks, M. & Sejnowski, T. Associative memory and hippocampal place cells. Int. J. Neural Syst. 6, 81-86 (1995).
9. McNaughton, B. L. et al. Deciphering the hippocampal polyglot: the hippocampus as a path integration system. J. Exp. Biol. 199, 173-185 (1996).
10. Buzsáki, G. & Moser, E. I. Memory, navigation and theta rhythm in the hippocampal-entorhinal system. Nat. Neurosci. 16, 130-138 (2013).
11. Hollup, S. A., Molden, S., Donnett, J. G., Moser, M. B. & Moser, E. I. Accumulation of hippocampal place fields at the goal location in an annular watermaze task. J. Neurosci. 21, 1635-1644 (2001).
12. Pfeiffer, B. E. & Foster, D. J. Hippocampal place-cell sequences depict future paths to remembered goals. Nature 497, 74-79 (2013).
13. Xu, H., Baracskay, P., O'Neill, J. & Csicsvari, J. Assembly responses of hippocampal CA1 place cells predict learned behavior in goal-directed spatial tasks on the radial eight-arm maze. Neuron 101, 119-132 (2019).
14. Mehta, M. R., Barnes, C. A. & McNaughton, B. L. Experience-dependent, asymmetric expansion of hippocampal place fields. Proc. Natl Acad. Sci. USA 94, 8918-8921 (1997).
15. Mehta, M. R. & McNaughton, B. L. Expansion and shift of hippocampal place fields: evidence for synaptic potentiation during behavior. Comput. Neurosci. Trends Res. 741-745 (1997).
16. Mehta, M. R. From synaptic plasticity to spatial maps and sequence learning. Hippocampus 25, 756-762 (2015).
17. Tulving, E. Episodic memory: from mind to brain. Annu. Rev. Psychol. 53, 1-25 (2002).
18. Baraduc, P. & Wirth, S. Schema cells in the macaque hippocampus. Science 363, 635-639 (2019).
19. Pastalkova, E., Itskov, V., Amarasingham, A. & Buzsiki, G. Internally generated cell assembly sequences in the rat hippocampus. Science 321, 1322-1327 (2008).
20. Ravassard, P. et al. Multisensory control of hippocampal spatiotemporal selectivity. Science 340, 1342-1346 (2013).
21. Aghajan, Z. M. et al. Impaired spatial selectivity and intact phase precession in two-dimensional virtual reality. Nat. Neurosci. 18, 121-128 (2015).
22. Villette, V., Malvache, A., Tressard, T., Dupuy, N. & Cossart, R. Internally recurring hippocampal sequences as a population template of spatiotemporal information. Neuron 88, 357-366 (2015).
23. Sarel, A., Finkelstein, A., Las, L. & Ulanovsky, N. Vectorial representation of spatial goals in the hippocampus of bats. Science 355, 176-180 (2017).
24. Markram, H., LUbke, J. & Frotscher, M. Regulation of synaptic efficacy by coincidence of postsynaptic APs and EPSPs. Science 275, 213-215 (1997).
25. Bi, G. & Poo, M. Synaptic modifications in cultured hippocampal neurons: dependence on spike timing, synaptic strength, and postsynaptic cell type. J. Neurosci. 18, 10464-10472 (1998).
26. Mehta, M. R. & Wilson, M. A. From hippocampus to V1: effect of LTP on spatio-temporal dynamics of receptive fields. Neurocomputing 32-33, 905-911 (2000).
27. Kentros, C. et al. Abolition of long-term stability of new hippocampal place cell maps by NMDA receptor blockade. Science 280, 2121-2126 (1998).
28. Ekstrom, A. D., Meltzer, J., McNaughton, B. L. & Barnes, C. A. NMDA receptor antagonism blocks experience-dependent expansion of hippocampal ‘place fields’. Neuron 31, 631-638 (2001).
29. Sato, M. et al. Hippocampus-dependent goal localization by head-fixed mice in virtual reality. eNeuro 4, ENEURO.0369-16.2017 (2017).
30. Rowland, L. H. et al. Selective cognitive impairments associated with NMDA receptor blockade in humans. Neuropsychopharmacology 30, 633-639 (2005).
31. Dupret, D., O'Neill, J., Pleydell-Bouverie, B. & Csicsvari, J. The reorganization and reactivation of hippocampal maps predict spatial memory performance. Nat. Neurosci. 13, 995-1002 (2010).
32. Gothard, K. M., Skaggs, W. E. & McNaughton, B. L. Dynamics of mismatch correction in the hippocampal ensemble code for space: interaction between path integration and environmental cues. J. Neurosci. 16, 8027-8040 (1996).
33. Acharya, L., Aghajan, Z. M., Vuong, C., Moore, J. J. & Mehta, M. R. Causal influence of visual cues on hippocampal directional selectivity. Cell 164, 197-207 (2016).
34. Ziv, Y. et al. Long-term dynamics of CA1 hippocampal place codes. Nat. Neurosci. 16, 264-266 (2013).
35. Howard, L. R. et al. The hippocampus and entorhinal cortex encode the path and euclidean distances to goals during navigation. Curr. Biol. 24, 1331-1340 (2014).
36. MacDonald, C. J., Lepage, K. Q., Eden, U. T. & Eichenbaum, H. Hippocampal ‘time cells’ bridge the gap in memory for discontiguous events. Neuron 71, 737-749 (2011).
37. Gauthier, J. L. & Tank, D. W. A dedicated population for reward coding in the hippocampus. Neuron 99, 179-193 (2018).
38. Leutgeb, S. et al. Independent codes for spatial and episodic memory in hippocampal neuronal ensembles. Science 309, 619-623 (2005).
39. Rolls, E. T., Treves, A., Robertson, R. G., Georges-Frangois, P. & Panzeri, S. Information about spatial view in an ensemble of primate hippocampal cells. J. Neurophysiol. 79, 1797-1813 (1998).
40. Miller, J. F. et al. Neural activity in human hippocampal formation reveals the spatial context of retrieved memories. Science 342, 1111-1114 (2013).
41. Jacobs, J., Kahana, M. J., Ekstrom, A. D., Mollison, M. V. & Fried, I. A sense of direction in human entorhinal cortex. Proc. Nat Acad. Sci. USA 107, 6487-6492 (2010).
42. Aronov, D. & Tank, D. W. Engagement of neural circuits underlying 2D spatial navigation in a rodent virtual reality system. Neuron 84, 442-456 (2014).
43. Chen, G., King, J. A., Lu, Y., Cacucci, F. & Burgess, N. Spatial cell firing during virtual navigation of open arenas by head-restrained mice. eLife 7, e34789 (2018).
44. Resnik, E., McFarland, J. M., Sprengel, R., Sakmann, B. & Mehta, M. R. The effects of GluA1 deletion on the hippocampal population code for position. J. Neurosci. 32, 8952-8968 (2012).
45. Tse, D. et al. Schemas and memory consolidation. Science 316, 76-82 (2007).
46. Rubin, A., Yartsev, M. M. & Ulanovsky, N. Encoding of head direction by hippocampal place cells in bats. J. Neurosci. 34, 1067-1080 (2014).
47. Shahi, M. et al. A generalized linear model approach to dissociate object-centric and allocentric directional responses in hippocampal place cells. Soc. Neurosci. Abstr. 1, (2017).
48. Jercog, P. E. et al. Heading direction with respect to a reference point modulates place-cell activity. Nat. Commun. 10, 2333 (2019).
49. Cabral, H. O., Fouquet, C., Rondi-Reig, L., Pennartz, C. M. A. & Battaglia, F. P. Single-trial properties of place cells in control and CA1 NMDA receptor subunit 1-KO mice. J. Neurosci. 34, 15861-15869 (2014).
50. Mehta, M. R. Neuronal dynamics of predictive coding. Neurosci. 7, 490-495 (2001).
51. Harvey, C. D., Collman, F., Dombeck, D. A. & Tank, D. W. Intracellular dynamics of hippocampal place cells during virtual navigation. Nature 461, 941-946 (2009).
52. Safaryan, K. & Mehta, M. Enhanced hippocampal theta rhythmicity and emergence of eta oscillation in virtual reality. Nat. Neurosci. 24, 1065-1070 (2021).
53. Kumar, A. & Mehta, M. R. Frequency-dependent changes in NMDAR-dependent synaptic plasticity. Front. Comput. Neurosci. 5, 38 (2011).
54. Wang, S. H. & Morris, R. G. M. Hippocampal-neocortical interactions in memory formation, consolidation, and reconsolidation. Annu. Rev. Psychol. 61, 49-79 (2010).
55. Mehta, M. R. Cortico-hippocampal interaction during up-down states and memory consolidation. Nat. Neurosci. 10, 13-15 (2007).
56. Brun, V. H. et al. Place cells and place recognition maintained by direct entorhinal-hippocampal circuitry. Science 296, 2243-2246 (2002).
57. Ahmed, O. J. & Mehta, M. R. The hippocampal rate code: anatomy, physiology and theory. Trends Neurosci. 32, 329-338 (2009).
58. Mehta, M. R. Contribution of Ih to LTP, place cells, and grid cells. Cell 147, 968-970 (2011).
59. Moore, J. J. et al. Dynamics of cortical dendritic membrane potential and spikes in freely behaving rats. Science 355, eaaj1497 (2017).
60. Mehta, M. R. Cooperative LTP can map memory sequences on dendritic branches. Trends Neurosci. 27, 69-72 (2004).
61. Wolbers, T., Wiener, J. M., Mallot, H. A. & BUchel, C. Differential recruitment of the hippocampus, medial prefrontal cortex, and the human motion complex during path integration in humans. J. Neurosci. 27, 9408-9416 (2007).
62. Hahn, T. T. G., McFarland, J. M., Berberich, S., Sakmann, B. & Mehta, M. R. Spontaneous persistent activity in entorhinal cortex modulates cortico-hippocampal interaction in vivo. Nat. Neurosci. 15, 1531-1538 (2012).
63. Wang, C. et al. Egocentric coding of external items in the lateral entorhinal cortex. Science 362, 945-949 (2018).

### Methods

### Brief Methods

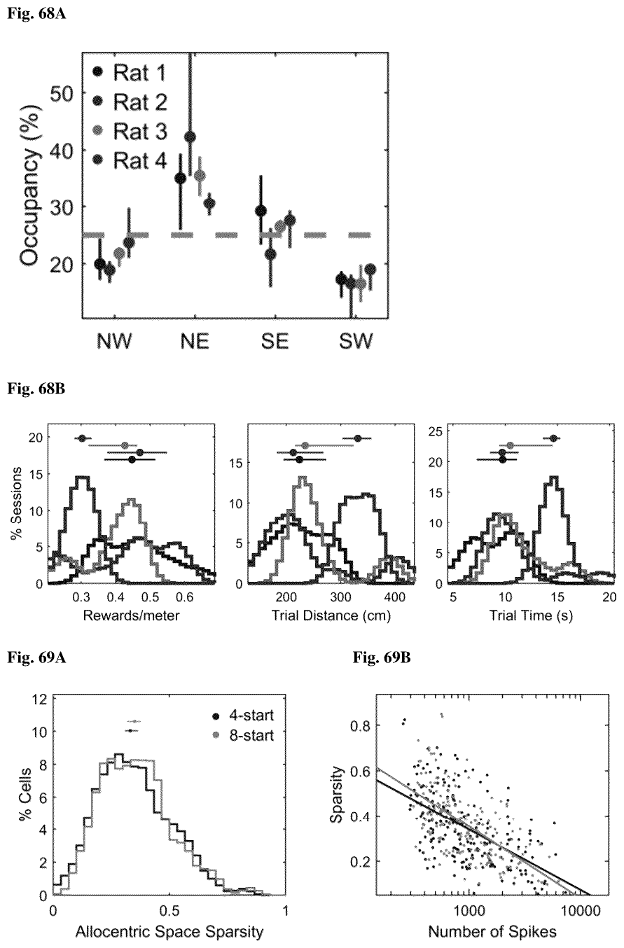

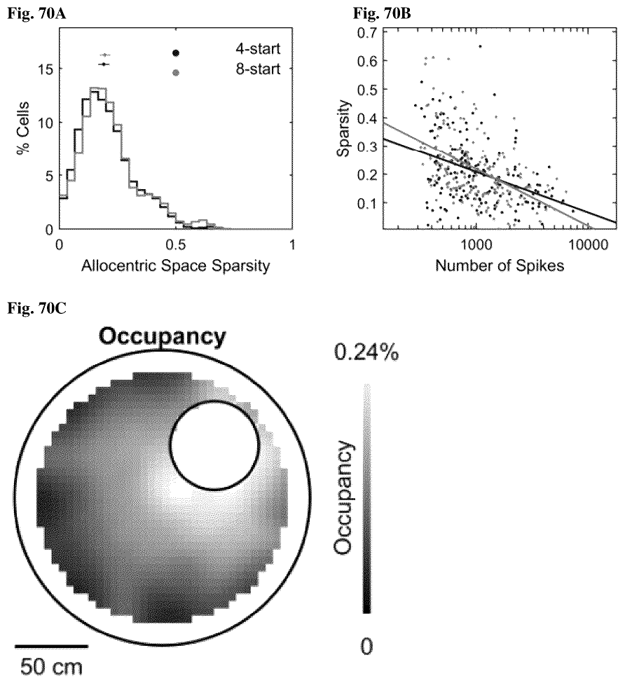

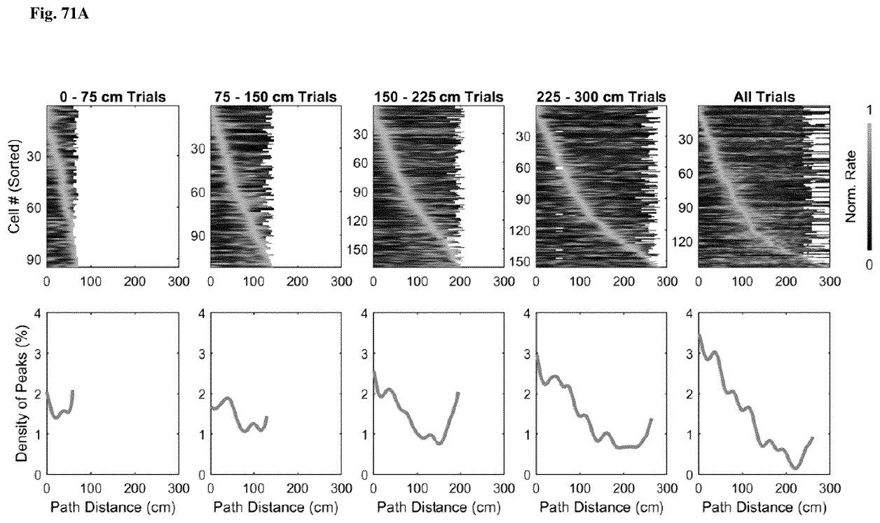

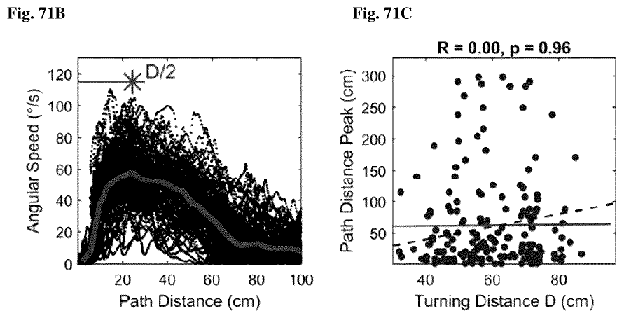

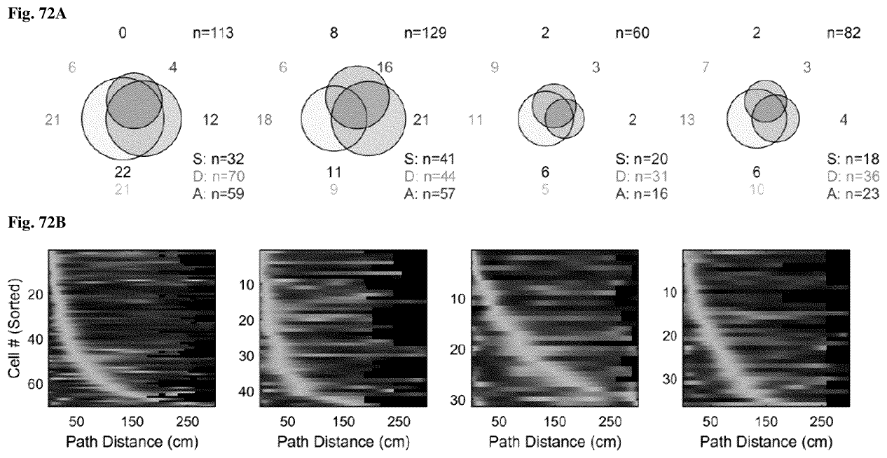

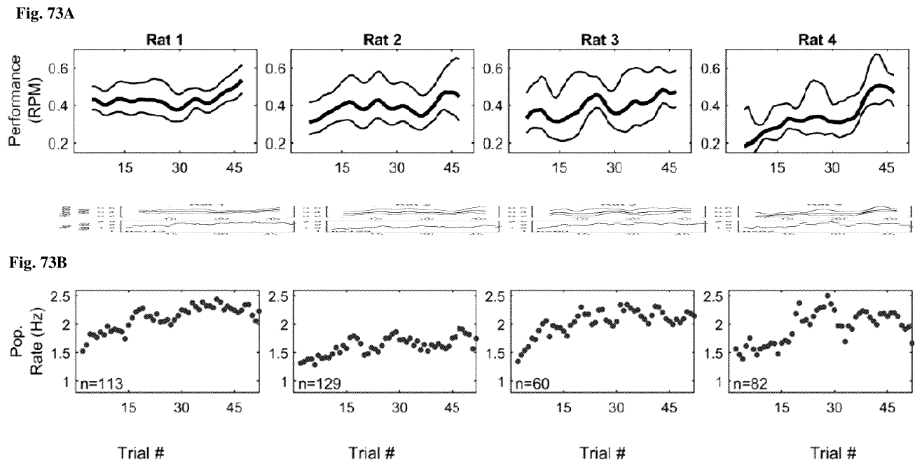

A body-fixed VR system was used in which rats were trained to run to a hidden reward location in the virtual space, as described previously⁵. Single units were recorded from bilateral CA1 in well-trained rats (n=4). Units were manually clustered offline, and well-separated pyramidal units with a mean firing rate during movement greater than 0.5 Hz were included in all analyses. A total of 384 units meeting these criteria were recorded across 34 behavioural sessions. No systematic differences were observed among data from different rats or from sessions with four start positions or eight start positions, so all units were pooled together for analysis, except for direct comparisons between the two conditions. Selectivity maps for allocentric position, path distance and head angle were simultaneously estimated using a GLM framework³³. The degree of selectivity was quantified by sparsity, and statistical significance for each unit was assessed using its own shuffled data. Rats Four adult (7-16-month-old) male Long-Evans rats were implanted with bilateral hyperdrives each containing up to 12 tetrodes per hemisphere. Rats were food and water restricted to motivate performance. Six additional unimplanted adult (10-14-month-old) male Long-Evans rats were trained to perform the behavioural task alone, after which they were injected with NMDA antagonists (see below). All experimental procedures were approved by the University of California Los Angeles Chancellor's Animal Research Committee and were conducted in accordance with US federal guidelines. Behavioural task Animals navigated in a virtual space using a body-fixed VR system, as previously described^{5,20}. The circular virtual table was 100 cm in radius, placed in the centre of a room measuring 400×400 cm. Each wall had a unique visual design to provide a rich visual environment (FIG. 49A). The table had a finely textured pattern to give optic flow without providing spatial information. The table was placed 100 cm above a floor with a black and white grid pattern so rats were able to visually detect and turn away from the edge of the table⁵. Trials began with the rat in one of four (or eight) start positions, at a distance of 5 cm from the table edge facing radially outwards. These start positions corresponded to those directly facing the walls (defined as north, east, south and west) for sessions with four start positions and angles in the middle of these for sessions with eight start positions. The hidden reward zone (radius of 20-30 cm) was always located in the northeast quadrant with its centre at coordinates (35.3, 35.3). Rats freely moved around the virtual space until they entered the reward zone. Upon entry, the reward zone turned white, and pulses of sugar water were delivered at 500-ms intervals, accompanied by auditory tones for each pulse. This continued until five rewards were delivered or the rat exited the reward zone, ending the trial. At trial end, the visual scene was turned off, and a blackout period of 2-5 s ensued. Rats were teleported to a new randomly chosen start position during this period. The visual scene was then restored, and a new trial began. The VR environment and virtual position tracking was done using custom-written software in C++. To encourage the use of a navigational strategy rather than a stereotyped motor response, experimental conditions were occasionally changed to define ‘session blocks’ unique to each rat. Specifically, the number of start positions, the reward zone size or the set of wall cues was changed every 2-4 d, yielding a total of 12 session blocks (FIG. 50A). The primary performance measure was rewards per meter travelled (RPM), equivalent to the inverse of the mean path length. To quantify the improvement in behaviour across sessions, we calculated the percent change in RPM relative to the first day in a session block (FIG. 50A, bottom). To test for statistical significance of the change in performance, the difference between the percent change on consecutive days within a session block was computed (27 such differences) and was analysed by a two-sided Wilcoxon sign-rank test. This is a conservative estimate, as we include data from up to 4 d in the same condition, whereas the biggest improvement tended to occur between day 1 and day 2 within a session block. Position in the virtual environment was sampled at a rate of 55 Hz. Throughout the paper, ‘angle’ refers to the viewing angle of the virtual avatar. Angle is purely defined by visual cues, as rats are body-fixed to point in the same direction in the RW frame of reference at all times. Electrophysiology Neural activity was recorded extracellularly from dorsal CA1 using tetrodes. Tetrodes were made from a nickel-chromium alloy and insulated with polyimide. Data were recorded using the Digital Lynx SX acquisition system (Neuralynx), controlled using Cheetah 5.0 software (Neuralynx). Action potentials were detected as described previously and manually sorted into putative neurons or units20,21 using a customized program written in Python 2.7. Only putative pyramidal neurons were used for analysis, identified by having a high complex spike index (>15) and waveform with a width at half-maximum for at least 0.4 ms. Only units with a mean firing rate greater than 0.5 Hz during movement (speed>5 cm s⁻¹) were included. Tetrodes were adjusted daily to increase the total number of independent neurons. NMDAR block in vivo To test whether our VNT involved NMDA-dependent plasticity, we trained a separate group of six unimplanted rats to perform this task. These rats were subjected to multiple environments and reward zone locations with application of either the NMDAR antagonist (R)-CPPene or saline vehicle, according to the following schedule. Rats were trained to navigate in the VR on a similar schedule as that for the implanted rats. On day 1 of week 1, the distal visual cues of the virtual environment were changed, and the reward zone was relocated. Rats were given an intraperitoneal injection of 3.5 mg kg⁻¹ of (R)-CPPene27,28 and then allowed to rest for 1 h in a sleep box before a 30-min VNT session. Immediately after this session, a probe trial of 1-3 min was run in which the reward zone was disabled. This process was repeated for a total of 5 d in the same environment, with injection only on day 1. This protocol was repeated the following week with a new environment and reward location, with an intraperitoneal injection of an equivalent volume of saline on day 1. Sample sizes Sample sizes for the number of cells to be used in analyses were not pre-determined and were constrained by the number of viable units recorded using available tetrode technology. Six rats were chosen for the NMDAR block experiments to be able to provide sufficient statistical power using non-parametric tests should a clear effect (all performances impaired) be observed. Data exclusions No data were excluded from analyses. Replication Recordings were performed in parallel across all rats over the span of many weeks. Primary findings were largely maintained in individual animals (FIGS. 68A, 68B, 72A, 72B, and 73A-73D), serving as biological replicates. Randomization and blinding Different experimental groups were not established for the primary findings, so randomization and blinding were not performed. Animals served as their own controls in the NMDAR block experiments.

### Binning Method of Computing Rate Maps

Similar methods as described previously²¹ were used to compute rate maps using the binning method. In all analyses, only behaviour and neural activity when the rat was running faster than 5 cm s⁻¹ outside the reward zone were included. Additionally, only data with a path distance less than 300 cm were included, because longer run distances were rare (<5% of trials). Additional details are provided in the Supplementary Information. GLM A GLM with logarithmic link function, similar to the one used in previous work³³, was further developed to estimate the simultaneous contribution of space, distance and angle to neural firing, including regularization⁶⁴. For each neuron, spike counts were binned using 100-ms bins. For allocentric space, between 5 and 32 Zernike polynomials were used as basis functions³³. For path distance, basis functions were the first 10 Chebyshev polynomials of the first kind⁶⁵. For angle, basis functions were sine and cosine functions with frequencies n/2π for n={1, 2, 3, 4, 5}. These choices of basis functions provided orthonormal sets that spanned the entire parameter range. Additional details are provided in the Supplementary Information.

### Quantifying Tuning

A sparsity to quantify the degree of tuning of each rate map. Maps were divided into N bins as defined above, and sparsity was computed as

$$Sparsity ( s ) = 1 - \frac{{( {\sum }_{i = 1}^{N} {R}_{i} )}^{2}}{N {\sum }_{i = 1}^{N} {R}_{i}^{2}} ,$$

where Ri is the value of the rate map in the ith bin.

### Statistics

All analyses were performed using custom-written code in MATLAB version 9.5 (R2018b). For determining the statistical significance of rate maps, each unit served as its own control. For each parameter (space, distance and angle), the relevant behavioural vector was time reversed and shifted by n×s seconds, for n=1-60, where s is the duration of the session divided by 61. Rate maps were re-estimated for this altered dataset, and the resulting sparsity values formed a null distribution for the shifted parameter. Rate maps were considered statistically significantly tuned if the original sparsity exceeded all 60 control sparsity values, yielding an effective statistical criterion of P<0.017. This procedure is non-parametric and does not make any assumptions about the nature of the null distributions. Offsets were generated from a continuous range rather than using randomly drawn offsets to eliminate the possibility of randomly selecting two offset values that are near each other. This ensures that the shuffled distribution is composed of independent data points.

### Weighted Correlations and Linear Fits

To robustly estimate the relationship between performance and neural tuning (FIG. 61A-61E, 66C), both unweighted and weighted correlation coefficients and linear fits were computed. For weighted calculations, each session was weighted by the number of units in that session. To determine statistical significance, we performed a bootstrapping procedure. The data was resampled 10,000 times with replacement to generate a distribution of weighted correlation coefficients. The P value was the fraction of resampled datasets with a correlation coefficient R≤0 for FIGS. 61C and 66C or R≥0 for FIGS. 61A, 61B.

### Stability of GLM Maps

To demonstrate the robustness of the GLM fitting procedure, a stability analysis was performed (FIGS. 54A-54E). Tuning curves for the first and second half of sessions were estimated using the GLM procedure described above using data only from trials 1-26 and 27-52, respectively. Valid bins in these restricted rate maps had to have a minimum occupancy more than 50 ms for allocentric space and more than 500 ms for path distance and angle.

Stability is defined as the correlation coefficient between first-half and second-half maps. Additionally, as stated above, all GLM fitting was performed using fivefold cross-validation to mitigate the effects of overfitting. Cells were classified as ‘tuned’ or ‘untuned’ based on their maps estimated from the entire session, as described above (‘Statistics’ section). Null distributions in FIGS. 54A-54E were obtained by computing the correlation coefficients between random first-half and second-half maps by shuffling cell identities once.

### Episodic Relationship Among Distance, Angle and Position Codes

To compute the percentage of cells significantly tuned for space, distance or angle as a function of the progression of a trial (FIGS. 55H and 60A-60C), a centre coordinate was assigned for each cell as the location of the peak of its path distance field derived from the GLM. Percentage tuned as a function of distance was then computed using the distance bins defined for binned rate maps and smoothed with a Gaussian kernel with a sigma of 15 cm. The significance of the ordering of tuning to the three variables was tested using a cross-correlation analysis (FIG. 60C). To compute significance while controlling for the non-uniform distribution of peak locations, a resampling procedure was performed. All units, significant or not, were assigned a new peak location by resampling with replacement from the original pool of peak locations; the population of these surrogate cells then went through the same procedure described above to generate null curves and null cross-correlations. This was repeated 5,000 times, and the dotted lines in FIG. 60C represent the 95% range at each lag for the control data.

### Analysis of Variance for Sparsity Between Conditions

Sparsity is typically negatively correlated with the logarithm of the number of spikes that a neuron fires in a session³³. Thus, we used a two-way ANOVA in MATLAB to compare sparsity among VNT, random foraging in RW and random foraging in VR (FIG. 49D) or between four- and eight-start VNTs (Supplementary Information, FIGS. 57A-57J and 59A-59J). Recording condition (VNT, RW, VR and four-start or eight-start) was a categorical predictor, and log 10 (number of spikes) was a continuous predictor of the sparsity. The P values reported in the identified figures are for the main effect of recording condition on sparsity. Population vector overlap and population vector decoding Population vector overlap and population vector decoding were computed using binned rate maps for path distance or angle (FIGS. 65A-65D and 67A-67F; additional details in Supplementary Information).

### ANOVA for Decoding Accuracy Across Trials and Bins

A two-way ANOVA in MATLAB was used to compare decoding accuracy in different distance or angle bins across different trials (FIG. 67D, right, and FIG. 67F, right, respectively). Trial number was set to have random effects, and bin number was a continuous predictor of decoding accuracy.

### Average Paths

To construct the average path from each start position to the goal location (FIG. 50A), individual trials were grouped according to start position and then interpolated as follows. For a given start position, define Dmax as the distance travelled in the longest path (in cm). For each trial originating from that start position, define P as the X and Y coordinates of the path and D1 as the cumulative distance travelled in that trial only, normalized to have a maximum value of 1. D2 is defined as a linearly spaced vector from 0 to 1 with Dmax data points. Pinterp is then computed using the MATLAB function interp1(D1, P, D2). The average path is then computed as the median of all interpolated paths. Path correlation To estimate across-position path correlations (FIGS. 50C and 61A-61E), we computed correlation coefficients of rotated occupancy maps for each start position. Rotated occupancy maps were computed by rotating all paths originating from a given start position such that the initial heading was −90° (south) and then discarding the first three position bins (to reduce spurious correlation from low speed at the beginning of trials). Occupancy in 10×10-cm bins was then computed without smoothing to make the rotated occupancy map for each start position. The larger bin size was used to compensate for the reduction in the amount of data resulting from restricting the analysis to individual start positions. The ‘across-position path correlation’ was then the mean correlation coefficient across all pairs of maps from different start positions. Within-position path correlations were calculated in a similar manner as across-position correlations. First, the rotated occupancy map for a start position was computed. Then, the process was repeated by resampling individual trials with replacement to construct a new occupancy map. This was repeated 100 times. The ‘within-position path correlation’ was then defined as the first percentile (lowest) correlation coefficient between the original occupancy map and the resampled maps.

### Inclusion Criteria for Different Path Lengths

To test whether the aggregation of distance field peaks was the result of shorter distances being oversampled, the distribution of path distance field peaks for trials of different lengths was compared (Supplementary Information). Data were separated into four groups, comprising data from trials of 0-75 cm, 75-150 cm, 150-225 cm and 225-300 cm. Binned rate maps were computed as described above but with a larger smoothing kernel (sigma=7.5 cm) and a minimum occupancy of only 1 s, to adjust for the inclusion of less data. Within each distance range, only cells that met occupancy criteria in at least 75% of bins were included. To compute the distributions of distance peaks for the above analysis, peaks were binned using 80 equally spaced bins from 0 cm to the maximum distance for that range and smoothed with a Gaussian smoothing kernel with a sigma of 3 bins. Here, the smoothing window was defined using bins rather than centimetres, to allow unbiased comparison of these distributions. Rate modulation index Rate modulation index (FIGS. 62A-62G) was computed as (R2−R1)/(R2+R1), where R1 is the mean firing rate of a cell across trials 1-26, and R2 is the mean firing rate of a cell across trials 27-52. Experience dependence Details for analyses of experience dependence (FIGS. 62A-62G, 63A-63D, and 67C, 67E) are provided in the Supplementary Information. Temporal relation between neural and behavioural changes To assess the temporal relation between changes in neural coding and changes in behaviour, we computed the cross-correlation between the neural clustering measures and the behavioural clustering measures (FIGS. 64A-64D, bottom rows). Data were split into two groups containing sessions with high (top 50%) or low (bottom 50%) behavioural performance. Statistical significance was assessed by a shuffling procedure. The neural and behavioural curves were shuffled with respect to trial number to create control cross-correlations. This was repeated 5,000 times to create the 99% range indicated by the dotted lines in FIGS. 64A-64D, bottom rows.

### Supplementary Information

### Binning Method of Computing Rate Maps

Spatial rate maps were computed using bins of size 5×5 cm spanning from −100 to 100 cm in both X and Y coordinates. Occupancy maps and spike count maps were computed, smoothed with a 2-dimensional Gaussian kernel, and then divided to compute the rate. In FIGS. 49A-49D, 53A, and 53B, the 2D smoothing kernel had a sigma of 7.5 cm, to directly compare values to previous work. In all other figures, the 2D smoothing kernel had a sigma of 5 cm. Bins with less than 250 ms of occupancy were excluded.

Path distance maps were computed in a similar fashion, with 80 bins of width 3.75 cm spanning 0 to 300 cm, and smoothed with a Gaussian kernel with a sigma of 3.75 cm. Bins with occupancy less than 2 seconds were excluded.

Angular rate maps used bins of size 4.5 degrees from −71 to 7, circularly smoothed with a Gaussian kernel with a sigma of 4.5 degrees.

Temporal rate maps (FIGS. 58A-58Bb) were constructed in a similar fashion. The moment a rat began moving in a trial was defined as time 0.80 bins of width 250 ms spanning 0 to 20 seconds were used and smoothed with a Gaussian kernel with a sigma of 250 ms.

Goal distance maps (FIGS. 58C, 58D) were constructed in a similar fashion to path distance maps using the same sized bins, but from −300 cm to 0 cm, with 0 cm indicating the moment the rat entered the reward zone.

### Generalized Linear Model

Coefficients for the GLM basis functions were fit with the Matlab function glmnet( )⁷⁴ with an alpha parameter of 0 using 5-fold cross validation and 100 lambda values generated by glmnet( ). This was repeated for models containing 5 to 32 Zernike polynomials, yielding 2800 possible models for each neuron. The fitness F of each model was computed as the mean cross-validated error. To bias model selection towards smooth maps, this fitness was then divided by the coherence of the spatial rate map for each model to get the final fitness F*. The coefficients corresponding to the model with the lowest F* value were then used to reconstruct the rate maps (see below).

For plotting purposes and calculation of tuning, GLM-derived maps for space, distance, and angle were constructed by evaluating the GLM at the center of the bins described above for binned rate maps. Minimum occupancy values were identical to those for binned rate maps.

Specifically, GLM allocentric space maps R_{Space}(X,Y) were calculated as

exp(Σᵢ₌₁^{N}βᵢ^{S}Zᵢ(X,Y)),

where βᵢ^{S} is the coefficient for the ith spatial component, Zᵢ is the i^{th} Zernike basis function, and (X, Y) are the centers of the 5 cm×5 cm spatial bins. Here, N is the number of basis functions selected by the above cross-validation process.

Path distance maps R_{Distance}(D) were calculated as

exp(Σᵢ₌₁^{N}βᵢ^{D}Cᵢ(D)), where βᵢ^{D}

is the coefficient for the i^{th} distance component, Cᵢ is the i^{th} Chebyshev basis function⁷⁵, and D represents the centers of 3.75 cm-wide distance bins from 0 to 300 cm. Here, N=10.

Allocentric angle maps R_{Angle}(A) were calculated as

exp(Σᵢ₌₁^{N}βᵢ^{A}Sᵢ(A)), where βᵢ^{A}

is the coefficient for the i^{th} angle component, Sᵢ is the i^{th} sinusoidal function, and A represents the centers of 4.5 degree-wide angle bins from −π to π. Here, N=10.

The predicted firing rate of a neuron from these GLM-derived maps is the product of all 3 maps with a constant scaling factor:

R(t)=c*R_{Space}(X(t),Y(t))*R_{Distance}(D(t))*R_{Angle}(A(t)).

Each individual map can be considered akin to an individual “risk factor” for firing. The spatial, distance, and angle curves along with the constant term must be considered simultaneously to derive a predicted firing rate at any time. Consequently, the maximum rate for any individual GLM-derived curve is arbitrary. These maps are presented in terms of normalized rates. As sparsity is a scale-invariant measure (see below), the sparsity of normalized rate maps is identical to the sparsity of non-normalized rate maps. When individual GLM-derived maps are presented (FIGS. 54A-54E, 55A-55H, 57A-57J, and 59A-59J), the mean firing rate (p) of the unit is noted, to give an idea of the general activity rate of the unit.

### Statistics

Unless otherwise noted, statistical comparisons were made using a two-sided Wilcoxon rank-sum test for nonmatched data and a two-sided Wilcoxon sign-rank test for matched data. Significance of correlation values were assessed on the linear correlation coefficient using a T-test.

Unless otherwise specified, all values are reported as the median and 95% confidence interval of the median, in the form M [L, U], where M is the median of the data and L and U are the lower and upper bounds, respectively, of the 95% confidence interval of the median. Unless otherwise stated, error bars in all figures indicate the 95% confidence interval of the median. Confidence intervals for percentages were obtained using the Matlab function binofit( ).

### Population Vector Overlap and Population Vector Decoding

Population vector overlap and population vector decoding (FIGS. 65A-A-65D, 67D, and 67F) were done using binned rate maps for path distance or angle. The “template” vectors in FIGS. 65B, left and 65D, left were constructed from all data in trials 1-52 across all sessions and rats. The “true” vectors in FIGS. 65B, left and 65D, left were computed for each trial. The correlation coefficient between each pair of distances (or angles) was then computed for each trial. The matrices plotted in FIGS. 65B, left and 65D, left were constructed by averaging the trial-wise matrices for trials 1-15 (left). To construct the population vector overlap matrices in Extended Data FIGS. 65B, right and 65D, right, population rate maps were constructed using bins twice the width as defined for binned rate maps, to compensate for the smaller amount of data in single trials (7.5 cm for distance, 9 degrees for angle; minimum occupancy of 200 ms in a given trial). “Template” rate maps were constructed from all data in trials 16-30, and “True” rate maps were constructed from all data in trials 1-15.

Population vector decoding (FIGS. 65A-65D, 67D, and 67F) was done for each bin by picking the distance (or angle) corresponding to the highest correlation in that bin. In FIGS. 65B, right, and 65D, right, the decoded value was then smoothed with a Gaussian kernel with a sigma of 1 bin (7.5 cm for distance, 9 degrees for angle).

### Occupancy Index, Speed Index, and Goal Heading Index

To quantify behavioral performance, the occupancy index and speed index was calculated (FIGS. 55E, 55F) as follows. Position was binned using 5×5 cm bins. For each session, the occupancy as a function of radial distance from the reward was calculated as the mean occupancy time of bins falling within 6 cm radial bins. Speed as a function of radial distance was computed in a similar manner.

To compute occupancy and speed indices, null behavioral data was generated for each session. For sessions with 4 start positions, the path for each trial was rotated by 90, 180, and 270 degrees. For sessions with 8 start positions, each path was rotated by 45, 90, 135, 180, 225, 270, and 315 degrees. Rotated paths were truncated after first crossing into the reward zone, if applicable. Null radial occupancy and speed were then computed from these rotated data sets. Occupancy index was then defined as:

$$100 * \frac{Occ - {Occ}_{Null}}{Occ + {Occ}_{Null}}$$

where Occ is the original occupancy distribution and OCC_{Null} is the null occupancy distribution.

Speed index was defined in a similar way.

### Goal Heading Index.

Goal Heading Index was defined as: T_{Towards}−T_{Away}/T_{Towards}+T_{Away}, where T_{Towards} is the amount of time spent moving towards the reward zone, and T_{Away} is the amount of time spent moving away from the reward zone. For this calculation, only data where the rat was moving at least 0.5 cm/s was included.

### Decomposing Maps, Goodness of Fit, Peak Index, and Peak Width

To fit a mixture of Gaussians to a path distance map, the rate map was smoothed with a Gaussian kernel with a sigma of 7.5 cm (2 bins). Then, a mixture of N Gaussians with a constant offset was fitted to the curve where N is defined as the number of distinct peaks above 25% of the maximum value. Distinct peaks were defined as peaks with a value below the 25% threshold between them. The reconstructed curve was then re-estimated using the same procedure (without smoothing), and this was repeated until the number of components did not change. The results were robust to small changes in the specific values of the above parameters.

The procedure for decomposing allocentric angular rate maps (FIGS. 59A-59J) was similar, except mixtures of Von Mises functions were used, and the original curve was circularly smoothed with a sigma of 13.5 degrees (3 bins).

Goodness of fit (FIGS. 57A-57J and 59A-59J) was calculated as the correlation coefficient between the original rate map and the map reconstructed from its fit components.

Peak index (FIGS. 57A-57J and 59A-59J) was computed for each distance peak, and calculated as the ratio of A/C, where A is the amplitude of the fitted component and C is the constant offset of the fitted curve.

For path distance, peak width (FIGS. 57A-57J) was defined as the sigma value for each fitted component.

For allocentric angle, peak width (FIGS. 59A-59J) was defined as the width of each fitted component at 50% of the component's amplitude (i.e., full width at half max).

### Experience Dependence

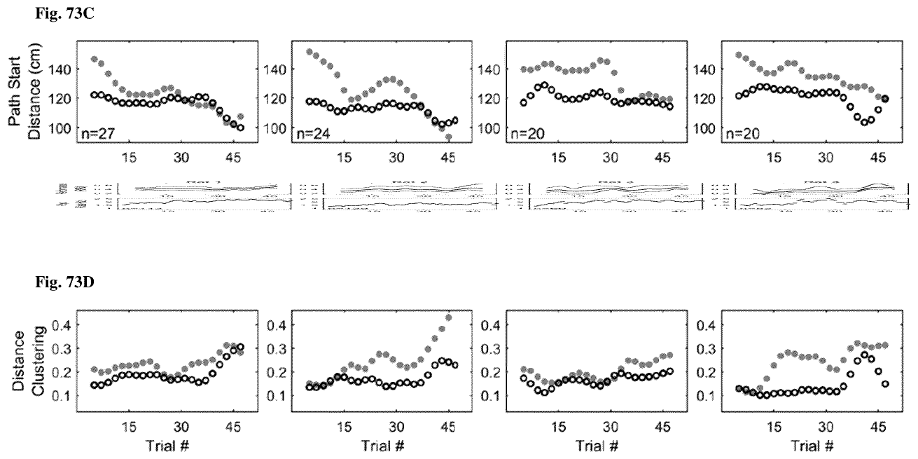

For analyses of experience dependence (FIGS. 63A-63D, 67C, 67E FIGS. 73A-73D, but not FIG. 62A-62G, see below), occupancy and spikes were binned for individual trials using bins as defined in “Binning method of computing rate maps” above, and smoothed across trials with a Gaussian kernel with a sigma of 4 trials. The smoothed spike maps were divided by the smoothed occupancy maps to produce trial-by-trial rate maps. Minimum occupancy was 40 ms for path distance and allocentric angle, and 20 ms for allocentric space.

Trial-by-trial rate maps for path distance and allocentric angle were decomposed as above to identify peaks, which were then analyzed for FIGS. 63A-63D. Allocentric space peaks in FIGS. 63A-63D were defined as local maxima greater than 20% of the peak rate.

Peak density plots in FIGS. 63A, 67C, and 67E were constructed by binning peaks using bins as defined in “Binning method of computing rate maps” above for each trial. Values in each bin in this distribution were then divided by the total number of cells with defined rates in that bin. This normalization ensures that experience-dependent changes in the distribution of rate map peaks are not artificially driven by experience dependent changes in the distribution of occupancy. Finally, the distribution for each trial was normalized to sum to 100%.

Single unit shifts in distance tuning curves (FIGS. 62A-62G) were evaluated on GLM-derived rate maps for trials 1-26 and 27-52, as in FIGS. 54A-54E. To isolate the effect of experience on distance tuning only, we selected cells which were i) significantly tuned for distance; ii) not significantly tuned for angle; and iii) included sufficient spiking data in both halves to construct rate maps. 91 cells met criteria (i) and (ii), and 88 cells met all three criteria.

### Experience-Dependent Clustering Analyses

To quantify the experience dependent changes in both neural responses and behavior for allocentric space, we computed two measures: distance from reward and spatial clustering. To compute distance from reward, the distributions shown in FIGS. 63A-63D were reparametrized by computing the radial distance between any position and the reward zone (FIG. 63A, top). This measure was binned using 40 bins of 3 cm width from 0 to 120 cm to generate a distribution of radial distance. Distance from reward was defined as the center of mass of this distribution (FIG. 63B, left). Spatial clustering was defined as the sparsity (defined above) of this distribution (FIG. 63B, right). The advantage of these measures is that they can be applied to both behavioral and neural data to allow not just qualitative, but quantitative, comparisons between them. These same measures were applied to the distribution of the peaks of the allocentric spatial rate maps (FIG. 63A, bottom) to estimate their distance from reward and clustering. Similarly, we computed the distribution of path distance for both behavior and peaks of path distance rate maps (FIG. 67C), and defined distance from start and distance clustering (63C) in the same way as above. For angular tuning, we reparametrized the angle distributions (FIG. 67E) as the absolute difference between any angle and 45 degrees (corresponding to the northeast quadrant where the hidden reward zone is located). This angular difference was binned using 40 bins of width 4.5° from 0 to 180° to generate a distribution which was quantified as above to compute angular distance from goal and angular clustering (FIG. 63D).

What is claimed is:

1. A system comprising:
a virtual or augmented reality system adapted to provide a virtual environment to a user;
at least one sensor coupled to the user;
a computing node comprising a computer readable storage medium having program instructions embodied therewith, the program instructions executable by a processor of the computing node to cause the processor to perform a method comprising:
presenting a first visual stimulus to the user within the virtual environment, the first visual stimulus having a high spatial frequency; and
presenting a second visual stimulus to the user within the virtual environment, the second visual stimulus having a low spatial frequency.

2. The system of claim 1, wherein the method further comprises:
measuring at least one electrical activity of a brain of the user by the at least one sensor;
providing the measured at least one electrical activity of the brain to a learning system and determining therefrom an updated first visual stimulus and an updated second visual stimulus adapted to induce a change in the at least one electrical activity of the brain; and
presenting the updated first visual stimulus and the updated second visual stimulus to the user within the virtual environment.

3. The system of claim 1, wherein the first visual stimulus is presented on a floor of the virtual environment.

4. The system of claim 1, wherein the second visual stimulus is presented on a wall of the virtual environment.

5. The system of claim 4, wherein the second visual stimulus is presented on one or more of: a forward surface, peripheral surfaces, and a rear surface.

6. The system of claim 3, wherein the first visual stimulus comprises a virtual platform and a virtual floor.

7. The system of claim 6, wherein the virtual platform comprises a different shape and/or pattern from the virtual floor.

8. The system of claim 3, wherein the first visual stimulus and the second visual stimulus comprises a size based on a visual acuity of the user.

9. The system of claim 8, wherein a size of the first visual stimulus corresponds to the visual acuity of the user.

10. The system of claim 8, wherein a size of the second visual stimulus is greater than the visual acuity of the user.

11. The system of claim 2, wherein the at least one electrical activity of the brain comprises theta waves.

12. The system of claim 2, wherein the at least one electrical activity of the brain comprises hippocampal activity.

13. The system of claim 2, wherein the learning system comprises an artificial neural network.

14. A method comprising:
providing a virtual environment to a user via a virtual or augmented reality system;
presenting a first visual stimulus to the user within the virtual environment, the first visual stimulus having a high spatial frequency; and
presenting a second visual stimulus to the user within the virtual environment, the second visual stimulus having a low spatial frequency.

15. The method of claim 14, further comprising:
measuring at least one electrical activity of a brain of the user by at least one sensor;
providing the measured at least one electrical activity of the brain to a learning system and determining therefrom an updated first visual stimulus and an updated second visual stimulus adapted to induce a change in the at least one electrical activity of the brain; and
presenting the updated first visual stimulus and the updated second visual stimulus to the user within the virtual environment.

16. The method of claim 14, wherein the first visual stimulus is presented on a floor of the virtual environment.

17. The method of claim 14, wherein the second visual stimulus is presented on a wall of the virtual environment.

18. The method of claim 17, wherein the second visual stimulus is presented on one or more of: a forward surface, peripheral surfaces, and a rear surface.

19. The method of claim 16, wherein the first visual stimulus comprises a virtual platform and a virtual floor.

20. The method of claim 19, wherein the virtual platform comprises a different shape and/or pattern from the virtual floor.

21. The method of claim 16, wherein the first visual stimulus and the second visual stimulus comprises a size based on a visual acuity of the user.

22. The method of claim 21, wherein a size of the first visual stimulus corresponds to the visual acuity of the user.

23. The method of claim 21, wherein a size of the second visual stimulus is greater than the visual acuity of the user.

24. The method of claim 15, wherein the at least one electrical activity of the brain comprises theta waves.

25. The method of claim 15, wherein the at least one electrical activity of the brain comprises hippocampal activity.

26. The method of claim 15, wherein the learning system comprises an artificial neural network.

27. A computer program product comprising a computer readable storage medium having program instructions embodied therewith, the program instructions executable by a processor to cause the processor to perform a method comprising:
providing a virtual environment to a user via a virtual or augmented reality system;
presenting a first visual stimulus to the user within the virtual environment, the first visual stimulus having a high spatial frequency; and
presenting a second visual stimulus to the user within the virtual environment, the second visual stimulus having a low spatial frequency.
